SEQUENCE LISTING (139 sequences)

SEQ ID NO: 1 - DNA, 36 bp, synthetic construct
  source 1..36: mol_type = other DNA
gagtctaagtacggaccgccctgccccccttgccct

SEQ ID NO: 2 - DNA, 36 bp, synthetic construct
  source 1..36: mol_type = other DNA
gagtctaagtacggaccgccttgcccaccgtgccca

SEQ ID NO: 3 - DNA, 321 bp, synthetic construct
  source 1..321: mol_type = other DNA
ggccagcctagagaaccccaggtgtacaccctgcctcccagccaggaagagatgaccaagaaccaggtgtccctgacctgcctggtcaaaggcttctaccccagcgatatcgccgtggaatgggagagcaacggccagcccgagaacaactacaagaccaccccccctgtgctggacagcgacggcagcttcttcctgtactcccggctgaccgtggacaagagccggtggcaggaaggcaacgtcttcagctgcagcgtgatgcacgaggccctgcacaaccactacacccagaagtccctgagcctgagcctgggcaag

SEQ ID NO: 4 - DNA, 648 bp, synthetic construct
  source 1..648: mol_type = other DNA
gcaccacctgtggcaggaccgtcagtcttcctcttcccaccaaaacccaaggacaccctgatgatcagccggacccccgaggtgacctgcgtggtggtggacgtgagccaggaagatcccgaggtccagttcaattggtacgtggacggcgtggaagtgcacaacgccaagaccaagcccagagaggaacagttccaaagcacctaccgggtggtgtctgtgctgaccgtgctgcaccaggactggctgaacggcaaagaatacaagtgcaaggtgtccaacaagggcctgcccagcagcatcgaaaagaccatcagcaaggccaagggccagcctcgcgagccccaggtgtacaccctgcctccctcccaggaagagatgaccaagaaccaggtgtccctgacctgcctggtgaagggcttctaccccagcgacatcgccgtggagtgggagagcaacggccagcctgagaacaactacaagaccacccctcccgtgctggacagcgacggcagcttcttcctgtacagccggctgaccgtggacaagagccggtggcaggaaggcaacgtctttagctgcagcgtgatgcacgaggccctgcacaaccactacacccagaagagcctgagcctgtccctgggcaag

SEQ ID NO: 5 - DNA, 336 bp, Homo sapiens
  source 1..336: mol_type = other DNA
cgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 6 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - protein, 114 aa, Homo sapiens
ELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - DNA, 126 bp, synthetic construct
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 9 - DNA, 129 bp, synthetic construct
  source 1..129: mol_type = other DNA
gtgaaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 10 - protein, 43 aa, synthetic construct
VKRGRKKLLYIFKQPFMRPVQTTQEEDGCSCRFPEEEEGGCEL

SEQ ID NO: 11 - protein, 41 aa, synthetic construct
VKRGRKKLLYIFKQPFMRPVQTTQEEDGCSCRFPEEEEGGC

SEQ ID NO: 12 - DNA, 84 bp, synthetic construct
  source 1..84: mol_type = other DNA
atgttctgggtgctggtggtggtcggaggcgtgctggcctgctacagcctgctggtcaccgtggccttcatcatcttttgggtg

SEQ ID NO: 13 - DNA, 81 bp, synthetic construct
  source 1..81: mol_type = other DNA
atgttctgggtgctggtggtggtcggaggcgtgctggcctgctacagcctgctggtcaccgtggccttcatcatcttttgg

SEQ ID NO: 14 - DNA, 81 bp, synthetic construct
  source 1..81: mol_type = other DNA
atgttctgggtgctggtggtggtgggcggggtgctggcctgctacagcctgctggtgacagtggccttcatcatcttttgg

SEQ ID NO: 15 - protein, 27 aa, synthetic construct
MFWVLVVVGGVLACYSLLVTVAFIIFW

SEQ ID NO: 16 - protein, 26 aa, synthetic construct
FWVLVVVGGVLACYSLLVTVAFIIFW

SEQ ID NO: 17 - protein, 26 aa, synthetic construct
  REGION 1..3: note = (GSG) residues at the 5' end of the peptide improve cleavage efficiency
GSGATNFSLLKQAGDVEENPGPGASG

SEQ ID NO: 18 - protein, 21 aa, synthetic construct
  REGION 1..3: note = (GSG) residues at the 5' end of the peptide improve cleavage efficiency
GSGEGRGSLLTCGDVEENPGP

SEQ ID NO: 19 - protein, 24 aa, synthetic construct
  REGION 1..3: note = (GSG) residues at the 5' end of the peptide improve cleavage efficiency
GSGQCTNYALLKLAGDVESNPGPP

SEQ ID NO: 20 - protein, 25 aa, synthetic construct
  REGION 1..3: note = (GSG) residues at the 5' end of the peptide improve cleavage efficiency
GSGVKQTLNFDLLKLAGDVESNPGP

SEQ ID NO: 21 - protein, 257 aa, Homo sapiens
MAQRMTTQLLLLLVWVAVVGEAQTRIAWARTELLNVCMNAKHHKEKPGPEDKLHEQCRPWRKNACCSTNTSQEAHKDVSYLYRENWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMSGAGPWAAWPFLLSLALMLLWLLS

SEQ ID NO: 22 - protein, 249 aa, synthetic construct
DIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIKGGGGSGGGGSGGGGSGGGGSEVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSS

SEQ ID NO: 23 - protein, 249 aa, synthetic construct
EVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSSGGGGSGGGGSGGGGSGGGGSDIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIK

SEQ ID NO: 24 - protein, 5 aa, synthetic construct
GYGLS

SEQ ID NO: 25 - protein, 17 aa, synthetic construct
MISSGGSYTYYADSVKG

SEQ ID NO: 26 - protein, 10 aa, synthetic construct
HGDDPAWFAY

SEQ ID NO: 27 - protein, 12 aa, synthetic construct
SVSSSISSNNLH

SEQ ID NO: 28 - protein, 7 aa, synthetic construct
GTSNLAS

SEQ ID NO: 29 - protein, 11 aa, synthetic construct
QQWSSYPYMYT

SEQ ID NO: 30 - protein, 119 aa, synthetic construct
EVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSS

SEQ ID NO: 31 - protein, 110 aa, synthetic construct
DIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIK

SEQ ID NO: 32 - protein, 5 aa, synthetic construct
GYFMN

SEQ ID NO: 33 - protein, 17 aa, synthetic construct
RIHPYDGDTFYNQKFQG

SEQ ID NO: 34 - protein, 9 aa, synthetic construct
YDGSRAMDY

SEQ ID NO: 35 - protein, 15 aa, synthetic construct
KASQSVSFAGTSLMH

SEQ ID NO: 36 - protein, 7 aa, synthetic construct
RASNLEA

SEQ ID NO: 37 - protein, 9 aa, synthetic construct
QQSREYPYT

SEQ ID NO: 38 - protein, 118 aa, synthetic construct
QVQLVQSGAEVVKPGASVKISCKASGYTFTGYFMNWVKQSPGQSLEWIGRIHPYDGDTFYNQKFQGKATLTVDKSSNTAHMELLSLTSEDFAVYYCTRYDGSRAMDYWGQGTTVTVSS

SEQ ID NO: 39 - protein, 112 aa, synthetic construct
DIVLTQSPLSLAVSLGQPAIISCKASQSVSFAGTSLMHWYHQKPGQQPRLLIYRASNLEAGVPDRFSGSGSKTDFTLNISPVEAEDAATYYCQQSREYPYTFGGGTKLEIKR

SEQ ID NO: 40 - protein, 118 aa, synthetic construct; identical to SEQ ID NO: 38

SEQ ID NO: 41 - protein, 112 aa, synthetic construct
DIVLTQSPLSLAVSLGQPAIISCKASQSVSFAGTSLMHWYHQKPGQQPRLLIYRASNLEAGVPDRFSGSGSKTDFTLTISPVEAEDAATYYCQQSREYPYTFGGGTKLEIKR

SEQ ID NO: 42 - protein, 5 aa, synthetic construct
DFYMN

SEQ ID NO: 43 - protein, 19 aa, synthetic construct
FIRNKANGYTTEFNPSVKG

SEQ ID NO: 44 - protein, 13 aa, synthetic construct
TLYGYAYYYVMDA

SEQ ID NO: 45 - protein, 11 aa, synthetic construct
RTSEDIFRNLA

SEQ ID NO: 46 - protein, 7 aa, synthetic construct
DTNRLAD

SEQ ID NO: 47 - protein, 9 aa, synthetic construct
QQYDNYPLT

SEQ ID NO: 48 - protein, 124 aa, synthetic construct
EVQLVESGGGLVQPGGSLRLSCAASGFTFTDFYMNWVRQPPGKAPEWLGFIRNKANGYTTEFNPSVKGRFTISRDNSKNSLYLQMNSLKTEDTATYYCARTLYGYAYYYVMDAWGQGTLVTVSS

SEQ ID NO: 49 - protein, 107 aa, synthetic construct
DIQMTQSPSSLSASLGDRVTITCRTSEDIFRNLAWYQQKPGKAPKLLIYDTNRLADGVPSRFSGSGSGTDYTLTISSLQPEDFATYFCQQYDNYPLTFGQGTKLEIK

SEQ ID NO: 50 - protein, 5 aa, synthetic construct
NYWMQ

SEQ ID NO: 51 - protein, 17 aa, synthetic construct
AIYPGNGDSRYTQKFQG

SEQ ID NO: 52 - protein, 8 aa, synthetic construct
RDGNYAAY

SEQ ID NO: 53 - protein, 11 aa, synthetic construct
RASENIYSNLA

SEQ ID NO: 54 - protein, 7 aa, synthetic construct
AATNLAD

SEQ ID NO: 55 - protein, 9 aa, synthetic construct
QHFWASPYT

SEQ ID NO: 56 - protein, 117 aa, synthetic construct
QVQLVQSGAEVAKPGASVKLSCKASGYTFTNYWMQWIKQRPGQGLEWIGAIYPGNGDSRYTQKFQGKATLTADKSSSTAYMQVSSLTSEDSAVYYCARRDGNYAAYWGQGTLVTVSA

SEQ ID NO: 57 - protein, 108 aa, synthetic construct
DIQMTQSPSSLSVSVGERVTITCRASENIYSNLAWYQQKPGKSPKLLVYAATNLADGVPSRFSGSESGTDYSLKINSLQPEDFGSYYCQHFWASPYTFGQGTKLEIKR

SEQ ID NO: 58 - protein, 5 aa, synthetic construct
NYWMY

SEQ ID NO: 59 - protein, 17 aa, synthetic construct
AIYPGNSDTTYNQKFQG

SEQ ID NO: 60 - protein, 9 aa, synthetic construct
RHDYGAMDY

SEQ ID NO: 61 - protein, 11 aa, synthetic construct
RASENIYTNLA

SEQ ID NO: 62 - protein, 7 aa, synthetic construct
TASNLAD

SEQ ID NO: 63 - protein, 9 aa, synthetic construct
QHFWVSPYT

SEQ ID NO: 64 - protein, 118 aa, synthetic construct
QVQLQQSGAVVAKPGASVKMSCKASGYTFTNYWMYWIKQRPGQGLELIGAIYPGNSDTTYNQKFQGKATLTAVTSANTVYMEVSSLTSEDSAVYYCTKRHDYGAMDYWGQGTSVTVSS

SEQ ID NO: 65 - protein, 108 aa, synthetic construct
DIQMTQSPSSLSVSVGERVTITCRASENIYTNLAWYQQKPGKSPKLLVYTASNLADGVPSRFSGSGSGTDYSLKINSLQPEDFGTYYCQHFWVSPYTFGQGTKLEIKR

SEQ ID NO: 66 - protein, 5 aa, synthetic construct
SFGMH

SEQ ID NO: 67 - protein, 17 aa, synthetic construct
YISSGSSTISYADSVKG

SEQ ID NO: 68 - protein, 9 aa, synthetic construct
EAYGSSMEY

SEQ ID NO: 69 - protein, 11 aa, synthetic construct
RASQNINNNLH

SEQ ID NO: 70 - protein, 7 aa, synthetic construct
YVSQSVS

SEQ ID NO: 71 - protein, 10 aa, synthetic construct
QQSNSWPHYT

SEQ ID NO: 72 - protein, 118 aa, synthetic construct
EVQLVESGGGLVQPGGSRRLSCAASGFTFSSFGMHWVRQAPGKGLEWVAYISSGSSTISYADSVKGRFTISRDNSKKTLLLQMTSLRAEDTAMYYCAREAYGSSMEYWGQGTLVTVSS

SEQ ID NO: 73 - protein, 109 aa, synthetic construct
EIVLTQSPATLSVTPGDRVSLSCRASQNINNNLHWYQQKPGQSPRLLIKYVSQSVSGIPDRFSGSGSGTDFTLSISSVEPEDFGMYFCQQSNSWPHYTFGQGTKLEIKR

SEQ ID NO: 74 - protein, 5 aa, synthetic construct
SYTMH

SEQ ID NO: 75 - protein, 17 aa, synthetic construct
YINPISGYTNYNQKFQG

SEQ ID NO: 76 - protein, 11 aa, synthetic construct
GGAYGRKPMDY

SEQ ID NO: 77 - protein, 11 aa, synthetic construct
KASQNVGPNVA

SEQ ID NO: 78 - protein, 7 aa, synthetic construct
SASYRYS

SEQ ID NO: 79 - protein, 9 aa, synthetic construct
QQYNSYPYT

SEQ ID NO: 80 - protein, 120 aa, synthetic construct
QVQLVQSGAEVAKPGASVKMSCKASGYTFTSYTMHWVKQRPGQGLAWIGYINPISGYTNYNQKFQGKATLTADKSSSTAYMQLNSLTSEDSAVYYCASGGAYGRKPMDYWGQGTSVTVSS

SEQ ID NO: 81 - protein, 108 aa, synthetic construct
EIVMTQSPATMSTSPGDRVSVTCKASQNVGPNVAWYQQKPGQSPRALIYSASYRYSGVPARFTGSGSGTDFTLTISNMQSEDLAEYFCQQYNSYPYTFGQGTKLEIKR

SEQ ID NO: 82 - protein, 1140 aa, Homo sapiens
MVISLNSCLSFICLLLCHWIGTASPLNLEDPNVCSHWESYSVTVQESYPHPFDQIYYTSCTDILNWFKCTRHRVSYRTAYRHGEKTMYRRKSQCCPGFYESGEMCVPHCADKCVHGRCIAPNTCQCEPGWGGTNCSSACDGDHWGPHCTSRCQCKNGALCNPITGACHCAAGFRGWRCEDRCEQGTYGNDCHQRCQCQNGATCDHVTGECRCPPGYTGAFCEDLCPPGKHGPQCEQRCPCQNGGVCHHVTGECSCPSGWMGTVCGQPCPEGRFGKNCSQECQCHNGGTCDAATGQCHCSPGYTGERCQDECPVGTYGVLCAETCQCVNGGKCYHVSGACLCEAGFAGERCEARLCPEGLYGIKCDKRCPCHLENTHSCHPMSGECACKPGWSGLYCNETCSPGFYGEACQQICSCQNGADCDSVTGKCTCAPGFKGIDCSTPCPLGTYGINCSSRCGCKNDAVCSPVDGSCTCKAGWHGVDCSIRCPSGTWGFGCNLTCQCLNGGACNTLDGTCTCAPGWRGEKCELPCQDGTYGLNCAERCDCSHADGCHPTTGHCRCLPGWSGVHCDSVCAEGRWGPNCSLPCYCKNGASCSPDDGICECAPGFRGTTCQRICSPGFYGHRCSQTCPQCVHSSGPCHHITGLCDCLPGFTGALCNEVCPSGRFGKNCAGICTCTNNGTCNPIDRSCQCYPGWIGSDCSQPCPPAHWGPNCIHTCNCHNGAFCSAYDGECKCTPGWTGLYCTQRCPLGFYGKDCALICQCQNGADCDHISGQCTCRTGFMGRHCEQKCPSGTYGYGCRQICDCLNNSTCDHITGTCYCSPGWKGARCDQAGVIIVGNLNSLSRTSTALPADSYQIGAIAGIIILVLVVLFLLALFIIYRHKQKGKESSMPAVTYTPAMRVVNADYTISGTLPHSNGGNANSHYFTNPSYHTLTQCATSPHVNNRDRMTVTKSKNNQLFVNLKNVNPGKRGPVGDCTGTLPADWKHGGYLNELGAFGLDRSYMGKSLKDLGKNSEYNSSNCSLSSSENPYATIKDPPVLIPKSSECGYVEMKSPARRDSPYAEINNSTSANRNVYEVEPTVSVVQGVFSNNGRLSQDPYDLPKNSHIPCHYDLLPVRDSSSSPKQEDSGGSSSNSSSSSE

SEQ ID NO: 83 - protein, 592 aa, Homo sapiens [sequence omitted]

SEQ ID NO: 84 - protein, 207 aa, Homo sapiens
MENEDGYMTLSFKNRCKSKQKSKDFSLYPQYYCLLLIFGCIVILIFIMTGIDLKFWHKKMDFSQNVNVSSLSGHNYLCPNDWLLNEGKCYWFSTSFKTWKESQRDCTQLQAHLLVIQNLDELEFIQNSLKPGHFGWIGLYVTFQGNLWMWIDEHFLVPELFSVIGPTDDRSCAVITGNWVYSEDCSSTFKGICQRDAILTHNGTSGV

SEQ ID NO: 85 - protein, 1148 aa, Homo sapiens
MESLLLPVLLLLAILWTQAAALINLKYSVEEEQRAGTVIANVAKDAREAGFALDPRQASAFRVVSNSAPHLVDINPSSGLLVTKQKIDRDLLCRQSPKCIISLEVMSSSMEICVIKVEIKDLNDNAPSFPAAQIELEISEAASPGTRIPLDSAYDPDSGSFGVQTYELTPNELFGLEIKTRGDGSRFAELVVEKSLDRETQSHYSFRITALDGGDPPRLGTVGLSIKVTDSNDNNPVFSESTYAVSVPENSPPNTPVIRLNASDPDEGTNGQVVYSFYGYVNDRTRELFQIDPHSGLVTVTGALDYEEGHVYELDVQAKDLGPNSIPAHCKVTVSVLDTNDNPPVINLLSVNSELVEVSESAPPGYVIALVRVSDRDSGLNGRVQCRLLGNVPFRLQEYESFSTILVDGRLDREQHDQYNLTIQARDGGVPMLQSAKSFTVLITDENDNHPHFSKPYYQVIVQENNTPGAYLLSVSARDPDLGLNGSVSYQIVPSQVRDMPVFTYVSINPNSGDIYALRSFNHEQTKAFEFKVLAKDGGLPSLQSNATVRVIILDVNDNTPVITAPPLINGTAEVYIPRNSGIGYLVTVVKAEDYDEGENGRVTYDMTEGDRGFFEIDQVNGEVRTTRTFGESSKSSYELIVVAHDHGKTSLSASALVLIYLSPALDAQESMGSVNLSLIFIIALGSIAGILFVTMIFVAIKCKRDNKEIRTYNCSNCLTITCLLGCFIKGQNSKCLHCISVSPISEEQDKKTEEKVSLRGKRIAEYSYGHQKKSSKKKKISKNDIRLVPRDVEETDKMNVVSCSSLTSSLNYFDYHQQTLPLGCRRSESTFLNVENQNTRNTSANHIYHHSFNSQGPQQPDLIINGVPLPETENYSFDSNYVNSRAHLIKSSSTFKDLEGNSLKDSGHEESDQTDSEHDVQRSLYCDTAVNDVLNTSVTSMGSQMPDHDQNEGFHCREECRILGHSDRCWMPRNPMPIRSKSPEHVRNIIALSIEATAADVEAYDDCGPTKRTFATFGKDVSDHPAEERPTLKGKRTVDVTICSPKVNSVIREAGNGCEAISPVTSPLHLKSSLPTKPSVSYTIALAPPARDLEQYVNNVNNGPTRPSEAEPRGADSEKVMHEVSPILKEGRNKESPGVKRLKDIVL

SEQ ID NO: 86 - protein, 317 aa, Homo sapiens [sequence omitted]

SEQ ID NO: 87 - protein, 5 aa, synthetic construct
  REPEAT 1..5: note = GGGGS is repeated n times, wherein n is an integer including 1, 2, 3, 4, 5, 6, 7, 8, 9 or 10.
GGGGS

SEQ ID NO: 88 - protein, 9 aa, synthetic construct
  REPEAT 1..4: note = GGGS is repeated n times, wherein n is an integer including 1, 2, 3, 4, 5, 6, 7, 8, 9 or 10
  REPEAT 5..9: note = GGGGS is repeated n times, wherein n is an integer including 1, 2, 3, 4, 5, 6, 7, 8, 9 or 10
GGGSGGGGS

SEQ ID NO: 89 - protein, 7 aa, synthetic construct
  REPEAT 1..4: note = GGGS is repeated n times, wherein n is an integer including 1, 2, 3, 4, 5, 6, 7, 8, 9 or 10
  REPEAT 5..7: note = GGS is repeated n times, wherein n is an integer including 1, 2, 3, 4, 5, 6, 7, 8, 9 or 10
GGGSGGS

SEQ ID NO: 90 - protein, 9 aa, synthetic construct
  REPEAT 1..4: note = GGGS is repeated n times, wherein n is an integer including 1, 2, 3, 4, 5, 6, 7, 8, 9 or 10
GGGSGGGGS

SEQ ID NO: 91 - protein, 20 aa, synthetic construct
GGGGSGGGGSGGGGSGGGGS

SEQ ID NO: 92 - protein, 15 aa, synthetic construct
GGGGSGGGGSGGGGS

SEQ ID NO: 93 - protein, 10 aa, synthetic construct
GGGGSGGGGS

SEQ ID NO: 94 - protein, 5 aa, synthetic construct
GGGGS

SEQ ID NO: 95 - protein, 8 aa, synthetic construct
GGGSGGGS

SEQ ID NO: 96 - protein, 4 aa, synthetic construct
GGGS

SEQ ID NO: 97 - protein, 6 aa, synthetic construct
GGSGGS

SEQ ID NO: 98 - protein, 11 aa, synthetic construct
GGSGGGSGGSG

SEQ ID NO: 99 - protein, 10 aa, synthetic construct
GGSGGGSGSG

SEQ ID NO: 100 - protein, 8 aa, synthetic construct
GGSGGGSG

SEQ ID NO: 101 - protein, 5 aa, synthetic construct
  REPEAT 1..5: note = EAAAK is repeated n times, wherein n is an integer including 1, 2, 3, 4, 5, 6, 7, 8, 9, or more times
EAAAK

SEQ ID NO: 102 - protein, 6 aa, synthetic construct
HHHHHH

SEQ ID NO: 103 - protein, 8 aa, synthetic construct
DYKDDDDK

SEQ ID NO: 104 - protein, 8 aa, synthetic construct
DLYDDDDK

SEQ ID NO: 105 - protein, 15 aa, synthetic construct
GLNDIFEAQKIEWHE

SEQ ID NO: 106 - protein, 26 aa, synthetic construct
KRRWKKNFIAVSAANRFKKISSSGAL

SEQ ID NO: 107 - protein, 9 aa, synthetic construct
YPYDVPDYA

SEQ ID NO: 108 - protein, 10 aa, synthetic construct
EQKLISEEDL

SEQ ID NO: 109 - protein, 8 aa, synthetic construct
WRHPQFGG

SEQ ID NO: 110 - protein, 8 aa, synthetic construct
WSHPQFEK

SEQ ID NO: 111 - protein, 13 aa, synthetic construct
SLAELLNAGLGGS

SEQ ID NO: 112 - protein, 8 aa, synthetic construct
TQDPSRVG

SEQ ID NO: 113 - protein, 14 aa, synthetic construct
GKPIPNPLLGLDST

SEQ ID NO: 114 - DNA, 19 bp, synthetic construct
  source 1..19: mol_type = other DNA
ccctgacggtcatcaacca

SEQ ID NO: 115 - DNA, 21 bp, synthetic construct
  source 1..21: mol_type = other DNA
caccatccaaatagcgcagtg

SEQ ID NO: 116 - DNA, 16 bp, synthetic construct
  source 1..16: mol_type = other DNA
cagcgaggacttccag

SEQ ID NO: 117 - DNA, 972 bp, synthetic construct
  source 1..972: mol_type = other DNA
atgccacctccaagactcctcttcttcctcctcttcctgacaccaatggaagtcaggcctgaggaacctctagtggtgaaggtggaagagggagataacgctgtgttacagtgcctcaagggaacctcagatggacccactcagcagctgacctggtctcgggagtctccgcttaaacccttcctgaaactcagccttggactgccaggtctgggaatccacatgaggccactggctatctggctgttcatcttcaacgtctctcaacagatgggaggcttctacctgtgtcagcctggaccaccttctgagaaggcatggcagcctggttggacagtcaatgtggagggttctggtgagctgttccggtggaatgtttcggacctaggtggactgggatgtggtctgaagaacaggtcctcagagggacctagctctccttccgggaagctcatgagccccaagctgtatgtgtgggccaaagaccgccctgagatctgggagggagagcctccgtgtgtcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgccctgccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagatga

SEQ ID NO: 118 - DNA, 113 bp, synthetic construct
  source 1..113: mol_type = other DNA
ctcggcttgctgaagcgcgcacggcaagaggcgaggggcggcgactggtgagtacgccaaaaattttgactagcggaggctagaaggagagagatgggtgcgagagcgtcagt

SEQ ID NO: 119 - DNA, 333 bp, synthetic construct
  source 1..333: mol_type = other DNA
gcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggatc

SEQ ID NO: 120 - DNA, 179 bp, synthetic construct
  source 1..179: mol_type = other DNA
tacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttgg

SEQ ID NO: 121 - DNA, 544 bp, synthetic construct
  source 1..544: mol_type = other DNA
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 122 - DNA, 23 bp, synthetic construct
  source 1..23: mol_type = other DNA
gaccctgcttgctcaactctacg

SEQ ID NO: 123 - DNA, 24 bp, synthetic construct
  source 1..24: mol_type = other DNA
gtgtacaccctgcctcccagccag

SEQ ID NO: 124 - DNA, 66 bp, synthetic construct
  source 1..66: mol_type = other DNA
atgctgctgctcgtgaccagcctgctgctgtgcgaactgccccaccctgcctttctgctgatcccc

SEQ ID NO: 125 - DNA, 60 bp, synthetic construct
  source 1..60: mol_type = other DNA
ggcggcggaggaagcggaggcggaggatctggcggagggggctccggagggggaggctct

SEQ ID NO: 126 - DNA, 601 bp, synthetic construct
  source 1..601: mol_type = other DNA
atcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgat

SEQ ID NO: 127 - DNA, 105 bp, synthetic construct
  source 1..105: mol_type = other DNA
gtacctttaagaccaatgacttacaaggcagctgtagatcttagccactttttaaaagaaaaggggggactggaagggctaattcactcccaaagaagacaagat

SEQ ID NO: 128 - DNA, 81 bp, synthetic construct
  source 1..81: mol_type = other DNA
ctgctttttgcctgtactgggtctctctggttagaccagatctgagcctgggagctctctggctaactagggaacccactg

SEQ ID NO: 129 - DNA, 114 bp, synthetic construct
  source 1..114: mol_type = other DNA
cttaagcctcaataaagcttgccttgagtgcttcaagtagtgtgtgcccgtctgttgtgtgactctggtaactagagatccctcagacccttttagtcagtgtggaaaatctct

SEQ ID NO: 130 - DNA, 859 bp, synthetic construct
  source 1..859: mol_type = other DNA
atgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggt

SEQ ID NO: 131 - DNA, 882 bp, synthetic construct
  source 1..882: mol_type = other DNA
aaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagag

SEQ ID NO: 132 - DNA, 200 bp, synthetic construct
  source 1..200: mol_type = other DNA
ggtcgagatccggtcgaccagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaa

SEQ ID NO: 133 - DNA, 600 bp, synthetic construct
  source 1..600: mol_type = other DNA
atcgattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggaattc

SEQ ID NO: 134 - DNA, 9608 bp, synthetic construct
  source 1..9608: mol_type = other DNA
gttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggatctacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggggatcaattgcatgaagaatctgcttagggttaggcgttttgcgctgcttcgcgaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacggctagccaccatgctgctgctcgtgaccagcctgctgctgtgcgaactgccccaccctgcctttctgctgatccccgatattcagcttactcaaagtccgagtagtctgtctgcctcagttggcgatagggtgaccatcacttgctccgtaagtagttctatttcttccaacaacctgcattggtatcaacagaaaccaggtaaagcacctaagccgtggatctacggaacgtccaaccttgcgtctggcgtaccaagccggttctccgggagtgggagtggtacagattacacatttactatcagttctcttcaaccggaagacattgccacatattattgccagcaatggtcatcttacccctatatgtacacatttggtcagggtacaaaggttgaaataaaaggcggcggaggaagcggaggcggaggatctggcggagggggctccggagggggaggctctgaggtacagcttgtcgagagcggtggtggagtagtccaaccgggtcgaagtcttaggctttcctgtagcgcatctgggttcacttttagtggctacggcctctcctgggtgagacaggcgcctgggaaggggctggagtgggtagccatgatttcatctggtggctcatatacttattatgccgactccgtaaagggaagattcgcaatatcacgcgataacgctaaaaatacactcttcttgcagatggattctttgagacctgaggataccggggtttacttttgcgccagacacggggatgaccccgcctggtttgcctattggggacagggaacccctgtgacggtatcctctgagtctaagtacggaccgccctgccccccttgccctggccagcctagagaaccccaggtgtacaccctgcctcccagccaggaagagatgaccaagaaccaggtgtccctgacctgcctggtcaaaggcttctaccccagcgatatcgccgtggaatgggagagcaacggccagcccgagaacaactacaagaccaccccccctgtgctggacagcgacggcagcttcttcctgtactcccggctgaccgtggacaagagccggtggcaggaaggcaacgtcttcagctgcagcgtgatgcacgaggccctgcacaaccactacacccagaagtccctgagcctgagcctgggcaagatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatccaggccctaggatgccacctccaagactcctcttcttcctcctcttcctgacaccaatggaagtcaggcctgaggaacctctagtggtgaaggtggaagagggagataacgctgtgttacagtgcctcaagggaacctcagatggacccactcagcagctgacctggtctcgggagtctccgcttaaacccttcctgaaactcagccttggactgccaggtctgggaatccacatgaggccactggctatctggctgttcatcttcaacgtctctcaacagatgggaggcttctacctgtgtcagcctggaccaccttctgagaaggcatggcagcctggttggacagtcaatgtggagggttctggtgagctgttccggtggaatgtttcggacctaggtggactgggatgtggtctgaagaacaggtcctcagagggacctagctctccttccgggaagctcatgagccccaagctgtatgtgtgggccaaagaccgccctgagatctgggagggagagcctccgtgtgtcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgccctgccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagatgagcggccgctctagacccgggctgcaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagccgtacctttaagaccaatgacttacaaggcagctgtagatcttagccactttttaaaagaaaaggggggactggaagggctaattcactcccaaagaagacaagatctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagaattcgatatcaagcttatcgataccgtcgacctcgagggggggcccggtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggagctccaccgcggtggcggcctcgaggtcgagatccggtcgaccagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttcgacggtatcgattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggaattcggagtggcgagccctcagatcctgcatataagcagctgctttttgcctgtactgggtctctctg

SEQ ID NO: 135 - DNA, 24 bp, synthetic construct
  source 1..24: mol_type = other DNA
gagtctaagtacggaccgccctgc

SEQ ID NO: 136 - DNA, 333 bp, synthetic construct
  source 1..333: mol_type = other DNA
cccccttgccctggccagcctagagaaccccaggtgtacaccctgcctcccagccaggaagagatgaccaagaaccaggtgtccctgacctgcctggtcaaaggcttctaccccagcgatatcgccgtggaatgggagagcaacggccagcccgagaacaactacaagaccaccccccctgtgctggacagcgacggcagcttcttcctgtactcccggctgaccgtggacaagagccggtggcaggaaggcaacgtcttcagctgcagcgtgatgcacgaggccctgcacaaccactacacccagaagtccctgagcctgagcctgggcaag

SEQ ID NO: 137 - DNA, 72 bp, synthetic construct
  source 1..72: mol_type = other DNA
ctcgagggcggcggagagggcagaggaagtcttctaacatgcggtgacgtggaggagaatccaggccctagg

SEQ ID NO: 138 - DNA, 357 bp, synthetic construct
  source 1..357: mol_type = other DNA
gaggtacagcttgtcgagagcggtggtggagtagtccaaccgggtcgaagtcttaggctttcctgtagcgcatctgggttcacttttagtggctacggcctctcctgggtgagacaggcgcctgggaaggggctggagtgggtagccatgatttcatctggtggctcatatacttattatgccgactccgtaaagggaagattcgcaatatcacgcgataacgctaaaaatacactcttcttgcagatggattctttgagacctgaggataccggggtttacttttgcgccagacacggggatgaccccgcctggtttgcctattggggacagggaacccctgtgacggtatcctct

SEQ ID NO: 139 - DNA, 330 bp, synthetic construct
  source 1..330: mol_type = other DNA
gatattcagcttactcaaagtccgagtagtctgtctgcctcagttggcgatagggtgaccatcacttgctccgtaagtagttctatttcttccaacaacctgcattggtatcaacagaaaccaggtaaagcacctaagccgtggatctacggaacgtccaaccttgcgtctggcgtaccaagccggttctccgggagtgggagtggtacagattacacatttactatcagttctcttcaaccggaagacattgccacatattattgccagcaatggtcatcttacccctatatgtacacatttggtcagggtacaaaggttgaaataaaa

(19) United States
(12) Patent Application Publication
(10) Pub. No.: US 2025/0025557 A1
(43) Pub. Date: Jan. 23, 2025

(54) TREATMENTS FOR CANCERS UTILIZING CELL-TARGETED THERAPIES AND ASSOCIATED RESEARCH PROTOCOLS

(71) Applicant: Fred Hutchinson Cancer Center, Seattle, WA (US)

(72) Inventors: Soheil Meshinchi, Seattle, WA (US); Quy Le, Puyallup, WA (US); Rhonda Ries, Tacoma, WA (US); Sommer Castro, Maple Valley, WA (US)

(73) Assignee: Fred Hutchinson Cancer Center, Seattle, WA (US)

(21) Appl. No.: 18/707,099

(22) PCT Filed: Nov. 2, 2022
(86) PCT No.: PCT/US2022/079180

§ 371 Date: May 2, 2024

Related U.S. Application Data
(60) Provisional application No. 63/371,265, filed on Aug. 12, 2022.
(60) Provisional application No. 63/274,914, filed on Nov. 2, 2021.

(51) Int. Cl. A61K 39/00; A61K 47/68; A61K 51/10; A61P 35/02; C07K 14/705; C07K 14/725; C07K 16/18; C07K 16/28; C07K 16/40; C12N 5/0783

(52) U.S. Cl. CPC A61K 39/4631; A61K 39/4611; A61K 39/464402; A61K 39/464411; A61K 47/68031; A61K 47/68033; A61K 47/68035; A61K 47/68037; A61K 47/6805; A61K 47/6809; A61K 47/6849; A61K 51/1027; A61P 35/02; C07K 14/70503; C07K 14/7051; C07K 14/70521; C07K 14/70578; C07K 16/18; C07K 16/28; C07K 16/40; C12N 5/0636; A61K 2121/00; C07K 2317/622; C07K 2319/03

(57) ABSTRACT

Targeted therapeutics for the treatment of cancers expressing FOLR1, MEGF10, HPSE2, KLRF2, PCDH19, and/or FRAS1 are described. The targeted therapeutics can include a chimeric antigen receptor (CAR) expressed by an immune cell or an antibody-targeted therapeutic. The targeted therapeutics can be used to treat a variety of cancers including solid tumors and blood cancers, such as CBFA2T3-GLIS2 acute myeloid leukemia (C/G AML).

### CROSS-REFERENCE TO RELATED APPLICATIONS

This application is a U.S. National Phase patent application based on International Patent Application No. PCT/US2022/079180, filed on Nov. 2, 2022, which claims priority to U.S. Provisional Patent Application No. 63/274,914 filed Nov. 2, 2021 and U.S. Provisional Patent Application No. 63/371,265 filed Aug. 12, 2022, each of which are incorporated herein by reference in their entirety as if fully set forth herein.

### REFERENCE TO SEQUENCE LISTING

The Sequence Listing associated with this application is provided in XML format in lieu of a paper copy and is hereby incorporated by reference into the specification. The name of the XML file containing the Sequence Listing is 31D9382.xml. The XML file is 151,552 bytes, was created on Apr. 2, 2024, and is being submitted electronically via Patent Center.

### FIELD OF THE DISCLOSURE

The current disclosure provides targeted cancer treatments for cancer cells expressing FOLR1, MEGF10, HPSE2, KLRF2, PCDH19, or FRAS1. The targeted therapeutic can include a chimeric antigen receptor (CAR) expressed by an immune cell, such as a T cell. Treated cancers include a variety of solid tumor cancers and blood cancers.

### BACKGROUND OF THE DISCLOSURE

According to the World Health Organization, cancer is a leading cause of death globally, and was responsible for nearly 10 million deaths in 2020. Beyond traditional cancer treatments such as surgery, chemotherapy, and radiation therapy, more targeted therapies have emerged to specifically target cancer cells by identifying and exploiting specific molecular changes seen primarily in those cells. For example, immune cells can be genetically engineered to target and kill cancer cells. Many of these immune cells are T cells have been genetically engineered to express a chimeric antigen receptor (CAR) which recognizes a protein or molecule expressed on the surface of the cancer cell so that the genetically modified T cell can recognize and kill the cancer cells. Furthermore, antibodies or binding fragments thereof that bind a protein or molecule expressed on the surface of the cancer cell can be used to trigger immune reactions against cancer cells. These antibodies or binding fragments thereof can be conjugated to cytotoxic drugs to further enhance their cytotoxic effects.

Numerous cancer types would benefit from the development of additional CAR-based therapies, such as leukemias, peritoneal cancer, fallopian tube cancer, ovarian cancer, endometrial cancer, cervical cancer, breast cancer, bladder cancer, renal cell carcinoma, pituitary tumors, lung cancer, uterine cancer, squamous cell carcinoma, ureter cancer, urethral cancer, osteosarcoma, and transitional cell carcinoma.

Pediatric acute myeloid leukemia (AML), for example, is a diverse group of diseases classified based on morphology, lineage, and genetics (Rubnitz, Blood 119:5980-8, 2012) and its prognosis depends on several cytogenetic and molecular characteristics. Despite improved survival and remission induction rates, outcomes vary significantly amongst the different biological subtypes of AML (Kim, Blood Res. 55(Suppl): S5-S13, 2020). To better stratify risk and survival outcomes, genomic investigations of AML has led to new genomic classifications and predictive biomarkers (Arber, Semin Hematol. 56: 90-5, 2019; and Arber et al., Blood 127: 2391-405, 2016). One such genetic prognostic marker includes the CBFA2T3-GLIS2 (C/G) fusion gene. The C/G fusion gene characterizes a subtype of leukemia that is extremely aggressive and specific to pediatrics. This subtype of AML is highly refractory to conventional therapies, resulting in survival rates as low as 15-30% (Masetti et al., Br J Haematol. 184(3): 337-347, 2019). Because of the significant morbidity and mortality rates for C/G AML, efforts to identify new therapies is under continual investigation.

### SUMMARY OF THE DISCLOSURE

The current disclosure provides targeted therapies against cancer cells expressing FOLR1, MEGF10, HPSE2, KLRF2, PCDH19, and/or FRAS1. Pediatric acute myeloid leukemia (AML) provides an example of a cancer type that can be treated with targeted therapies against cancer cells expressing FOLR1, MEGF10, HPSE2, KLRF2, PCDH19, and/or FRAS1. Leukemias, peritoneal cancer, fallopian tube cancer, ovarian cancer, endometrial cancer, cervical cancer, breast cancer, bladder cancer, renal cell carcinoma, pituitary tumors, lung cancer, uterine cancer, squamous cell carcinoma, ureter cancer, urethral cancer, osteosarcoma, and transitional cell carcinoma provide examples of cancer types that can be treated with targeted therapies against cancer cells expressing FOLR1.

In particular embodiments, a targeted therapeutic disclosed herein includes a chimeric antigen receptor (CAR) expressed by an immune cell, such as a T cell. In certain examples, the CAR includes a binding domain that binds FOLR1, an IgG4 spacer, a CD28 transmembrane domain, and a 4-1BB/CD3ζ intracellular effector domain. Targeted therapeutics can also include antibody conjugates, such as antibody-drug conjugates, antibody-radioisotope conjugates, or antibody-nanoparticle conjugates.

### BRIEF DESCRIPTION OF THE FIGURES

Some of the drawings submitted herein may be better understood in color. Applicant considers the color versions of the drawings as part of the original submission and reserves the right to present color images of the drawings in later proceedings.

FIGS. 1A-1J. CBFA2T3-GLIS2 (C/G)-cord blood (CB) cells induce leukemia recapitulating primary disease. 1A. Diagram of experimental design. 1B. Kaplan-Meier survival curves of NSG-SGM3 mice transplanted with green fluorescent protein (GFP)-CB control and C/G-CB cells. Statistical differences in survival were evaluated using Logrank Mantel-Cox. 1C. Representative histology of hematoxylin and eosin (H&E) stain of femurs taken from mice transplanted with C/G-CB cells (top) and a C/G positive patient sample (bottom) after development of leukemia. PDX stands for C/G patient-derived leukemia cells. Magnification: left (2.5×), middle (5×), right (C/G-CB 40×; PDX, 20×). 1D. Expression of the RAM immunophenotype in C/G-CB cells harvested from the bone marrow of a representative mouse at necropsy compared to a primary patient sample and PDX marrow xenograft cells. In all three samples, malignant cells were gated based on human CD45 expression and side scatter (SSC). 1E. Left and middle, representative immunohistochemistry showing high expression of ERG (10× magnification) and CD56 (5× magnification) in the femur of a representative mouse transplanted with C/G-CB cells. Right, small aggregates of blasts with high CD56 expression detected in a bone marrow biopsy of a chemotherapy refractory C/G fusion positive patient, consistent with residual, adherent, patchy disease distribution (100× magnification). 1F. Kaplan-Meier plot showing survival in primary (1°), secondary (2°) and tertiary (3°) transplantations of C/G-CB cells. 1G. Engraftment of C/G-CB cells in the bone marrow at time of symptomatic leukemia, shown as percent human CD45+. 1H. Quantification of CD56+ cells amongst human CD45+ cells isolated from the bone marrow (BM) at necropsy following development of symptomatic leukemia. 1I. Expression of acute megakaryocytic leukemia (AMKL) markers, CD41 and CD42, in C/G-CB and PDX cells harvested from the bone marrow at necropsy. C/G-CB cells were gated on human CD45+ cells. PDX cells were gated on human CD45+CD56+ cells. 1J. Quantification of CD41/CD42 subsets described in FIG. 1I. Bars indicate mean+/−standard error of mean (SEM).

FIG. 2. C/G-CB cells form tight clusters in mouse bone marrow. (related to FIGS. 1A-1J). Histology of femurs taken from primary, secondary and tertiary transplants of C/G-CB cells.

FIGS. 3A-3C. Expression of CD56 and AMKL markers in C/G-CB xenograft cells following development of symptomatic leukemia in NSG-SGM3 mice. 3A. Percent human CD45+ cells in the bone marrow, spleen, liver and peripheral blood (PB) from mice transplanted with C/G-CB cells in primary (1°), secondary (2°) and tertiary (3°) transplants. 3B, 3C. Percent CD56+ and CD41/CD42 subsets in mouse tissues described in FIG. 3A.

FIGS. 4A-4J. Endothelial cells (ECs) enhance the proliferative potential and promote leukemic progression of C/G-CB cells. 4A. Diagram of experimental design. 4B. Growth kinetics of C/G-CB and GFP-CB cells in EC co-culture or myeloid promoting conditions (MC). 4C. C/GCB cells expanded in EC co-culture for 9 weeks were reseeded in EC co-culture either directly (direct contact) or in EC transwells (indirect contact) or placed in liquid culture containing serum free expansion medium (SFEM) II (+SCF, FLT3L, and TPO). After 7 days, the number of GFP+ cells was quantified by flow cytometry. 4D. At 6 and 12 weeks, a fraction of each culture was transferred to MegaCult cultures. Colonies derived from megakaryocytic (Mk) progenitors were scored and enumerated. Data are normalized to the 500 input cells at the start of the EC co-culture or MC culture. A representative colony stained with anti-human CD41 and an alkaline phosphotase detection system is shown. 4E. Equivalent number of C/G-CB and GFPCB cells were transplanted into NSG-SGM3 mice (5-10×10⁶/mouse) at indicated timepoints. Due to insufficient expansion, GFP-CB cells were not transplanted after 3 weeks in either condition, similarly for C/G-CB cells after 6 weeks in MC culture. Median survival and Kaplan-Meier survival curves are shown. C/G-CB (N=3 mice/group), GFP-CB (N=2 mice/group) 4F, 4G. Quantification of CD56+ cells (4F) and CD41/CD42 subsets (4G) amongst human CD45+ cells over weeks in culture. 4H. Unsupervised clustering by uniform manifold and projection (UMAP) analysis of C/G-CB and GFP-CB cells in reference to primary AML samples. Dashed circle indicates C/G-CB cells co-cultured with ECs at week 6 and 12 timepoints. NBM=normal bone marrow. 4I. Heatmap of differentially expressed genes in C/G-CB versus GFP-CB cells in EC co-culture or MC. 4J. GSEA plots of C/G and HSC signature genes comparing C/G-CB cells in EC co-culture versus MC. (4B-4E, 4G-4I) Data presented as mean+/−standard deviation from 3 technical replicates.

FIGS. 5A-5C. Assessment of RAM and AMKL markers in C/G-CB cells isolated from mice transplanted with engineered cells cultured in EC co-culture or MC. 5A. Percent human CD45+ cells in the bone marrow, spleen liver and peripheral blood from mice transplanted with C/G-CB and GFP-CB cells at indicated timepoints in EC co-culture or MC. 5B, 5C. Percent CD41/CD42 subsets (5B) and CD56+ cells (5C) among live human CD45+ in mouse tissues described in FIG. 5A. Data analyzing CB cells in the liver for mice transplanted with GFP-CB cells from MC culture are not included as not enough cells were present in the samples. Peripheral blood data from 2 mice transplanted with C/G-CB cells grown in MC are also not included as enough cells were not present in the samples.

FIGS. 6A-6C. C/G-CB cells cultured with ECs recapitulate the immunophenotype and morphology of C/G fusion positive AML. 6A. Expression of the RAM immunophenotype in C/G-CB cells after 6 weeks in EC co-culture or MC. 6B. Quantification of CD41/CD42 subsets at indicated timepoints in EC co-culture or MC. 6C. Morphological evaluation of the C/G-CB cells cultured with ECs or in MC for 9 weeks showed features of megakaryocytic differentiation, including open chromatin, prominent nucleoli, and abundant focally, basophilic and vacuolated cytoplasm with cytoplasmic blebbing.

FIGS. 7A-7D. ECs promote transformation of C/G-CB cells. 7A. Schematic of transduction and long-term cultures of cord blood CD34+ HSPCs from a second donor. 7B. Growth kinetics of transduced cells over days in EC or MC as determined by the cumulative number of GFP+ cells. Mean+/−standard deviation from 3 technical replicates are shown. Growth rate constant k was determined by regression analysis using the formula N(t)=N(0)ekt where t is measured in days. 7C. Following 6 and 12 weeks of culture, a fraction of each culture was transferred to megacult to enumerate Mk colonies. Data are normalized to the CD34+ input cells at the start of the culture and presented as mean+/−standard deviation from 3 technical replicates. 7D. Expression of the RAM immunophenotype in C/G-CB cells after 6 weeks in either EC co-culture or MC.

FIGS. 8A, 8B. C/G-specific genes and pathways that are recapitulated in C/G-CB cells cultured with ECs versus in MC. 8A. The expression (labeled Expression (Log 2 cpm)) of ERG, BMP2 and GATA1 in GFP-CB versus C/G-CB cells over weeks in EC and MC conditions as well as in C/G fusion positive primary versus normal marrow samples. Single-sample gene-set enrichment (ssGSEA) scores (labeled Enrichment Score) of Hedgehog, TGFB, and WNT signaling pathways for GFP-CB versus C/G-CB cells and normal bone marrow samples versus primary fusion positive samples. 8B. Pathways that are upregulated (left) and downregulated (right) in C/G-CB cells in EC co-culture compared to MC.

FIGS. 9A-9C. Expression of C/G-specific genes. Heat maps showing expression of C/G-specific focal adhesion and cell adhesion molecule genes (9A), genes associated with primary C/G fusion positive AML (9E), and HSC signature genes (9C). Unsupervised hierarchical clustering demonstrates clustering of C/G-CB cells cultured with ECs for 6 and 12 weeks with primary C/G samples.

FIGS. 10A-10G. Integrative transcriptomics of primary samples and C/G-CB identify FOLR1 therapeutic target. 10A. Diagram of computational workflow to identify C/G-specific CAR targets. See Methods and FIG. 11 for details. Normal tissues include bulk bone marrow (BM) samples and peripheral blood (PB) CD34+ samples. 10B, 10C. Expression of C/G-specific CAR targets in primary fusion positive patients versus normal bone marrow (NBM) (10B) and C/G-CB versus GFP-CB cells (10C). 10D. Top, gating strategies used to identify AML cells and normal lymphocytes, monocytes and myeloid cells in 4 representative patients based on CD45 expression and SSC. Bottom, FOLR1 expression in the AML blast subpopulation versus normal cells. 10E. Quantification of FOLR1 expression (geometric mean fluorescent intensity, MFI) among AML blasts and their normal counterparts across N=15 patients. Autofluorescence was used as control. ***, p<0.0005 (paired Student t-test) 10F, 10G. Expression of FOLR1 (10F) and quantification of FOLR1+ cells (10G) amongst GFP-CB and C/G-CB over weeks in EC co-culture.

FIG. 11. Identification of C/G fusion-specific CAR targets. (Related to FIG. 10A) Flow diagram of AML-restricted gene and CAR-T target identification. The procedure involves three main steps: 1) Determine the ratio of expression for AML primary samples versus healthy normal hematopoietic tissue samples (bulk marrows and CD34+ peripheral blood) from log 10 transformed normalized gene expression. The ratio is calculated per gene from mean AML expression and mean normal hematopoietic tissue expression, where normal tissue values are the divisor, which acts as a measure of over or under expression. A normal curve is fit to the ratios and this procedure is completed for all heterogenous AML samples as a group, and iteratively within fusion and mutation subtypes; genes with ratios greater than +2 standard deviations and with absent expression in normal hematopoietic tissues were retained (N=607) for further analysis. 2) The AML restricted genes were further selected if found to be significantly overexpressed in fusion positive patient samples compared to healthy marrows and were likewise overexpressed in C/G-CB at weeks 6 and 12 in EC co-culture with absent expression in GFPCB controls, providing several candidate (N=42) targets. 3) Optimal CAR-T targets were selected by the identification of candidate genes with cell surface localization potential, and those with an absence of expression in healthy tissue controls as noted in step 1, but expression in >75% of C/G patient samples, and with moderate to high expression levels (N=6).

FIG. 12. Expression of FOLR1 transcript in C/G-CB cells cultured on ECs. RT-PCR analysis of FOLR1 expression in engineered CB cells and in fusion positive cell lines M07e and WSU-AML. Expression is normalized as fold-change relative to GFP-CB/EC Wk 3 samples.

FIGS. 13A-13D. Pre-clinical efficacy of FOLR1 CAR T cells against C/G AML cells. 13A. Cytolytic activity of CD8 T cells unmodified or transduced with FOLR1 CAR following 6 hours of co-culture with C/G-CB, WSU-AML, Kasumi-1 FOLR1+ and Kasumi-1 parental cells. Data presented are mean leukemia specific lysis+/−SD from 3 technical replicates at indicated effector:target (E:T) ratios. Data are representative of 2 donors (see related data in FIG. 16). 13B. Concentration of secreted IL-2, IFN-γ, and TNF-α in the supernatant following 24 hour of T cell/AML co-culture at 1:1 E:T ratio as measured by ELISA. Data are representative of 2 donors and are presented as mean+/−SD from 3 technical replicates (see related data in FIGS. 17A-17F). Where concentrations of cytokines are too low to discern, the number above the x-axis indicates the average concentration. Statistical significance was determined by unpaired Student's t test, assuming unequal variances. p<0.05 (*), p<0.005 (**), p<0.0005 (***). 13C. Representative flow cytometric analysis of cell proliferation of Cell Proliferation Dye (Celltrace)-labeled unmodified and FOLR1 CAR T cells after 4-day co-culture with target cells at 1:1 E:T ratio. CAR T cells divided rapidly and diluted their Celltrace fluorescence after 4-hour co-incubation with FOLR1-positive AML cells. Data are representative of 2 donors. 13D. Bioluminescent imaging of C/G-CB, WSU-AML, Kasumi-1 FOLR1+ and Kasumi-1 leukemias in mice treated with unmodified or FOLR1 CAR T cells at 5×10⁶ T cells per mouse. N=5 mice/group. Radiance scale indicates an increase in leukemia from blue to red; X indicates death.

FIG. 14A-14C. In vivo efficacy of FOLR1-directed CAR T. (Related to FIGS. 13A-13D) 14A. Quantification of leukemia burden over time based on IVIS radiance in CBFA2T3-GLIS2-transduced HSPCs, WSU-AML, Kasumi-1 FOLR1+ and Kasumi-1 xenografts treated with unmodified or FOLR1 CAR T cells at 5×10⁶/mouse. Leukemia burden is shown for each mouse. N=5 mice per group. 14B. Quantification of human T cells in the mouse peripheral blood at indicated time points after T cell injection. Shown is human CD45+CD3+ frequency amongst DAPI− cells. N=5 mice per group. Data presented are the average+/−standard deviation from 5 mice. *, p<0.05 (unpaired Student's t-test) 14C. Kaplan-Meier survival curves of xenografts treated with unmodified or FOLR1 CAR T cells. N=5 per group. Statistical differences in survival were evaluated using Log-rank Mantel-Cox. Note: 2 C/G-CB bearing mice treated with CAR T cells died without leukemia and T cells present in bone marrow, spleen and liver tissues and in peripheral blood as determined by flow cytometric analysis

FIGS. 15-15F. FORL1-directed CAR T effectively eliminate C/G-CB cells without impacting viability of HSPCs. 15A. Gating strategy used to identify HPSC subsets from a representative CD34-enriched marrow sample from a healthy donor. Shown is representative of 3 donors. Immunophenotype of the HSPCs is as follows: CD34+CD38−CD90+CD45RA− (hematopoietic stem cell, HSC); CD34+CD38−CD90−CD45RA− (multipotent progenitors, MPP); CD34+CD38−CD90−CD45RA+ multi-lymphoid progenitors, MLP); CD34+CD38+CD10+(Common lymphoid progenitor, CLP); CD34+CD38+CD10−CD123−CD45RA− (megakaryocyte-erythroid progenitor, MEP); CD34+CD38+CD10−CD123+CD45RA− (common myeloid progenitor, CMP); CD34+CD38+CD10−CD123+CD45RA+ (granulocyte monocyte progenitor, GMP). 15B. Histogram of FOLR1 expression in normal HSPC subsets. 15C. Quantification of percent FOLR1+ in C/G-CB cells (>12 weeks of EC co-culture) and HSPC subsets from three CD34-enriched samples from healthy donors. 15D. Percent specific lysis in C/G-CB cells and the HSPC subsets shown in FIG. 15C following 4-hour incubation with unmodified or FOLR1 CAR T cells at 2:1 E:T ratio. Note that data points for C/G-CB cells are from 2 technical replicates. Only two out of three normal CD34+ samples were used in this experiment. 15E, 15F. After 4 hours, co-cultures of healthy donor CD34+ or C/G-CB cells with either unmodified or MSLN CAR T cells at 2:1 E:T ratio were transferred to methylcellulose with cytokines for colony-forming cell (CFC) assay. Colonies derived from erythroid (E), granulocyte-macrophage (G, M, and GM) and multipotential granulocyte, erythroid, macrophage, megakaryocyte (GEMM) progenitors were scored and enumerated after 7-10 days (15E). Total colonies from C/G-CB cells are tabulated (15F). Data are presented as mean+/−SD from 3 technical replicates for each donor. No significant difference in the total number of colonies was detected between co-cultures with unmodified T cells versus FOLR1 CAR T cells for normal HSPCs as determined by unpaired Student's t test, assuming unequal variances. Statistical significance was determined by unpaired Student's t test, assuming unequal variances. P<0.05 (*), p<0.005 (**), p<0.0005 (***).

FIG. 16. Expression of C/G transcript in C/G-CB cells. RT-PCR analysis of C/G expression in engineered CB cells and in fusion positive cell lines M07e and WSU-AML.

FIGS. 17A-17F. FOLR1 CAR constructs and reactivity of short, intermediate and long FOLR1 CAR T cells. 17A. Schematic diagram of second-generation FOLR1 CAR constructs with different IgG4 spacer lengths. SP=GM-CSFR signal peptide; scFv=single-chain variable fragment; TM=transmembrane domain; CD=costimulatory domain; SD=stimulatory domain; tCD19=transduced marker truncated CD19. The anti-FOLR1 scFv could be replaced with a different binding domain including binding domains that bind to MEGF10, HPSE2, KLRF2, PCDH19, FRAS1, or other binding domains that bind to FOLR1. 17B. Expression of FOLR1 in C/G-CB, M07e, WSU-AML, Kasumi-1 FOLR1+ and Kasumi-1 parental cells. 17C. Cytolytic activity of CD8 T cells unmodified or transduced with short, intermediate or long FOLR1 CAR construct against C/G-CB, M07e, WSU-AML, Kasumi-1 FOLR1+ and Kasumi-1 parental cells in a 6-hour assay. Shown is mean percent specific lysis+/−SD from 3 technical replicates at indicated Effector:Target (E:T) ratios. 17D. Concentration of secreted IL-2, IFN-γ, and TNF-α in the supernatant following 24 hour of CD8 T cell/AML co-culture at 1:1 E:T ratio. Mean+/−SD from 3 technical replicates is shown. 17E. Representative flow plots showing expression of NFAT, NF-kB and AP-1 in Jurkat Nur77 reporter transduced with FOLR1 CAR constructs cultured alone (top) or co-incubated with Kasumi-1 FOLR1+ target cells for 24 hours at 1:1 E:T ratio (bottom). Kasumi-1 FOLR1+ cells were labeled with Violet Cell Proliferation Dye to differentiate from Jurkat cells. Transduced Jurkat cells were gated based on tCD19 expression. Number in top right corner indicates the percentage of positive cells. Analysis was performed on day 4 post transduction. 17F. Quantification of percent NFAT+, NF-kB+ and AP-1+ cells in FIG. 17E.

FIG. 18. Information for antibodies.

FIG. 19. Sequences supporting the disclosure include IgG4 hinge coding sequence-A (SEQ ID NO: 1); IgG4 hinge coding sequence-B (SEQ ID NO: 2); IgG4 hinge S10P (SEQ ID NO: 135); Hinge+intermediate spacer (DS) (SEQ ID NO: 136); IgG4-int(DS) coding sequence (SEQ ID NO: 3); IgG4-long coding sequence (SEQ ID NO: 4); CD3ζ coding sequence (SEQ ID NO: 5); CD3 protein-A (SEQ ID NO: 6); CD3ζ protein-B (SEQ ID NO: 7); 4-1BB signaling coding sequence-A (SEQ ID NO: 8); 4-1BB signaling coding sequence-B (SEQ ID NO: 9); 4-1BB protein-A (SEQ ID NO: 10); 4-1BB protein-B (SEQ ID NO: 11); CD28TM coding sequence-A (SEQ ID NO: 12); CD28TM coding sequence-B (SEQ ID NO: 13); CD28TM coding sequence-C(SEQ ID NO: 14); CD28TM protein-A (SEQ ID NO: 15); CD28TM protein-B (SEQ ID NO: 16); T2A coding sequence (SEQ ID NO: 137); P2A (SEQ ID NO: 17); T2A (SEQ ID NO: 18); E2A (SEQ ID NO: 19); F2A (SEQ ID NO: 20); tCD19 coding sequence (SEQ ID NO: 117); Psi (SEQ ID NO: 118); RRE (SEQ ID NO: 119); and Flap (SEQ ID NO: 120).

### DETAILED DESCRIPTION

For many years, the chosen treatments for cancer were surgery, chemotherapy, and/or radiation therapy. In recent years, more targeted therapies have emerged to specifically target cancer cells by identifying and exploiting specific molecular and/or immunophenotypic changes seen primarily in those cells. For example, many cancer cells preferentially express particular antigens on their cellular surfaces and these antigens have provided targets for successful antibody- and cell-based therapeutics.

Although targeted therapies have been successful in treating many cancers, the targeted therapy of acute myeloid leukemia (AML) remains a challenge given significant overlap of target antigens expressed on AML and normal hematopoietic cells.

Pediatric acute myeloid leukemia (AML) is a diverse group of diseases classified based on morphology, lineage, and genetics (Rubnitz, Blood 119:5980-8, 2012) and its prognosis depends on several cytogenetic and molecular characteristics. Although, the overall survival and remission-induction rates of children with AML have improved over the past three decades, outcomes vary significantly amongst the different biological subtypes of AML (Kim, Blood Res. 55(Suppl): S5-S13, 2020). To better stratify risk and survival outcomes, genomic investigations of AML have led to new genomic classifications and predictive biomarkers (Arber, Semin Hematol. 56: 90-5, 2019; and Arber et al., Blood 127: 2391-405, 2016). One such genetic prognostic marker includes the CBFA2T3-GLIS2 (C/G) fusion gene. The C/G fusion gene characterizes a subtype of leukemia that is extremely aggressive and specific to pediatrics. This subtype of AML is highly refractory to conventional therapies, resulting in survival rates as low as 15-30% (Masetti et al., Br J Haematol. 184(3): 337-347, 2019).

C/G AML and other AML-restricted genes were discovered through an expansive target discovery effort through TARGET and Target Pediatric AML (TpAML). These genes were further filtered to include those that are upregulated in both C/G AML and in C/G-cord blood (CB) cells cultured with endothelial cells and to those genes that encode proteins that localize to the plasma membrane. This resulted in seven C/G fusion-specific targets: FOLR1, MEGF10, HPSE2, KLRF2, PCDH19, and FRAS1 which were identified to be highly expressed in C/G patients and in C/G-CB cells but entirely silent in normal hematopoiesis. The current disclosure provides targeted therapeutic treatments with binding domains that bind FOLR1, MEGF10, HPSE2, KLRF2, PCDH19, or FRAS1 for the treatment of AML including C/G AML.

Targeted therapeutics disclosed herein that bind FOLR1 can additionally be used to treat other cancers including other leukemias, peritoneal cancer, fallopian tube cancer, ovarian cancer (e.g., epithelial ovarian cancer), endometrial cancer, cervical cancer, breast cancer (e.g., triple-negative breast cancer, HER2-breast cancer), bladder cancer, renal cell carcinoma, pituitary tumors, lung cancer (e.g., lung adenocarcinoma or epithelial lung cancer such as non-small cell lung cancer), uterine cancer, squamous cell carcinoma, ureter cancer, urethral cancer, osteosarcoma, or transitional cell carcinoma.

Particular examples of targeted therapeutics disclosed herein include chimeric antigen receptors (CAR). In particular embodiments, the CAR include a binding domain that binds FOLR1. In particular embodiments, the binding domain that binds FOLR1 is a Farletuzumab scFv. In particular embodiments, the CAR include a binding domain that binds MEGF10. In particular embodiments, the CAR include a binding domain that binds HPSE2. In particular embodiments, the CAR include a binding domain that binds KLRF2. In particular embodiments, the CAR include a binding domain that binds PCDH19. In particular embodiments, the CAR include a binding domain that binds FRAS1.

In particular embodiments, the current disclosure provides CAR having an intermediate spacer region. In particular embodiments, the intermediate spacer region includes the hinge region and the CH3 domain of IgG4. In particular embodiments, the spacer is a short spacer. In particular embodiments, the spacer is a long spacer.

In particular embodiments the current disclosure provides CAR having a transmembrane domain including the CD28 transmembrane domain. In particular embodiments, the current disclosure provides CAR having an intracellular effector domain including the 4-1BB and CD3ζ signaling domains.

In particular embodiments, the CAR including a binding domain that binds FOLR1 is encoded by SEQ ID NO: 134.

The current disclosure also provides targeted therapeutics for the treatment of cancer based on antibody formats, such as antibody-drug conjugates, antibody-radioisotope conjugates, antibody-immunotoxin conjugates, or antibody-nanoparticle conjugates.

The current disclosure also provides methods and assays to further study the cancer biology of C/G AML. The cancer biology of C/G AML can be studied by the development of a model for C/G AML cells prepared by transduction of a C/G fusion gene into target cells. In particular embodiments, the cells include cord blood (CB) hematopoietic stem and progenitor cells (HSPCs). CB-HSPC cells transduced with the C/G fusion gene are referred to herein as C/G-CB cells. Furthermore, to model C/G AML cells, the microenvironment of C/G AML is recreated by either culturing the transduced cells in an animal model or in micro-environment stimulating conditions in monoculture. In particular embodiments, micro-environment stimulating conditions include co-culture with endothelial cells. In particular embodiments, micro-environment stimulating conditions include myeloid promoting conditions.

Aspects of the current disclosure are now described with additional detail and options as follows: (i) Immune Cells; (ii) Cell Sample Collection and Cell Enrichment; (iii) Genetically Modifying Cell Populations to Express Chimeric Antigen Receptors (CAR); (iii-a) Genetic Engineering Techniques; (iii-b) CAR Subcomponents; (iii-b-i) Binding Domains; (iii-b-ii) Spacer Regions; (iii-b-iii) Transmembrane Domains; (iii-b-iv) Intracellular Effector Domains; (iii-b-v) Linkers; (iii-b-vi) Control Features Including Tag Cassettes, Transduction Markers, and/or Suicide Switches; (iv) Cell Activating Culture Conditions; (v) Ex Vivo Manufactured Cell Formulations; (vi) Antibody Conjugates; (vii) Compositions; (viii) Methods of Use; (ix) Reference Levels Derived from Control Populations; (x) Cell Transformation Methods; (xi) Exemplary Embodiments; (xii) Experimental Examples; and (xiii) Closing Paragraphs. These headings are provided for organizational purposes only and do not limit the scope or interpretation of the disclosure.

(i) Immune Cells. The present disclosure describes cells genetically modified to express CAR. Genetically modified cells can include T-cells, B cells, natural killer (NK) cells, NK-T cells, monocytes/macrophages, lymphocytes, hematopoietic stem cells (HSCs), hematopoietic progenitor cells (HPC), and/or a mixture of HSC and HPC (i.e., HSPC). In particular embodiments, genetically modified cells include T-cells.

Several different subsets of T-cells have been discovered, each with a distinct function. For example, a majority of T-cells have a T-cell receptor (TCR) existing as a complex of several proteins. The actual T-cell receptor is composed of two separate peptide chains, which are produced from the independent T-cell receptor alpha and beta (TCRα and TCRβ) genes and are called α- and β-TCR chains.

γδ T-cells represent a small subset of T-cells that possess a distinct T-cell receptor (TCR) on their surface. In γδ T-cells, the TCR is made up of one γ-chain and one δ-chain. This group of T-cells is much less common (2% of total T-cells) than the αβ T-cells.

CD3 is expressed on all mature T cells. Activated T-cells express 4-1BB (CD137), CD69, and CD25. CD5 and transferrin receptor are also expressed on T-cells.

T-cells can further be classified into helper cells (CD4+ T-cells) and cytotoxic T-cells (CTLs, CD8+ T-cells), which include cytolytic T-cells. T helper cells assist other white blood cells in immunologic processes, including maturation of B cells into plasma cells and activation of cytotoxic T-cells and macrophages, among other functions. These cells are also known as CD4+ T-cells because they express the CD4 protein on their surface. Helper T-cells become activated when they are presented with peptide antigens by MHC class II molecules that are expressed on the surface of antigen presenting cells (APCs). Once activated, they divide rapidly and secrete small proteins called cytokines that regulate or assist in the active immune response.

Cytotoxic T-cells destroy virally infected cells and tumor cells and are also implicated in transplant rejection. These cells are also known as CD8+ T-cells because they express the CD8 glycoprotein on their surface. These cells recognize their targets by binding to antigen associated with MHC class I, which is present on the surface of nearly every cell of the body.

“Central memory” T-cells (or “TCM”) as used herein refers to an antigen experienced CTL that expresses CD62L or CCR7 and CD45RO on the surface thereof and does not express or has decreased expression of CD45RA as compared to naive cells. In particular embodiments, central memory cells are positive for expression of CD62L, CCR7, CD25, CD127, CD45RO, and CD95, and have decreased expression of CD45RA as compared to naive cells.

“Effector memory” T-cell (or “TEM”) as used herein refers to an antigen experienced T-cell that does not express or has decreased expression of CD62L on the surface thereof as compared to central memory cells and does not express or has decreased expression of CD45RA as compared to a naive cell. In particular embodiments, effector memory cells are negative for expression of CD62L and CCR7, compared to naive cells or central memory cells, and have variable expression of CD28 and CD45RA. Effector T-cells are positive for granzyme B and perforin as compared to memory or naive T-cells.

“Naive” T-cells as used herein refers to a non-antigen experienced T cell that expresses CD62L and CD45RA and does not express CD45RO as compared to central or effector memory cells. In particular embodiments, naive CD8+ T lymphocytes are characterized by the expression of phenotypic markers of naive T-cells including CD62L, CCR7, CD28, CD127, and CD45RA.

Natural killer cells (also known as NK cells, K cells, and killer cells) are activated in response to interferons or macrophage-derived cytokines. They serve to contain viral infections while the adaptive immune response is generating antigen-specific cytotoxic T cells that can clear the infection. NK cells express CD8, CD16 and CD56 but do not express CD3.

NK cells include NK-T cells. NK-T cells are a specialized population of T cells that express a semi-invariant T cell receptor (TCR ab) and surface antigens typically associated with natural killer cells. NK-T cells contribute to antibacterial and antiviral immune responses and promote tumor-related immunosurveillance or immunosuppression. Like natural killer cells, NK-T cells can also induce perforin-, Fas-, and TNF-related cytotoxicity. Activated NK-T cells are capable of producing IFN-γ and IL-4. In particular embodiments, NK-T cells are CD3+/CD56+.

Macrophages (and their precursors, monocytes) reside in every tissue of the body (in certain instances as microglia, Kupffer cells and osteoclasts) where they engulf apoptotic cells, pathogens and other non-self-components. Monocytes/macrophages express CD11b, F4/80; CD68; CD11c; IL-4Rα; and/or CD163.

Immature dendritic cells (i.e., pre-activation) engulf antigens and other non-self-components in the periphery and subsequently, in activated form, migrate to T-cell areas of lymphoid tissues where they provide antigen presentation to T cells. Dendritic cells express CD1a, CD1b, CD1c, CD1d, CD21, CD35, CD39, CD40, CD86, CD101, CD148, CD209, and DEC-205.

Hematopoietic Stem/Progenitor Cells or HSPC refer to a combination of hematopoietic stem cells and hematopoietic progenitor cells.

Hematopoietic stem cells refer to undifferentiated hematopoietic cells that are capable of self-renewal either in vivo, essentially unlimited propagation in vitro, and capable of differentiation to all other hematopoietic cell types.

A hematopoietic progenitor cell is a cell derived from hematopoietic stem cells or fetal tissue that is capable of further differentiation into mature cell types. In certain embodiments, hematopoietic progenitor cells are CD24^{lo} Lin⁻ CD117⁺ hematopoietic progenitor cells. HPC can differentiate into (i) myeloid progenitor cells which ultimately give rise to monocytes and macrophages, neutrophils, basophils, eosinophils, erythrocytes, megakaryocytes/platelets, or dendritic cells; or (ii) lymphoid progenitor cells which ultimately give rise to T-cells, B-cells, and NK-cells.

HSPC can be positive for a specific marker expressed in increased levels on HSPC relative to other types of hematopoietic cells. For example, such markers include CD34, CD43, CD45RO, CD45RA, CD59, CD90, CD109, CD117, CD133, CD166, HLA DR, or a combination thereof. Also, the HSPC can be negative for an expressed marker relative to other types of hematopoietic cells. For example, such markers include Lin, CD38, or a combination thereof. Preferably, the HSPC are CD34⁺ cells.

A statement that a cell or population of cells is “positive” for or expressing a particular marker refers to the detectable presence on or in the cell of the particular marker. When referring to a surface marker, the term can refer to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

A statement that a cell or population of cells is “negative” for a particular marker or lacks expression of a marker refers to the absence of substantial detectable presence on or in the cell of a particular marker. When referring to a surface marker, the term can refer to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

Cells to be genetically modified according to the teachings of the current disclosure can be patient-derived cells (autologous) or allogeneic when appropriate and can also be in vivo or ex vivo. In particular embodiments, cells to be genetically modified include CD4+ or CD8+ T cells.

(ii) Cell Sample Collection and Cell Enrichment. Methods of sample collection and enrichment are known by those skilled in the art. In some embodiments, cells are derived from cell lines. In particular embodiments, cells are derived from humans. In some embodiments, cells are obtained from a xenogeneic source, for example, from mouse, rat, non-human primate, or pig.

In some embodiments, T cells are derived or isolated from samples such as whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. In particular embodiments, cells from the circulating blood of a subject are obtained, e.g., by apheresis or leukapheresis. The samples, in particular embodiments, contain lymphocytes, including T cells, monocytes, granulocytes, B cells, other nucleated white blood cells, HSC, HPC, HSPC, red blood cells, and/or platelets, and in some aspects, contains cells other than red blood cells and platelets and further processing is necessary.

In some embodiments, blood cells collected from a subject are washed, e.g., to remove the plasma fraction and to place the cells in an appropriate buffer or media for subsequent processing steps. In particular embodiments, the cells are washed with phosphate buffered saline (PBS). In some embodiments, the wash solution lacks calcium and/or magnesium and/or many or all divalent cations. Washing can be accomplished using a semi-automated “flow-through” centrifuge (for example, the Cobe 2991 cell processor, Baxter) according to the manufacturer's instructions. Tangential flow filtration (TFF) can also be performed. In particular embodiments, cells can be re-suspended in a variety of biocompatible buffers after washing, such as, Ca++/Mg++ free PBS.

The isolation can include one or more of various cell preparation and separation steps, including separation based on one or more properties, such as size, density, sensitivity or resistance to particular reagents, and/or affinity, e.g., immunoaffinity, to antibodies or other binding partners. In particular embodiments, the isolation is carried out using the same apparatus or equipment sequentially in a single process stream and/or simultaneously. In particular embodiments, the isolation, culture, and/or engineering of the different populations is carried out from the same starting material, such as from the same sample.

In particular embodiments, a sample can be enriched for T cells by using density-based cell separation methods and related methods. For example, white blood cells can be separated from other cell types in the peripheral blood by lysing red blood cells and centrifuging the sample through a Percoll or Ficoll gradient.

In particular embodiments, a bulk T cell population can be used that has not been enriched for a particular T cell type. In particular embodiments, a selected T cell type can be enriched for and/or isolated based on cell-marker based positive and/or negative selection. In positive selection, cells having bound cellular markers are retained for further use. In negative selection, cells not bound by a capture agent, such as an antibody to a cellular marker are retained for further use. In some examples, both fractions can be retained for a further use.

The separation need not result in 100% enrichment or removal of a particular cell population or cells expressing a particular marker. For example, positive selection of or enrichment for cells of a particular type refers to increasing the number or percentage of such cells but need not result in a complete absence of cells not expressing the marker. Likewise, negative selection, removal, or depletion of cells of a particular type refers to decreasing the number or percentage of such cells but need not result in a complete removal of all such cells.

In some examples, multiple rounds of separation steps are carried out, where the positively or negatively selected fraction from one step is subjected to another separation step, such as a subsequent positive or negative selection.

In some embodiments, an antibody or binding domain for a cellular marker is bound to a solid support or matrix, such as a magnetic bead or paramagnetic bead, to allow for separation of cells for positive and/or negative selection. For example, in some embodiments, the cells and cell populations are separated or isolated using immunomagnetic (or affinity magnetic) separation techniques (reviewed in Methods in Molecular Medicine, vol. 58: Metastasis Research Protocols, Vol. 2: Cell Behavior In Vitro and In Vivo, p 17-25 Edited by: S. A. Brooks and U. Schumacher © Humana Press Inc., Totowa, NJ); see also U.S. Pat. Nos. 4,452,773; 4,795,698; 5,200,084; and EP 452342.

In some embodiments, affinity-based selection is via magnetic-activated cell sorting (MACS) (Miltenyi Biotec, Auburn, CA). MACS systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1(5):355-376). In both cases, cells can be labeled with multiple markers, allowing for the isolation of well-defined cell subsets at high purity.

Cell-markers for different T cell subpopulations are described above. In particular embodiments, specific subpopulations of T cells, such as cells positive or expressing high levels of one or more surface markers, e.g., CCR7, CD45RO, CD8, CD27, CD28, CD62L, CD127, CD4, and/or CD45RA T cells, are isolated by positive or negative selection techniques. CD3+, CD28+ T cells can be positively selected for and expanded using anti-CD3/anti-CD28 conjugated magnetic beads (e.g., DYNABEADS® M-450 CD3/CD28 T Cell Expander).

In particular embodiments, a CD8+ or CD4+ selection step is used to separate CD4+ helper and CD8+ cytotoxic T cells. Such CD8+ and CD4+ populations can be further sorted into sub-populations by positive or negative selection for markers expressed or expressed to a relatively higher degree on one or more naive, memory, and/or effector T cell subpopulations.

In some embodiments, enrichment for central memory T (TCM) cells is carried out. In particular embodiments, memory T cells are present in both CD62L subsets of CD8+ peripheral blood lymphocytes. PBMC can be enriched for or depleted of CD62L, CD8 and/or CD62L+CD8+ fractions, such as by using anti-CD8 and anti-CD62L antibodies.

In some embodiments, the enrichment for central memory T (TCM) cells is based on positive or high surface expression of CCR7, CD45RO, CD27, CD62L, CD28, CD3, and/or CD127; in some aspects, it is based on negative selection for cells expressing or highly expressing CD45RA and/or granzyme B. In some aspects, isolation of a CD8+ population enriched for TCM cells is carried out by depletion of cells expressing CD4, CD14, CD45RA, and positive selection or enrichment for cells expressing CCR7, CD45RO, and/or CD62L. In one aspect, enrichment for central memory T (TCM) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8+ cell population or subpopulation, also is used to generate the CD4+ cell population or sub-population, such that both the positive and negative fractions from the CD4-based separation are retained, optionally following one or more further positive or negative selection steps.

In a particular example, a sample of PBMCs or other white blood cell sample is subjected to selection of CD4+ cells, where both the negative and positive fractions are retained. The negative fraction then is subjected to negative selection based on expression of CD14 and CD45RA or RORI, and positive selection based on a marker characteristic of central memory T cells, such as CCR7, CD45RO, and/or CD62L, where the positive and negative selections are carried out in either order.

In particular embodiments, PBMCs are isolated over Lymphoprep (StemCell Technologies, Cat #07851). In particular embodiments CD4+ and/or CD8+ T cells are isolated from PBMCs using negative magnetic selection. In particular embodiments, negative magnetic selection includes using Easy Sep Human CD4+ T cell Isolation Kit II (StemCell Technologies, Cat #17952) and Easy Sep Human CD8+ T cell Isolation Kit II (StemCell Technologies, Cat #17953).

Other cell types can be enriched based on known marker profiles and techniques. For example, CD34+ HSC, HSP, and HSPC can be enriched using anti-CD34 antibodies directly or indirectly conjugated to magnetic particles in connection with a magnetic cell separator, for example, the CliniMACS® Cell Separation System (Miltenyi Biotec, Bergisch Gladbach, Germany).

(iii) Genetically Modifying Cell Populations to Express Chimeric Antigen Receptors (CAR). Cell populations are genetically modified to express chimeric antigen receptors (CAR) described herein.

(iii-a) Genetic Engineering Techniques. Desired genes encoding CAR disclosed herein can be introduced into cells by any method known in the art, including transfection, electroporation, microinjection, lipofection, calcium phosphate mediated transfection, infection with a viral or bacteriophage vector including the gene sequences, cell fusion, chromosome-mediated gene transfer, microcell-mediated gene transfer, spheroplast fusion, in vivo nanoparticle-mediated delivery, etc. Numerous techniques are known in the art for the introduction of foreign genes into cells (see e.g., Loeffler and Behr, 1993, Meth. Enzymol. 217:599-618; Cohen, et al., 1993, Meth. Enzymol. 217:618-644; Cline, 1985, Pharmac. Ther. 29:69-92) and may be used, provided that the necessary developmental and physiological functions of the recipient cells are not unduly disrupted. The technique can provide for the stable transfer of the gene to the cell, so that the gene is expressible by the cell and, in certain instances, preferably heritable and expressible by its cell progeny.

The term “gene” refers to a nucleic acid sequence (used interchangeably with polynucleotide or nucleotide sequence) that encodes a CAR. This definition includes various sequence polymorphisms, mutations, and/or sequence variants wherein such alterations do not substantially affect the function of the encoded CAR. The term “gene” may include not only coding sequences but also regulatory regions such as promoters, enhancers, and termination regions. The term further can include all introns and other DNA sequences spliced from an mRNA transcript, along with variants resulting from alternative splice sites. Gene sequences encoding the molecule can be DNA or RNA that directs the expression of the chimeric molecule. These nucleic acid sequences may be a DNA strand sequence that is transcribed into RNA or an RNA sequence that is translated into protein. The nucleic acid sequences include both the full-length nucleic acid sequences as well as non-full-length sequences derived from the full-length protein. The sequences can also include degenerate codons of the native sequence or sequences that may be introduced to provide codon preference in a specific cell type. Portions of complete gene sequences are referenced throughout the disclosure as is understood by one of ordinary skill in the art.

Gene sequences encoding CAR are provided herein and can also be readily prepared by synthetic or recombinant methods from the relevant amino acid sequences and other description provided herein. In embodiments, the gene sequence encoding any of these sequences can also have one or more restriction enzyme sites at the 5′ and/or 3′ ends of the coding sequence in order to provide for easy excision and replacement of the gene sequence encoding the sequence with another gene sequence encoding a different sequence. In embodiments, the gene sequence encoding the sequences can be codon optimized for expression in mammalian cells.

“Encoding” refers to the property of specific sequences of nucleotides in a gene, such as a cDNA, or an mRNA, to serve as templates for synthesis of other macromolecules such as a defined sequence of amino acids. Thus, a gene codes for a protein if transcription and translation of mRNA corresponding to that gene produces the protein in a cell or other biological system. A “gene sequence encoding a protein” includes all nucleotide sequences that are degenerate versions of each other and that code for the same amino acid sequence or amino acid sequences of substantially similar form and function.

Polynucleotide gene sequences encoding more than one portion of an expressed CAR can be operably linked to each other and relevant regulatory sequences. For example, there can be a functional linkage between a regulatory sequence and an exogenous nucleic acid sequence resulting in expression of the latter. For another example, a first nucleic acid sequence can be operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary or helpful, join coding regions, into the same reading frame.

In any of the embodiments described herein, a polynucleotide can include a polynucleotide that encodes a self-cleaving polypeptide, wherein the polynucleotide encoding the self-cleaving polypeptide is located between the polynucleotide encoding the CAR construct and a polynucleotide encoding a transduction marker (e.g., tCD19 or tEGFR). Exemplary self-cleaving polypeptides include 2A peptide from porcine teschovirus-1 (P2A), Thosea asigna virus (T2A), equine rhinitis A virus (E2A), foot-and-mouth disease virus (F2A), or variants thereof (see FIG. 36). Further exemplary nucleic acid and amino acid sequences of 2A peptides are set forth in, for example, Kim et al. (PLOS One 6:e18556 (2011).

A “vector” is a nucleic acid molecule that is capable of transporting another nucleic acid. Vectors may be, e.g., plasmids, cosmids, viruses, or phage. An “expression vector” is a vector that is capable of directing the expression of a protein encoded by one or more genes carried by the vector when it is present in the appropriate environment.

“Lentivirus” refers to a genus of retroviruses that are capable of infecting dividing and non-dividing cells. Several examples of lentiviruses include HIV (human immunodeficiency virus: including HIV type 1, and HIV type 2); equine infectious anemia virus; feline immunodeficiency virus (FIV); bovine immune deficiency virus (BIV); and simian immunodeficiency virus (SIV).

“Retroviruses” are viruses having an RNA genome. “Gammaretrovirus” refers to a genus of the retroviridae family. Exemplary gammaretroviruses include mouse stem cell virus, murine leukemia virus, feline leukemia virus, feline sarcoma virus, and avian reticuloendotheliosis viruses.

Retroviral vectors (see Miller, et al., 1993, Meth. Enzymol. 217:581-599) can be used. In such embodiments, the gene to be expressed is cloned into the retroviral vector for its delivery into cells. In particular embodiments, a retroviral vector includes all of the cis-acting sequences necessary for the packaging and integration of the viral genome, i.e., (a) a long terminal repeat (LTR), or portions thereof, at each end of the vector; (b) primer binding sites for negative and positive strand DNA synthesis; and (c) a packaging signal, necessary for the incorporation of genomic RNA into virions. More detail about retroviral vectors can be found in Boesen, et al., 1994, Biotherapy 6:291-302; Clowes, et al., 1994, J. Clin. Invest. 93:644-651; Kiem, et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114. Adenoviruses, adeno-associated viruses (AAV) and alphaviruses can also be used. See Kozarsky and Wilson, 1993, Current Opinion in Genetics and Development 3:499-503, Rosenfeld, et al., 1991, Science 252:431-434; Rosenfeld, et al., 1992, Cell 68:143-155; Mastrangeli, et al., 1993, J. Clin. Invest. 91:225-234; Walsh, et al., 1993, Proc. Soc. Exp. Bioi. Med. 204:289-300; and Lundstrom, 1999, J. Recept. Signal Transduct. Res. 19: 673-686. Other methods of gene delivery include use of mammalian artificial chromosomes (Vos, 1998, Curr. Op. Genet. Dev. 8:351-359); liposomes (Tarahovsky and Ivanitsky, 1998, Biochemistry (Mosc) 63:607-618); ribozymes (Branch and Klotman, 1998, Exp. Nephrol. 6:78-83); and triplex DNA (Chan and Glazer, 1997, J. Mol. Med. 75:267-282).

There are a large number of available viral vectors suitable within the current disclosure, including those identified for human gene therapy applications (see Pfeifer and Verma, 2001, Ann. Rev. Genomics Hum. Genet. 2:177). Methods of using retroviral and lentiviral viral vectors and packaging cells for transducing mammalian host cells with viral particles including CAR transgenes are described in, e.g., U.S. Pat. No. 8,119,772; Walchli, et al., 2011, PLoS One 6:327930; Zhao, et al., 2005, J. Immunol. 174:4415; Engels, et al., 2003, Hum. Gene Ther. 14:1155; Frecha, et al., 2010, Mol. Ther. 18:1748; and Verhoeyen, et al., 2009, Methods Mol. Biol. 506:97. Retroviral and lentiviral vector constructs and expression systems are also commercially available.

Targeted genetic engineering approaches may also be utilized. The CRISPR (Clustered Regularly Interspaced Short Palindromic Repeats)/Cas (CRISPR-associated protein) nuclease system is an engineered nuclease system used for genetic engineering that is based on a bacterial system. Information regarding CRISPR-Cas systems and components thereof are described in, for example, U.S. Pat. Nos. 8,697,359, 8,771,945, 8,795,965, 8,865,406, 8,871,445, 8,889,356, 8,889,418, 8,895,308, 8,906,616, 8,932,814, 8,945,839, 8,993,233 and 8,999,641 and applications related thereto; and WO2014/018423, WO2014/093595, WO2014/093622, WO2014/093635, WO2014/093655, WO2014/093661, WO2014/093694, WO2014/093701, WO2014/093709, WO2014/093712, WO2014/093718, WO2014/145599, WO2014/204723, WO2014/204724, WO2014/204725, WO2014/204726, WO2014/204727, WO2014/204728, WO2014/204729, WO2015/065964, WO2015/089351, WO2015/089354, WO2015/089364, WO2015/089419, WO2015/089427, WO2015/089462, WO2015/089465, WO2015/089473 and WO2015/089486, WO2016205711, WO2017/106657, WO2017/127807 and applications related thereto.

Particular embodiments utilize zinc finger nucleases (ZFNs) as gene editing agents. ZFNs are a class of site-specific nucleases engineered to bind and cleave DNA at specific positions. ZFNs are used to introduce double stranded breaks (DSBs) at a specific site in a DNA sequence which enables the ZFNs to target unique sequences within a genome in a variety of different cells. For additional information regarding ZFNs and ZFNs useful within the teachings of the current disclosure, see, e.g., U.S. Pat. Nos. 6,534,261; 6,607,882; 6,746,838; 6,794,136; 6,824,978; 6,866,997; 6,933,113; 6,979,539; 7,013,219; 7,030,215; 7,220,719; 7,241,573; 7,241,574; 7,585,849; 7,595,376; 6,903,185; 6,479,626; US 2003/0232410 and US 2009/0203140 as well as Gaj et al., Nat Methods, 2012, 9(8):805-7; Ramirez et al., Nucl Acids Res, 2012, 40(12):5560-8; Kim et al., Genome Res, 2012, 22(7): 1327-33; Urnov et al., Nature Reviews Genetics, 2010, 11:636-646; Miller, et al. Nature biotechnology 25, 778-785 (2007); Bibikova, et al. Science 300, 764 (2003); Bibikova, et al. Genetics 161, 1169-1175 (2002); Wolfe, et al. Annual review of biophysics and biomolecular structure 29, 183-212 (2000); Kim, et al. Proceedings of the National Academy of Sciences of the United States of America 93, 1156-1160 (1996); and Miller, et al. The EMBO journal 4, 1609-1614 (1985).

Particular embodiments can use transcription activator like effector nucleases (TALENs) as gene editing agents. TALENs refer to fusion proteins including a transcription activator-like effector (TALE) DNA binding protein and a DNA cleavage domain. TALENs are used to edit genes and genomes by inducing double DSBs in the DNA, which induce repair mechanisms in cells. Generally, two TALENs must bind and flank each side of the target DNA site for the DNA cleavage domain to dimerize and induce a DSB. For additional information regarding TALENs, see U.S. Pat. Nos. 8,440,431; 8,440,432; 8,450,471; 8,586,363; and 8,697,853; as well as Joung and Sander, Nat Rev Mol Cell Biol, 2013, 14(I):49-55; Beurdeley et al., Nat Commun, 2013, 4: 1762; Scharenberg et al., Curr Gene Ther, 2013, 13(4):291-303; Gaj et al., Nat Methods, 2012, 9(8):805-7; Miller, et al. Nature biotechnology 29, 143-148 (2011); Christian, et al. Genetics 186, 757-761 (2010); Boch, et al. Science 326, 1509-1512 (2009); and Moscou, & Bogdanove, Science 326, 1501 (2009).

Particular embodiments can utilize MegaTALs as gene editing agents. MegaTALs have a sc rare-cleaving nuclease structure in which a TALE is fused with the DNA cleavage domain of a meganuclease. Meganucleases, also known as homing endonucleases, are single peptide chains that have both DNA recognition and nuclease function in the same domain. In contrast to the TALEN, the megaTAL only requires the delivery of a single peptide chain for functional activity.

Nanoparticles that result in selective in vivo genetic modification of targeted cell types have been described and can be used within the teachings of the current disclosure. In particular embodiments, the nanoparticles can be those described in WO2014153114, WO2017181110, and WO201822672.

In particular embodiments, T cells are transduced with a lentivirus encoding CAR.

(iii-b) CAR Subcomponents. As described previously, CAR molecules include several distinct subcomponents that allow genetically modified cells to recognize and kill unwanted cells, such as cancer cells. The subcomponents include at least an extracellular component and an intracellular component. The extracellular component includes a binding domain that specifically binds a marker that is preferentially present on the surface of unwanted cells. When the binding domain binds such markers, the intracellular component activates the cell to destroy the bound cell. CAR additionally include a transmembrane domain that links the extracellular component to the intracellular component, and other subcomponents that can increase the CAR's function. For example, the inclusion of a spacer region and/or one or more linker sequences can allow the CAR to have additional conformational flexibility, often increasing the binding domain's ability to bind the targeted cell marker.

(iii-b-i) Binding Domains. The current disclosure provides CAR with binding domains that bind FOLR1, MEGF10, HPSE2, KLRF2, PCDH19, or FRAS1.

Binding domains include any substance that binds to a cellular marker to form a complex. The choice of binding domain can depend upon the type and number of cellular markers that define the surface of a target cell. Examples of binding domains include cellular marker ligands, receptor ligands, antibodies, peptides, peptide aptamers, receptors (e.g., T cell receptors), or combinations and engineered fragments or formats thereof.

Antibodies are one example of binding domains and include whole antibodies or binding fragments of an antibody, e.g., Fv, Fab, Fab′, F(ab′)2, and single chain (sc) forms and fragments thereof that bind specifically a cellular marker (such as FOLR1). Antibodies or antigen binding fragments can include all or a portion of polyclonal antibodies, monoclonal antibodies, human antibodies, humanized antibodies, synthetic antibodies, non-human antibodies, recombinant antibodies, chimeric antibodies, bispecific antibodies, mini bodies, and linear antibodies.

Antibodies are produced from two genes, a heavy chain gene and a light chain gene. Generally, an antibody includes two identical copies of a heavy chain, and two identical copies of a light chain. Within a variable heavy chain and variable light chain, segments referred to as complementary determining regions (CDRs) dictate epitope binding. Each heavy chain has three CDRs (i.e., CDRH1, CDRH2, and CDRH3) and each light chain has three CDRs (i.e., CDRL1, CDRL2, and CDRL3). CDR regions are flanked by framework residues (FR). The precise amino acid sequence boundaries of a given CDR or FR can be readily determined using any of a number of well-known schemes, including those described by: Kabat et al. (1991) “Sequences of Proteins of Immunological Interest,” 5th Ed. Public Health Service, National Institutes of Health, Bethesda, Md. (Kabat numbering scheme); Al-Lazikani et al. (1997) J Mol Biol 273: 927-948 (Chothia numbering scheme); Maccallum et al. (1996) J Mol Biol 262: 732-745 (Contact numbering scheme); Martin et al. (1989) Proc. Natl. Acad. Sci., 86: 9268-9272 (AbM numbering scheme); North et al. (2011) J. Mol. Biol. 406(2):228-56 (North numbering scheme); Lefranc M P et al. (2003) Dev Comp Immunol 27(1): 55-77 (IMGT numbering scheme); and Honegger and Pluckthun (2001) J Mol Biol 309(3): 657-670 (“Aho” numbering scheme). The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. In particular embodiments, the antibody CDR sequences disclosed herein are according to Kabat numbering. North numbering uses longer sequences in the structural analysis of the conformations of CDR loops. CDR residues can be identified using software programs such as ABodyBuilder.

The folate receptor 1 (FOLR1) is encoded by the FOLR1 gene. In particular embodiments, the binding domain binds FOLR1. In particular embodiments, the amino acid sequence for human FOLR1 includes the sequence:

(SEQ ID NO: 21)
MAQRMTTQLLLLLVWVAVVGEAQTRIAWARTELLNVCMNAKHHKEKPGP
EDKLHEQCRPWRKNACCSTNTSQEAHKDVSYLYRENWNHCGEMAPACKR
HFIQDTCLYECSPNLGPWIQQVDQSWRKERVLNVPLCKEDCEQWWEDCR
TSYTCKSNWHKGWNWTSGFNKCAVGAACQPFHFYFPTPTVLCNEIWTHS
YKVSNYSRGSGRCIQMWFDPAQGNPNEEVARFYAAAMSGAGPWAAWPFL
LSLALMLLWLLS.

In particular embodiments, the FOLR1-binding domain includes the Farletuzumab scFv. In particular embodiments, the Farletuzumab scFv includes the sequence:

(SEQ ID NO: 22)
DIQLTQSPSSLSASVGDRVTITCSVSSSISSNNLHWYQQKPGKAPKPWI
YGTSNLASGVPSRFSGSGSGTDYTFTISSLQPEDIATYYCQQWSSYPYM
YTFGQGTKVEIKGGGGSGGGGSGGGGSGGGGSEVQLVESGGGVVQPGRS
LRLSCSASGFTFSGYGLSWVRQAPGKGLEWVAMISSGGSYTYYADSVKG
RFAISRDNAKNTLFLQMDSLRPEDTGVYFCARHGDDPAWFAYWGQGTPV
TVSS.

In particular embodiments, the FOLR1-binding domain includes the Farletuzumab scFv. In particular embodiments, the Farletuzumab scFv includes the sequence:

(SEQ ID NO: 23)
EVQLVESGGGVVQPGRSLRLSCSASGFTFSGYGLSWVRQAPGKGLEWVA
MISSGGSYTYYADSVKGRFAISRDNAKNTLFLQMDSLRPEDTGVYFCAR
HGDDPAWFAYWGQGTPVTVSSGGGGSGGGGSGGGGSGGGGSDIQLTQSP
SSLSASVGDRVTITCSVSSSISSNNLHWYQQKPGKAPKPWIYGTSNLAS
GVPSRFSGSGSGTDYTFTISSLQPEDIATYYCQQWSSYPYMYTFGQGTK
VEIK.

In particular embodiments, the FOLR1-binding domain includes the Farletuzumab antibody (MorAb-003). In particular embodiments, the FOLR1-binding domain is a human or humanized binding domain including a variable heavy chain including a CDRH1 sequence including GYGLS (SEQ ID NO: 24), a CDRH2 sequence including MISSGGSYTYYADSVKG (SEQ ID NO: 25), and a CDRH3 sequence including HGDDPAWFAY (SEQ ID NO: 26), and a variable light chain including a CDRL1 sequence including SVSSSISSNNLH (SEQ ID NO: 27), a CDRL2 sequence including GTSNLAS (SEQ ID NO: 28), and a CDRL3 sequence including QQWSSYPYMYT (SEQ ID NO: 29), according to Kabat numbering scheme.

In particular embodiments, the FOLR1-binding domain includes the Farletuzumab antibody. In particular embodiments, a sequence that binds human FOLR1 includes a heavy chain region including sequence: EVQLVESGGGVVQPGRSLRLSCSASGFTFSGYGLSWVRQAPGKGLEWVAMISSGGSYTYYA DSVKGRFAISRDNAKNTLFLQMDSLRPEDTGVYFCARHGDDPAWFAYWGQGTPVTVSS (SEQ ID NO: 30), and a light chain region including sequence:

(SEQ ID NO: 31)
DIQLTQSPSSLSASVGDRVTITCSVSSSISSNNLHWYQQKPGKAPKPWI
YGTSNLASGVPSRFSGSGSGTDYTFTISSLQPEDIATYYCQQWSSYPYM
YTFGQGTKVEIK.

In particular embodiments, the FOLR1-binding domain includes a variable heavy chain region encoded by the sequence: GAGGTACAGCTTGTCGAGAGCGGTGGTGGAGTAGTCCAACCGGGTCGAAGTCTTAGGCTT TCCTGTAGCGCATCTGGGTTCACTTTTAGTGGCTACGGCCTCTCCTGGGTGAGACAGGCG CCTGGGAAGGGGCTGGAGTGGGTAGCCATGATTTCATCTGGTGGCTCATATACTTATTATG CCGACTCCGTAAAGGGAAGATTCGCAATATCACGCGATAACGCTAAAAATACACTCTTCTT GCAGATGGATTCTTTGAGACCTGAGGATACCGGGGTTTACTTTTGCGCCAGACACGGGGA TGACCCCGCCTGGTTTGCCTATTGGGGACAGGGAACCCCTGTGACGGTATCCTCT (SEQ ID NO: 138), and a variable light chain region encoded by the sequence:

(SEQ ID NO: 139)
GATATTCAGCTTACTCAAAGTCCGAGTAGTCTGTCTGCCTCAGTTGGCG
ATAGGGTGACCATCACTTGCTCCGTAAGTAGTTCTATTTCTTCCAACAA
CCTGCATTGGTATCAACAGAAACCAGGTAAAGCACCTAAGCCGTGGATC
TACGGAACGTCCAACCTTGCGTCTGGCGTACCAAGCCGGTTCTCCGGGA
GTGGGAGTGGTACAGATTACACATTTACTATCAGTTCTCTTCAACCGGA
AGACATTGCCACATATTATTGCCAGCAATGGTCATCTTACCCCTATATG
TACACATTTGGTCAGGGTACAAAGGTTGAAATAAAA

In particular embodiments, the FOLR1-binding domain includes the huMOV19 (M9346A) antibody. In particular embodiments, the FOLR1-binding domain is a human or humanized binding domain including a variable heavy chain including a CDRH1 sequence including GYFMN (SEQ ID NO: 32), a CDRH2 sequence including RIHPYDGDTFYNQKFQG (SEQ ID NO: 33), and a CDRH3 sequence including YDGSRAMDY (SEQ ID NO: 34), and a variable light chain including a CDRL1 sequence including KASQSVSFAGTSLMH (SEQ ID NO: 35), a CDRL2 sequence including RASNLEA (SEQ ID NO: 36), and a CDRL3 sequence including QQSREYPYT (SEQ ID NO: 37), according to Kabat numbering scheme.

In particular embodiments, the FOLR1-binding domain includes the huMOV19 version 1.00. In particular embodiments, a sequence that binds human FOLR1 includes a variable heavy chain region including sequence: QVQLVQSGAEVVKPGASVKISCKASGYTFTGYFMNWVKQSPGQSLEWIGRIHPYDGDTFYNQ KFQGKATLTVDKSSNTAHMELLSLTSEDFAVYYCTRYDGSRAMDYWGQGTTVTVSS (SEQ ID NO: 38), and a variable light chain region including sequence:

(SEQ ID NO: 39)
DIVLTQSPLSLAVSLGQPAIISCKASQSVSFAGTSLMHWYHQKPGQQPR
LLIYRASNLEAGVPDRFSGSGSKTDFTLNISPVEAEDAATYYCQQSREY
PYTFGGGTKLEIKR.

In particular embodiments, the FOLR1-binding domain includes the huMOV19 version 1.60. In particular embodiments, a sequence that binds human FOLR1 includes a variable heavy chain region including sequence: QVQLVQSGAEVVKPGASVKISCKASGYTFTGYFMNWVKQSPGQSLEWIGRIHPYDGDTFYNQ KFQGKATLTVDKSSNTAHMELLSLTSEDFAVYYCTRYDGSRAMDYWGQGTTVTVSS (SEQ ID NO: 40), and a variable light chain region including sequence:

(SEQ ID NO: 41)
DIVLTQSPLSLAVSLGQPAIISCKASQSVSFAGTSLMHWYHQKPGQQPR
LLIYRASNLEAGVPDRFSGSGSKTDFTLTISPVEAEDAATYYCQQSREY
PYTFGGGTKLEIKR.

In particular embodiments, the FOLR1-binding domain includes the RA15-7 antibody. In particular embodiments, the FOLR1-binding domain is a human or humanized binding domain including a variable heavy chain including a CDRH1 sequence including DFYMN (SEQ ID NO: 42), a CDRH2 sequence including FIRNKANGYTTEFNPSVKG (SEQ ID NO: 43), and a CDRH3 sequence including TLYGYAYYYVMDA (SEQ ID NO: 44), and a variable light chain including a CDRL1 sequence including RTSEDIFRNLA (SEQ ID NO: 45), a CDRL2 sequence including DTNRLAD (SEQ ID NO: 46), and a CDRL3 sequence including QQYDNYPLT (SEQ ID NO: 47), according to Kabat numbering scheme.

In particular embodiments, the FOLR1-binding domain includes the RA15-7 antibody. In particular embodiments, a sequence that binds human FOLR1 includes a variable heavy chain region including sequence: EVQLVESGGGLVQPGGSLRLSCAASGFTFTDFYMNWVRQPPGKAPEWLGFIRNKANGYTTEF NPSVKGRFTISRDNSKNSLYLQMNSLKTEDTATYYCARTLYGYAYYYVMDAWGQGTLVTVSS (SEQ ID NO: 48), and a variable light chain region including sequence:

(SEQ ID NO: 49)
DIQMTQSPSSLSASLGDRVTITCRTSEDIFRNLAWYQQKPGKAPKLLIY
DTNRLADGVPSRFSGSGSGTDYTLTISSLQPEDFATYFCQQYDNYPLTF
GQGTKLEIK

In particular embodiments, the FOLR1-binding domain includes the huFR1-48. In particular embodiments, the FOLR1-binding domain is a human or humanized binding domain including a variable heavy chain including a CDRH1 sequence including NYWMQ (SEQ ID NO: 50), a CDRH2 sequence including AIYPGNGDSRYTQKFQG (SEQ ID NO: 51), and a CDRH3 sequence including RDGNYAAY (SEQ ID NO: 52), and a variable light chain including a CDRL1 sequence including RASENIYSNLA (SEQ ID NO: 53), a CDRL2 sequence including AATNLAD (SEQ ID NO: 54), and a CDRL3 sequence including QHFWASPYT (SEQ ID NO: 55), according to Kabat numbering scheme.

In particular embodiments, the FOLR1-binding domain includes the huFR1-48. In particular embodiments, a sequence that binds human FOLR1 includes a variable heavy chain region including sequence: QVQLVQSGAEVAKPGASVKLSCKASGYTFTNYWMQWIKQRPGQGLEWIGAIYPGNGDSRYT QKFQGKATLTADKSSSTAYMQVSSLTSEDSAVYYCARRDGNYAAYWGQGTLVTVSA (SEQ ID NO: 56), and a variable light chain region including sequence:

(SEQ ID NO: 57)
DIQMTQSPSSLSVSVGERVTITCRASENIYSNLAWYQQKPGKSPKLLVY
AATNLADGVPSRFSGSESGTDYSLKINSLQPEDFGSYYCQHFWASPYTF
GQGTKLEIKR.

In particular embodiments, the FOLR1-binding domain includes the huFR1-49. In particular embodiments, the FOLR1-binding domain is a human or humanized binding domain including a variable heavy chain including a CDRH1 sequence including NYWMY (SEQ ID NO: 58), a CDRH2 sequence including AIYPGNSDTTYNQKFQG (SEQ ID NO: 59), and a CDRH3 sequence including RHDYGAMDY (SEQ ID NO: 60), and a variable light chain including a CDRL1 sequence including RASENIYTNLA (SEQ ID NO: 61), a CDRL2 sequence including TASNLAD (SEQ ID NO: 62), and a CDRL3 sequence including QHFWVSPYT (SEQ ID NO: 63), according to Kabat numbering scheme.

In particular embodiments, the FOLR1-binding domain includes the huFR1-49. In particular embodiments, a sequence that binds human FOLR1 includes a variable heavy chain region including sequence: QVQLQQSGAVVAKPGASVKMSCKASGYTFTNYWMYWIKQRPGQGLELIGAIYPGNSDTTYNQ KFQGKATLTAVTSANTVYMEVSSLTSEDSAVYYCTKRHDYGAMDYWGQGTSVTVSS (SEQ ID NO: 64), and a variable light chain region including sequence:

(SEQ ID NO: 64)
DIQMTQSPSSLSVSVGERVTITCRASENIYTNLAWYQQKPGKSPKLLVY
TASNLADGVPSRFSGSGSGTDYSLKINSLQPEDFGTYYCQHFWVSPYTF
GQGTKLEIKR.

In particular embodiments, the FOLR1-binding domain includes the huFR1-57. In particular embodiments, the FOLR1-binding domain is a human or humanized binding domain including a variable heavy chain including a CDRH1 sequence including SFGMH (SEQ ID NO: 66), a CDRH2 sequence including YISSGSSTISYADSVKG (SEQ ID NO: 67), and a CDRH3 sequence including EAYGSSMEY (SEQ ID NO: 68), and a variable light chain including a CDRL1 sequence including RASQNINNNLH (SEQ ID NO: 69), a CDRL2 sequence including YVSQSVS (SEQ ID NO: 70), and a CDRL3 sequence including QQSNSWPHYT (SEQ ID NO: 71), according to Kabat numbering scheme.

In particular embodiments, the FOLR1-binding domain includes the huFR1-57. In particular embodiments, a sequence that binds human FOLR1 includes a variable heavy chain region including sequence: EVQLVESGGGLVQPGGSRRLSCAASGFTFSSFGMHWVRQAPGKGLEWVAYISSGSSTISYAD SVKGRFTISRDNSKKTLLLQMTSLRAEDTAMYYCAREAYGSSMEYWGQGTLVTVSS (SEQ ID NO: 72), and a variable light chain region including sequence:

(SEQ ID NO: 73)
EIVLTQSPATLSVTPGDRVSLSCRASQNINNNLHWYQQKPGQSPRLLIK
YVSQSVSGIPDRFSGSGSGTDFTLSISSVEPEDFGMYFCQQSNSWPHYT
FGQGTKLEIKR.

In particular embodiments, the FOLR1-binding domain includes the huFR1-65. In particular embodiments, the FOLR1-binding domain is a human or humanized binding domain including a variable heavy chain including a CDRH1 sequence including SYTMH (SEQ ID NO: 74), a CDRH2 sequence including YINPISGYTNYNQKFQG (SEQ ID NO: 75), and a CDRH3 sequence including GGAYGRKPMDY (SEQ ID NO: 76), and a variable light chain including a CDRL1 sequence including KASQNVGPNVA (SEQ ID NO: 77), a CDRL2 sequence including SASYRYS (SEQ ID NO: 78), and a CDRL3 sequence including QQYNSYPYT (SEQ ID NO: 79), according to Kabat numbering scheme.

In particular embodiments, the FOLR1-binding domain includes the huFR1-65. In particular embodiments, a sequence that binds human FOLR1 includes a variable heavy chain region including sequence: QVQLVQSGAEVAKPGASVKMSCKASGYTFTSYTMHWVKQRPGQGLAWIGYINPISGYTNYNQ KFQGKATLTADKSSSTAYMQLNSLTSEDSAVYYCASGGAYGRKPMDYWGQGTSVTVSS (SEQ ID NO: 80), and a variable light chain region including sequence:

(SEQ ID NO: 81)
EIVMTQSPATMSTSPGDRVSVTCKASQNVGPNVAWYQQKPGQSPRALIY
SASYRYSGVPARFTGSGSGTDFTLTISNMQSEDLAEYFCQQYNSYPYTF
GQGTKLEIKR.

In particular embodiments, the FOLR1-binding domain includes a sequence having at least 90% sequence identity to SEQ ID NOs: 22-81. In particular embodiments, the FOLR1-binding domain includes a sequence having at least 95%, at least 96%, at least 97%, at least 98%, or at least 99% sequence identity to SEQ ID NOs: 22-81. In certain embodiments, the FOLR1-binding domain is an antibody and/or the polypeptide that specifically binds FOLR1.

The multiple EGF like domain 10 (MEGF10) protein is encoded by the MEGF10 gene. In particular embodiments, the binding domain binds MEGF10. In particular embodiments, the amino acid sequence for human MEGF10 includes the sequence:

(SEQ ID NO: 82)
MVISLNSCLSFICLLLCHWIGTASPLNLEDPNVCSHWESYSVTVQESYPHPFDQIYYTSCTDILN
WFKCTRHRVSYRTAYRHGEKTMYRRKSQCCPGFYESGEMCVPHCADKCVHGRCIAPNTCQC
EPGWGGTNCSSACDGDHWGPHCTSRCQCKNGALCNPITGACHCAAGFRGWRCEDRCEQG
TYGNDCHQRCQCQNGATCDHVTGECRCPPGYTGAFCEDLCPPGKHGPQCEQRCPCQNGG
VCHHVTGECSCPSGWMGTVCGQPCPEGRFGKNCSQECQCHNGGTCDAATGQCHCSPGYT
GERCQDECPVGTYGVLCAETCQCVNGGKCYHVSGACLCEAGFAGERCEARLCPEGLYGIKC
DKRCPCHLENTHSCHPMSGECACKPGWSGLYCNETCSPGFYGEACQQICSCQNGADCDSVT
GKCTCAPGFKGIDCSTPCPLGTYGINCSSRCGCKNDAVCSPVDGSCTCKAGWHGVDCSIRCP
SGTWGFGCNLTCQCLNGGACNTLDGTCTCAPGWRGEKCELPCQDGTYGLNCAERCDCSHA
DGCHPTTGHCRCLPGWSGVHCDSVCAEGRWGPNCSLPCYCKNGASCSPDDGICECAPGFR
GTTCQRICSPGFYGHRCSQTCPQCVHSSGPCHHITGLCDCLPGFTGALCNEVCPSGRFGKNC
AGICTCTNNGTCNPIDRSCQCYPGWIGSDCSQPCPPAHWGPNCIHTCNCHNGAFCSAYDGEC
KCTPGWTGLYCTQRCPLGFYGKDCALICQCQNGADCDHISGQCTCRTGFMGRHCEQKCPSG
TYGYGCRQICDCLNNSTCDHITGTCYCSPGWKGARCDQAGVIIVGNLNSLSRTSTALPADSYQI
GAIAGIIILVLVVLFLLALFIIYRHKQKGKESSMPAVTYTPAMRVVNADYTISGTLPHSNGGNANSH
YFTNPSYHTLTQCATSPHVNNRDRMTVTKSKNNQLFVNLKNVNPGKRGPVGDCTGTLPADWK
HGGYLNELGAFGLDRSYMGKSLKDLGKNSEYNSSNCSLSSSENPYATIKDPPVLIPKSSECGY
VEMKSPARRDSPYAEINNSTSANRNVYEVEPTVSVVQGVFSNNGRLSQDPYDLPKNSHIPCHY
DLLPVRDSSSSPKQEDSGGSSSNSSSSSE.

In particular embodiments, binding domains that bind MEGF10 include the LS-C678634, LS-C668447, LSC497216, or PA5-76556 antibodies or binding fragments thereof.

The heparinase-2 (HPSE2) enzyme is encoded by the HPSE2 gene. In particular embodiments, the binding domain binds HPSE2. In particular embodiments, the amino acid sequence for human HPSE2 includes the sequence:

(SEQ ID NO: 83)
MRVLCAFPEAMPSSNSRPPACLAPGALYLALLLHLSLSSQAGDRRPLPVDRAAGLKEKTLILLD
VSTKNPVRTVNENFLSLQLDPSIIHDGWLDFLSSKRLVTLARGLSPAFLRFGGKRTDFLQFQNL
RNPAKSRGGPGPDYYLKNYEDDIVRSDVALDKQKGCKIAQHPDVMLELQREKAAQMHLVLLKE
QFSNTYSNLILTARSLDKLYNFADCSGLHLIFALNALRRNPNNSWNSSSALSLLKYSASKKYNIS
WELGNEPNNYRTMHGRAVNGSQLGKDYIQLKSLLQPIRIYSRASLYGPNIGRPRKNVIALLDGF
MKVAGSTVDAVTWQHCYIDGRVVKVMDFLKTRLLDTLSDQIRKIQKVVNTYTPGKKIWLEGVVT
TSAGGTNNLSDSYAAGFLWLNTLGMLANQGIDVVIRHSFFDHGYNHLVDQNFNPLPDYWLSLL
YKRLIGPKVLAVHVAGLQRKPRPGRVIRDKLRIYAHCTNHHNHNYVRGSITLFIINLHRSRKKIKL
TGTLRDKLVHQYLLQPYGQEGLKSKSVQLNGQPLVMVDDGTLPELKPRPLRAGRTLVIPPVTM
GFFVVKNVNALACRYR.

In particular embodiments, binding domains that bind HPSE2 include the LS-B14593, LS-C322089, LS-C378319, or HPA044603 antibodies or binding fragments thereof.

The killer cell lectin like receptor F2 (KLRF2) protein is encoded by the KLRF2 gene. In particular embodiments, the binding domain binds KLRF2. In particular embodiments, the amino acid sequence for human KLRF2 includes the sequence:

(SEQ ID NO: 84)
MENEDGYMTLSFKNRCKSKQKSKDFSLYPQYYCLLLIFGCIVILIFIMT
GIDLKFWHKKMDFSQNVNVSSLSGHNYLCPNDWLLNEGKCYWFSTSFKT
WKESQRDCTQLQAHLLVIQNLDELEFIQNSLKPGHFGWIGLYVTFQGNL
WMWIDEHFLVPELFSVIGPTDDRSCAVITGNWVYSEDCSSTFKGICQRD
AILTHNGTSGV.

In particular embodiments, binding domains that bind KLRF2 include the LS-C329740, LS-C203747, SAB2108513, SAB2108684, HPA055964, SAB2108320, or SAB2108355 antibodies or binding fragments thereof.

The protocadherin-19 (PCDH19) protein is encoded by the PCDH19 gene. In particular embodiments, the binding domain binds PCDH19. In particular embodiments, the amino acid sequence for human PCDH19 includes the sequence:

(SEQ ID NO: 85)
MESLLLPVLLLLAILWTQAAALINLKYSVEEEQRAGTVIANVAKDAREAGFALDPRQASAFRVVS
NSAPHLVDINPSSGLLVTKQKIDRDLLCRQSPKCIISLEVMSSSMEICVIKVEIKDLNDNAPSFPA
AQIELEISEAASPGTRIPLDSAYDPDSGSFGVQTYELTPNELFGLEIKTRGDGSRFAELVVEKSL
DRETQSHYSFRITALDGGDPPRLGTVGLSIKVTDSNDNNPVFSESTYAVSVPENSPPNTPVIRL
NASDPDEGTNGQVVYSFYGYVNDRTRELFQIDPHSGLVTVTGALDYEEGHVYELDVQAKDLG
PNSIPAHCKVTVSVLDTNDNPPVINLLSVNSELVEVSESAPPGYVIALVRVSDRDSGLNGRVQC
RLLGNVPFRLQEYESFSTILVDGRLDREQHDQYNLTIQARDGGVPMLQSAKSFTVLITDENDNH
PHFSKPYYQVIVQENNTPGAYLLSVSARDPDLGLNGSVSYQIVPSQVRDMPVFTYVSINPNSG
DIYALRSFNHEQTKAFEFKVLAKDGGLPSLQSNATVRVIILDVNDNTPVITAPPLINGTAEVYIPR
NSGIGYLVTVVKAEDYDEGENGRVTYDMTEGDRGFFEIDQVNGEVRTTRTFGESSKSSYELIV
VAHDHGKTSLSASALVLIYLSPALDAQESMGSVNLSLIFIIALGSIAGILFVTMIFVAIKCKRDNKEI
RTYNCSNCLTITCLLGCFIKGQNSKCLHCISVSPISEEQDKKTEEKVSLRGKRIAEYSYGHQKKS
SKKKKISKNDIRLVPRDVEETDKMNVVSCSSLTSSLNYFDYHQQTLPLGCRRSESTFLNVENQN
TRNTSANHIYHHSFNSQGPQQPDLIINGVPLPETENYSFDSNYVNSRAHLIKSSSTFKDLEGNSL
KDSGHEESDQTDSEHDVQRSLYCDTAVNDVLNTSVTSMGSQMPDHDQNEGFHCREECRILG
HSDRCWMPRNPMPIRSKSPEHVRNIIALSIEATAADVEAYDDCGPTKRTFATFGKDVSDHPAEE
RPTLKGKRTVDVTICSPKVNSVIREAGNGCEAISPVTSPLHLKSSLPTKPSVSYTIALAPPARDLE
QYVNNVNNGPTRPSEAEPRGADSEKVMHEVSPILKEGRNKESPGVKRLKDIVL.

In particular embodiments, binding domains that bind PCDH19 include the LS-C676224, LS-C496779, LS-C761991, HPA027533, an HPA001461 antibodies or binding fragments thereof.

The Fraser extracellular matrix complex subunit 1 (FRAS1) protein is encoded by the FRAS1 gene. In particular embodiments, the binding domain binds FRAS1. In particular embodiments, the amino acid sequence for human FRAS1 includes the sequence:

(SEQ ID NO: 86)
MGVLKVWLGLALALAEFAVLPHHSEGACVYQGSLLADATIWKPDSCQSC
RCHGDIVICKPAVCRNPQCAFEKGEVLQIAANQCCPECVLRTPGSCHHE
KKIHEHGTEWASSPCSVCSCNHGEVRCTPQPCPPLSCGHQELAFIPEGS
CCPVCVGLGKPCSYEGHVFQDGEDWRLSRCAKCLCRNGVAQCFTAQCQP
LFCNQDETVVRVPGKCCPQCSARSCSAAGQVYEHGEQWSENACTTCICD
RGEVRCHKQACLPLRCGKGQSRARRHGQCCEECVSPALASQSVGIAGMS
HHAQSLLGPFLTQIKKPHFSCLE.

In particular embodiments, binding domains that bind FRAS1 include the LS-C763132, LS-B5486, LS-C754337, HPA011281, or HPA051601 antibodies or binding fragments thereof.

In some instances, additional scFvs based on the binding domains described herein and for use in a CAR can be prepared according to methods known in the art (see, for example, Bird et al., (1988) Science 242:423-426 and Huston et al., (1988) Proc. Natl. Acad. Sci. USA 85:5879-5883). ScFv molecules can be produced by linking VH and VL regions of an antibody together using flexible polypeptide linkers. If a short polypeptide linker is employed (e.g., between 5-10 amino acids) intrachain folding is prevented. Interchain folding is also required to bring the two variable regions together to form a functional epitope binding site. For examples of linker orientations and sizes see, e.g., Hollinger et al. 1993 Proc Natl Acad. Sci. U.S.A. 90:6444-6448, US 2005/0100543, US 2005/0175606, US 2007/0014794, and WO2006/020258 and WO2007/024715. More particularly, linker sequences that are used to connect the VL and VH of an scFv are generally five to 35 amino acids in length. In particular embodiments, a VL-VH linker includes from five to 35, ten to 30 amino acids or from 15 to 25 amino acids. Variation in the linker length may retain or enhance activity, giving rise to superior efficacy in activity studies. scFv are commonly used as the binding domains of CAR.

Other binding fragments, such as Fv, Fab, Fab′, F(ab′)2, can also be used within the CAR disclosed herein. Additional examples of antibody-based binding domain formats for use in a CAR include scFv-based grababodies and soluble VH domain antibodies. These antibodies form binding regions using only heavy chain variable regions. See, for example, Jespers et al., Nat. Biotechnol. 22:1161, 2004; Cortez-Retamozo et al., Cancer Res. 64:2853, 2004; Baral et al., Nature Med. 12:580, 2006; and Barthelemy et al., J. Biol. Chem. 283:3639, 2008.

In particular embodiments, the binding domain includes a humanized antibody or an engineered fragment thereof. In particular embodiments, a non-human antibody is humanized, where one or more amino acid residues of the antibody are modified to increase similarity to an antibody naturally produced in a human or fragment thereof. These nonhuman amino acid residues are often referred to as “import” residues, which are typically taken from an “import” variable domain. As provided herein, humanized antibodies or antibody fragments include one or more CDRs from nonhuman immunoglobulin molecules and framework regions wherein the amino acid residues including the framework are derived completely or mostly from human germline. A humanized antibody can be produced using a variety of techniques known in the art, including CDR-grafting (see, e.g., European Patent No. EP 239,400; WO 91/09967; and U.S. Pat. Nos. 5,225,539, 5,530,101, and 5,585,089), veneering or resurfacing (see, e.g., EP 592,106 and EP 519,596; Padlan, 1991, Molecular Immunology, 28(4/5):489-498; Studnicka et al., 1994, Protein Engineering, 7(6):805-814; and Roguska et al., 1994, PNAS, 91:969-973), chain shuffling (see, e.g., U.S. Pat. No. 5,565,332), and techniques disclosed in, e.g., US 2005/0042664, US 2005/0048617, U.S. Pat. Nos. 6,407,213, 5,766,886, WO 9317105, Tan et al., J. Immunol., 169:1119-25 (2002), Caldas et al., Protein Eng., 13(5):353-60 (2000), Morea et al., Methods, 20(3):267-79 (2000), Baca et al., J. Biol. Chem., 272(16): 10678-84 (1997), Roguska et al., Protein Eng., 9(10):895-904 (1996), Couto et al., Cancer Res., 55 (23 Supp):5973s-5977s (1995), Couto et al., Cancer Res., 55(8):1717-22 (1995), Sandhu J S, Gene, 150(2):409-10 (1994), and Pedersen et al., J. Mol. Biol., 235(3):959-73 (1994). Often, framework residues in the framework regions will be substituted with the corresponding residue from the CDR donor antibody to alter, for example improve, cellular marker binding. These framework substitutions are identified by methods well-known in the art, e.g., by modeling of the interactions of the CDR and framework residues to identify framework residues important for cellular marker binding and sequence comparison to identify unusual framework residues at particular positions. (See, e.g., U.S. Pat. No. 5,585,089; and Riechmann et al., 1988, Nature, 332:323).

Functional variants include one or more residue additions or substitutions that do not substantially impact the physiological effects of the protein. Functional fragments include one or more deletions or truncations that do not substantially impact the physiological effects of the protein. A lack of substantial impact can be confirmed by observing experimentally comparable results in an activation study or a binding study. Functional variants and functional fragments of intracellular domains (e.g., intracellular signaling domains) transmit activation or inhibition signals comparable to a wild-type reference when in the activated state of the current disclosure. Functional variants and functional fragments of binding domains bind their cognate antigen or ligand at a level comparable to a wild-type reference.

In particular embodiments, a VL region in a binding domain of the present disclosure is derived from or based on a VL of an antibody disclosed herein and contains one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions), or a combination of the above-noted changes, when compared with the VL of the antibody disclosed herein. An insertion, deletion or substitution may be anywhere in the VL region, including at the amino- or carboxy-terminus or both ends of this region, provided that each CDR includes zero changes or at most one, two, or three changes and provided a binding domain containing the modified VL region can still specifically bind its target with an affinity similar to the wild type binding domain.

In particular embodiments, a binding domain VH region of the present disclosure can be derived from or based on a VH of an antibody disclosed herein and can contain one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions or non-conservative amino acid substitutions), or a combination of the above-noted changes, when compared with the VH of the antibody disclosed herein. An insertion, deletion or substitution may be anywhere in the VH region, including at the amino- or carboxy-terminus or both ends of this region, provided that each CDR includes zero changes or at most one, two, or three changes and provided a binding domain containing the modified VH region can still specifically bind its target with an affinity similar to the wild type binding domain.

In particular embodiments, a binding domain includes or is a sequence that is at least 90%, at least 91%, at least 92%, at least 93%, at least 94%, at least 95%, at least 96%, at least 97%, at least 98%, at least 99%, at least 99.5%, or 100% identical to an amino acid sequence of a light chain variable region (VL) or to a heavy chain variable region (VH), or both, wherein each CDR includes zero changes or at most one, two, or three changes, from an antibody disclosed herein or fragment or derivative thereof that specifically binds to a cellular marker of interest.

(iii-b-ii) Spacer Regions. Spacer regions are used to create appropriate distances and/or flexibility from other CAR sub-components. In particular embodiments, the length of a spacer region is customized for binding targeted cells and mediating destruction. In particular embodiments, a spacer region length can be selected based upon the location of a cellular marker epitope, affinity of a binding domain for the epitope, and/or the ability of the targeting agent to mediate cell destruction following target binding.

Spacer regions typically include those having 10 to 250 amino acids, 10 to 200 amino acids, 10 to 150 amino acids, 10 to 100 amino acids, 10 to 50 amino acids, or 10 to 25 amino acids.

In particular embodiments, a spacer region is 5 amino acids, 8 amino acids, 10 amino acids, 12 amino acids, 14 amino acids, 20 amino acids, 21 amino acids, 26 amino acids, 27 amino acids, 45 amino acids, or 50 amino acids. These lengths qualify as short spacer regions.

In particular embodiments, a spacer region is 100 amino acids, 110 amino acids, 120 amino acids, 125 amino acids, 128 amino acids, 131 amino acids, 135 amino acids, 140 amino acids, 150 amino acids, 160 amino acids, or 170 amino acids. These lengths qualify as intermediate spacer regions.

In particular embodiments, a spacer region is 180 amino acids, 190 amino acids, 200 amino acids, 210 amino acids, 212 amino acids, 214 amino acids, 216 amino acids, 218 amino acids, 220 amino acids, 230 amino acids, 240 amino acids, or 250 amino acids. These lengths qualify as long spacer regions.

Exemplary spacer regions include all or a portion of an immunoglobulin hinge region. An immunoglobulin hinge region may be a wild-type immunoglobulin hinge region or an altered wild-type immunoglobulin hinge region. In certain embodiments, an immunoglobulin hinge region is a human immunoglobulin hinge region. As used herein, a “wild type immunoglobulin hinge region” refers to a naturally occurring upper and middle hinge amino acid sequences interposed between and connecting the CH1 and CH2 domains (for IgG, IgA, and IgD) or interposed between and connecting the CH1 and CH3 domains (for IgE and IgM) found in the heavy chain of an antibody.

An immunoglobulin hinge region may be an IgG, IgA, IgD, IgE, or IgM hinge region. An IgG hinge region may be an IgG1, IgG2, IgG3, or IgG4 hinge region. Sequences from IgG1, IgG2, IgG3, IgG4 or IgD can be used alone or in combination with all or a portion of a CH2 region; all or a portion of a CH3 region; or all or a portion of a CH2 region and all or a portion of a CH3 region.

In particular embodiments, the spacer is a short spacer including an IgG4 hinge region. In particular embodiments the short spacer is encoded by either of SEQ ID NOs: 1 or 2. In particular embodiments, the spacer is an IgG4 hinge S10P. In particular embodiments, the IgG4 hinge S10P is encoded by SEQ ID NO: 135. In particular embodiments, the spacer is an intermediate spacer including an IgG4 hinge region and an IgG4 hinge CH3 region. In particular embodiments the intermediate spacer is encoded by SEQ ID NO: 3. In particular embodiments, the spacer is a hinge and intermediate spacer (DS). In particular embodiments, the hinge and intermediate spacer (DS) is encoded by SEQ ID NO: 136. In particular embodiments, the spacer is a long spacer including an IgG4 hinge region, an IgG4 CH3 region, and an IgG4 CH2 region. In particular embodiments the long spacer is encoded by SEQ ID NO: 4.

Other examples of hinge regions that can be used in CAR described herein include the hinge region present in the extracellular regions of type 1 membrane proteins, such as CD8a, CD4, CD28 and CD7, which may be wild-type or variants thereof.

In particular embodiments, a spacer region includes a hinge region that includes a type II C-lectin interdomain (stalk) region or a cluster of differentiation (CD) molecule stalk region. A “stalk region” of a type II C-lectin or CD molecule refers to the portion of the extracellular domain (ECD) of the type II C-lectin or CD molecule that is located between the C-type lectin-like domain (CTLD; e.g., similar to CTLD of natural killer cell receptors) and the hydrophobic portion (transmembrane domain). For example, the ECD of human CD94 (GenBank Accession No. AAC50291.1) corresponds to amino acid residues 34-179, but the CTLD corresponds to amino acid residues 61-176, so the stalk region of the human CD94 molecule includes amino acid residues 34-60, which are located between the hydrophobic portion (transmembrane domain) and CTLD (see Boyington et al., Immunity 10:15, 1999; for descriptions of other stalk regions, see also Beavil et al., Proc. Nat'l. Acad. Sci. USA 89:153, 1992; and Figdor et al., Nat. Rev. Immunol. 2:11, 2002). These type II C-lectin or CD molecules may also have junction amino acids (described below) between the stalk region and the transmembrane region or the CTLD. In another example, the 233 amino acid human NKG2A protein (GenBank Accession No. P26715.1) has a hydrophobic portion (transmembrane domain) ranging from amino acids 71-93 and an ECD ranging from amino acids 94-233. The CTLD includes amino acids 119-231 and the stalk region includes amino acids 99-116, which may be flanked by additional junction amino acids. Other type II C-lectin or CD molecules, as well as their extracellular ligand-binding domains, stalk regions, and CTLDs are known in the art (see, e.g., GenBank Accession Nos. NP 001993.2; AAH07037.1; NP 001773.1; AAL65234.1; CAA04925.1; for the sequences of human CD23, CD69, CD72, NKG2A, and NKG2D and their descriptions, respectively).

(iii-b-iii) Transmembrane Domains. As indicated, transmembrane domains within a CAR serve to connect the extracellular component and intracellular component through the cell membrane. The transmembrane domain can anchor the expressed molecule in the modified cell's membrane.

The transmembrane domain can be derived either from a natural and/or a synthetic source. When the source is natural, the transmembrane domain can be derived from any membrane-bound or transmembrane protein. Transmembrane domains can include at least the transmembrane region(s) of the α, β or ζ chain of a T-cell receptor, CD28, CD27, CD3 epsilon, CD45, CD4, CD5, CD8, CD9, CD16, CD22; CD33, CD37, CD64, CD80, CD86, CD134, CD137 and CD154. In particular embodiments, a transmembrane domain may include at least the transmembrane region(s) of, e.g., KIRDS2, OX40, CD2, CD27, LFA-1 (CD 11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2Rβ, IL2Rγ, IL7R a, ITGA1, VLA1, CD49a, ITGA4, IA4, CD49D, ITGA6, VLA-6, CD49f, ITGAD, CDI Id, ITGAE, CD103, ITGAL, CDI Ia, ITGAM, CDI Ib, ITGAX, CDI Ic, ITGB1, CD29, ITGB2, CD18, ITGB7, TNFR2, DNAM1(CD226), SLAMF4 (CD244, 2B4), CD84, CD96 (Tactile), CEACAM1, CRT AM, Ly9(CD229), PSGL1, CD100 (SEMA4D), SLAMF6 (NTB-A, LyI08), SLAM (SLAMF1, CD150, IPO-3), BLAME (SLAMF8), SELPLG (CD162), LTBR, PAG/Cbp, NKG2D, or NKG2C. In particular embodiments, a variety of human hinges can be employed as well including the human Ig (immunoglobulin) hinge (e.g., an IgG4 hinge, an IgD hinge), a GS linker (e.g., a GS linker described herein), a KIR2DS2 hinge or a CD8a hinge.

In particular embodiments, a transmembrane domain has a three-dimensional structure that is thermodynamically stable in a cell membrane, and generally ranges in length from 15 to 30 amino acids. The structure of a transmembrane domain can include an a helix, a β barrel, a β sheet, a β helix, or any combination thereof.

A transmembrane domain can include one or more additional amino acids adjacent to the transmembrane region, e.g., one or more amino acid within the extracellular region of the CAR (e.g., up to 15 amino acids of the extracellular region) and/or one or more additional amino acids within the intracellular region of the CAR (e.g., up to 15 amino acids of the intracellular components). In one aspect, the transmembrane domain is from the same protein that the signaling domain, co-stimulatory domain or the hinge domain is derived from. In another aspect, the transmembrane domain is not derived from the same protein that any other domain of the CAR is derived from. In some instances, the transmembrane domain can be selected or modified by amino acid substitution to avoid binding of such domains to the transmembrane domains of the same or different surface membrane proteins to minimize interactions with other unintended members of the receptor complex. In particular embodiments, the transmembrane domain is encoded by the nucleic acid sequence encoding the CD28 transmembrane domain (SEQ ID NOs: 12-14). In particular embodiments, the transmembrane domain includes the amino acid sequence of the CD28 transmembrane domain (SEQ ID NOs: 15 and 16).

(iii-b-iv) Intracellular Effector Domains. The intracellular effector domains of a CAR are responsible for activation of the cell in which the CAR is expressed. The term “effector domain” is thus meant to include any portion of the intracellular domain sufficient to transduce an activation signal. An effector domain can directly or indirectly promote a biological or physiological response in a cell when receiving the appropriate signal. In certain embodiments, an effector domain is part of a protein or protein complex that receives a signal when bound, or it binds directly to a target molecule, which triggers a signal from the effector domain. An effector domain may directly promote a cellular response when it contains one or more signaling domains or motifs, such as an immunoreceptor tyrosine-based activation motif (ITAM). In other embodiments, an effector domain will indirectly promote a cellular response by associating with one or more other proteins that directly promote a cellular response, such as co-stimulatory domains.

Effector domains can provide for activation of at least one function of a modified cell upon binding to the cellular marker expressed by a cancer cell. Activation of the modified cell can include one or more of differentiation, proliferation and/or activation or other effector functions. In particular embodiments, an effector domain can include an intracellular signaling component including a T cell receptor and a co-stimulatory domain which can include the cytoplasmic sequence from co-receptor or co-stimulatory molecule.

An effector domain can include one, two, three or more intracellular signaling components (e.g., receptor signaling domains, cytoplasmic signaling sequences), co-stimulatory domains, or combinations thereof. Exemplary effector domains include signaling and stimulatory domains selected from: 4-1BB (CD137), CARD11, CD3γ, CD3δ, CD3ε, CD3ζ, CD27, CD28, CD79A, CD79B, DAP10, FcRα, FcRβ (FcεR1b), FcRγ, Fyn, HVEM (LIGHTR), ICOS, LAG3, LAT, Lck, LRP, NKG2D, NOTCH1, pTa, PTCH2, OX40, ROR2, Ryk, SLAMF1, Slp76, TCRα, TCRβ, TRIM, Wnt, Zap70, or any combination thereof. In particular embodiments, exemplary effector domains include signaling and co-stimulatory domains selected from: CD86, FcγRIIa, DAP12, CD30, CD40, PD-1, lymphocyte function-associated antigen-1 (LFA-1), CD2, CD7, LIGHT, NKG2C, B7-H3, a ligand that specifically binds with CD83, CDS, ICAM-1, GITR, BAFFR, SLAMF7, NKp80 (KLRF1), CD127, CD160, CD19, CD4, CD8α, CD8β, IL2Rβ, IL2Rγ, IL7Rα, ITGA4, VLA1, CD49a, IA4, CD49D, ITGA6, VLA-6, CD49f, ITGAD, CD11d, ITGAE, CD103, ITGAL, CD11a, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), PSGL1, CD100 (SEMA4D), CD69, SLAMF6 (NTB-A, Ly108), SLAM (CD150, IPO-3), BLAME (SLAMF8), SELPLG (CD162), LTBR, GADS, PAG/Cbp, NKp44, NKp30, or NKp46.

Intracellular signaling component sequences that act in a stimulatory manner may include iTAMs. Examples of iTAMs including primary cytoplasmic signaling sequences include those derived from CD3γ, CD3δ, CD3ε, CD3ζ, CD5, CD22, CD66d, CD79a, CD79b, and common FcRγ (FCER1G), FcγRIIa, FcRβ (Fcε Rib), DAP10, and DAP12. In particular embodiments, variants of CD3ζ retain at least one, two, three, or all ITAM regions.

In particular embodiments, an effector domain includes a cytoplasmic portion that associates with a cytoplasmic signaling protein, wherein the cytoplasmic signaling protein is a lymphocyte receptor or signaling domain thereof, a protein including a plurality of ITAMs, a co-stimulatory domain, or any combination thereof.

Additional examples of intracellular signaling components include the cytoplasmic sequences of the CD3ζ chain, and/or co-receptors that act in concert to initiate signal transduction following binding domain engagement.

A co-stimulatory domain is a domain whose activation can be required for an efficient lymphocyte response to cellular marker binding. Some molecules are interchangeable as intracellular signaling components or co-stimulatory domains. Examples of costimulatory domains include CD27, CD28, 4-1BB (CD 137), OX40, CD30, CD40, PD-1, ICOS, lymphocyte function-associated antigen-1 (LFA-1), CD2, CD7, LIGHT, NKG2C, B7-H3, and a ligand that specifically binds with CD83. For example, CD27 co-stimulation has been demonstrated to enhance expansion, effector function, and survival of human CART cells in vitro and augments human T cell persistence and anti-cancer activity in vivo (Song et al. Blood. 2012; 119(3):696-706). Further examples of such co-stimulatory domain molecules include CDS, ICAM-1, GITR, BAFFR, HVEM (LIGHTR), SLAMF7, NKp80 (KLRF1), NKp44, NKp30, NKp46, CD160, CD19, CD4, CD8α, CD8β, IL2Rβ, IL2Rγ, IL7Rα, ITGA4, VLA1, CD49a, ITGA4, IA4, CD49D, ITGA6, VLA-6, CD49f, ITGAD, CDIId, ITGAE, CD103, ITGAL, CDIIa, ITGAM, CDI Ib, ITGAX, CDIIc, ITGBI, CD29, ITGB2, CD18, ITGB7, TNFR2, TRANCE/RANKL, DNAM1 (CD226), SLAMF4 (CD244, 2B4), CD84, CD96 (Tactile), NKG2D, CEACAM1, CRTAM, Ly9 (CD229), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, and CD19a.

In particular embodiments, the nucleic acid sequences encoding the intracellular signaling components includes CD3ζ encoding sequence (SEQ ID NO: 5) and 4-1BB signaling encoding sequence (SEQ ID NOs: 8 and 9). In particular embodiments, the amino acid sequence of the intracellular signaling component includes a CD3ζ (SEQ ID NOs: 6 and 7) and a portion of the 4-1BB (SEQ ID NO: 10 and 11) intracellular signaling component.

In particular embodiments, the intracellular signaling component includes (i) all or a portion of the signaling domain of CD3ζ, (ii) all or a portion of the signaling domain of 4-1BB, or (iii) all or a portion of the signaling domain of CD3ζ and 4-1BB.

Intracellular components may also include one or more of a protein of a Wnt signaling pathway (e.g., LRP, Ryk, or ROR2), NOTCH signaling pathway (e.g., NOTCH1, NOTCH2, NOTCH3, or NOTCH4), Hedgehog signaling pathway (e.g., PTCH or SMO), receptor tyrosine kinases (RTKs) (e.g., epidermal growth factor (EGF) receptor family, fibroblast growth factor (FGF) receptor family, hepatocyte growth factor (HGF) receptor family, insulin receptor (IR) family, platelet-derived growth factor (PDGF) receptor family, vascular endothelial growth factor (VEGF) receptor family, tropomycin receptor kinase (Trk) receptor family, ephrin (Eph) receptor family, AXL receptor family, leukocyte tyrosine kinase (LTK) receptor family, tyrosine kinase with immunoglobulin-like and EGF-like domains 1 (TIE) receptor family, receptor tyrosine kinase-like orphan (ROR) receptor family, discoidin domain (DDR) receptor family, rearranged during transfection (RET) receptor family, tyrosine-protein kinase-like (PTK7) receptor family, related to receptor tyrosine kinase (RYK) receptor family, or muscle specific kinase (MuSK) receptor family); G-protein-coupled receptors, GPCRs (Frizzled or Smoothened); serine/threonine kinase receptors (BMPR or TGFR); or cytokine receptors (IL1R, IL2R, IL7R, or IL15R).

(iii-b-v) Linkers. As used herein, a linker can include any chemical moiety that serves to connect two other subcomponents of the molecule. Some linkers serve no purpose other than to link components while many linkers serve an additional purpose. Linkers can, for example, link VL and VH of antibody derived binding domains of scFvs and serve as junction amino acids between subcomponent portions of a CAR.

Linkers can be flexible, rigid, or semi-rigid, depending on the desired function of the linker. Linkers can include junction amino acids. For example, in particular embodiments, linkers provide flexibility and room for conformational movement between different components of CAR. Commonly used flexible linkers include Gly-Ser linkers. In particular embodiments, the linker sequence includes sets of glycine and serine repeats such as from one to ten repeats of (GlyₓSer_{y})ₙ, wherein x and y are independently an integer from 0 to 10 provided that x and y are not both 0 and wherein n is an integer of 1, 2, 3, 4, 5, 6, 7, 8, 9 or 10). Particular examples include (Gly₄Ser)ₙ (SEQ ID NO: 87), (Gly₃Ser)ₙ(Gly₄Ser)ₙ (SEQ ID NO: 88), (Gly₃Ser)ₙ(Gly₂Ser)ₙ (SEQ ID NO: 89), or (Gly3Ser)n(Gly₄Ser)1 (SEQ ID NO: 90). In particular embodiments, the linker is (Gly₄Ser)₄ (SEQ ID NO: 91), (Gly₄Ser)₃ (SEQ ID NO: 92), (Gly₄Ser)₂ (SEQ ID NO: 93), (Gly₄Ser)₁ (SEQ ID NO: 94), (Gly₃Ser)₂ (SEQ ID NO: 95), (Gly₃Ser)₁ (SEQ ID NO: 96), (Gly₂Ser)₂ (SEQ ID NO: 97) or (Gly₂Ser)₁, GGSGGGSGGSG (SEQ ID NO: 98), GGSGGGSGSG (SEQ ID NO: 99), or GGSGGGSG (SEQ ID NO: 100). In particular embodiments, a (Gly₄Ser)₄ linker is encoded by the sequence as set forth in SEQ ID NO: 91.

In particular embodiments, a linker region is (GGGGS)ₙ (SEQ ID NO: 87) wherein n is an integer including, 1, 2, 3, 4, 5, 6, 7, 8, 9, or more. In particular embodiments, the spacer region is (EAAAK)ₙ (SEQ ID NO: 101) wherein n is an integer including 1, 2, 3, 4, 5, 6, 7, 8, 9, or more.

In some situations, flexible linkers may be incapable of maintaining a distance or positioning of CAR needed for a particular use. In these instances, rigid or semi-rigid linkers may be useful. Examples of rigid or semi-rigid linkers include proline-rich linkers. In particular embodiments, a proline-rich linker is a peptide sequence having more proline residues than would be expected based on chance alone. In particular embodiments, a proline-rich linker is one having at least 30%, at least 35%, at least 36%, at least 39%, at least 40%, at least 48%, at least 50%, or at least 51% proline residues. Particular examples of proline-rich linkers include fragments of proline-rich salivary proteins (PRPs).

Linkers can be susceptible to cleavage (cleavable linker), such as, acid-induced cleavage, photo-induced cleavage, peptidase-induced cleavage, esterase-induced cleavage, and disulfide bond cleavage. Alternatively, linkers can be substantially resistant to cleavage (e.g., stable linker or non-cleavable linker). In some aspects, the linker is a pro-charged linker, a hydrophilic linker, or a dicarboxylic acid-based linker.

Junction amino acids can be a linker which can be used to connect sequences when the distance provided by a spacer region is not needed and/or wanted. For example, junction amino acids can be short amino acid sequences that can be used to connect co-stimulatory intracellular signaling components. In particular embodiments, junction amino acids are 9 amino acids or less (e.g., 2, 3, 4, 5, 6, 7, 8, or 9 amino acids). In particular embodiments, a glycine-serine doublet can be used as a suitable junction amino acid linker. In particular embodiments, a single amino acid, e.g., an alanine, a glycine, can be used as a suitable junction amino acid.

(iii-b-vi) Control Features Including Tag Cassettes, Transduction Markers, and/or Suicide Switches. In particular embodiments, CAR constructs can include one or more tag cassettes and/or transduction markers. Tag cassettes and transduction markers can be used to activate, promote proliferation of, detect, enrich for, isolate, track, deplete and/or eliminate genetically modified cells in vitro, in vivo and/or ex vivo. “Tag cassette” refers to a unique synthetic peptide sequence affixed to, fused to, or that is part of a CAR, to which a cognate binding molecule (e.g., ligand, antibody, or other binding partner) is capable of specifically binding where the binding property can be used to activate, promote proliferation of, detect, enrich for, isolate, track, deplete and/or eliminate the tagged protein and/or cells expressing the tagged protein. Transduction markers can serve the same purposes but are derived from naturally occurring molecules and are often expressed using a skipping element that separates the transduction marker from the rest of the CAR molecule.

In particular embodiments, CAR include a T2A ribosomal skip element that separates the expressed CAR from a truncated CD19 (tCD19) transduction marker. In particular embodiments, the T2A ribosomal skip element is encoded by SEQ ID NO: 137.

Tag cassettes that bind cognate binding molecules include, for example, His tag (HHHHHH; SEQ ID NO: 102), Flag tag (DYKDDDDK; SEQ ID NO: 103), Xpress tag (DLYDDDDK; SEQ ID NO: 104), Avi tag (GLNDIFEAQKIEWHE; SEQ ID NO: 105), Calmodulin tag (KRRWKKNFIAVSAANRFKKISSSGAL; SEQ ID NO: 106), Polyglutamate tag, HA tag (YPYDVPDYA; SEQ ID NO: 107), Myc tag (EQKLISEEDL; SEQ ID NO: 108), Strep tag (which refers the original STREP® tag (WRHPQFGG; SEQ ID NO: 109), STREP® tag II (WSHPQFEK SEQ ID NO: 110 (IBA Institut fur Bioanalytik, Germany); see, e.g., U.S. Pat. No. 7,981,632), Softag 1 (SLAELLNAGLGGS; SEQ ID NO: 111), Softag 3 (TQDPSRVG; SEQ ID NO: 112), and V5 tag (GKPIPNPLLGLDST; SEQ ID NO: 113).

Conjugate binding molecules that specifically bind tag cassette sequences disclosed herein are commercially available. For example, His tag antibodies are commercially available from suppliers including Life Technologies, Pierce Antibodies, and GenScript.Flag tag antibodies are commercially available from suppliers including Pierce Antibodies, GenScript, and Sigma-Aldrich. Xpress tag antibodies are commercially available from suppliers including Pierce Antibodies, Life Technologies and GenScript. Avi tag antibodies are commercially available from suppliers including Pierce Antibodies, IsBio, and Genecopoeia. Calmodulin tag antibodies are commercially available from suppliers including Santa Cruz Biotechnology, Abcam, and Pierce Antibodies. HA tag antibodies are commercially available from suppliers including Pierce Antibodies, Cell Signal and Abcam. Myc tag antibodies are commercially available from suppliers including Santa Cruz Biotechnology, Abcam, and Cell Signal. Strep tag antibodies are commercially available from suppliers including Abcam, Iba, and Qiagen.

Transduction markers may be selected from at least one of a truncated CD19 (tCD19; see Budde et al., Blood 122: 1660, 2013); a truncated human EGFR (tEGFR; see Wang et al., Blood 118: 1255, 2011); an ECD of human CD34; and/or RQR8 which combines target epitopes from CD34 (see Fehse et al, Mol. Therapy 1(5 Pt 1); 448-456, 2000) and CD20 antigens (see Philip et al, Blood 124: 1277-1278).

In particular embodiments, a polynucleotide encoding an iCaspase9 construct (iCasp9) may be inserted into a CAR construct as a suicide switch.

Control features may be present in multiple copies in a CAR or can be expressed as distinct molecules with the use of a skipping element (SEQ ID NOs: 17-20). For example, a CAR can have one, two, three, four or five tag cassettes and/or one, two, three, four, or five transduction markers could also be expressed. For example, embodiments can include a CAR construct having two Myc tag cassettes, or a His tag and an HA tag cassette, or a HA tag and a Softag 1 tag cassette, or a Myc tag and a SBP tag cassette. Exemplary transduction markers and cognate pairs are described in U.S. Ser. No. 13/463,247.

One advantage of including at least one control feature in a CAR is that cells expressing CAR administered to a subject can be increased or depleted using the cognate binding molecule to a tag cassette. In certain embodiments, the present disclosure provides a method for depleting a modified cell expressing a CAR by using an antibody specific for the tag cassette, using a cognate binding molecule specific for the control feature, or by using a second modified cell expressing a CAR and having specificity for the control feature. Elimination of modified cells may be accomplished using depletion agents specific for a control feature. For example, if tEGFR is used, then an anti-tEGFR binding domain (e.g., antibody, scFv) fused to or conjugated to a cell-toxic reagent (such as a toxin, radiometal) may be used, or an anti-tEGFR/anti-CD3 bispecific scFv, or an anti-tEGFR CAR T cell may be used.

In certain embodiments, modified cells expressing a chimeric molecule may be detected or tracked in vivo by using antibodies that bind with specificity to a control feature (e.g., anti-Tag antibodies), or by other cognate binding molecules that specifically bind the control feature, which binding partners for the control feature are conjugated to a fluorescent dye, radio-tracer, iron-oxide nanoparticle or other imaging agent known in the art for detection by X-ray, CT-scan, MRI-scan, PET-scan, ultrasound, flow-cytometry, near infrared imaging systems, or other imaging modalities (see, e.g., Yu, et al., Theranostics 2:3, 2012).

Thus, modified cells expressing at least one control feature with a CAR can be, e.g., more readily identified, isolated, sorted, induced to proliferate, tracked, and/or eliminated as compared to a modified cell without a tag cassette.

(iv) Cell Activating Culture Conditions. Cell populations can be incubated in a culture-initiating media to expand genetically modified cell populations. The incubation can be carried out in a culture vessel, such as a bag, cell culture plate, flask, chamber, chromatography column, cross-linked gel, cross-linked polymer, column, culture dish, hollow fiber, microtiter plate, silica-coated glass plate, tube, tubing set, well, vial, or other container for culture or cultivating cells.

Culture conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some aspects, incubation is carried out in accordance with techniques such as those described in U.S. Pat. No. 6,040,177, Klebanoff et al. (2012) J Immunother. 35(9): 651-660, Terakura et al. (2012) Blood. 1:72-82, and/or Wang et al. (2012) J Immunother. 35(9):689-701.

Exemplary culture media for culturing T cells include (i) RPMI supplemented with non-essential amino acids, sodium pyruvate, and penicillin/streptomycin; (ii) RPMI with HEPES, 5-15% human serum, 1-3% L-Glutamine, 0.5-1.5% penicillin/streptomycin, and 0.25×10-4-0.75×10-4 M β-MercaptoEthanol; (iii) RPMI-1640 supplemented with 10% fetal bovine serum (FBS), 2 mM L-glutamine, 10 mM HEPES, 100 U/ml penicillin and 100 m/mL streptomycin; (iv) DMEM medium supplemented with 10% FBS, 2 mM L-glutamine, 10 mM HEPES, 100 U/ml penicillin and 100 m/mL streptomycin; and (v) X-Vivo 15 medium (Lonza, Walkersville, MD) supplemented with 5% human AB serum (Gemcell, West Sacramento, CA), 1% HEPES (Gibco, Grand Island, NY), 1% Pen-Strep (Gibco), 1% GlutaMax (Gibco), and 2% N-acetyl cysteine (Sigma-Aldrich, St. Louis, MO). T cell culture media are also commercially available from Hyclone (Logan, UT). Additional T cell activating components that can be added to such culture media are described in more detail below.

In some embodiments, the T cells are expanded by adding to the culture-initiating media feeder cells (e.g., such that the resulting population of cells contains at least 5, 10, 20, or 40 or more feeder cells for each T lymphocyte in the initial population to be expanded); and incubating the culture (e.g., for a time sufficient to expand the numbers of T cells). In some aspects, the non-dividing feeder cells can include gamma-irradiated feeder cells. In some embodiments, the feeder cells are irradiated with gamma rays in the range of 3000 to 3600 rads to prevent cell division. In some aspects, the feeder cells are added to culture medium prior to the addition of the populations of T cells. In particular embodiments, a time sufficient to expand the numbers of T cells includes 24 hours. In particular embodiments, the ratio of T cells to feeder cells is 1:1, 2:1, or 1:2. In particular embodiments, the feeder cells include cells expressing FOLR1, MEGF10, HPSE2, KLRF2, PCDH19, or FRAS1. In particular embodiments, the feeder cells include cancer cells. In particular embodiments, the feeder cells include AML feeder cells.

In some embodiments, the stimulating conditions include temperature suitable for the growth of human T lymphocytes, for example, at least 25° C., at least 30° C., or 37° C.

The activating culture conditions for T cells include conditions whereby T cells of the culture-initiating media proliferate or expand. T cell activating conditions can include one or more cytokines, for example, interleukin (IL)-2, IL-7, IL-15 and/or IL-21. IL-2 can be included at a range of 10-100 ng/ml (e.g., 40, 50, or 60 ng/ml). IL-7, IL-15, and/or IL-21 can be individually included at a range of 0.1-50 ng/ml (e.g., 5, 10, or 15 ng/ml).

In particular embodiments, T cell activating culture condition conditions can include T cell stimulating epitopes. T cell stimulating epitopes include CD3, CD27, CD2, CD4, CD5, CD7, CD8, CD28, CD30, CD40, CD56, CD83, CD90, CD95, 4-1BB (CD 137), B7-H3, CTLA-4, Frizzled-1 (FZD1), FZD2, FZD3, FZD4, FZD5, FZD6, FZD7, FZD8, FZD9, FZD10, HVEM, ICOS, IL-1R, LAT, LFA-1, LIGHT, MHCI, MHCII, NKG2D, OX40, ROR2 and RTK.

CD3 is a primary signal transduction element of T cell receptors. As indicated previously, CD3 is expressed on all mature T cells. In particular embodiments, the CD3 stimulating molecule (i.e., CD3 binding domain) can be derived from the OKT3 antibody (see U.S. Pat. Nos. 5,929,212; 4,361,549; ATCC® CRL-8001™; and Arakawa et al., J. Biochem. 120, 657-662 (1996)), the 20G6-F3 antibody, the 4B4-D7 antibody, the 4E7-C9, or the 18F5-H10 antibody.

In particular embodiments, CD3 stimulating molecules can be included within culture media at a concentration of at least 0.25 or 0.5 ng/ml or at a concentration of 2.5-10 μg/ml. Particular embodiments utilize a CD3 stimulating molecule (e.g., OKT3) at 5 μg/ml.

In particular embodiments, activating molecules associated with avi-tags can be biotinylated and bound to streptavidin beads. This approach can be used to create, for example, a removable T cell epitope stimulating activation system.

An exemplary binding domain for CD28 can include or be derived from TGN1412, CD80, CD86 or the 9D7 antibody. Additional antibodies that bind CD28 include 9.3, KOLT-2, 15E8, 248.23.2, EX5.3D10, and CD28.3 (deposited as a synthetic single chain Fv construct under GenBank Accession No. AF451974.1; see also Vanhove et al., BLOOD, 15 Jul. 2003, Vol. 102, No. 2, pages 564-570). Further, 1YJD provides a crystal structure of human CD28 in complex with the Fab fragment of a mitogenic antibody (5.11A1). In particular embodiments, antibodies that do not compete with 9D7 are selected.

4-1BB binding domains can be derived from LOB12, IgG2a, LOB12.3, or IgG1 as described in Taraban et al. Eur J Immunol. 2002 December; 32(12):3617-27. In particular embodiments a 4-1BB binding domain is derived from a monoclonal antibody described in U.S. Pat. No. 9,382,328. Additional 4-1BB binding domains are described in U.S. Pat. Nos. 6,569,997, 6,303,121, and Mittler et al. Immunol Res. 2004; 29(1-3):197-208.

OX40 (CD134) and/or ICOS activation may also be used. OX40 binding domains are described in US20100196359, US 20150307617, WO 2015/153513, WO2013/038191 and Melero et al. Clin Cancer Res. 2013 Mar. 1; 19(5):1044-53. Exemplary binding domains that can bind and activate ICOS are described in e.g., US20080279851 and Deng et al. Hybrid Hybridomics. 2004 June; 23(3):176-82.

When in soluble form, T-cell activating agents can be coupled with another molecule, such as polyethylene glycol (PEG) molecule. Any suitable PEG molecule can be used. Typically, PEG molecules up to a molecular weight of 1000 Da are soluble in water or culture media. In some cases, such PEG based reagent can be prepared using commercially available activated PEG molecules (for example, PEG-NHS derivatives available from NOF North America Corporation, Irvine, Calif., USA, or activated PEG derivatives available from Creative PEGWorks, Chapel Hills, N.C., USA).

In particular embodiments, cell stimulating agents are immobilized on a solid phase within the culture media. In particular embodiments, the solid phase is a surface of the culture vessel (e.g., bag, cell culture plate, chamber, chromatography column, cross-linked gel, cross-linked polymer, column, culture dish, hollow fiber, microtiter plate, silica-coated glass plate, tube, tubing set, well, vial, other structure or container for culture or cultivation of cells).

In particular embodiments, a solid phase can be added to a culture media. Such solid phases can include, for example, beads, hollow fibers, resins, membranes, and polymers.

Exemplary beads include magnetic beads, polymeric beads, and resin beads (e.g., Strep-Tactin® Sepharose, Strep-Tactin® Superflow, and Strep-Tactin® MacroPrep IBA GmbH, Gottingen)). Anti-CD3/anti-CD28 beads are commercially available reagents for T cell expansion (Invitrogen). These beads are uniform, 4.5 μm superparamagnetic, sterile, non-pyrogenic polystyrene beads coated with a mixture of affinity purified monoclonal antibodies against the CD3 and CD28 cell surface molecules on human T cells. Hollow fibers are available from TerumoBCT Inc. (Lakewood, Colo., USA). Resins include metal affinity chromatography (IMAC) resins (e.g., TALON® resins (Westburg, Leusden)). Membranes include paper as well as the membrane substrate of a chromatography matrix (e.g., a nitrocellulose membrane or a polyvinylidene difluoride (PVDF) membrane).

Exemplary polymers include polysaccharides, such as polysaccharide matrices. Such matrices include agarose gels (e.g., Superflow™ agarose or a Sepharose® material such as Superflow™ Sepharose® that are commercially available in different bead and pore sizes) or a gel of crosslinked dextran(s). A further illustrative example is a particulate cross-linked agarose matrix, to which dextran is covalently bonded, that is commercially available (in various bead sizes and with various pore sizes) as Sephadex® or Superdex®, both available from GE Healthcare.

Synthetic polymers that may be used include polyacrylamide, polymethacrylate, a co-polymer of polysaccharide and agarose (e.g. a polyacrylamide/agarose composite) or a polysaccharide and N,N′-methylenebisacrylamide. An example of a copolymer of a dextran and N,N′-methylenebisacrylamide is the Sephacryl® (Pharmacia Fine Chemicals, Inc., Piscataway, NJ) series of materials.

Particular embodiments may utilize silica particles coupled to a synthetic or to a natural polymer, such as polysaccharide grafted silica, polyvinylpyrrolidone grafted silica, polyethylene oxide grafted silica, poly(2-hydroxyethylaspartamide) silica and poly(N-isopropylacrylamide) grafted silica.

Cell activating agents can be immobilized to solid phases through covalent bonds or can be reversibly immobilized through non-covalent attachments.

In particular embodiments, T cells are activated with anti-CD3/CD28 beads (3:1 beads: cell, Gibco, 11131D) on Retronectin-coated plates. In particular embodiments, CAR T cells are sorted with CD19 microbeads 8 to 10 days post activation. In particular embodiments, sorted cells are further expanded in CTL (+50 U/mL IL-2) media.

Culture conditions for HSC/HSP can include expansion with a Notch agonist (see, e.g., U.S. Pat. Nos. 7,399,633; 5,780,300; 5,648,464; 5,849,869; and 5,856,441 and growth factors present in the culture condition as follows: 25-300 ng/ml SCF, 25-300 ng/ml Flt-3L, 25-100 ng/ml TPO, 25-100 ng/ml IL-6 and 10 ng/ml IL-3. In more specific embodiments, 50, 100, or 200 ng/ml SCF; 50, 100, or 200 ng/ml of Flt-3L; 50 or 100 ng/ml TPO; 50 or 100 ng/ml IL-6; and 10 ng/ml IL-3 can be used.

(v) Ex Vivo Manufactured Cell Formulations. In particular embodiments, genetically modified cells can be harvested from a culture medium and washed and concentrated into a carrier in a therapeutically-effective amount. Exemplary carriers include saline, buffered saline, physiological saline, water, Hanks' solution, Ringer's solution, Nonnosol-R (Abbott Labs), PLASMA-LYTE A® (Baxter Laboratories, Inc., Morton Grove, IL), glycerol, ethanol, and combinations thereof.

In particular embodiments, carriers can be supplemented with human serum albumin (HSA) or other human serum components or fetal bovine serum. In particular embodiments, a carrier for infusion includes buffered saline with 5% HSA or dextrose. Additional isotonic agents include polyhydric sugar alcohols including trihydric or higher sugar alcohols, such as glycerin, erythritol, arabitol, xylitol, sorbitol, or mannitol.

Carriers can include buffering agents, such as citrate buffers, succinate buffers, tartrate buffers, fumarate buffers, gluconate buffers, oxalate buffers, lactate buffers, acetate buffers, phosphate buffers, histidine buffers, and/or trimethylamine salts.

Stabilizers refer to a broad category of excipients which can range in function from a bulking agent to an additive which helps to prevent cell adherence to container walls. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol; PEG; amino acid polymers; sulfur-containing reducing agents, such as urea, glutathione, thioctic acid, sodium thioglycolate, thioglycerol, alpha-monothioglycerol, and sodium thiosulfate; low molecular weight polypeptides (i.e., <10 residues); proteins such as HSA, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran.

Where necessary or beneficial, compositions and/or formulations can include a local anesthetic such as lidocaine to ease pain at a site of injection.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Therapeutically effective amounts of cells within compositions and/or formulations can be greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹.

In compositions and formulations disclosed herein, cells are generally in a volume of a liter or less, 500 mls or less, 250 mls or less or 100 mls or less. Hence the density of administered cells is typically greater than 10⁴ cells/ml, 10⁷ cells/ml or 10⁸ cells/ml.

As indicated, formulations include at least one genetically modified cell type (e.g., modified T cells, NK cells, or stem cells). Formulations can include different types of genetically-modified cells (e.g., T cells, NK cells, and/or stem cells in combination).

Different types of genetically-modified cells or cell subsets (e.g., modified T cells, NK cells, and/or stem cells) can be provided in different ratios e.g., a 1:1:1 ratio, 2:1:1 ratio, 1:2:1 ratio, 1:1:2 ratio, 5:1:1 ratio, 1:5:1 ratio, 1:1:5 ratio, 10:1:1 ratio, 1:10:1 ratio, 1:1:10 ratio, 2:2:1 ratio, 1:2:2 ratio, 2:1:2 ratio, 5:5:1 ratio, 1:5:5 ratio, 5:1:5 ratio, 10:10:1 ratio, 1:10:10 ratio, 10:1:10 ratio, etc. These ratios can also apply to numbers of cells expressing the same or different CAR components. If only two of the cell types are combined or only 2 combinations of expressed CAR components are included within a formulation, the ratio can include any 2-number combination that can be created from the 3 number combinations provided above. In embodiments, the combined cell populations are tested for efficacy and/or cell proliferation in vitro, in vivo and/or ex vivo, and the ratio of cells that provides for efficacy and/or proliferation of cells is selected.

The cell-based formulations disclosed herein can be prepared for administration by, e.g., injection, infusion, perfusion, or lavage. The formulations and formulations can further be formulated for bone marrow, intravenous, intradermal, intraarterial, intranodal, intralymphatic, intraperitoneal, intralesional, intratumoral, intravesicular, and/or subcutaneous injection.

(vi) Antibody Conjugates. An antibody conjugate refers to a binding domain as disclosed herein linked to another entity. The other entity can be, for example, a toxin, a drug, label, a radioisotope, or a nanoparticle. In particular examples, an antibody conjugate is an immunotoxin, an antibody-drug conjugate (ADC), an antibody-radioisotope conjugate, or an antibody-nanoparticle conjugate.

Immunotoxins include a binding domain (e.g., an antibody or binding fragment thereof) disclosed herein conjugated to one or more cytotoxins (e.g., protein toxins, enzymatically active toxins of bacterial, fungal, plant, or animal origin, or fragments thereof). A toxin can be any agent that is detrimental to cells. Frequently used plant toxins are divided into two classes: (1) holotoxins (or class II ribosome inactivating proteins), such as ricin, abrin, mistletoe lectin, and modeccin, and (2) hemitoxins (class I ribosome inactivating proteins), such as pokeweed antiviral protein (PAP), saporin, Bryodin 1, bouganin, and gelonin. Commonly used bacterial toxins include diphtheria toxin (DT) and Pseudomonas exotoxin (PE). Kreitman, Current Pharmaceutical Biotechnology 2:313-325 (2001). The toxin may be obtained from essentially any source and can be a synthetic or a natural product.

Immunotoxins with multiple (e.g., four) cytotoxins per binding domain can be prepared by partial reduction of the binding domain with an excess of a reducing reagent such as dithiothreitol (DTT) or tris(2-carboxyethyl)phosphine (TCEP) at 37° C. for 30 min, then the buffer can be exchanged by elution through SEPHADEX G-25 resin with 1 mM DTPA (diethylene triamine penta-acetic acid) in Dulbecco's phosphate-buffered saline (DPBS). The eluent can be diluted with further DPBS, and the thiol concentration of the binding domain can be measured using 5,5′-dithiobis(2-nitrobenzoic acid) [Ellman's reagent]. An excess, for example 5-fold, of a linker-cytotoxin conjugate can be added at 4° C. for 1 hr, and the conjugation reaction can be quenched by addition of a substantial excess, for example 20-fold, of cysteine. The resulting immunotoxin mixture can be purified on SEPHADEX G-25 equilibrated in PBS to remove unreacted linker-cytotoxin conjugate, desalted if desired, and purified by size-exclusion chromatography. The resulting immunotoxin can then be sterile filtered, for example, through a 0.2 μm filter, and can be lyophilized if desired for storage.

In particular embodiments, immunotoxins can include binding domains conjugated to toxins for targeted cell killing.

Antibody-drug conjugates (ADC) allow for the targeted delivery of a drug moiety to a cell expressing the target cellular marker. In particular embodiments, the drug moiety can include a cytotoxic drug or a therapeutic drug or agent.

In particular embodiments, ADC refer to targeted chemotherapeutic molecules which combine properties of both binding domains and cytotoxic drugs by targeting potent cytotoxic drugs to antigen-expressing cancer cells (Teicher, B. A. (2009) Current Cancer Drug Targets 9:982-1004), thereby enhancing the therapeutic index by maximizing efficacy and minimizing off-target toxicity (Carter, P. J. and Senter P. D. (2008) The Cancer Jour. 14(3):154-169; Chari, R. V. (2008) Acc. Chem. Res. 41:98-107). See also Kamath & lyer (Pharm Res. 32(11): 3470-3479, 2015), which describes considerations for the development of ADCs.

The cytotoxic drug moiety of the ADC may include any compound, moiety or group that has a cytotoxic or cytostatic effect. Cytotoxic drug moieties may impart their cytotoxic and cytostatic effects by mechanisms including tubulin binding, DNA binding or intercalation, and inhibition of RNA polymerase, protein synthesis, and/or topoisomerase. Exemplary drugs include actinomycin D, anthracycline, auristatin, calicheamicin, camptothecin, CC1065, colchicin, cytochalasin B, daunorubicin, 1-dehydrotestosterone, dihydroxy anthracinedione, dolastatin, doxorubicin, duocarmycin, elinafide, emetine, ethidium bromide, etoposide, gramicidin D, glucocorticoids, lidocaine, maytansinoid (including monomethyl auristatin E [MMAE]; vedotin), mithramycin, mitomycin, mitoxantrone, nemorubicin, PNU-159682, procaine, propranolol, puromycin, pyrrolobenzodiazepine (PBD), taxane, taxol, tenoposide, tetracaine, trichothecene, vinblastine, vinca alkaloid, vincristine, and stereoisomers, isosteres, analogs, and derivatives thereof that have cytotoxic activity.

In particular embodiments, the ADC compounds include a binding domain conjugated, i.e., covalently attached, to the drug moiety. In particular embodiments, the binding domain is covalently attached to the drug moiety through a linker. A linker can include any chemical moiety that is capable of linking a binding domain, an antibody, antibody fragment (e.g., antigen binding fragments) or functional equivalent to another moiety, such as a drug moiety. Linkers can be susceptible to cleavage (cleavable linker), such as, acid-induced cleavage, photo-induced cleavage, peptidase-induced cleavage, esterase-induced cleavage, and disulfide bond cleavage, at conditions under which the compound or the binding domain remains active. Alternatively, linkers can be substantially resistant to cleavage (e.g., stable linker or noncleavable linker). In some aspects, the linker is a procharged linker, a hydrophilic linker, or a dicarboxylic acid-based linker. The ADCs selectively deliver an effective dose of a drug to cancer cells whereby greater selectivity, i.e., a lower efficacious dose, may be achieved while increasing the therapeutic index (“therapeutic window”).

To prepare ADCs, linker-drug conjugates can be made by conventional methods analogous to those described by Doronina et al. (Bioconjugate Chem. 17: 114-124, 2006). Antibody-drug conjugates with multiple (e.g., four) drugs per binding domain can be prepared by partial reduction of the binding domain with an excess of a reducing reagent such as dithiothreitol (DTT) or tris(2-carboxyethyl)phosphine (TCEP) at 37° C. for 30 min, then the buffer can be exchanged by elution through SEPHADEX G-25 resin with 1 mM DTPA in Dulbecco's phosphate-buffered saline (DPBS). The eluent can be diluted with further DPBS, and the thiol concentration of the binding domain can be measured using 5,5′-dithiobis(2-nitrobenzoic acid) [Ellman's reagent]. An excess, for example 5-fold, of a linker-drug conjugate can be added at 4° C. for 1 hr, and the conjugation reaction can be quenched by addition of a substantial excess, for example 20-fold, of cysteine. The resulting ADC mixture can be purified on SEPHADEX G-25 equilibrated in PBS to remove unreacted linker-drug conjugate, desalted if desired, and purified by size-exclusion chromatography. The resulting ADC can then be sterile filtered, for example, through a 0.2 μm filter, and can be lyophilized if desired for storage.

Antibody-radioisotope conjugates include a binding domain linked to a radioisotope for use in nuclear medicine. Nuclear medicine refers to the diagnosis and/or treatment of conditions by administering radioactive isotopes (radioisotopes or radionuclides) to a subject. Therapeutic nuclear medicine is often referred to as radiation therapy or radioimmunotherapy (RIT).

Examples of radioactive isotopes that can be conjugated to binding domains of the present disclosure include actinium-225, iodine-131, arsenic-211, iodine-131, indium-111, yttrium-90, and lutetium-177, as well as alpha-emitting radionuclides such as astatine-211 or bismuth-212 or bismuth-213. Methods for preparing radioimmunoconjugates are established in the art. Examples of radioimmunoconjugates are commercially available, including Zevalin™ (DEC Pharmaceuticals), and similar methods can be used to prepare radioimmunoconjugates using the binding domains of the disclosure.

Examples of radionuclides that are useful for radiation therapy include ²²⁵Ac and ²²⁷Th. ²²⁵Ac is a radionuclide with the half-life of ten days. As ²²⁵Ac decays the daughter isotopes ²²¹Fr, ²¹³Bi, and ²⁰⁹Pb are formed. ²²⁷Th has a half-life of 19 days and forms the daughter isotope ²²³Ra.

Additional examples of useful radioisotopes include ²²⁸Ac, ¹¹¹Ag, ¹²⁴Am, ⁷⁴As, ²¹¹As, ²⁰⁹At, ¹⁹⁴Au, ¹²⁸Ba, ⁷Be, ²⁰⁶Bi, ²⁴⁵Bk, ²⁴⁶Bk, ⁷⁶Br, ¹¹C, ⁴⁷Ca, ²⁵⁴Cf, ²⁴²Cm, ⁵¹Cr, ⁶⁷Cu, ¹⁵³Dy, ¹⁵⁷Dy, ¹⁵⁹Dy, ¹⁶⁵Dy, ¹⁶⁶Dy, ¹⁷¹Er, ²⁵⁰Es, ²⁵⁴Es, ¹⁴⁷Eu, ¹⁵⁷Eu, ⁵²Fe, ⁵⁹Fe, ²⁵¹Fm, ²⁵²Fm, ²⁵³Fm, ⁶⁶Ga, ⁷²Ga, ¹⁴⁶Gd, ¹⁵³Gd ⁶⁸Ge, ¹⁷⁰Hf, ¹⁷¹Hf, ¹⁹³Hg, ¹⁹³mHg, ¹⁶⁰mHo, ¹³⁰I, ¹³¹I, ¹³⁵I, ¹¹⁴mIn, ¹⁸⁵Ir, ⁴²K, ⁴³K, ⁷⁶Kr, ⁷⁹Kr, ⁸¹mKr, ¹³²La, ²⁶²Lr, ¹⁶⁹Lu, ¹⁷⁴mLu, ¹⁷⁶mLu, ²⁵⁷Md, ²⁶⁰Md, ²⁸Mg, ⁵²Mn, ⁹⁰Mo, ²⁴Na, ⁹⁵Nb, ¹³⁸Nd, ⁵⁷Ni, ⁶⁶Ni, ²³⁴Np, ¹⁵O, ¹⁸²Os, ¹⁸⁹mOs, ¹⁹¹Os, ³²P ²⁰¹Pb, ¹⁰¹Pd, ¹⁴³Pr, ¹⁹¹Pt, ²⁴³Pu, ²²⁵Ra, ⁸¹Rb, ¹⁸⁸Re, ¹⁰⁵Rh, ²¹¹Rn, ¹⁰³Ru, ³⁵S, ⁴⁴Sc, ⁷²Se, ¹⁵³Sm, ¹²⁵Sn, ⁹¹Sr, ¹⁷³Ta, ¹⁵⁴Tb, ¹²⁷Te, ²³⁴Th, ⁴⁵Ti, ¹⁶⁶Tm, ²³⁰U, ²³⁷U, ²⁴⁰U, ⁴⁸V, ¹⁷⁸W, ¹⁸¹W, ¹⁸⁸W, ¹²⁵Xe, ¹²⁷Xe ¹³³Xe, ¹³³mXe, ¹³⁵Xe, ⁸⁵mY, ⁸⁶Y, ⁹⁰Y, ⁹³Y ¹⁶⁹Yb, ¹⁷⁵Yb, ⁶⁵Zn, ⁷¹mZn, ⁸⁶Zr, ⁹⁵Zr, and/or ⁹⁷Zr.

In particular embodiments, the antibody conjugate includes antibody-nanoparticle conjugates. Antibody-nanoparticle conjugates can function in the targeted delivery of a payload (e.g., small molecules or genetic engineering components) to a cell ex vivo or in vivo that expresses the target cell marker. For example, scFv or other binding fragments can be linked to the surface of nanoparticles to guide delivery to target cells. The linkage can be through, for example, covalent attachment.

Examples of nanoparticles include metal nanoparticles (e.g., gold, platinum, or silver), liposomes, and polymer-based nanoparticles.

Methods of forming liposomes are described in, for example, U.S. Pat. Nos. 4,229,360; 4,224,179; 4,241,046; 4,737,323; 4,078,052; 4,235,871; 4,501,728; and 4,837,028, as well as in Szoka et al., Ann. Rev. Biophys. Bioeng. 9:467 (1980) and Hope et al., Chem. Phys. Lip. 40:89 (1986). For additional information regarding nanoparticles, see Yetisgin et al., Molecules 2020, 25, 2193.

Examples of polymers that can be used within nanoparticles include polyglutamic acid (PGA); poly(lactic-co-glycolic acid) (PLGA); Polylactic acid (PLA); poly-D-lactic acid (PDLA); PLGA-dimethacrylate; polyamines; polyorganic amines (e.g., polyethyleneimine (PEI), polyethyleneimine celluloses); poly(amidoamines) (PAMAM); polyamino acids (e.g., polylysine (PLL), polyarginine); polysaccharides (e.g., cellulose, dextran, DEAE dextran, starch); spermine, spermidine, poly(vinylbenzyl trialkyl ammonium), poly(4-vinyl-N-alkyl-pyridiumiun), poly(acryloyl-trialkyl ammonium), and Tat proteins.

In particular embodiments, the nanoparticles can include a coating, particularly when used in vivo. A coating can serve to shield the encapsulated cargo and/or reduce or prevent off-target binding. Off-target binding is reduced or prevented by reducing the surface charge of the nanoparticles to neutral or negative. Coatings can include neutral or negatively charged polymer- and/or liposome-based coatings. In particular embodiments, the coating is a dense surface coating of hydrophilic and/or neutrally charged hydrophilic polymer sufficient to prevent the encapsulated cargo from being exposed to the environment before release into a target cell. In particular embodiments, the coating covers at least 80% or at least 90% of the surface of the nanoparticle.

Examples of neutrally charged polymers that can be used as a nanoparticle coating include polyethylene glycol (PEG); poly(propylene glycol); and polyalkylene oxide copolymers, (PLURONIC®, BASF Corp., Mount Olive, NJ).

The size of particles can vary over a wide range and can be measured in different ways. In preferred embodiments, nanoparticles are <130 nm in size. However, nanoparticles can also have a minimum dimension of equal to or less than 500 nm, less than 150 nm, less than 140 nm, less than 120 nm, less than 110 nm, less than 100 nm, less than 90 nm, less than 80 nm, less than 70 nm, less than 60 nm, less than 50 nm, less than 40 nm, less than 30 nm, less than 20 nm, or less than 10 nm. In particular embodiments, nanoparticles are 90 to 130 nm in size.

Dimensions of the particles can be determined using, e.g., conventional techniques, such as dynamic light scattering and/or electron microscopy.

(vii) Compositions. A composition as described herein includes (i) an antibody or antibody binding fragments; (ii) antibody conjugates; and/or (iii) nanoparticles (collectively referred to as “active ingredients” hereafter) and a pharmaceutically acceptable carrier. Any of the active ingredients described herein in any exemplary format or conjugation form can be formulated alone or in combination into compositions for administration to subjects. Salts and/or pro-drugs of the active ingredients can also be used.

A pharmaceutically acceptable salt includes any salt that retains the activity of the active ingredients and is acceptable for pharmaceutical use. A pharmaceutically acceptable salt also refers to any salt which may form in vivo as a result of administration of an acid, another salt, or a prodrug which is converted into an acid or salt.

Suitable pharmaceutically acceptable acid addition salts can be prepared from an inorganic acid or an organic acid. Examples of such inorganic acids are hydrochloric, hydrobromic, hydroiodic, nitric, carbonic, sulfuric and phosphoric acid. Appropriate organic acids can be selected from aliphatic, cycloaliphatic, aromatic, arylaliphatic, heterocyclic, carboxylic and sulfonic classes of organic acids.

Suitable pharmaceutically acceptable base addition salts include metallic salts made from aluminum, calcium, lithium, magnesium, potassium, sodium and zinc or organic salts made from N,N′-dibenzylethylene-diamine, chloroprocaine, choline, diethanolamine, ethylenediamine, N-methylglucamine, lysine, arginine and procaine.

A prodrug includes an active ingredient which is converted to a therapeutically active compound after administration, such as by cleavage or by hydrolysis of a biologically labile group.

Exemplary generally used pharmaceutically acceptable carriers include any and all absorption delaying agents, antioxidants, binders, buffering agents, bulking agents or fillers, chelating agents, coatings, disintegration agents, dispersion media, gels, isotonic agents, lubricants, preservatives, salts, solvents or co-solvents, stabilizers, surfactants, and/or delivery vehicles. Exemplary carriers include saline, buffered saline, physiological saline, water, Hanks' solution, Ringer's solution, Nonnosol-R (Abbott Labs), Plasma-Lyte A® (Baxter Laboratories, Inc., Morton Grove, IL), glycerol, ethanol, and combinations thereof.

Exemplary antioxidants include ascorbic acid, methionine, and vitamin E.

Exemplary buffering agents include citrate buffers, succinate buffers, tartrate buffers, fumarate buffers, gluconate buffers, oxalate buffers, lactate buffers, acetate buffers, phosphate buffers, histidine buffers, and/or trimethylamine salts.

An exemplary chelating agent is EDTA (ethylene-diamine-tetra-acetic acid).

Exemplary isotonic agents include polyhydric sugar alcohols including trihydric or higher sugar alcohols, such as glycerin, erythritol, arabitol, xylitol, sorbitol, or mannitol.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Stabilizers refer to a broad category of excipients which can range in function from a bulking agent to an additive which solubilizes the active ingredients or helps to prevent denaturation or adherence to the container wall. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol; PEG; amino acid polymers; sulfur-containing reducing agents, such as urea, glutathione, thioctic acid, sodium thioglycolate, thioglycerol, α-monothioglycerol, and sodium thiosulfate; low molecular weight polypeptides (i.e., <10 residues); proteins such as human serum albumin, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran. Stabilizers are typically present in the range of from 0.1 to 10,000 parts by weight based on therapeutic weight.

The compositions disclosed herein can be formulated for administration by, for example, injection, inhalation, infusion, perfusion, lavage, or ingestion. The compositions disclosed herein can further be formulated for intravenous, intradermal, intraarterial, intranodal, intralymphatic, intraperitoneal, intralesional, intraprostatic, intravaginal, intrarectal, topical, intrathecal, intratumoral, intramuscular, intravesicular, oral, sublingual, and/or subcutaneous administration.

For injection, compositions can be formulated as aqueous solutions, such as in buffers including Hanks' solution, Ringer's solution, or physiological saline. The aqueous solutions can include formulatory agents such as suspending, stabilizing, and/or dispersing agents. Alternatively, the composition can be in lyophilized and/or powder form for constitution with a suitable vehicle, e.g., sterile pyrogen-free water, before use.

Compositions can be formulated as an aerosol. In particular embodiments, the aerosol is provided as part of an anhydrous, liquid or dry powder inhaler. Aerosol sprays from pressurized packs or nebulizers can also be used with a suitable propellant, e.g., dichlorodifluoromethane, trichlorofluoromethane, dichlorotetrafluoroethane, carbon dioxide or other suitable gas.

Additionally, compositions can be formulated as sustained-release systems utilizing semipermeable matrices of solid polymers including at least one type of antibody conjugate or nanoparticle.

In particular embodiments, the compositions include active ingredients of at least 0.1% w/v or w/w of the composition; at least 1% w/v or w/w of composition; at least 10% w/v or w/w of composition; at least 20% w/v or w/w of composition; at least 30% w/v or w/w of composition; at least 40% w/v or w/w of composition; at least 50% w/v or w/w of composition; at least 60% w/v or w/w of composition; at least 70% w/v or w/w of composition; at least 80% w/v or w/w of composition; at least 90% w/v or w/w of composition; at least 95% w/v or w/w of composition; or at least 99% w/v or w/w of composition.

Any composition disclosed herein can advantageously include any other pharmaceutically acceptable carriers which include those that do not produce significantly adverse, allergic, or other untoward reactions that outweigh the benefit of administration. Exemplary pharmaceutically acceptable carriers are disclosed in Remington's Pharmaceutical Sciences, 18th Ed. Mack Printing Company, 1990. Moreover, compositions can be prepared to meet sterility, pyrogenicity, general safety, and purity standards as required by U.S. FDA Office of Biological Standards and/or other relevant foreign regulatory agencies.

(viii) Methods of Use. Methods disclosed herein include treating subjects (humans, veterinary animals (dogs, cats, reptiles, birds, etc.) livestock (horses, cattle, goats, pigs, chickens, etc.) and research animals (monkeys, rats, mice, fish, etc.) with formulations and/or compositions disclosed herein. Treating subjects includes delivering therapeutically effective amounts. Therapeutically effective amounts include those that provide effective amounts, prophylactic treatments and/or therapeutic treatments.

An “effective amount” is the amount of a formulation and/or composition necessary to result in a desired physiological change in the subject. For example, an effective amount can provide an immunogenic anti-cancer effect. Effective amounts are often administered for research purposes. Effective amounts disclosed herein can cause a statistically significant effect in an animal model or in vitro assay relevant to the assessment of a cancer's development or progression. An immunogenic amount can be provided in an effective amount, wherein the effective amount stimulates an immune response.

A “prophylactic treatment” includes a treatment administered to a subject who does not display signs or symptoms of a cancer or displays only early signs or symptoms of a cancer such that treatment is administered for the purpose of diminishing or decreasing the risk of developing the cancer further. Thus, a prophylactic treatment functions as a preventative treatment against a cancer.

A “therapeutic treatment” includes a treatment administered to a subject who displays symptoms or signs of a cancer and is administered to the subject for the purpose of diminishing or eliminating those signs or symptoms of the cancer. The therapeutic treatment can reduce, control, or eliminate the presence or activity of the cancer and/or reduce control or eliminate side effects of the cancer.

Function as an effective amount, prophylactic treatment or therapeutic treatment are not mutually exclusive, and in particular embodiments, administered dosages may accomplish more than one treatment type.

In particular embodiments, therapeutically effective amounts provide anti-cancer effects. Anti-cancer effects include a decrease in the number of cancer cells, decrease in tumor size, an increase in life expectancy, induced chemo- or radiosensitivity in cancer cells, inhibited cancer cell proliferation, prolonged subject life, reduced cancer-associated pain, and/or reduced relapse or re-occurrence of cancer following treatment.

In particular embodiments, therapeutically effective amounts induce an immune response. The immune response can be against a cancer cell.

In particular embodiments, the cancer cell expresses FOLR1, MEGF10, HPSE2, KLRF2, PCDH19, and/or FRAS1. In particular embodiments, the cancer includes leukemia, peritoneal cancer, fallopian tube cancer, ovarian cancer (e.g., epithelial ovarian cancer), endometrial cancer, cervical cancer, breast cancer (e.g., triple-negative breast cancer, HER2-breast cancer), bladder cancer, renal cell carcinoma, pituitary tumors, lung cancer (e.g., lung adenocarcinoma or epithelial lung cancer such as non-small cell lung cancer), uterine cancer, squamous cell carcinoma, ureter cancer, urethral cancer, osteosarcoma, transitional cell carcinoma. In particular embodiments, the leukemia is acute myeloid leukemia (AML). In particular embodiments, the AML is CBFA2T3/GLIS2 (C/G) AML. In particular embodiments, the cancer cell is a C/G AML cell, expressing FOLR1, MEGF10, HPSE2, KLRF2, PCDH19, and/or FRAS1. In particular embodiments, the cancer cell is a leukemia, peritoneal cancer, fallopian tube cancer, ovarian cancer, endometrial cancer, cervical cancer, breast cancer, bladder cancer, renal cell carcinoma, pituitary tumor, lung cancer, uterine cancer, squamous cell carcinoma, ureter cancer, urethral cancer, osteosarcoma, or transitional cell carcinoma cell expressing FOLR1.

The following clinical trials, by Trial Identifier No., provide further support for the efficacy of binding FOL1R in the treatment of various cancer types: GDCT0356356: Indications: Peritoneal Cancer (PC), Fallopian Tube Cancer (FTC), Epithelial Ovarian Cancer (EOC); GDCT0374537: Indications: Ovarian Cancer (OC), EOC, FTC, PC; GDCT0429750: Indications: OC, Solid Tumor, Endometrial Cancer (EC), Non-Small Cell Lung Cancer (NSCLC), FTC, PC, EOC, Triple-Negative Breast Cancer (TNBC); GDCT0026391: Indications: FTC, PC, OC, EOC; GDCT0447204: Indications: EOC, PC, FTC; GDCT0232423: Indications: EOC, PC, FTC, OC; GDCT0229058: Indications: NSCLC; GDCT0445760: Indications: OC; GDCT0001547: Indications: EOC, OC, PC, FTC; GDCT0198047: Indications: NSCLC; GDCT0201658: Indications: TNBC, Breast Cancer (BC), Human Epidermal Growth Factor Receptor 2 Negative Breast Cancer (HER2-Breast Cancer (HER2-BC)); GDCT0043006: Indications: OC; GDCT0423171: Indications: OC; GDCT0002756: Indications: EOC; GDCT0011290: Indications: Solid Tumor; GDCT0198051: Indications: OC; GDCT0286303: Indications: Metastatic BC, TNBC, BC, HER2-BC; GDCT0227787: Indications: OC, EOC, PC, FTC; GDCT0403589: Indications: FTC, PC, EOC; GDCT0004066: Indications: OC; GDCT0007040: Indications: Metastatic Renal Cell Carcinoma (RCC), RCC; GDCT0007042: Indications: OC, EC; GDCT0445900: Indications: OC; GDCT0041274: Indications: Adenomas, Pituitary Tumor; GDCT0016528: Indications: Lung Adenocarcinoma; GDCT0346710: Indications: EC, Uterine Cancer (UC); GDCT0347063: Indications: EC; GDCT0347076: Indications: EC; GDCT0014731: Indications: EOC, FTC, PC, OC; GDCT0144078: Indications: OC; GDCT0048904: Indications: Lung Adenocarcinoma, NSCLC; GDCT0152905: Indications: NSCLC, Squamous Cell Carcinoma; GDCT0010332: Indications: PC, EOC, FTC, OC; GDCT0380005: Indications: EOC, OC, FTC, PC, Metastatic OC; GDCT0433366: Indications: OC, PC, FTC, EOC; GDCT0291846: Indications: Bladder Cancer, Transitional Cell Carcinoma (Urothelial Cell Carcinoma), RCC, Ureter Cancer, Urethral Cancer, Metastatic Transitional (Urothelial) Tract Cancer, Bladder Carcinoma; GDCT0381573: Indications: TNBC, EC, OC, NSCLC, PC, FTC, EOC; GDCT0232424: Indications: EOC, PC, FTC, EC, OC; GDCT0325007: Indications: EC, OC, FTC, PC, Carcinomas, UC, Metastatic BC; GDCT0401603: Indications: BC, Lung Cancer (LC); GDCT0405347: Indications: OC; GDCT0429611: Indications: EOC, EC, Solid Tumor, PC, FTC, NSCLC; GDCT0209078: Indications: Solid Tumor; GDCT0158131: Indications: Solid Tumor, EC, EOC, NSCLC, OC, FTC, PC, Transitional Cell Carcinoma (Urothelial Cell Carcinoma), Cervical Cancer, RCC; GDCT0301058: Indications: Osteosarcoma; GDCT0456860: Indications: NSCLC, RCC, EOC, Solid Tumor, FTC, PC, EC, Squamous Non-Small Cell Lung Carcinoma; GDCT0450501: Indications: PC, NSCLC; GDCT0284998: Indications: OC, EOC, Carcinomas; GDCT0005507: Indications: OC, EOC; GDCT0006565: Indications: Metastatic Cancer Advanced Malignancy; GDCT0250249: Indications: Unspecified Cancer; GDCT0006477: Indications: Metastatic Cancer; GDCT0291176: Indications: Osteosarcoma; GDCT0429953: Indications: OC; GDCT0002840: Indications: Solid Tumor; GDCT0017012: Indications: Solid Tumor Lymphoma; GDCT0007464: Indications: Advanced Malignancy Solid Tumor, Metastatic OC, Unspecified Cancer; GDCT0205391: Indications: Solid Tumor OC, EC, NSCLC, TNBC; GDCT0452526: Indications: Osteosarcoma; GDCT0198062: Indications: Metastatic RCC, OC; GDCT0281679: Indications: TNBC; GDCT0243737: Indications: OC, FTC, Solid Tumor, Malignant Mesothelioma, EC; GDCT0170283: Indications: EC, Advanced Malignancy, Metastatic Cancer, Solid Tumor; GDCT0162335: Indications: Solid Tumor; GDCT0289943: Indications: Solid Tumor EC, TNBC, OC, FTC, NSCLC, PC; GDCT0279766: Indications: OC; GDCT0278064: Indications: FTC, OC, PC, UC, EC, Carcinomas, TNBC, BC; GDCT0250120: Indications: TNBC; GDCT0295131: Indications: OC, LC, TNBC; GDCT0391284: Indications: FTC, EC, EOC, Peritoneal Tumor, OC, PC; GDCT0003999: Indications: Metastatic Cancer, Metastatic RCC; GDCT0011743: Indications: FTC, PC, EOC; GDCT0232969: Indications: Solid Tumor, Hodgkin Lymphoma (B-Cell Hodgkin Lymphoma), Non-Hodgkin Lymphoma, OC, EC; GDCT0327878: Indications: OC, FTC, PC, EOC; GDCT0434405: Indications: Solid Tumor; GDCT0319681: Indications: OC, EC, EOC, FTC, PC, Gynecological Cancer.

Formulations and/or compositions disclosed herein can also be used to treat a complication or disease related to C/G AML. For example, complications relating to AML may include a preceding myelodysplastic syndrome (MDS, formerly known as “preleukemia”), secondary leukemia, in particular secondary AML, high white blood cell count, and absence of Auer rods. Among others, leukostasis and involvement of the central nervous system (CNS), hyperleukocytosis, residual disease, are also considered complications or diseases related to AML.

For administration, therapeutically effective amounts (also referred to herein as doses) can be initially estimated based on results from in vitro assays and/or animal model studies. Such information can be used to more accurately determine useful doses in subjects of interest. The actual dose amount administered to a particular subject can be determined by a physician, veterinarian or researcher taking into account parameters such as physical and physiological factors including target, body weight, severity of condition, stage of cancer, previous or concurrent therapeutic interventions, idiopathy of the subject and route of administration.

Therapeutically effective amounts of cell-based formulations can include 10⁴ to 10⁹ cells/kg body weight, or 10³ to 10¹¹ cells/kg body weight. Therapeutically effective amounts to administer can include greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹.

Therapeutically effective amounts of compositions can include 0.1 μg/kg to 5 mg/kg body weight, 0.5 μg/kg to 2 mg/kg, or 1 mg/kg to 4 mg/kg. Therapeutically effective amounts to administer can include greater than 0.1 μg/kg, greater than 0.6 μg/kg, greater than 1 mg/kg, greater than 2 mg/kg, greater than 3 mg/kg, greater than 4 mg/kg, or greater than 5 mg/kg.

Therapeutically effective amounts can be achieved by administering single or multiple doses during the course of a treatment regimen (e.g., daily, every other day, every 3 days, every 4 days, every 5 days, every 6 days, weekly, every 2 weeks, every 3 weeks, monthly, every 2 months, every 3 months, every 4 months, every 5 months, every 6 months, every 7 months, every 8 months, every 9 months, every 10 months, every 11 months or yearly). In particular embodiments, the treatment protocol may be dictated by a clinical trial protocol or an FDA-approved treatment protocol.

Therapeutically effective amounts can be administered by, e.g., injection, infusion, perfusion, or lavage. Routes of administration can include bolus intravenous, intradermal, intraarterial, intraparenteral, intranodal, intralymphatic, intraperitoneal, intralesional, intraprostatic, intrathecal, intratumoral, intravesicular, and/or subcutaneous.

In certain embodiments, formulations and/or compositions are administered to a patient in conjunction with (e.g., before, simultaneously or following) any number of relevant treatment modalities. In particular embodiments, cells may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycoplienolic acid, steroids, FR901228, cytokines, and irradiation.

(ix) Reference Levels Derived from Control Populations. Obtained values for parameters associated with a therapy described herein can be compared to a reference level derived from a control population, and this comparison can indicate whether a therapy described herein is effective for a subject in need thereof. Reference levels can be obtained from one or more relevant datasets from a control population. A “dataset” as used herein is a set of numerical values resulting from evaluation of a sample (or population of samples) under a desired condition. The values of the dataset can be obtained, for example, by experimentally obtaining measures from a sample and constructing a dataset from these measurements. As is understood by one of ordinary skill in the art, the reference level can be based on e.g., any mathematical or statistical formula useful and known in the art for arriving at a meaningful aggregate reference level from a collection of individual data points; e.g., mean, median, median of the mean, etc. Alternatively, a reference level or dataset to create a reference level can be obtained from a service provider such as a laboratory, or from a database or a server on which the dataset has been stored.

A reference level from a dataset can be derived from previous measures derived from a control population. A “control population” is any grouping of subjects or samples of like specified characteristics. The grouping could be according to, for example, clinical parameters, clinical assessments, therapeutic regimens, disease status, severity of condition, etc. In particular embodiments, the grouping is based on age range and non-immunocompromised status. In particular embodiments, a normal control population includes individuals that are age-matched to a test subject and non-immune compromised. In particular embodiments, age-matched includes, e.g., 0-1 years old; 1-2 years old, 2-4 years old, 4-5 years old, 5-18 years old, 18-25 years old, 25-50 years old, 50-80 years old, etc., as is clinically relevant under the circumstances. In particular embodiments, a control population can include those that have a cancer having cancer cells that express FOLR1, MEGF10, HPSE2, KLRF2, PCDH19, and/or FRAS1 and have not been administered a therapeutically effective amounts of compositions or formulations as described herein.

In particular embodiments, the relevant reference level for values of a particular parameter associated with a therapy described herein is obtained based on the value of a particular corresponding parameter associated with a therapy in a control population to determine whether a therapy disclosed herein has been therapeutically effective for a subject in need thereof.

In particular embodiments, conclusions are drawn based on whether a sample value is statistically significantly different or not statistically significantly different from a reference level. A measure is not statistically significantly different if the difference is within a level that would be expected to occur based on chance alone. In contrast, a statistically significant difference or increase is one that is greater than what would be expected to occur by chance alone. Statistical significance or lack thereof can be determined by any of various methods well-known in the art. An example of a commonly used measure of statistical significance is the p-value. The p-value represents the probability of obtaining a given result equivalent to a particular data point, where the data point is the result of random chance alone. A result is often considered significant (not random chance) at a p-value less than or equal to 0.05. In particular embodiments, a sample value is “comparable to” a reference level derived from a normal control population if the sample value and the reference level are not statistically significantly different.

(x) Cell Transformation Methods. The current disclosure also provides methods and assays to further study the cancer biology of C/G AML. A model of C/G AML cells is provided by expressing the C/G fusion construct in cells by any appropriate protein expression technology. In particular embodiments, the methods include inserting the C/G fusion construct into a vector, producing viral particles, and transducing a target cell type with the viral particle. In particular embodiments, the transduced cell type is cocultured with endothelial cells to recreate the microenvironment of C/G AML cells.

In particular embodiments, the C/G fusion construct can be inserted into a lentivirus vector. In particular embodiments, the C/G fusion construct is a MSCV-CBFA2T3-GLIS2-IRES-mCherry construct. In particular embodiments, the C/G fusion gene and MND promoter are inserted into a lentivirus vector. In particular embodiments, the lentivirus vector is a pRRLhPGK-GFP lentivirus vector.

In particular embodiments, the transduced cells include cord blood (CB) hematopoietic stem and progenitor cells (HSPCs). These cells are referred to herein as C/G-CB cells. In particular embodiments, transduced cells are grown on Notch ligand at 37° C. in 5% CO₂. In particular embodiments, transduced cells are transplanted into an animal or grown in micro-environment stimulating conditions in monoculture. In particular embodiments, micro-environment stimulating conditions include co-culture with endothelial cells. In particular embodiments, micro-environment stimulating conditions include myeloid promoting conditions. In particular embodiments, cells are in monoculture at 75,000 cells per well in a 6-well plate. In particular embodiments, cells are in monoculture at 300,000 cells per well in a 12-well plate.

Co-culture with endothelial cells or EC co-culture includes culture with endothelial cells in serum free expansion medium (SFEM) II supplemented with 50 ng/mL SCF, 50 ng/mL TPO, 50 ng/mL FLT3L, and 100U/mL Penicillin/Streptomycin. In particular embodiments, endothelial cells include human umbilical vein endothelial cells (HUVECs). In particular embodiments, endothelial cells are transduced with E4ORF1 construct and propagated. In particular embodiments, endothelial cells are seeded at 800,000 cells per well in a 6-well plate. In particular embodiments, endothelial cells are seeded at 300,000 cells per well in a 12-well plate. Endothelial cells can be cultured in medium 199 supplemented with FBS, endothelial mitogen, Heparin, HEPES, L-Glutamine, and Penicillin/Streptomycin. Before co-culture, endothelial cells can be washed with buffer (e.g., phosphate buffered saline). In EC co-culture, endothelial cells can be replaced every week. In particular embodiments, 3-20% of the cultures are replated every week.

Myeloid promoting conditions or MC include Iscove's Modified Dulbecco's Medium (IMDM, Gibco 12-440-053) supplemented with 15% fetal bovine serum (FBS, Corning, 35-010-CV), 100U/mL Penicillin-Streptomycin (Pen/Strep, Gibco, 15-140-122), 10 ng/mL SCF, 10 ng/mL TPO, 10 ng/mL FLT3L, 10 ng/mL IL-6 (Shenandoah Biotechnology, Cat #100-10), and 10 ng/mL IL3 (Shenandoah, Cat #100-80).

The Exemplary Embodiments and Examples below are included to demonstrate particular, non-limiting embodiments of the disclosure. Those of ordinary skill in the art will recognize in light of the present disclosure that many changes can be made to the specific embodiments disclosed herein and still obtain a like or similar result without departing from the spirit and scope of the disclosure.

### (xi) Exemplary Embodiments

1. A targeted therapeutic molecule including a binding domain that binds folate receptor 1 (FOLR1), multiple EGF like domain 10 (MEGF10), heparinase-2 enzyme (HPSE2), killer cell lectin like receptor F2 (KLRF2), protocadherin-19 (PCDH19), or Fraser extracellular matrix complex subunit 1 (FRAS1).
2. The targeted therapeutic molecule of embodiment 1, wherein the targeted therapeutic molecule is a chimeric antigen receptor (CAR) including, when expressed by a cell, an extracellular component including the binding domain that binds FOLR1, MEGF10, HPSE2, KLRF2, PCDH19, or FRAS1;
   an intracellular component including an effector domain; and
   a transmembrane domain linking the extracellular component to the intracellular component.
3. The targeted therapeutic molecule of embodiment 2, wherein the binding domain specifically binds FOLR1.
4. The targeted therapeutic molecule of embodiments 2 or 3, wherein the binding domain includes a single chain variable fragment (scFv).
5. The targeted therapeutic molecule of embodiment 4, wherein the scFv has the sequence as set forth in SEQ ID NO: 22 or SEQ ID NO: 23 or has a sequence with at least 95% sequence identity to the sequence as set forth in SEQ ID NO: 22 or SEQ ID NO: 23.
6. The targeted therapeutic molecule of any of embodiments 3-5, wherein the binding domain includes
   a variable heavy chain set forth in SEQ ID NO: 30 and a variable light chain set forth in SEQ ID NO: 31 or a variable heavy chain having at least 95% sequence identity to the sequence as set forth in SEQ ID NO: 30 and a variable light chain having at least 95% sequence identity to the sequence as set forth in SEQ ID NO: 31;
   a variable heavy chain set forth in SEQ ID NO: 38 and a variable light chain set forth in SEQ ID NO: 39 or a variable heavy chain having at least 95% sequence identity to the sequence as set forth in SEQ ID NO: 38 and a variable light chain having at least 95% sequence identity to the sequence as set forth in SEQ ID NO: 39;
   a variable heavy chain set forth in SEQ ID NO: 40 and a variable light chain set forth in SEQ ID NO: 41 or a variable heavy chain having at least 95% sequence identity to the sequence as set forth in SEQ ID NO: 40 and a variable light chain having at least 95% sequence identity to the sequence as set forth in SEQ ID NO: 41;
   a variable heavy chain set forth in SEQ ID NO: 48 and a variable light chain set forth in SEQ ID NO: 49 or a variable heavy chain having at least 95% sequence identity to the sequence as set forth in SEQ ID NO: 48 and a variable light chain having at least 95% sequence identity to the sequence as set forth in SEQ ID NO: 49;
   a variable heavy chain set forth in SEQ ID NO: 56 and a variable light chain set forth in SEQ ID NO: 57 or a variable heavy chain having at least 95% sequence identity to the sequence as set forth in SEQ ID NO: 56 and a variable light chain having at least 95% sequence identity to the sequence as set forth in SEQ ID NO: 57;
   a variable heavy chain set forth in SEQ ID NO: 64 and a variable light chain set forth in SEQ ID NO: 65 or a variable heavy chain having at least 95% sequence identity to the sequence as set forth in SEQ ID NO: 64 and a variable light chain having at least 95% sequence identity to the sequence as set forth in SEQ ID NO: 65;
   a variable heavy chain set forth in SEQ ID NO: 72 and a variable light chain set forth in SEQ ID NO: 73 or a variable heavy chain having at least 95% sequence identity to the sequence as set forth in SEQ ID NO: 72 and a variable light chain having at least 95% sequence identity to the sequence as set forth in SEQ ID NO: 73; or
   a variable heavy chain set forth in SEQ ID NO: 80 and a variable light chain set forth in SEQ ID NO: 81 or a variable heavy chain having at least 95% sequence identity to the sequence as set forth in SEQ ID NO: 80 and a variable light chain having at least 95% sequence identity to the sequence as set forth in SEQ ID NO: 81.
7. The targeted therapeutic molecule of any of embodiments 3-6, wherein the binding domain includes a variable heavy chain with complementarity determining regions (CDRH) 1 as set forth in SEQ ID NO: 24, a CDRH2 as set forth in SEQ ID NO: 25, and a CDRH3 as set forth in SEQ ID NO: 26, and
   a variable light chain complementarity determining region (CDRL) 1 as set forth in SEQ ID NO: 27, a CDRL2 as set forth in SEQ ID NO: 28, and a CDRL3 as set forth in SEQ ID NO: 29;
   a CDRH1 as set forth in SEQ ID NO: 32, a CDRH2 as set forth in SEQ ID NO: 33, and a CDRH3 as set forth in SEQ ID NO: 34, and
   a CDRL1 as set forth in SEQ ID NO: 35, a CDRL2 as set forth in SEQ ID NO: 36, and a CDRL3 as set forth in SEQ ID NO: 37;
   a CDRH1 as set forth in SEQ ID NO: 42, a CDRH2 as set forth in SEQ ID NO: 43, and a CDRH3 as set forth in SEQ ID NO: 44, and
   a CDRL1 as set forth in SEQ ID NO: 45, a CDRL2 as set forth in SEQ ID NO: 46, and a CDRL3 as set forth in SEQ ID NO: 47;
   a CDRH1 as set forth in SEQ ID NO: 50, a CDRH2 as set forth in SEQ ID NO: 51, and a CDRH3 as set forth in SEQ ID NO: 52, and
   a CDRL1 as set forth in SEQ ID NO: 53, a CDRL2 as set forth in SEQ ID NO: 54, and a CDRL3 as set forth in SEQ ID NO: 55;
   a CDRH1 as set forth in SEQ ID NO: 58, a CDRH2 as set forth in SEQ ID NO: 59, and a CDRH3 as set forth in SEQ ID NO:60, and
   a CDRL1 as set forth in SEQ ID NO: 61, a CDRL2 as set forth in SEQ ID NO: 62, and a CDRL3 as set forth in SEQ ID NO: 63;
   a CDRH1 as set forth in SEQ ID NO: 66, a CDRH2 as set forth in SEQ ID NO: 67, and a CDRH3 as set forth in SEQ ID NO: 68, and
   a CDRL1 as set forth in SEQ ID NO: 69, a CDRL2 as set forth in SEQ ID NO: 70, and a CDRL3 as set forth in SEQ ID NO: 71; and
   a CDRH1 as set forth in SEQ ID NO: 74, a CDRH2 as set forth in SEQ ID NO: 75, and a CDRH3 as set forth in SEQ ID NO: 76, and
   a CDRL1 as set forth in SEQ ID NO: 77, a CDRL2 as set forth in SEQ ID NO: 78, and a CDRL3 as set forth in SEQ ID NO: 79,
   according to the Kabat numbering scheme.
8. The targeted therapeutic molecule of embodiment 2, encoded by the sequence as set forth in SEQ ID NO: 134 or a sequence having at least 90% sequence identity to the sequence as set forth in SEQ ID NO: 134.
9. The targeted therapeutic molecule of embodiment 2, wherein the binding domain specifically binds MEGF10.
10. The targeted therapeutic molecule of embodiment 9, wherein the binding domain includes LS-C678634, LS-C668447, LSC497216, or PA5-76556, or a binding fragment thereof.
11. The targeted therapeutic molecule of embodiment 2, wherein the binding domain specifically binds HPSE2.
12. The targeted therapeutic molecule of embodiment 11, wherein the binding domain includes LS-B14593, LS-C322089, LS-C378319, or HPA044603, or a binding fragment thereof.
13. The targeted therapeutic molecule of embodiment 2, wherein the binding domain specifically binds KLRF2.
14. The targeted therapeutic molecule of embodiment 13, wherein the binding domain includes LS-C329740, LS-C203747, SAB2108513, SAB2108684, HPA055964, SAB2108320, or SAB2108355, or a binding fragment thereof.
15. The targeted therapeutic molecule of embodiment 2, wherein the binding domain specifically binds PCDH19.
16. The targeted therapeutic molecule of embodiment 15, wherein the binding domain includes LS-C676224, LS-C496779, LS-C761991, HPA027533, or HPA001461, or a binding fragment thereof.
17. The targeted therapeutic molecule of embodiment 2, wherein the binding domain specifically binds FRAS1.
18. The targeted therapeutic molecule of embodiment 17, wherein the binding domain includes LS-C763132, LS-B5486, LS-C754337, HPA011281, or HPA051601, or a binding fragment thereof.
19. The targeted therapeutic molecule of any of embodiments 2-18, wherein the extracellular component further includes a spacer region.
20. The targeted therapeutic molecule of embodiment 19, wherein the spacer region includes a long spacer region, intermediate spacer region, or short spacer region.
21. The targeted therapeutic molecule of embodiment 20, wherein the intermediate spacer region is 135 amino acids or less.
22. The targeted therapeutic molecule of embodiments 20 or 21, wherein the intermediate spacer region is 131 amino acids or less and includes a hinge region and a CH3 domain of IgG4.
23. The targeted therapeutic molecule of embodiment 22, wherein the intermediate spacer region is encoded by the sequence as set forth in SEQ ID NO: 136 or a sequence having at least 90% sequence identity to the sequence as set forth in SEQ ID NO: 136.
24. The targeted therapeutic molecule of any of embodiments 20-23, wherein the intermediate spacer region is encoded by the sequence as set forth in SEQ ID NO: 3 or a sequence having at least 90% sequence identity to the sequence as set forth in SEQ ID NO: 3.
25. The targeted therapeutic molecule of embodiment 20, wherein the long spacer region is greater than 200 amino acids and includes an IgG4 hinge, IgG4 CH3 region, and an IgG4 CH2 region.
26. The targeted therapeutic molecule of embodiments 20 or 25, wherein the long spacer region is encoded by the sequence as set forth in SEQ ID NO: 4 or a sequence having at least 90% sequence identity to the sequence as set forth in SEQ ID NO: 4.
27. The targeted therapeutic molecule of embodiment 20, wherein the short spacer region is less than 50 amino acids and includes an IgG4 hinge.
28. The targeted therapeutic molecule of embodiments 20 or 27, wherein the short spacer region is encoded by the sequence as set forth in SEQ ID NO: 1 or SEQ ID NO: 2 or a sequence having at least 90% sequence identity to the sequence as set forth in SEQ ID NO: 1 or SEQ ID NO: 2.
29. The targeted therapeutic molecule of any of embodiments 2-28, wherein the intracellular effector domain includes all or a portion of the signaling domain of CD3ζ and 4-1BB.
30. The targeted therapeutic molecule of embodiment 29, wherein the CD3ζ signaling domain is encoded by the CD3ζ coding sequence as set forth in SEQ ID NO: 5 or a sequence having at least 90% sequence identity to the sequence as set forth in SEQ ID NO: 5.
31. The targeted therapeutic molecule of embodiments 29 or 30, wherein the CD3ζ signaling domain includes the sequence as set forth in SEQ ID NO: 6 or SEQ ID NO: 7 or a sequence having at least 90% sequence identity to the sequence as set forth in SEQ ID NO: 6 or SEQ ID NO: 7.
32. The targeted therapeutic molecule of any of embodiments 29-31, wherein the 4-1BB signaling domain is encoded by SEQ ID NO: 8 or SEQ ID NO: 9 or a sequence having at least 90% sequence identity to the sequence as set forth in SEQ ID NO: 8 or SEQ ID NO: 9.
33. The targeted therapeutic molecule of any of embodiments 29-32, wherein the 4-1BB signaling domain includes the sequence as set forth in SEQ ID NO: 10 or SEQ ID NO: 11 or a sequence having at least 90% sequence identity to the sequence as set forth in SEQ ID NO: 10 or SEQ ID NO: 11.
34. The targeted therapeutic molecule of any of embodiments 2-33, wherein the transmembrane domain includes a CD28 transmembrane domain.
35. The targeted therapeutic molecule of embodiment 34, wherein the CD28 transmembrane domain is encoded by SEQ ID NO: 12, SEQ ID NO: 13, or SEQ ID NO: 14 or a sequence having at least 90% sequence identity to the sequence as set forth in SEQ ID NO: 12, SEQ ID NO: 13, or SEQ ID NO: 14.
36. The targeted therapeutic molecule of embodiments 34 or 35, wherein the CD28 transmembrane domain includes SEQ ID NO: 15 or SEQ ID NO: 16 or a sequence having at least 90% sequence identity to the sequence as set forth in SEQ ID NO: 15 or SEQ ID NO: 16.
37. The targeted therapeutic molecule of any of embodiments 2-36, further including a control feature selected from a tag cassette, a transduction marker, and/or a suicide switch.
38. The targeted therapeutic molecule of embodiment 37, wherein the transduction marker includes a truncated CD19.
39. The targeted therapeutic molecule of embodiment 38, wherein the truncated CD19 is encoded by SEQ ID NO: 117 or a sequence having at least 90% sequence identity to the sequence as set forth in SEQ ID NO: 117.
40. The targeted therapeutic molecule of any of embodiments 2-39, further including a ribosomal skip element.
41. The targeted therapeutic molecule of embodiment 40, wherein the ribosomal skip element includes T2A, P2A, E2A, or F2A.
42. The targeted therapeutic molecule of embodiments 40 or 41, wherein the ribosomal skip element includes T2A.
43. The targeted therapeutic molecule of embodiment 42, wherein T2A is encoded by SEQ ID NO: 137 or a sequence having at least 90% sequence identity to the sequence as set forth in SEQ ID NO: 137.
44. A genetic construct encoding the CAR of any of embodiments 2-43.
45. A nanoparticle encapsulating the genetic construct of embodiments 44.
46. A cell genetically modified to express the CAR of any of embodiments 2-43.
47. The cell of embodiment 46, wherein the cell is an autologous cell or an allogeneic cell in reference to a subject.
48. The cell of embodiments 46 or 47, wherein the cell is in vivo or ex vivo.
49. The cell of any of embodiments 46-48, wherein the cell is a T cell, B cell, natural killer (NK) cell, NK-T cell, monocyte/macrophage, hematopoietic stem cells (HSC), or a hematopoietic progenitor cell (HPC).
50. The cell of embodiment 49, wherein the cell is a T cell selected from a CD3+ T cell, a CD4+ T cell, a CD8+ T cell, a central memory T cell, an effector memory T cell, and/or a naive T cell.
51. The cell of embodiments 49 or 50, wherein the cell is a CD8+ T cell and/or a CD4+ T cell.
52. The targeted therapeutic molecule of any of embodiments 1-40, wherein the binding domain is conjugated to a cytotoxic payload.
53. The targeted therapeutic of any of embodiments 1-43, wherein the binding domain specifically binds FOLR1.
54. The targeted therapeutic of any of embodiments 1-43, wherein the binding domain includes a single chain variable fragment (scFv).
55. The targeted therapeutic of embodiment 54, wherein the scFv has the sequence as set forth in SEQ ID NO: 22 or SEQ ID NO: 23 or a sequence having at least 90% sequence identity to the sequence as set forth in SEQ ID NO: 22 or SEQ ID NO: 23.
56. The targeted therapeutic of any of embodiments 52-55, wherein the binding domain includes
   a variable heavy chain set forth in SEQ ID NO: 30 and a variable light chain set forth in SEQ ID NO: 31 or a variable heavy chain having at least 95% sequence identity to the sequence as set forth in SEQ ID NO: 30 and a variable light chain having at least 95% sequence identity to the sequence as set forth in SEQ ID NO: 31;
   a variable heavy chain set forth in SEQ ID NO: 38 and a variable light chain set forth in SEQ ID NO: 39 or a variable heavy chain having at least 95% sequence identity to the sequence as set forth in SEQ ID NO: 38 and a variable light chain having at least 95% sequence identity to the sequence as set forth in SEQ ID NO: 39;
   a variable heavy chain set forth in SEQ ID NO: 40 and a variable light chain set forth in SEQ ID NO: 41 or a variable heavy chain having at least 95% sequence identity to the sequence as set forth in SEQ ID NO: 40 and a variable light chain having at least 95% sequence identity to the sequence as set forth in SEQ ID NO: 41;
   a variable heavy chain set forth in SEQ ID NO: 48 and a variable light chain set forth in SEQ ID NO: 49 or a variable heavy chain having at least 95% sequence identity to the sequence as set forth in SEQ ID NO: 48 and a variable light chain having at least 95% sequence identity to the sequence as set forth in SEQ ID NO: 49;
   a variable heavy chain set forth in SEQ ID NO: 56 and a variable light chain set forth in SEQ ID NO: 57 or a variable heavy chain having at least 95% sequence identity to the sequence as set forth in SEQ ID NO: 56 and a variable light chain having at least 95% sequence identity to the sequence as set forth in SEQ ID NO: 57;
   a variable heavy chain set forth in SEQ ID NO: 64 and a variable light chain set forth in SEQ ID NO: 65 or a variable heavy chain having at least 95% sequence identity to the sequence as set forth in SEQ ID NO: 64 and a variable light chain having at least 95% sequence identity to the sequence as set forth in SEQ ID NO: 65;
   a variable heavy chain set forth in SEQ ID NO: 72 and a variable light chain set forth in SEQ ID NO: 73 or a variable heavy chain having at least 95% sequence identity to the sequence as set forth in SEQ ID NO: 72 and a variable light chain having at least 95% sequence identity to the sequence as set forth in SEQ ID NO: 73; or
   a variable heavy chain set forth in SEQ ID NO: 80 and a variable light chain set forth in SEQ ID NO: 81 or a variable heavy chain having at least 95% sequence identity to the sequence as set forth in SEQ ID NO: 80 and a variable light chain having at least 95% sequence identity to the sequence as set forth in SEQ ID NO: 81.
57. The targeted therapeutic of any of embodiments 52-55, wherein the binding domain includes
   a variable heavy chain with complementarity determining regions (CDRH) 1 as set forth in SEQ ID NO: 24, a CDRH2 as set forth in SEQ ID NO: 25, and a CDRH3 as set forth in SEQ ID NO: 26, and
   a variable light chain complementarity determining region (CDRL) 1 as set forth in SEQ ID NO: 27, a CDRL2 as set forth in SEQ ID NO: 28, and a CDRL3 as set forth in SEQ ID NO: 29;
   a CDRH1 as set forth in SEQ ID NO: 32, a CDRH2 as set forth in SEQ ID NO: 33, and a CDRH3 as set forth in SEQ ID NO: 34, and
   a CDRL1 as set forth in SEQ ID NO: 35, a CDRL2 as set forth in SEQ ID NO: 36, and a CDRL3 as set forth in SEQ ID NO: 37;
   a CDRH1 as set forth in SEQ ID NO: 42, a CDRH2 as set forth in SEQ ID NO: 43, and a CDRH3 as set forth in SEQ ID NO: 44, and
   a CDRL1 as set forth in SEQ ID NO: 45, a CDRL2 as set forth in SEQ ID NO: 46, and a CDRL3 as set forth in SEQ ID NO: 47;
   a CDRH1 as set forth in SEQ ID NO: 50, a CDRH2 as set forth in SEQ ID NO: 51, and a CDRH3 as set forth in SEQ ID NO: 52, and
   a CDRL1 as set forth in SEQ ID NO: 53, a CDRL2 as set forth in SEQ ID NO: 54, and a CDRL3 as set forth in SEQ ID NO: 55;
   a CDRH1 as set forth in SEQ ID NO: 58, a CDRH2 as set forth in SEQ ID NO: 59, and a CDRH3 as set forth in SEQ ID NO:60, and
   a CDRL1 as set forth in SEQ ID NO: 61, a CDRL2 as set forth in SEQ ID NO: 62, and a CDRL3 as set forth in SEQ ID NO: 63;
   a CDRH1 as set forth in SEQ ID NO: 66, a CDRH2 as set forth in SEQ ID NO: 67, and a CDRH3 as set forth in SEQ ID NO: 68, and
   a CDRL1 as set forth in SEQ ID NO: 69, a CDRL2 as set forth in SEQ ID NO: 70, and a CDRL3 as set forth in SEQ ID NO: 71; and
   a CDRH1 as set forth in SEQ ID NO: 74, a CDRH2 as set forth in SEQ ID NO: 75, and a CDRH3 as set forth in SEQ ID NO: 76, and
   a CDRL1 as set forth in SEQ ID NO: 77, a CDRL2 as set forth in SEQ ID NO: 78, and a CDRL3 as set forth in SEQ ID NO: 79,
   according to the Kabat numbering scheme.
58. The targeted therapeutic molecule of any of embodiments 1-43, wherein the binding domain specifically binds MEGF10.
59. The targeted therapeutic molecule of embodiment 58, wherein the binding domain includes LS-C678634, LS-C668447, LSC497216, or PA5-76556, or a binding fragment thereof.
60. The targeted therapeutic of any of embodiments 1-43, wherein the binding domain specifically binds HPSE2.
61. The targeted therapeutic molecule of embodiment 60, wherein the binding domain includes LS-B14593, LS-C322089, LS-C378319, or HPA044603, or a binding fragment thereof.
62. The targeted therapeutic molecule of any of embodiments 1-43, wherein the binding domain specifically binds KLRF2.
63. The targeted therapeutic molecule of embodiment 62, wherein the binding domain includes LS-C329740, LS-C203747, SAB2108513, SAB2108684, HPA055964, SAB2108320, or SAB2108355, or a binding fragment thereof.
64. The targeted therapeutic molecule of any of embodiments 1-43, wherein the binding domain specifically binds PCDH19.
65. The targeted therapeutic molecule of embodiment 64, wherein the binding domain includes LS-C676224, LS-C496779, LS-C761991, HPA027533, or HPA001461, or a binding fragment thereof.
66. The targeted therapeutic molecule of any of embodiments 1-43, wherein the binding domain specifically binds FRAS1.
67. The targeted therapeutic molecule of embodiment 66, wherein the binding domain includes LS-C763132, LS-B5486, LS-C754337, HPA011281, or HPA051601, or a binding fragment thereof.
68. The targeted therapeutic molecule of any of embodiments 52-67, wherein the cytotoxic payload includes a cytotoxin, a cytotoxic drug, a radioisotope, or a nanoparticle.
69. The targeted therapeutic molecule of embodiment 68, wherein the cytotoxin includes a holotoxin or a hemitoxin.
70. The targeted therapeutic molecule of embodiment 68, wherein the cytotoxic drug includes actinomycin D, anthracycline, auristatin, calicheamicin, camptothecin, CC1065, colchicin, cytochalasin B, daunorubicin, 1-dehydrotestosterone, dihydroxy anthracinedione, dolastatin, doxorubicin, duocarmycin, elinafide, emetine, ethidium bromide, etoposide, gramicidin D, glucocorticoids, lidocaine, maytansinoid, mithramycin, mitomycin, mitoxantrone, nemorubicin, PNU-159682, procaine, propranolol, puromycin, pyrrolobenzodiazepine (PBD), taxane, taxol, tenoposide, tetracaine, trichothecene, vinblastine, vinca alkaloid, vincristine, or stereoisomers, isosteres, analogs, or derivatives thereof.
71. The targeted therapeutic molecule of embodiment 68, wherein the radioisotope includes ²²⁸Ac, ¹¹¹Ag, ¹²⁴Am, ⁷⁴As, ²¹¹As, ²⁰⁹At, ¹⁹⁴Au, ¹²⁸Ba, ⁷Be, ²⁰⁶Bi, ²⁴⁵Bk, ²⁴⁶Bk, ⁷⁶Br, ¹¹C, ⁴⁷Ca, ²⁵⁴Cf, ²⁴²Cm, ⁵¹Cr, ⁶⁷Cu, ¹⁵³Dy, ¹⁵⁷Dy, ¹⁵⁹Dy, ¹⁶⁵Dy, ¹⁶⁶Dy, ¹⁷¹Er, ²⁵⁰Es, ²⁵⁴Es, ¹⁴⁷Eu, ¹⁵⁷Eu, ⁵²Fe, ⁵⁹Fe, ²⁵¹Fm, ²⁵²Fm, ²⁵³Fm, ⁶⁶Ga, ⁷²Ga, ¹⁴⁶Gd, ¹⁵³Gd, ⁶⁸Ge, ¹⁷⁰Hf, ¹⁷¹Hf, ¹⁹³Hg, ¹⁹³mHg, ¹⁶⁰mHo, ¹³⁰I, ¹³¹I, ¹³⁵I, ¹¹⁴mIn, ¹⁸⁵Ir, ⁴²K, ⁴³K, ⁷⁶Kr, ⁷⁹Kr, ⁸¹mKr, ¹³²La, ²⁶²Lr, ¹⁶⁹Lu, ¹⁷⁴mLu, ¹⁷⁶mLu, ²⁵⁷Md, ²⁶⁰Md, ²⁸Mg, ⁵²Mn, ⁹⁰Mo, ²⁴Na, ⁹⁵Nb, ¹³⁸Nd, ⁵⁷Ni, ⁶⁶Ni, ²³⁴Np, ¹⁵O, ¹⁸²Os, ¹⁸⁹mOs, ¹⁹¹Os, ³²P²⁰¹Pb, ¹⁰¹Pd, ¹⁴³Pr, ¹⁹¹Pt, ²⁴³Pu ²²⁵Ra, ⁸¹Rb, ¹⁸⁸Re, ¹⁰⁵Rh, ²¹¹Rn, ¹⁰³Ru, ³⁵S, ⁴⁴Sc, ⁷²Se, ¹⁵³Sm, ¹²⁵Sn, ⁹¹Sr, ¹⁷³Ta, ¹⁵⁴Tb, ¹²⁷Te, ²³⁴Th, ⁴⁵Ti, ¹⁶⁶Tm, ²³⁰U, ²³⁷U, ²⁴⁰U, ⁴⁸V, ¹⁷⁸W, ¹⁸¹W, ¹⁸⁸W, ¹²⁵Xe, ¹²⁷Xe, ¹³³Xe, ¹³³mXe, ¹³⁵Xe, ⁸⁵mY, ⁸⁶Y, ⁹⁰Y, ⁹³Y, ¹⁶⁹Yb, ¹⁷⁵Yb, ⁶⁵Zn, ⁷¹mZn, ⁸⁶Zr, ⁹⁵Zr, and/or ⁹⁷Zr.
72. The targeted therapeutic molecule of embodiment 68, wherein the nanoparticle includes a metal nanoparticle, a liposome, or a polymer nanoparticle.
73. A formulation including cells genetically modified to express the CAR system of any of embodiments 2-43.
74. The formulation of embodiment 73, wherein the cells are T cells, natural killer cells, monocyte/macrophages, hematopoietic stem cells or hematopoietic progenitor cells.
75. The formulation of embodiment 74, wherein the T cells are selected from CD3 T cells, CD4 T cells, CD8 T cells, central memory T cells, effector memory T cells, and/or naive T cells.
76. The formulation of embodiments 74 or 75, wherein the T cells are CD4 T cells and/or CD8 T cells.
77. The formulation of any of embodiments 73-76, further including a pharmaceutically acceptable carrier.
78. A composition including the targeted therapeutic of any of embodiments 52-72 and a pharmaceutically acceptable carrier.
79. A method of treating a subject in need thereof including administering a therapeutically effective amount of the formulation of any of embodiments 73-77 and/or the composition of embodiment 78 to the subject thereby treating the subject in need thereof.
80. The method of embodiment 79, wherein the subject in need thereof has cancer.
81. The method of embodiment 80, wherein the cancer includes cancer cells expressing FOLR1, MEGF10, HPSE2, KLRF2, PCDH19, or FRAS1.
82. The method of embodiment 81, wherein the cancer includes leukemia.
83. The method of embodiment 82, wherein the leukemia is acute myeloid leukemia (AML).
84. The method of embodiment 83, wherein the AML includes CBFA2T3/GLIS2 AML.
85. The method of any of embodiments 80-84, wherein the cancer includes cancer cells expressing FOLR1.
86. The method of embodiment 85, wherein the cancer includes leukemia, peritoneal cancer, fallopian tube cancer, ovarian cancer, endometrial cancer, cervical cancer, breast cancer, bladder cancer, renal cell carcinoma, pituitary tumors, lung cancer, uterine cancer, squamous cell carcinoma, ureter cancer, urethral cancer, osteosarcoma, or transitional cell carcinoma.
87. The method of embodiment 86, wherein the cancer is metastatic.
88. The method of embodiment 86 or 87, wherein the ovarian cancer includes epithelial ovarian cancer.
89. The method of embodiment 86 or 87, wherein the breast cancer includes triple-negative breast cancer or HER2-breast cancer.
90. The method of embodiment 86 or 87, wherein the lung cancer includes lung adenocarcinoma or epithelial lung cancer such as non-small cell lung cancer.
91. The method of any of embodiments 79-90, wherein the formulation includes autologous cells or allogeneic cells.
92. A method of treating a subject with CBFA2T3/GLIS2 acute myeloid leukemia (AML) including administering a therapeutically effective amount of the formulation of any of embodiments 73-77 and/or the composition of embodiment 78 to the subject thereby treating the subject with the CBFA2T3/GLIS2 AML.
93. The method of embodiment 92, wherein the formulation includes autologous cells or allogeneic cells.

(xii) Experimental Examples. Example 1. CBFA2T3-GLIS2 oncogenic fusion is sufficient for leukemic transformation.

Abstract. Fusion oncoproteins are the initiating event in acute myeloid leukemia (AML) pathogenesis, although they are thought to require additional cooperating mutations for leukemic transformation. CBFA2T3-GLIS2 (C/G) fusion occurs exclusively in infants and is associated with highly aggressive disease (de Rooij et al., Nat Genet 49: 451-456, 2017; Gruber et al., Cancer Cell 22, 683-697, 2012; Masetti et al., Blood 121: 3469-3472, 2013; and Smith et al., Clin Cancer Res 26: 726-737, 2020). Here it is reported that lentiviral transduction of C/G fusion is sufficient to induce malignant transformation of human cord blood hematopoietic stem and progenitor cells (CB HSPCs) that fully recapitulates C/G AML. Engineered CB HSPCs co-cultured with endothelial cells undergo complete malignant transformation with identical molecular, morphologic, phenotypic and disease characteristics observed in primary C/G AML. Interrogating the transcriptome of engineered cells identified a library of C/G fusion-specific targets that are candidates for chimeric antigen receptor (CAR) T cell therapy. CAR-T cells directed against one of the targets, FOLR1, were developed. These CAR-T cells demonstrated the pre-clinical efficacy against C/G AML while sparing normal hematopoiesis. The findings underscore the role of the endothelial niche in promoting leukemic transformation of C/G-transduced CB HSPCs. Moreover, this work has broad implications for studies of leukemogenesis applicable to a variety of oncogenic fusion-driven pediatric leukemias, providing a robust and tractable model system to characterize the molecular mechanisms of leukemogenesis and identify biomarkers for disease diagnosis and targets for therapy.

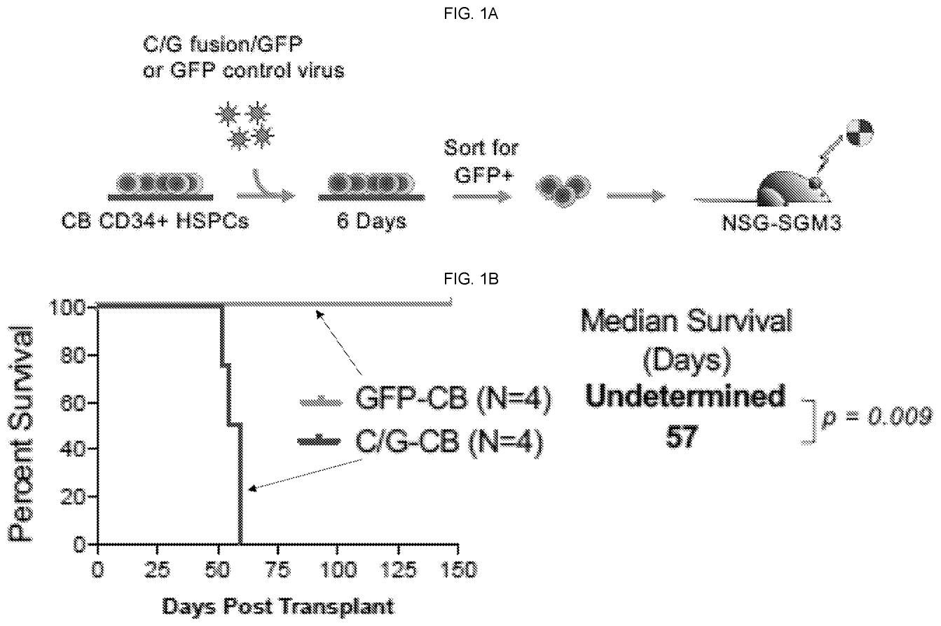

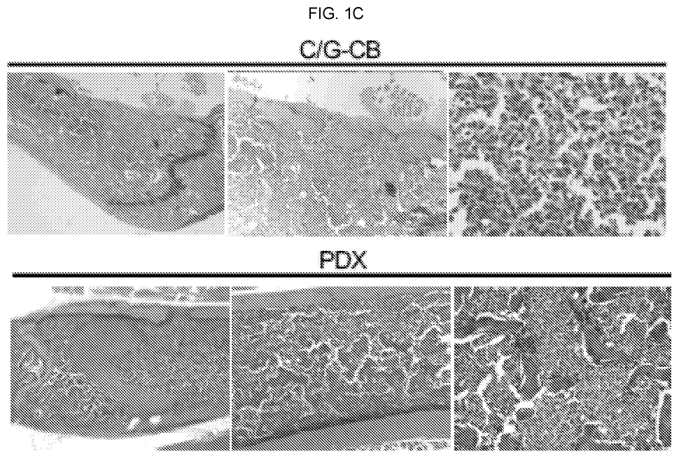

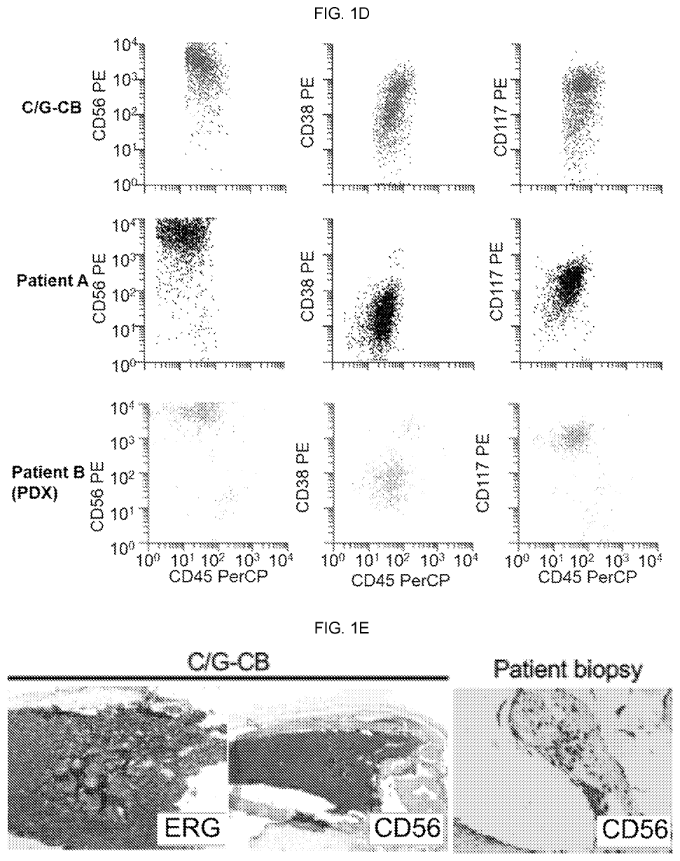

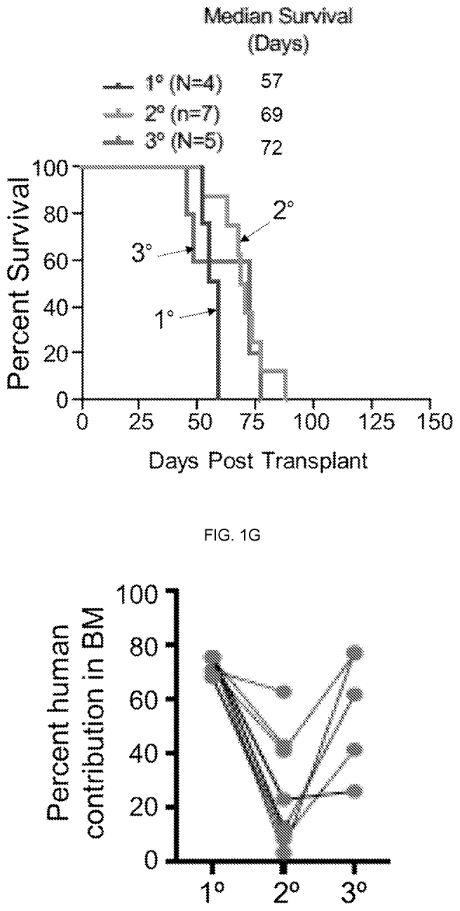

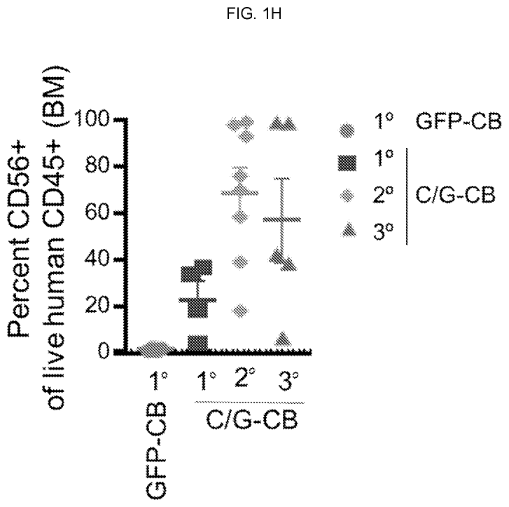

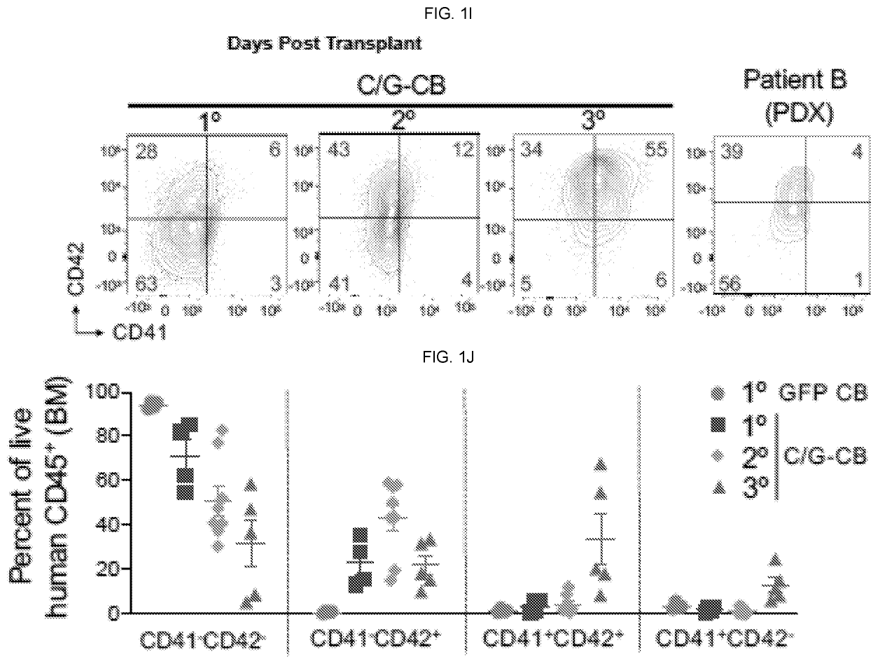

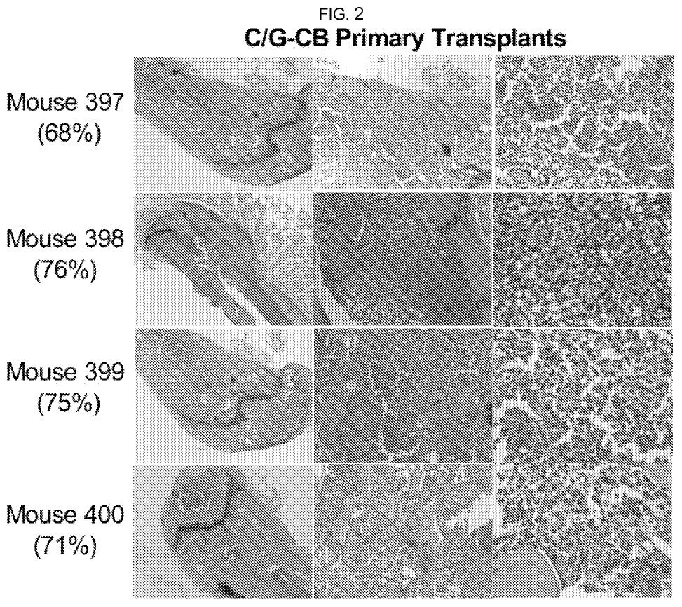

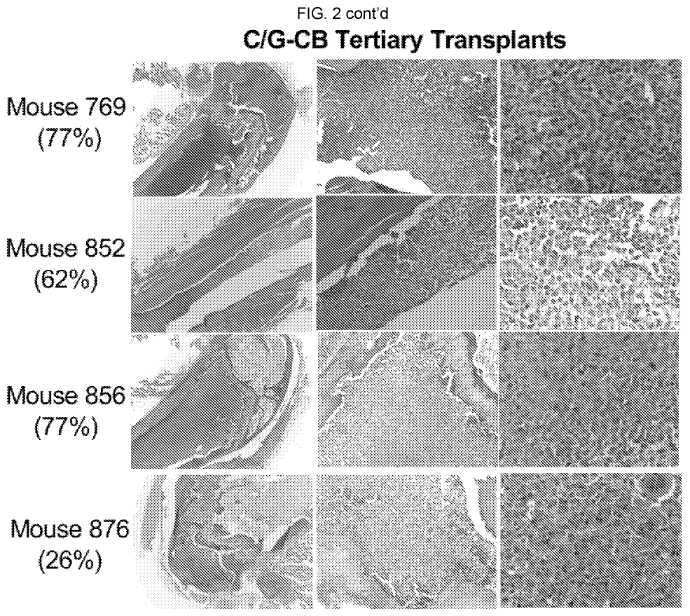

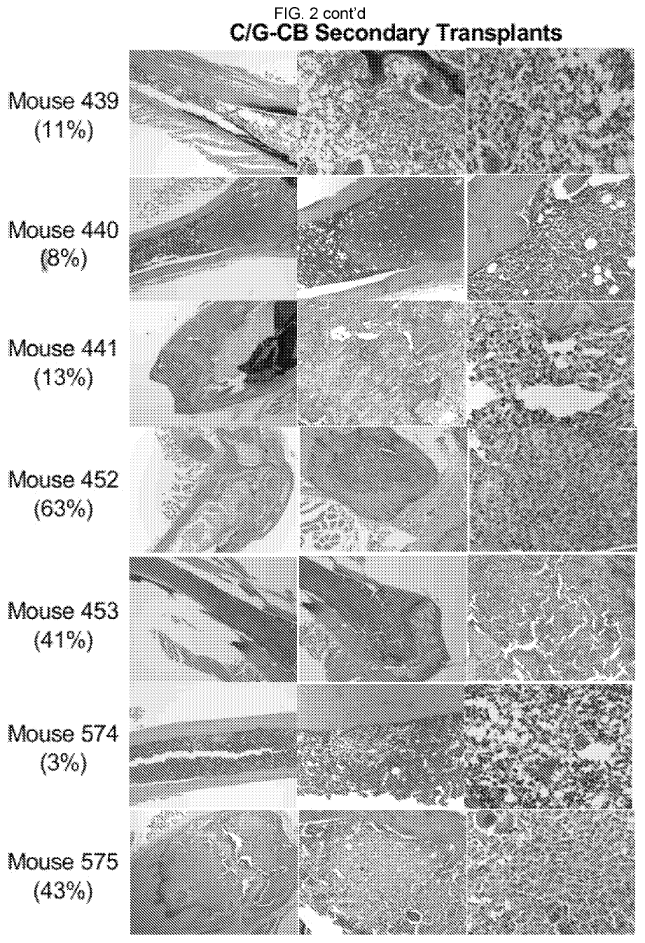

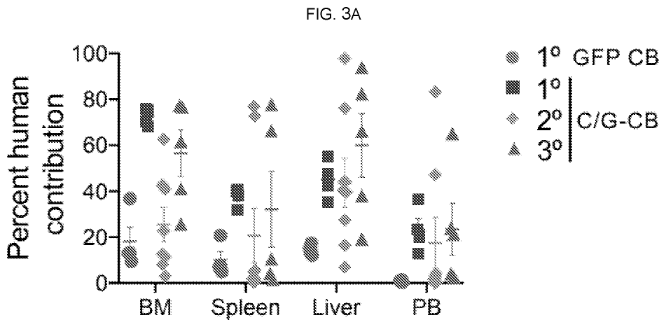

Results. C/G expression transforms human CB HSPCs. CEFA2T3 (ETO2) is a member of the ETO family of transcription factors. Its fusion partner GLIS2 is a zinc finger protein regulated by the Hedgehog pathway. C/G AML is devoid of recurrent cooperating mutations (Gruber et al., Cancer Cell 22, 683-697, 2012; Smith et al., Clin Cancer Res 26: 726-737, 2020; and Bolouri et al., Nat Med 25: 530, 2019), suggesting that the fusion might be sufficient for malignant transformation. To test this, the C/G fusion or GFP control were expressed in CB HSPCs (C/G-CB or GFP-CB) by lentiviral transduction and transplanted the transduced cells into NSG-SGM3 mice (FIG. 1A). Within 60 days of transplant, all mice (4/4) injected with C/G-CB cells developed florid leukemia, while all control mice (4/4) survived until study endpoint (FIG. 1B). Histology of the femur from C/G-CB xenograft mice revealed extensive leukemia with bone remodeling resembling the pathology observed in xenograft mice bearing C/G patient-derived leukemia cells (PDX, FIGS. 1C and 2). The malignant cells had a unique pattern of focal adhesion to neighboring cells characteristic of C/G AML. Flow cytometric analysis of marrow C/G-CB xenograft cells identified a malignant population that is of the RAM immunophenotype (CD56^{hi}, CD45^{dim}, and CD38^{dim/-}, FIG. 1D) previously reported in infants with C/G AML (Pardo et al., Cytometry B Clin Cytom 98: 52-56, 2020; and Eidenschink Brodersen et al., Leukemia 30: 2077-2080, 2016). Immunohistochemistry further showed high expression of ERG and CD56 (markers associated with C/G AML (Pardo et al., Cytometry B Clin Cytom 98: 52-56, 2020; Eidenschink Brodersen et al., Leukemia 30: 2077-2080, 2016; and Thirant et al., Cancer Cell 31: 452-465, 2017)) in the mouse bone marrow indicative of malignant transformation, similar to the high CD56 expression in leukemia aggregates present in a bone marrow biopsy from a C/G patient (FIG. 1E).

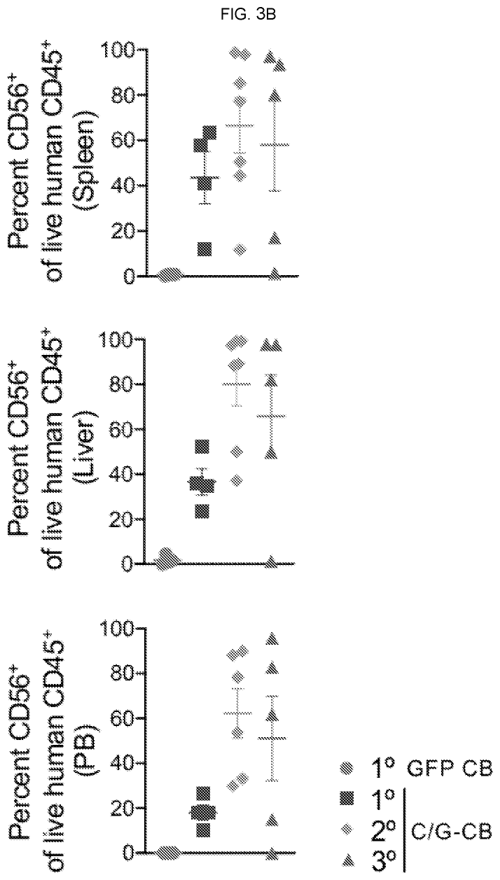

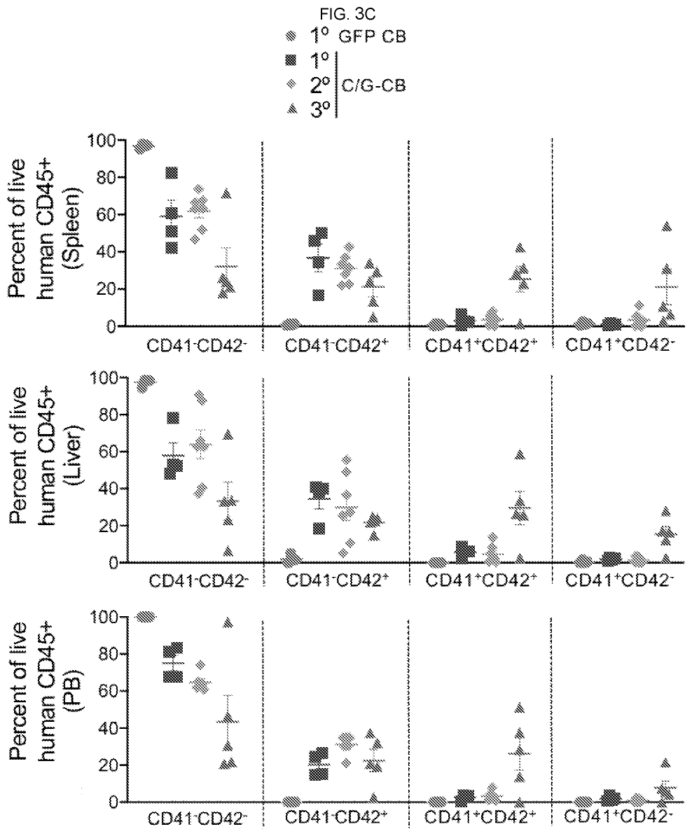

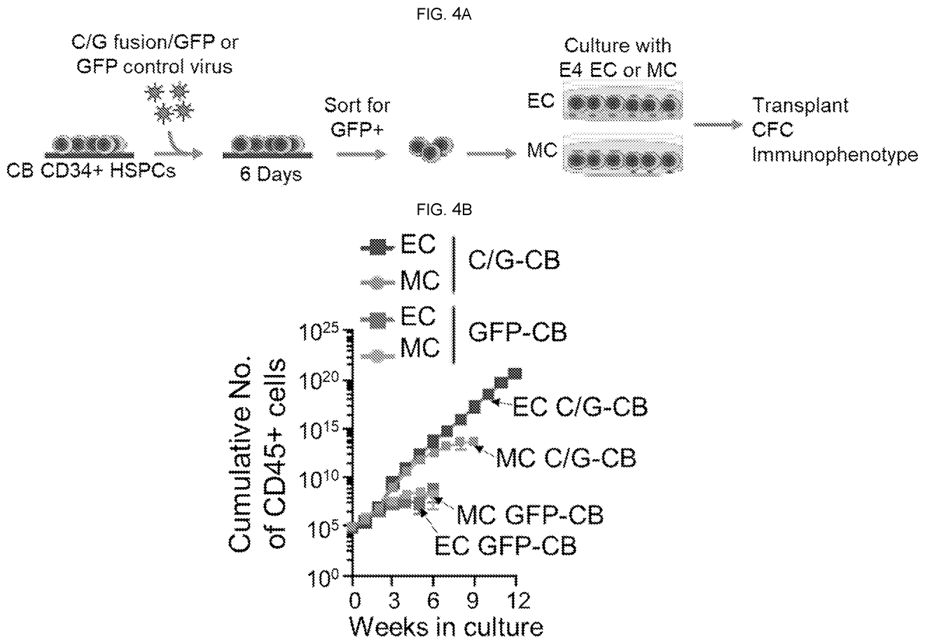

To evaluate whether C/G imparts enhanced self-renewal to leukemia-initiating cells (LICs), serial transplantation of C/G-CB cells was performed. All mice from secondary (8/8) and tertiary (5/5) transplants also developed AML, with a median survival of 69 and 72 days, respectively (FIG. 1F). Bone marrow engraftment of C/G-CB cells was variable in these mice at time of symptomatic disease (5-70%, FIG. 1G); focal clusters of leukemia cells were present in the femur in all mice (FIG. 2), resembling those of the primary transplant and the PDX model. Notably, there was immunophenotypic evolution during the serial transplants with expanded population of CD56+ cells (FIG. 1H). Similar observations were made in other tissues at necropsy (FIGS. 3A and 3B).

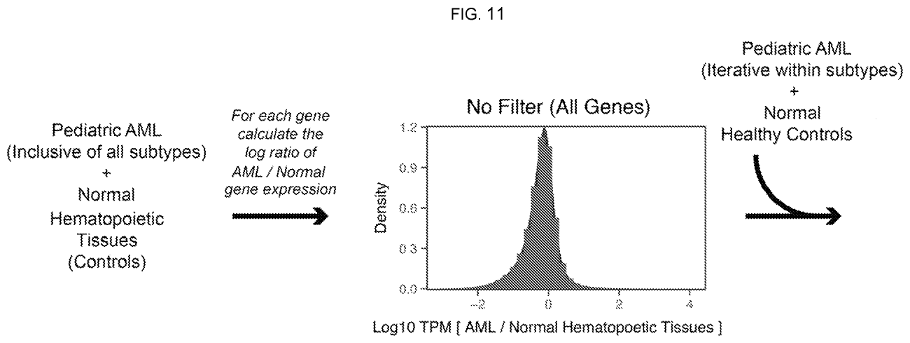

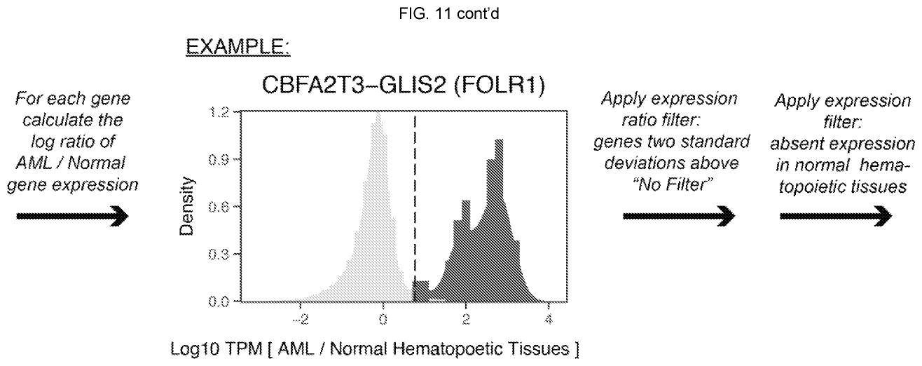

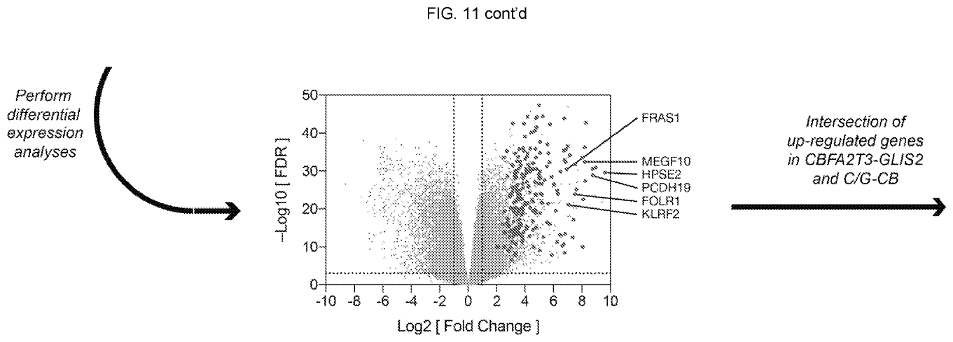

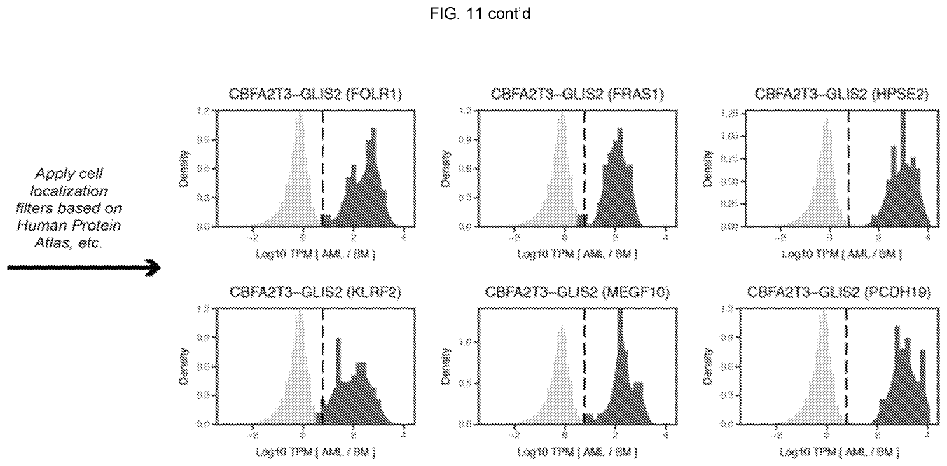

Acute megakaryocytic leukemia (AMKL) is a form of AML that is characterized by immature blasts expressing megakaryocytic markers CD41, CD42 or CD61 (Paredes-Aguilera et al., Am J Hematol 73: 71-80, 2003). Since AMKL is prevalent in C/G-positive patients (Smith et al., Clin Cancer Res 26: 726-737, 2020), CD41 and CD42 expression were assessed on C/G-CB cells. Immunophenotype analysis revealed an aberrant megakaryocytic subset (CD41⁻CD42⁺) in the primary and subsequent serial transplantations (FIGS. 11 and 3C). Bertuccio et. al. previously identified a similar subpopulation whose gene expression most closely matched that of human C/G leukemia (Bertuccio et al., Hemasphere 4: e319, 2020). Monitoring CD41 and CD42 expression during serial transplantation showed an immunophenotypic evolution from CD41⁻CD42⁺ to the mature CD41⁺CD42⁺ megakaryocytic subsets (FIGS. 1I, 1J, and 3C). Taken together, these results demonstrate that the expression of C/G induces transformation of CB HSPCs that faithfully recapitulates human C/G AMKL.

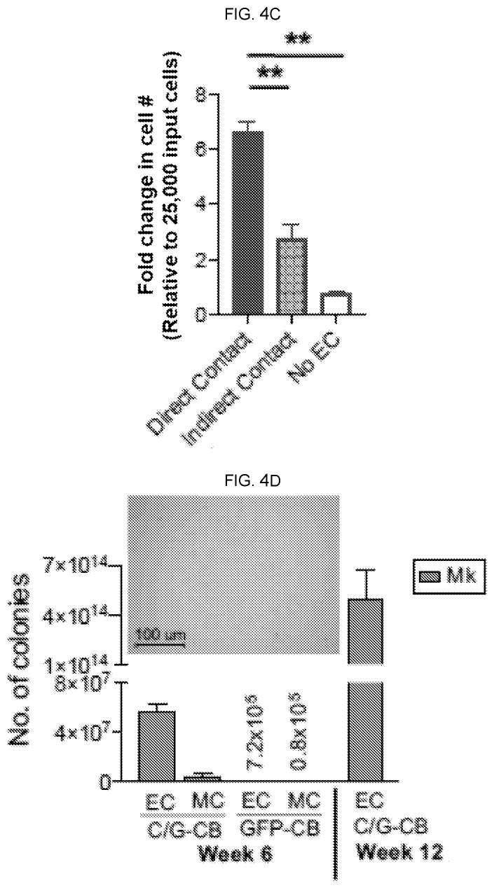

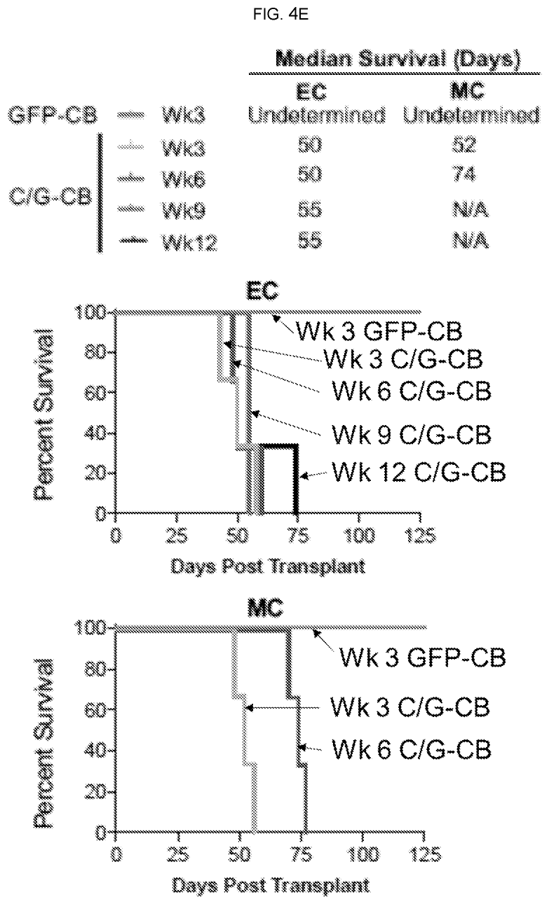

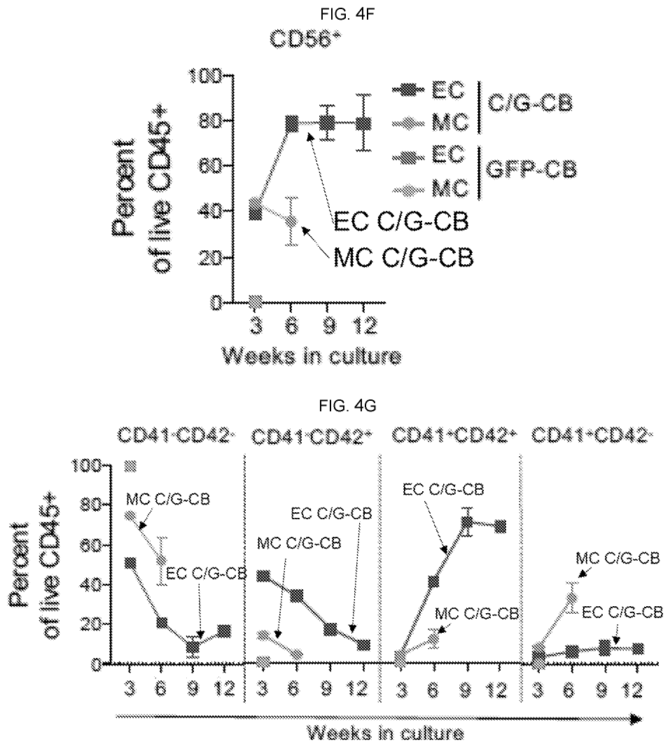

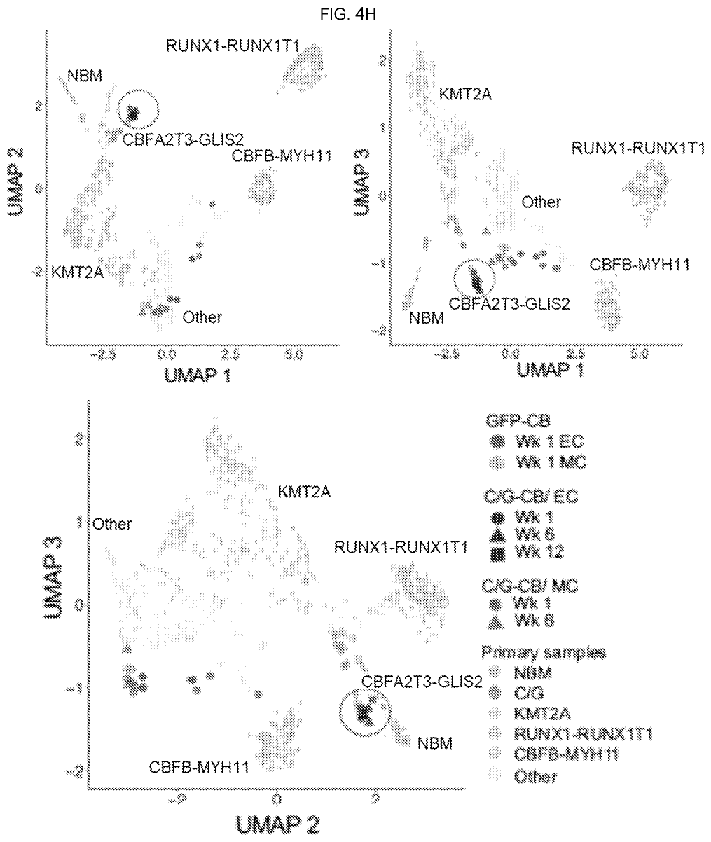

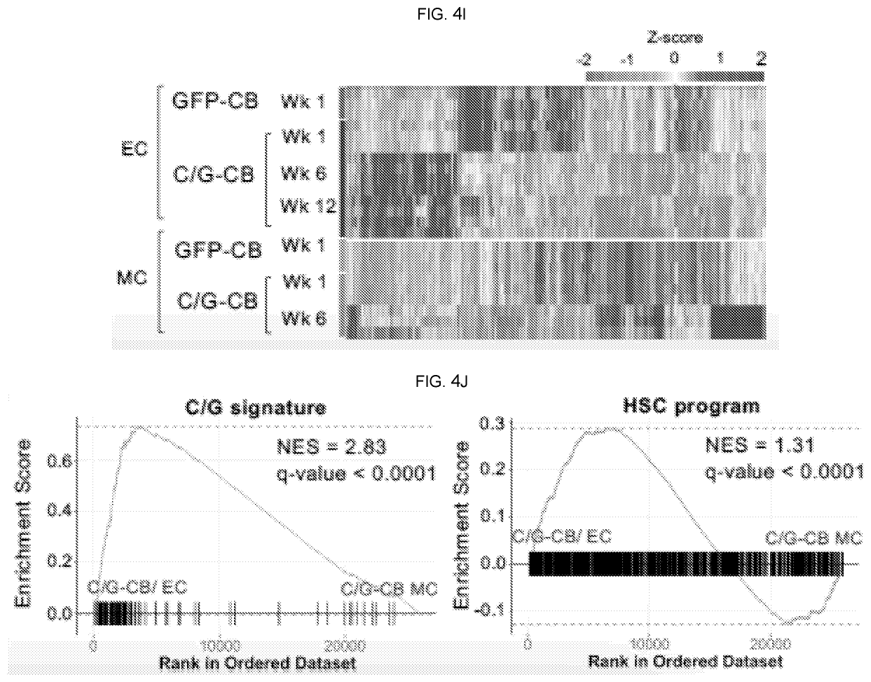

ECs promote leukemic progression ex vivo. Mounting evidence supports the role of the microenvironment in the leukemic process. Vascular niche endothelial cells (ECs), in particular, play a critical role in both normal and malignant hematopoiesis, contributing to maintenance and self-renewal of HSPCs as well as supporting leukemic progression, leukemia precursor survival and drug resistance (Pinho et al., Nat Rev Mol Cell Biol 20, 303-320, 2019; Poulos, M. G. et al. Exp Hematol 42: 976-986 e971-973, 2014; Walter, R. B. et al. Leukemia 28: 1969-1977, 2014; and Le et al., Leukemia: 35:601-605, 2021). Previous studies have demonstrated that human umbilical vein endothelial cells transduced with E4ORF1 virus (E4 ECs) support the expansion of CB HSPCs (Butler et al., Blood 120: 1344-1347, 2012) and provide efficient conditions for long-term culture of primary AML precursors (Walter, R. B. et al. Leukemia 28: 1969-1977, 2014), thus effectively recapitulating the EC niche ex vivo. To assess whether ECs support leukemic transformation of C/G fusion, C/G-CB cells were cultured in E4 EC co-culture (Butler et al., Blood 120: 1344-1347, 2012) or in myeloid-promoting conditions (Imren et al., Blood 124: 3608-3612, 2014) (MC, FIG. 4A). C/G-CB cells expanded faster with prolonged lifespan in EC co-culture compared to MC, as determined by the cumulative number of GFP+ cells (FIG. 4B). In contrast, GFP-CB cells exhibited limited, short-lived proliferation reaching exhaustion after 3 weeks in either condition. Proliferation of C/G-CB cells declined after transfer to either an EC trans-well culture or in suspension culture (FIG. 4C), suggesting that the growth promoting effect of the ECs is mediated by direct contact and secreted factors.

The C/G fusion has been previously shown to confer self-renewal to hematopoietic progenitors (Gruber et al., Cancer Cell 22, 683-697, 2012; and Thirant et al., Cancer Cell 31: 452-465, 2017). This property in C/G-CB cells was further enhanced by EC co-culture (or culture with endothelial cells). At 6 weeks, C/G-CB cells in EC co-culture formed significantly more megakaryocytic colonies than C/G-CB cells grown in MC or C/G-GFP cells grown in either condition. Strikingly, after 12 weeks C/G-CB cells cultured in EC co-culture produced a large number of megakaryocytic colonies (FIG. 4D), demonstrating long lived self-renewal of the C/G-CB cells co-cultured with ECs.

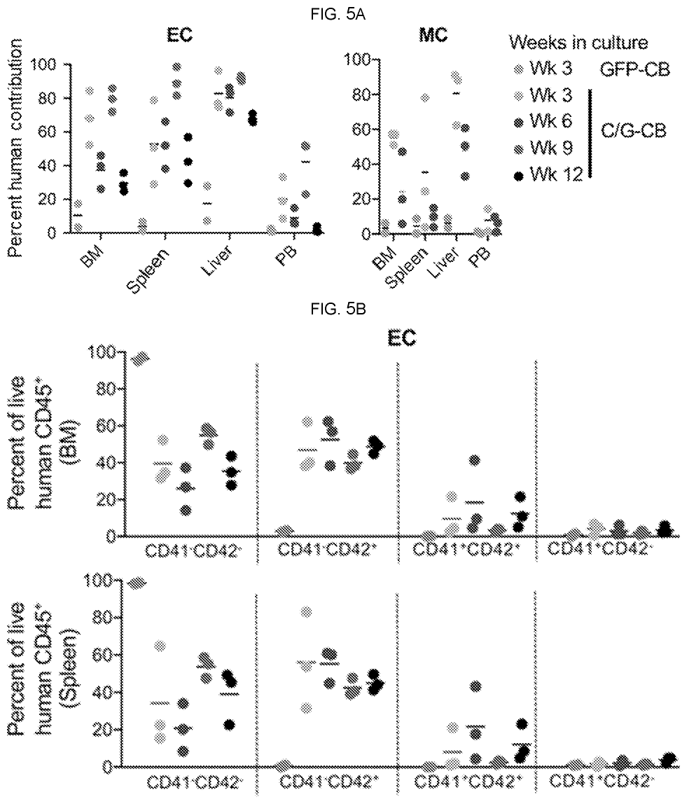

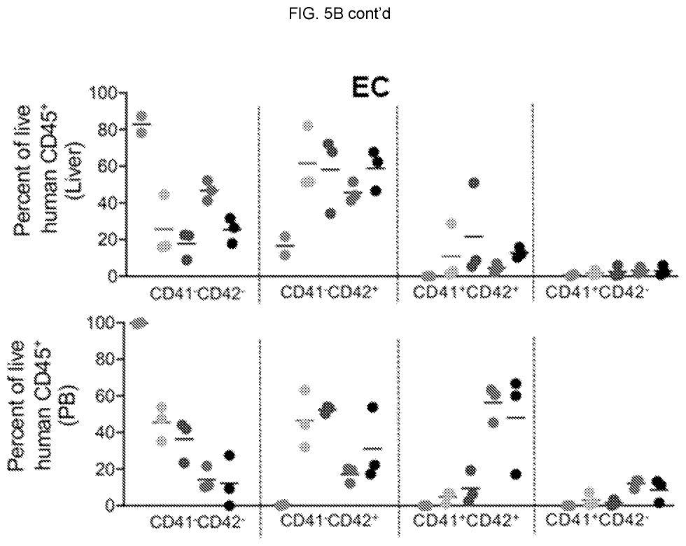

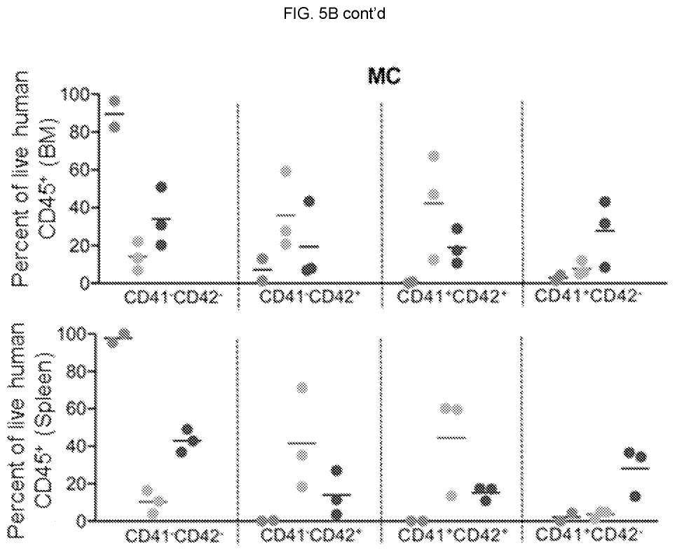

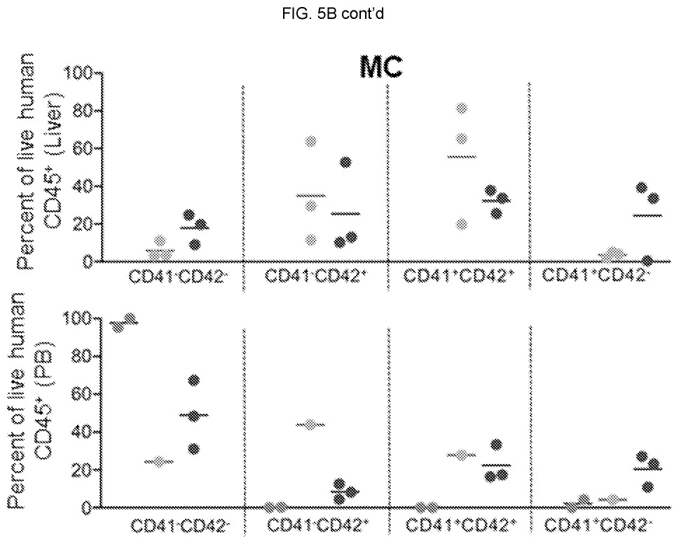

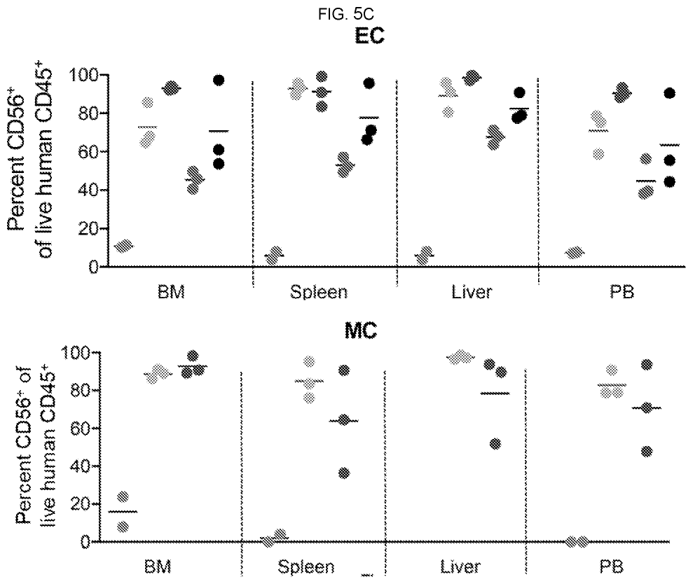

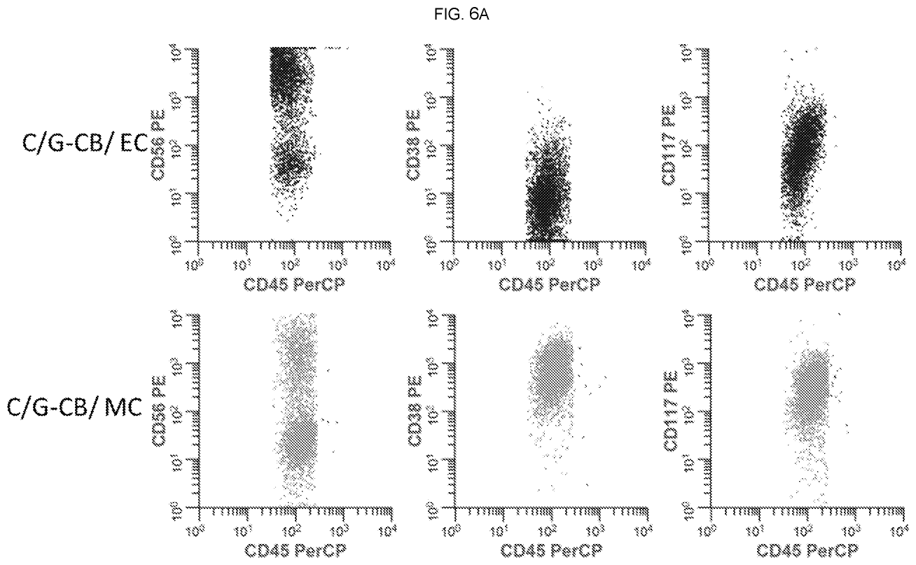

To determine whether the EC niche promotes the generation and propagation of LICs, the engraftment of C/G-CB cells expanded on ECs or in MC following 3, 6, 9 and 12 weeks of culture was evaluated. Remarkably, C/G-CB cells cultured in EC co-culture at each time point exhibited robust engraftment that progressed to frank leukemia in vivo (FIGS. 4E, and 5A-5C), demonstrating that EC co-culture promotes long-term maintenance of functional LICs. C/G-CB cells grown in the MC also induced leukemia from 3- and 6-week cultures but then became senescent at 9 and 12 weeks, suggesting limited preservation of the LICs.

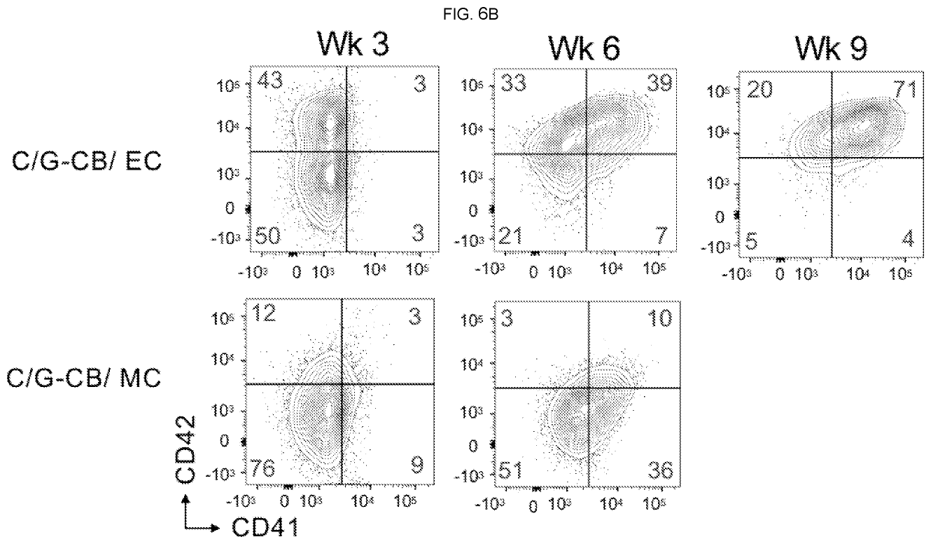

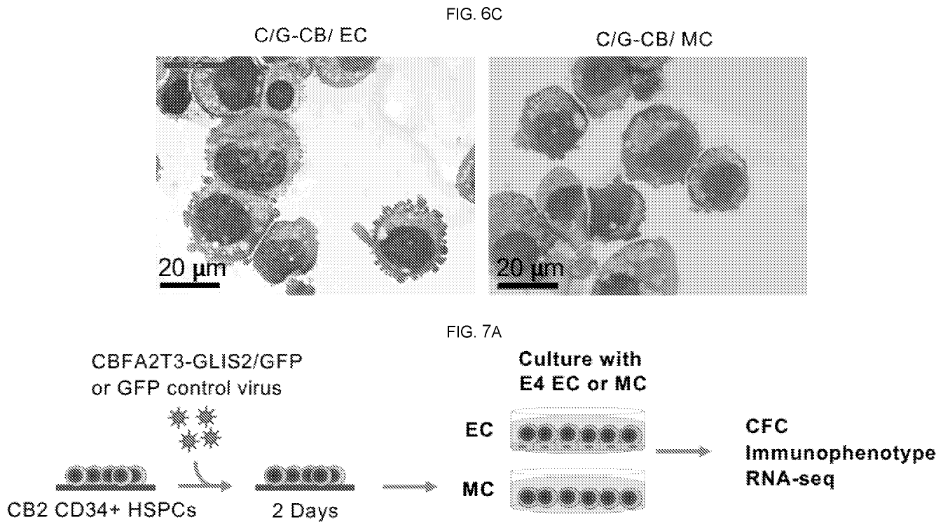

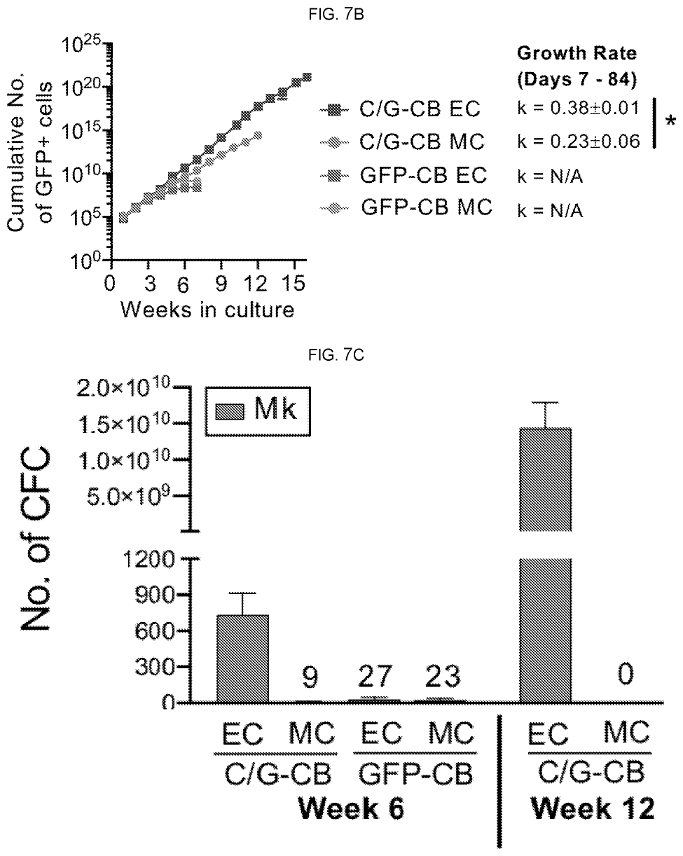

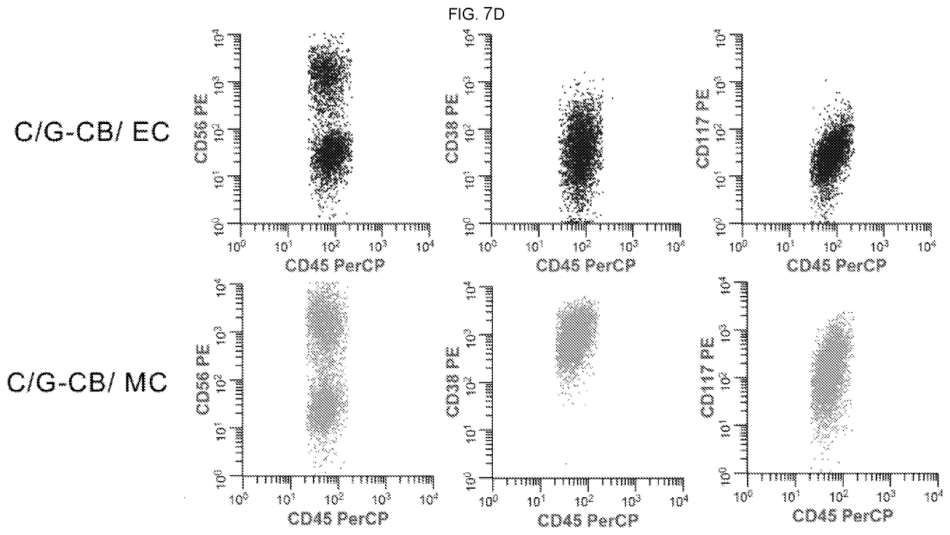

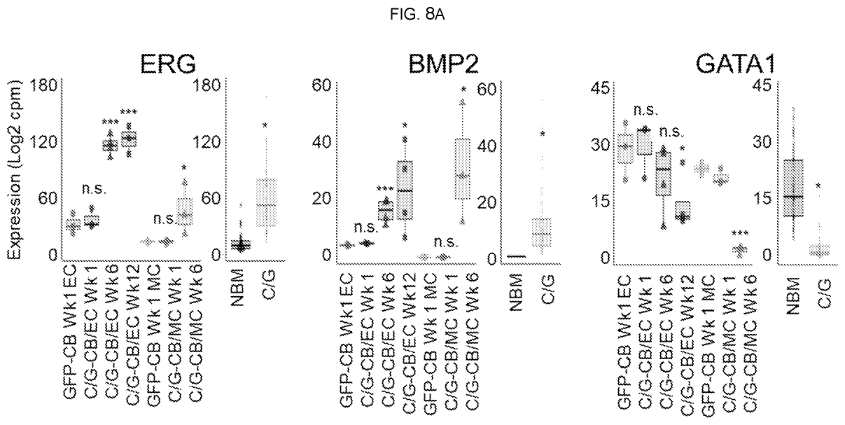

To monitor leukemic evolution, the expression of the RAM immunophenotype and AMKL markers on C/G-CB cells from EC co-culture and MC was assessed. C/G-CB cells in EC co-culture constituted an almost homogeneous population that expressed the RAM immunophenotype, whereas only a subset was detected in the MC at week 6 (FIG. 6A). A high percentage of CD56+ cells was maintained in EC co-culture for 6-12 weeks (FIG. 4F). Emergence of the aberrant CD41⁻CD42⁺ subset occurred by week 3 in both culture conditions, albeit more prominently in EC co-culture (FIGS. 4G and 6B), then progressed to the more mature immunophenotype. Morphological evaluation showed megakaryocytic features among C/G-CB cells in both culture conditions (FIG. 6C). These results were reproduced in a separate experiment with CB HSPCs from another donor (FIG. 7A-7D). Thus, EC co-culture supports the development of C/G-transformed CB HSPCs that recapitulate the series of immunophenotypic changes associated with transformation in primary C/G AML.

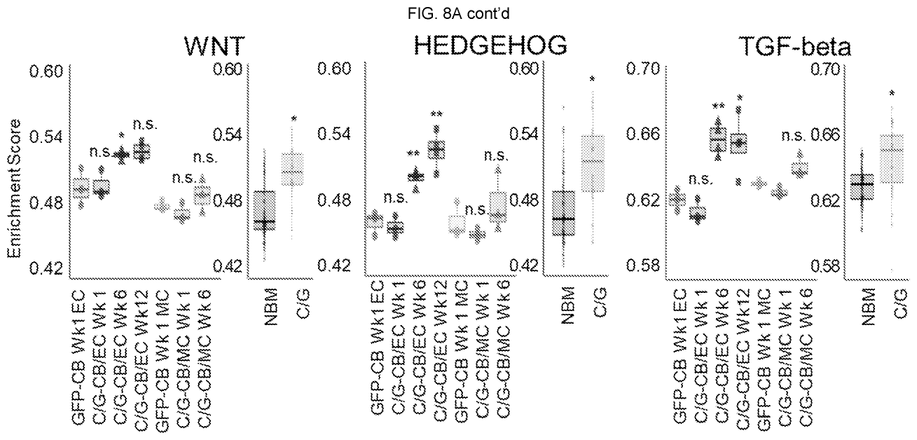

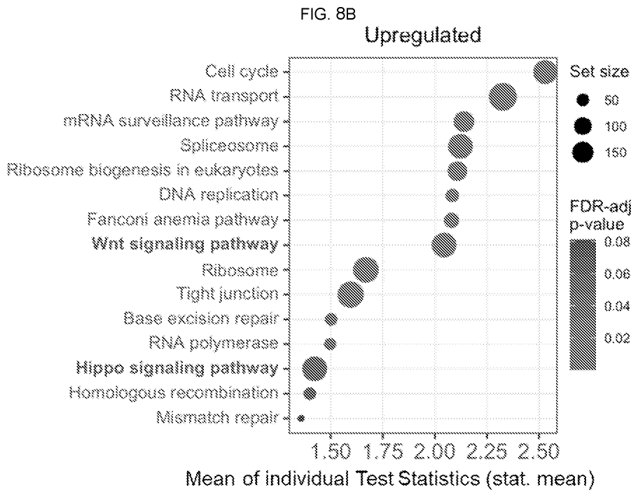

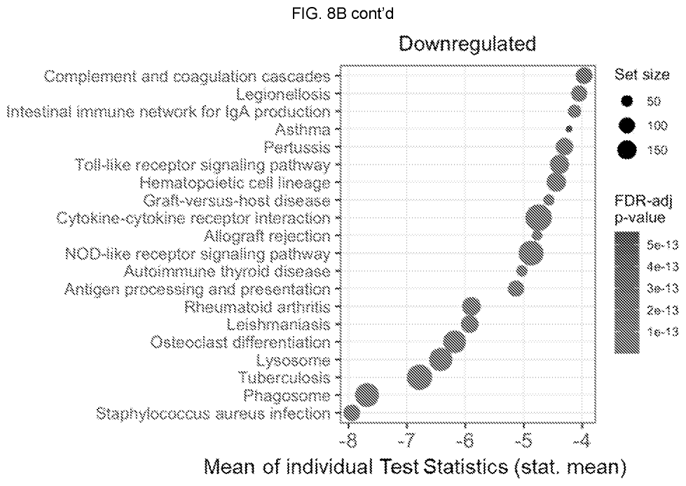

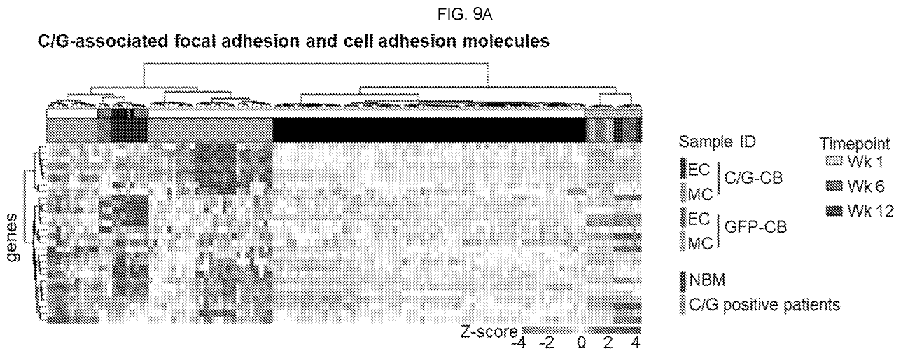

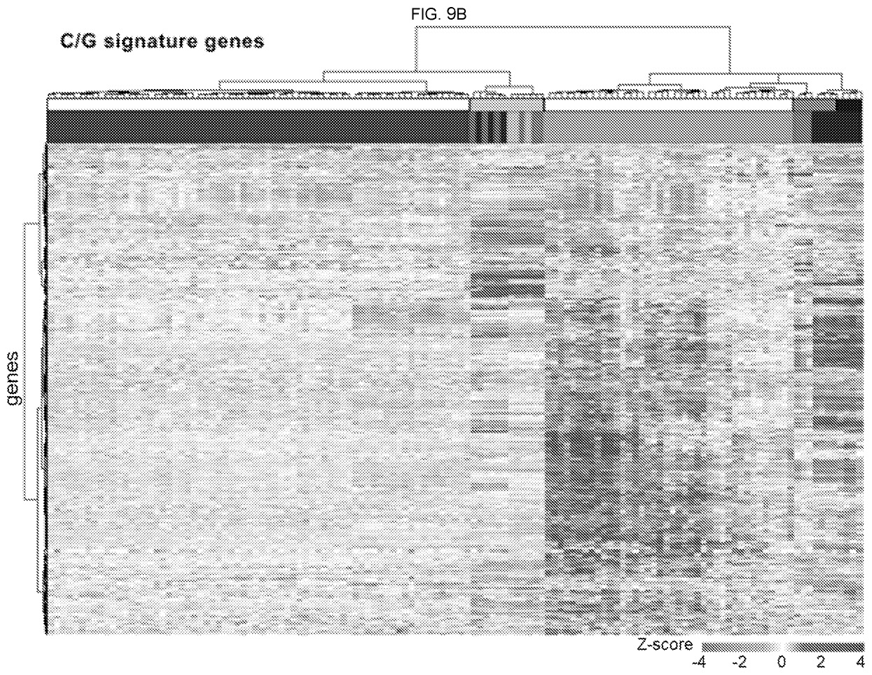

Fidelity of engineered cells to C/G AML. To determine the fidelity of transformation to primary leukemia, RNA-sequencing was performed of C/G-CB cells cultured with ECs or in MC. Remarkably, unsurpervised clustering analysis demonstrated that the C/G-CB cells from weeks 6 and 12 in EC co-culture clustered with primary C/G-positive patient samples, but not C/G-CB cells cultured in MC nor GFP controls (FIG. 4H). This suggested that the signaling pathways that are aberrantly dysregulated in primary C/G leukemia are faithfully recapitulated in C/G-CB cells co-cultured with ECs. Further transcriptome analysis revealed up-regulation of ERG and BMP2, downstream genes previously shown to be strongly upregulated in C/G AML (Gruber et al., Cancer Cell 22, 683-697, 2012; and Thirant et al., Cancer Cell 31: 452-465, 2017), and down-regulation of erythroid-megakaryocyte differentiation gene GATA1 (Welch et al., Blood 104: 3136-3147, 2004; Wang et al., EMBO J 21: 5225-5234, 2002; Vyas et al., Blood 93: 2867-2875, 1999; Shivdasani et al., EMBO J 16: 3965-3973, 1997; and Kuhl et al., Mol Cell Biol 25: 8592-8606, 2005), also down-regulated in C/G AML (Thirant et al, Cancer Cell 31: 452-465, 2017), in both EC co-culture and MC (FIG. 8A). However, there were significant differences in the global expression profiles of C/G-CB cells from EC co-culture compared to MC (FIG. 4I).

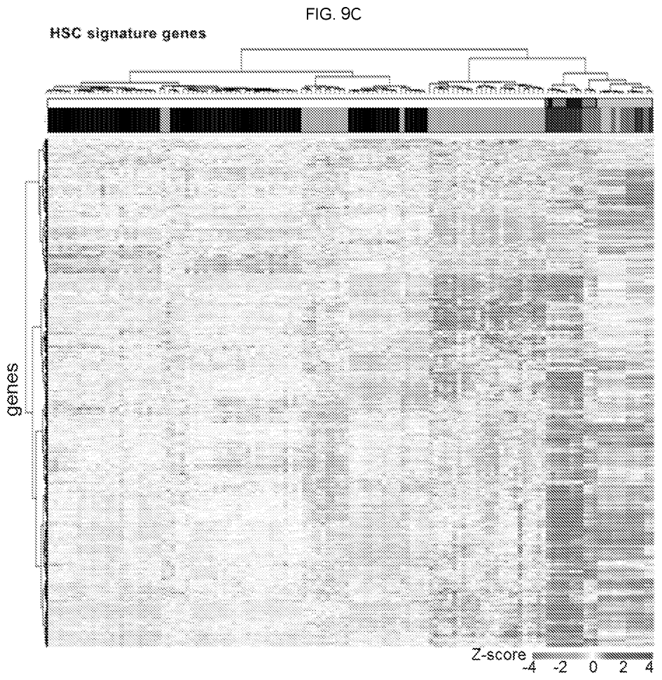

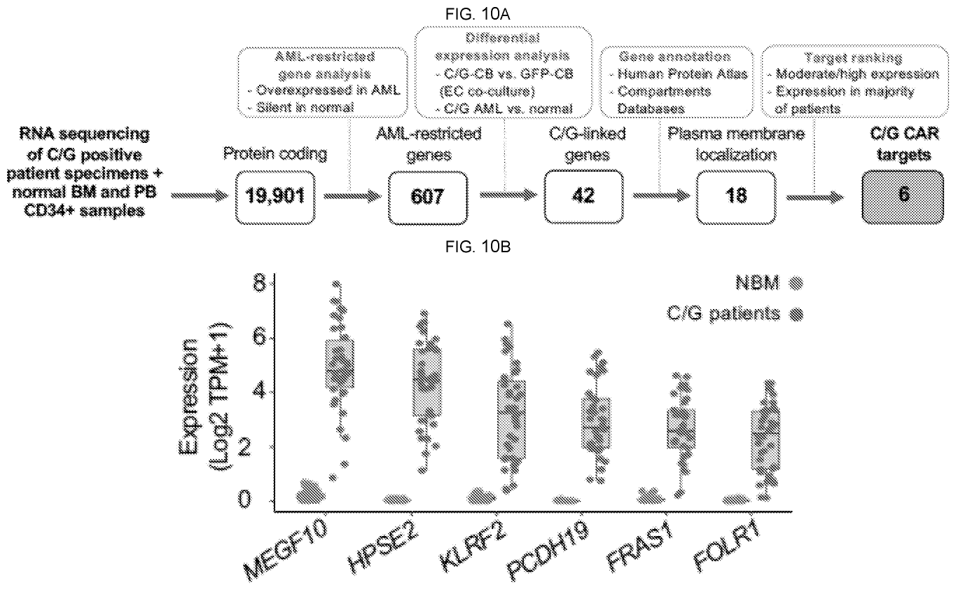

To determine the effects of ECs on malignant transformation, the status was assessed of the WNT, HEDGEHOG and TGF-beta pathways known to be dysregulated in C/G leukemia (Gruber et al., Cancer Cell 22, 683-697, 2012; and Smith et al., Clin Cancer Res 26: 726-737, 2020). These pathways were highly enriched in C/G-CB cells grown in EC co-culture but not in MC (FIG. 8A). It has been demonstrated that a number of cell adhesions and integrins are upregulated in C/G leukemia (Smith et al., Clin Cancer Res 26: 726-737, 2020). A majority of these genes were upregulated in C/G-CB cells independent of the culture condition (FIG. 9A), suggesting this pathway is determined by the fusion and not the microenvironment. The expression of cell adhesions and integrins presumably contributes to the focal distribution and adherent morphology identified in the C/G-CB xenograft mice (FIG. 1C).

Gene Set Enrichment Analysis (GSEA) also revealed that C/G and HSC signature genes, previously identified to be associated with C/G AML (Smith et al., Clin Cancer Res 26: 726-737, 2020; and Thirant et al., Cancer Cell 31: 452-465, 2017), were both significantly enriched in C/G-CB cells grown in EC culture relative to MC (FIGS. 4J, 9B, and 9C).

Hippo signaling pathway and tight junction are other C/G-specific pathways (see Smith et al., Clin Cancer Res 26: 726-737, 2020) that were also significantly enriched in the C/G-CB cells in EC co-culture compared to MC (FIG. 8B). Together, these results suggest that ECs induce transcriptional programs that synergize with the fusion to recapitulate the primary leukemia.

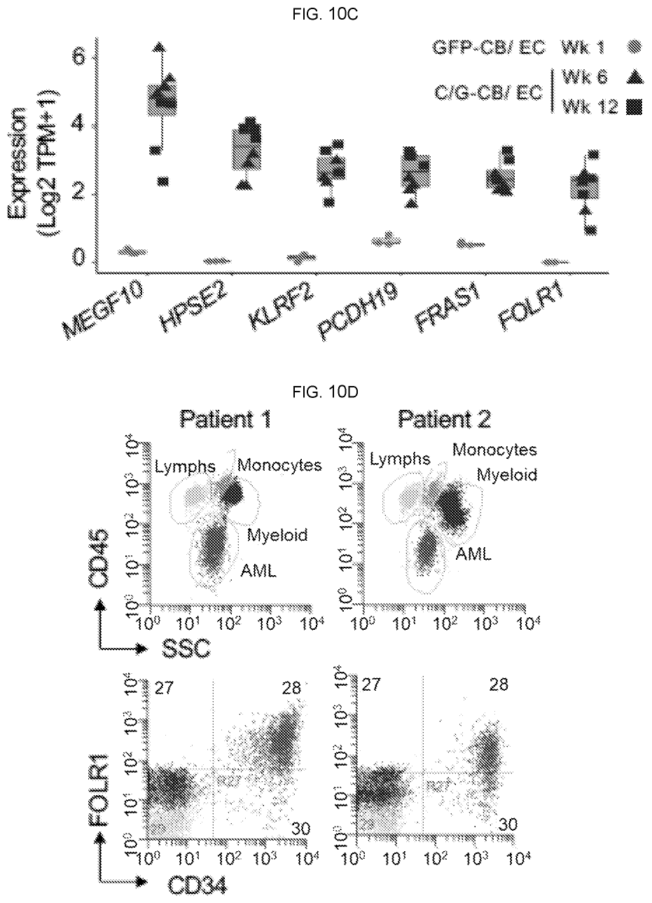

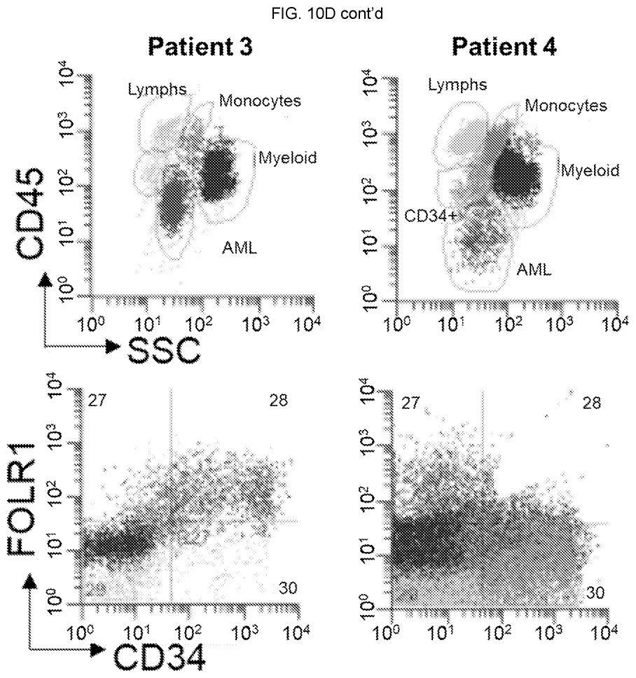

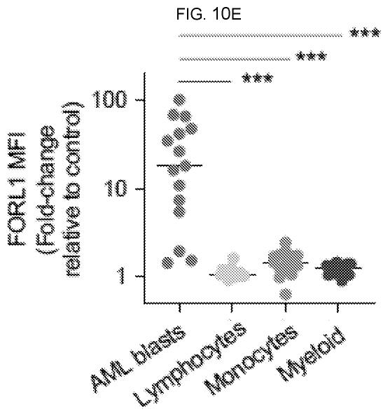

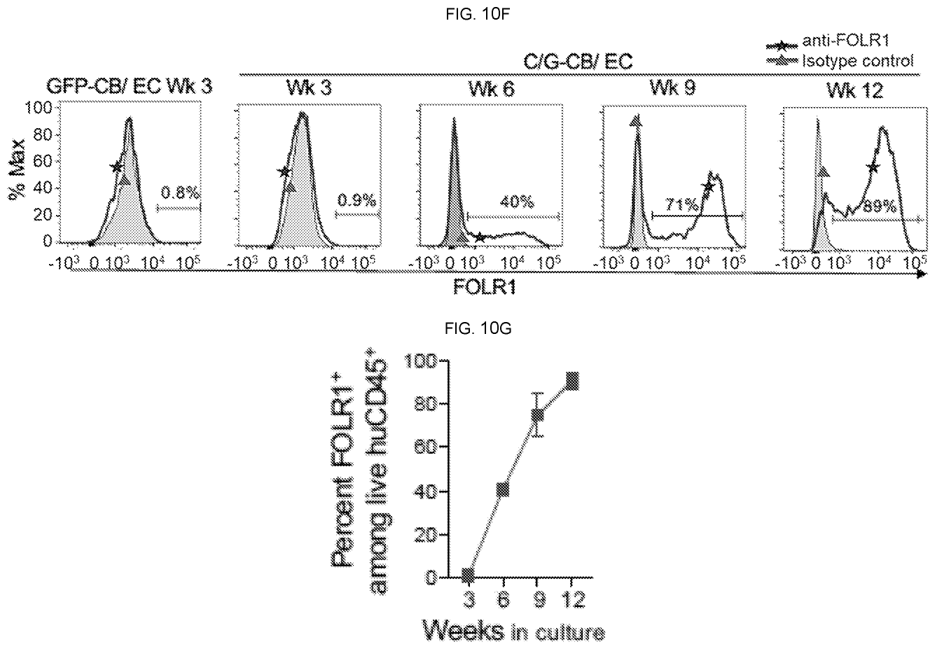

Upregulation of FOLR1 therapeutic target. Although CAR T therapy has proven successful in treating B-cell acute lymphoblastic leukemia (B-ALL), immunotherapeutic targeting of AML remains a challenge given significant overlap of target antigens expressed on AML and normal hematopoietic cells. The expansive target discovery effort through TARGET and Target Pediatric AML (TpAML) has identified a library of AML-restricted genes (expression in AML, silent in normal hematopoiesis) in one or more AML subtypes, including C/G AML (607 genes, FIGS. 10A and 11). Of these, 42 were upregulated in both C/G AML and in C/G-CB cells cultured with ECs, representing C/G fusion-linked genes. Eighteen of these encode proteins that localize to the plasma membrane, of which seven C/G fusion-specific CAR targets (FOLR1, MEGF10, HPSE2, KLRF2, PCDH19, and FRAS1) were identified to be highly expressed in C/G patients and in C/G-CB cells but entirely silent in normal hematopoesis (FIGS. 10B and 10C).

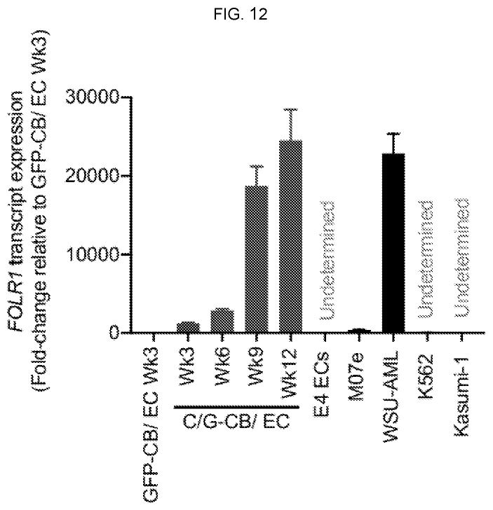

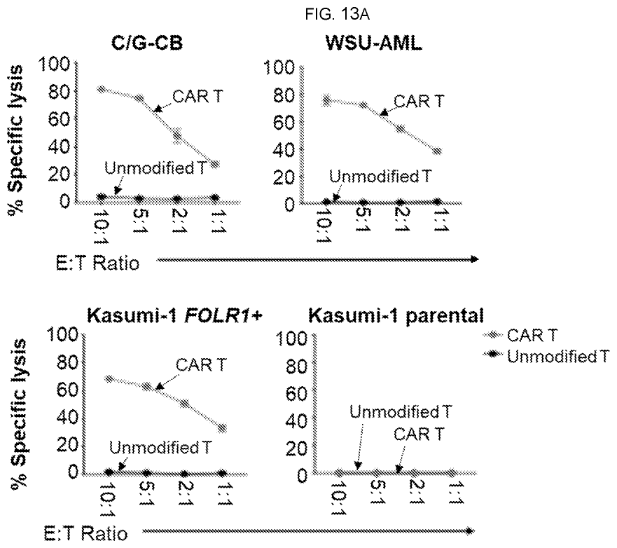

FOLR1 was prioritized for further development given its existing record as a target in solid tumors (Scaranti et al., Nat Rev Clin Oncol 17: 349-359, 2020). FOLR1 transcript expression was confirmed by qPCR (FIG. 12). Flow cytometric analysis of primary AML cells showed that FOLR1 was expressed on AML blasts but not on normal lymphocytes, monocytes, and myeloid cells within individual patients (FIGS. 10D and 10E). Surface FOLR1 protein was detected in C/G-CB cells as early as 6 weeks of EC co-culture, progressing to near uniform expression by week 12 (FIGS. 10f and 10G).

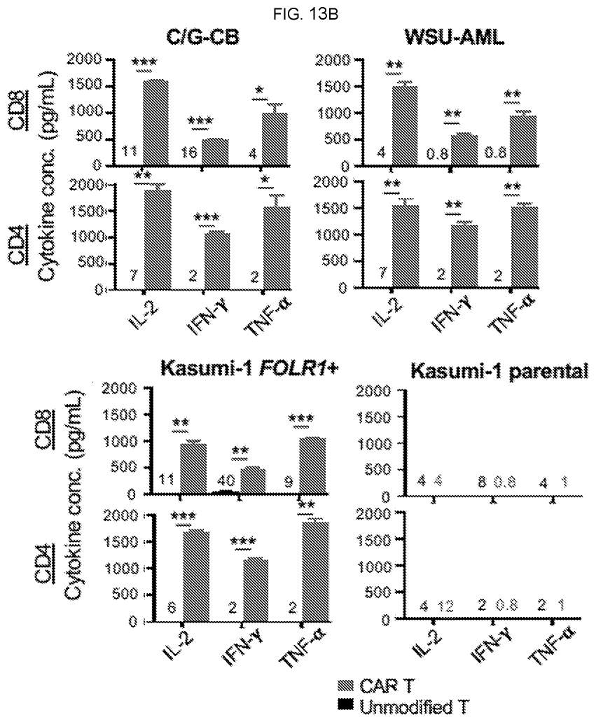

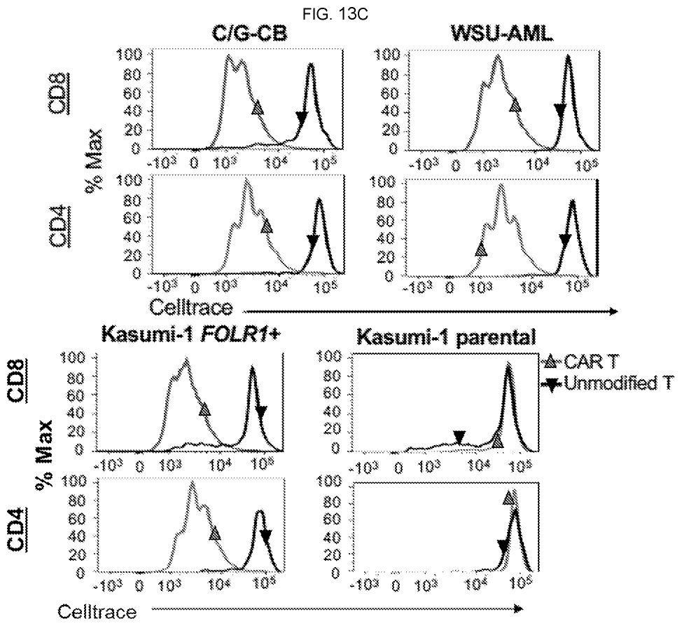

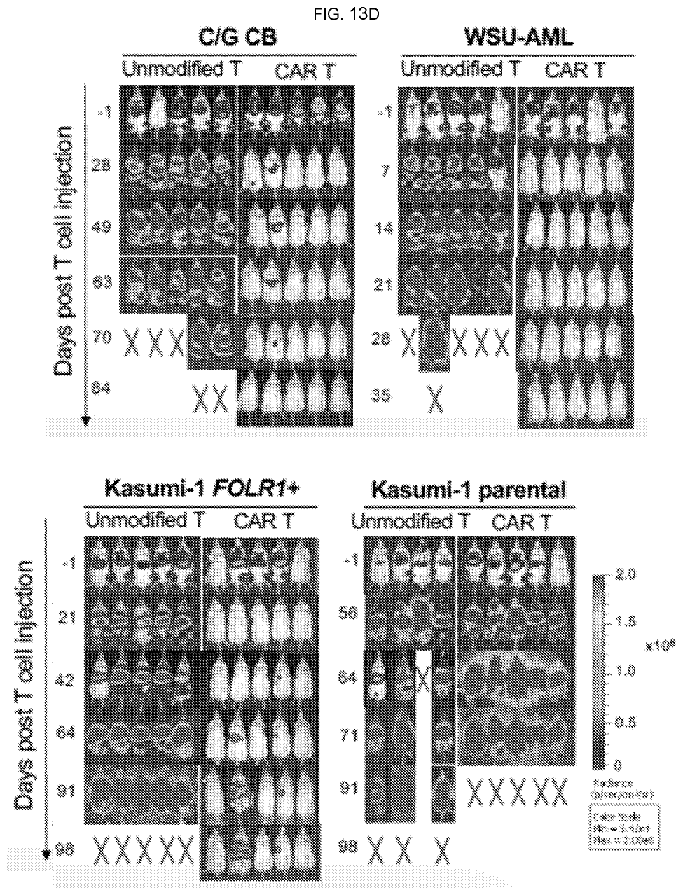

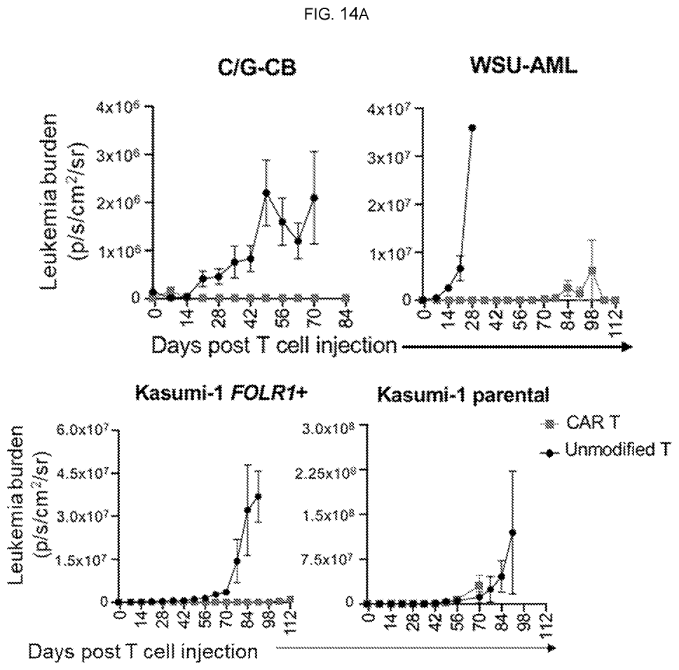

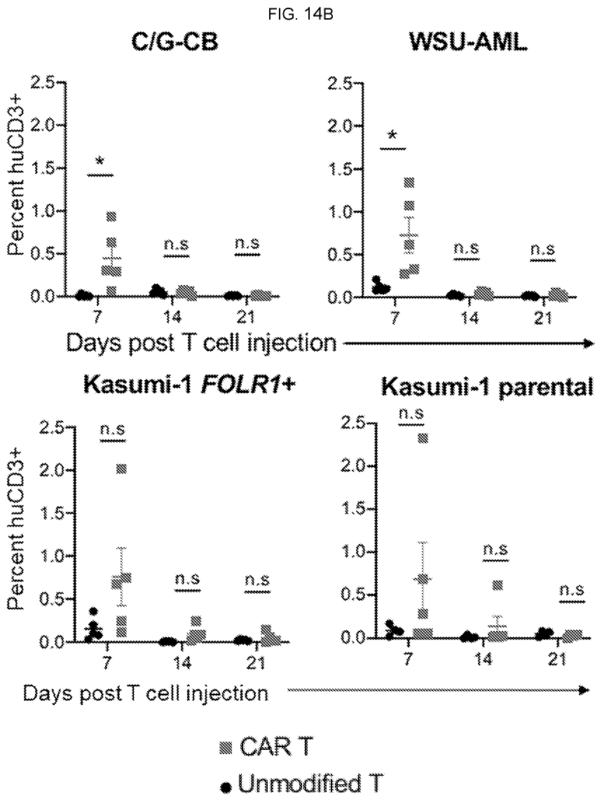

Targeting C/G AML with FOLR1 CAR T. The evidence that FOLR1 is causally linked to the C/G fusion and uniquely expressed in AML blasts suggested that targeting FOLR1 may provide a specific strategy to eliminate C/G leukemia without impacting normal hematopoiesis. To evaluate the therapeutic potential of targeting FOLR1, a FOLR1-directed CAR was generated using anti-FOLR1 binder (Farletuzumab), IgG4 intermediate spacer and 41-BB/CD3zeta signaling domains (see Methods). The target specificity of FOLR1-directed CAR T cells was tested against FOLR1-positive (C/G-CB, WSU-AML, Kasumi-1 FOLR1+) and FOLR1-negative (Kasumi-1) cells. CD8 FOLR1 CAR T cells demonstrated cytolytic activity against FOLR1 positive but not FOLR1 negative cells (FIG. 13A). Furthermore, both CD8 and CD4 FOLR1 CAR T cells produced higher levels of IL-2, IFN-γ, and TNF-α and proliferated more robustly than did unmodified T cells when co-incubated with FOLR1 positive but not FOLR1 negative cells (FIGS. 13B and 13C). These results indicate highly specific reactivity of FOLR1 CAR T cells against AML cells expressing FOLR1.

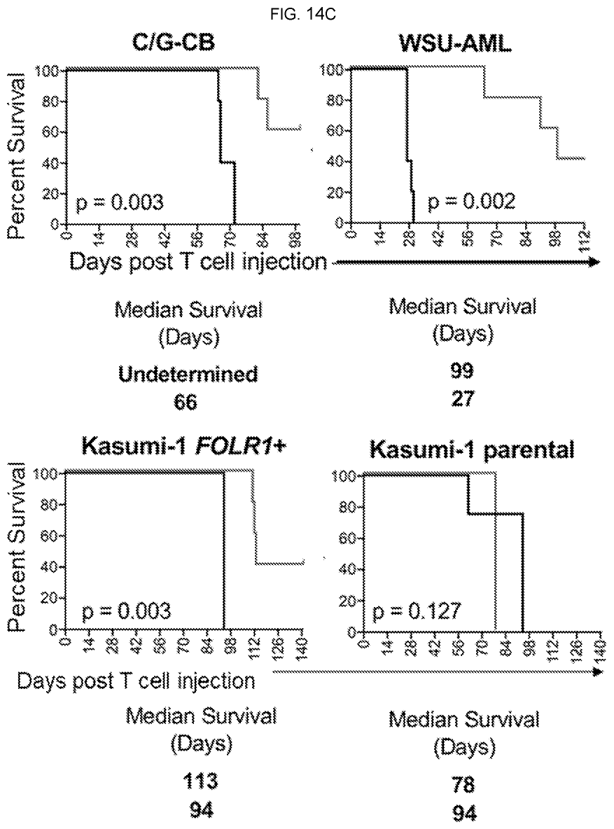

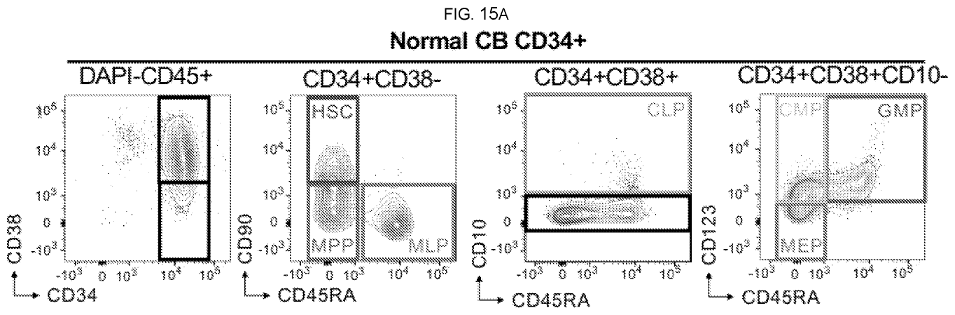

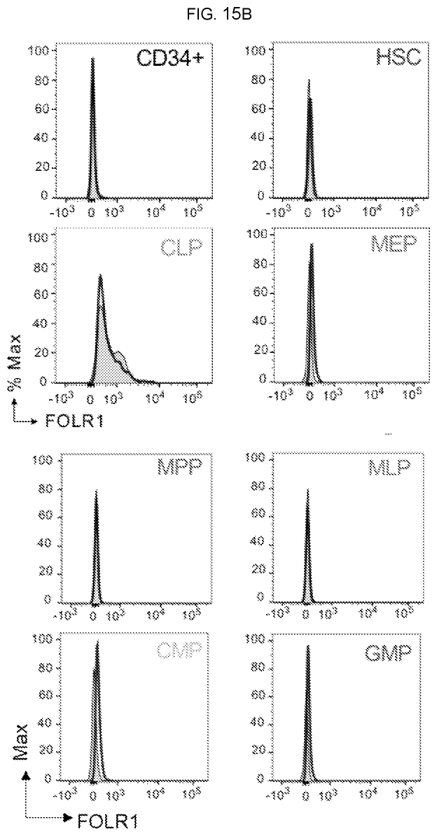

The in vivo efficacy of FOLR1-directed CAR T cells was next investigated. In C/G-CB, WSU-AML, and Kasumi-1 FOLR1+ xenograft models, treatment with FOLR1 CAR T cells induced leukemia clearance, while disease progression occurred in all mice that received unmodified T cells (FIGS. 13D and 14A). Leukemia clearance was associated with expansion of CAR T cells in the peripheral blood of C/G-CB and WSU-AML xenografts (FIG. 14B). Importantly, treatment with FOLR1 CAR T cells significantly extended the median survival in mice bearing C/G-CB, WSU-AML, Kasumi-1 FOLR1+ leukemias, respectively (FIG. 14C). Activity of FOLR1 CAR T cells in vivo was target specific, as they did not limit the leukemia progression nor extend the survival of Kasumi-1 xenografts (FIGS. 13D and 14C).

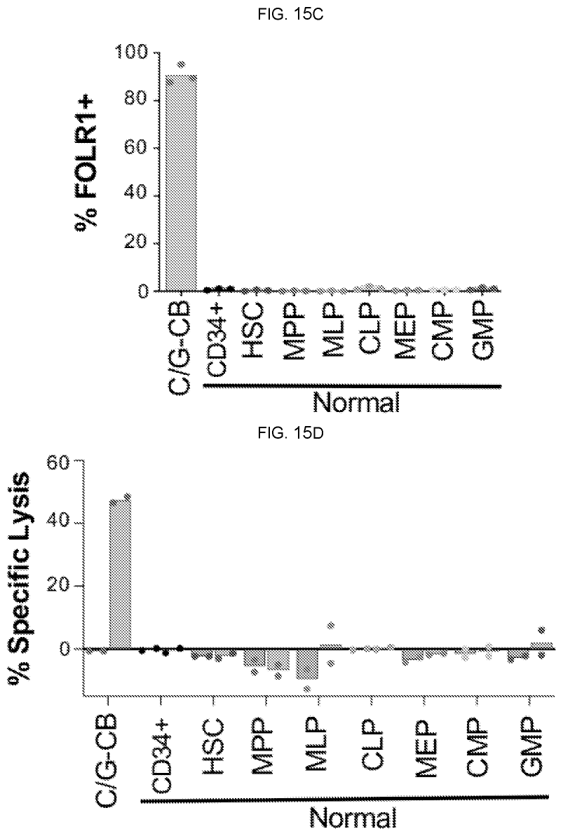

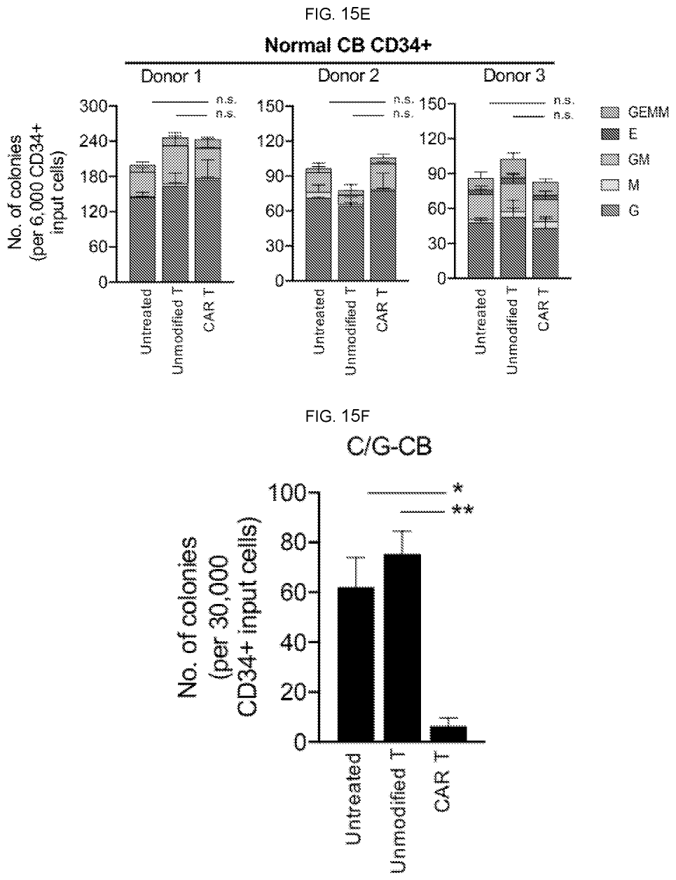

To determine whether FOLR1 is expressed on normal HSPCs, FOLR1 expression was characterized in CB CD34+ samples from three healthy donors. FOLR1 expression was entirely silent in HSPC subsets (FIGS. 15A-15c). Consistent with lack of expression, no cytolytic activity was detected against HPSCs after 4-hour co-incubation with CAR T cells (FIG. 15D). Moreover, FOLR1 CAR T cells did not affect the self-renewal and multilineage differentiation capacity of normal HSPCs as compared to unmodified control T cells (FIG. 15E), whereas significant eradication of colonies were detected in the C/G-CB cells (FIG. 15F). Taken together, these results suggest that FOLR1 CAR T can eradicate C/G AML cells without compromising normal HSPCs and may be a promising therapy for C/G AML.

Discussion Previous attempts to generate overt leukemia from C/G-transduced murine marrow hemopoietic cells have not been successful (Gruber et al., Cancer Cell 22, 683-697, 2012; and Dang et al., Leukemia 31: 2228-2234, 2017), leading to the notion that cooperating mutations are required for leukemic transformation. This example demonstrates that the C/G oncogenic fusion is sufficient to transform human CB HSPCs that faithfully recapitulates the transcriptome, morphology and immunophenotype of C/G AML observed in infants as well as highly aggressive leukemia in xenograft models. It is further demonstrated that direct interactions with EC niche are required for malignant transformation by this fusion protein. These results demonstrate that oncogenic fusions may be sufficient to induce frank AML phenotype given the appropriate developmental milieu (CB HSPCs) and the permissive microenvironment (EC niche). This contrasts with the widely accepted “cooperative” model of AML requiring synergy between a class II (fusion) and class I (SNVs) variants for recapitulating the AML phenotype (Gilliland et al., Curr Opin Hematol 8: 189-191, 2001).

Progress in elucidating mechanisms of disease and development of novel therapies for the C/G AML cohort is currently limited by a lack of relevant model systems that accurately recapitulate human disease. The EC co-culture platform overcomes this barrier and recapitulates the vascular EC niche that supports malignant transformation, self-renewal and LIC propagation in vitro. This platform is thus suited to interrogating AML-niche interactions and identifying novel therapeutic targets for C/G, and it should be extended to studies with other oncogenic fusions.

Finally, the results presented here address a fundamental challenge in immunotherapy for AML, as AML-restricted targets have been elusive. By integrating transcriptomics of primary C/G AML and engineered CB cells, seven C/G fusion-specific genes have been identified that represent potential high-value targets. The present disclosure provides validation for FOLR1, by showing that FOLR1-direct CAR T effectively eradicates C/G AML cells while sparing normal HSPCs. These results provide a pre-clinical foundation for further development of FOLR-directed CAR T in clinical trials for the treatment of C/G AML.

### Materials and Methods.

Animals. NOD/SCID/γc^{-/-} (NSG) and NOD.CG-Prkdc^{scid} Il2rg^{tm1Wjl} Tg(CMV-IL3, CSF2, KITLG) 1Eav/MloySzJ (NSG-SGM3) mice were obtained from the Jackson Laboratory. For all experiments, 6-10-week-old age-matched females were randomly assigned to experimental groups. Mice transplanted with engineered CB or AML cell lines were monitored and euthanized when they exhibited symptomatic leukemia (tachypnea, hunchback, persistent weight loss, fatigue or hind-limb paralysis). Experiments were performed after approval by Institutional Animal Care and Use Committee (protocol #51068) and in accordance with institutional and national guidelines and regulations.

Primary Specimens. Human umbilical cord blood samples were obtained from normal deliveries at Swedish Medical Center (Seattle, WA). Frozen aliquots of AML diagnostic bone marrow samples were obtained from the Children's Oncology Group. Cells were thawed in Iscove's Modified Dulbecoo's Medium (IMDM) supplemented with 20% fetal bovine serum (FBS) and 100 U/mL DNasel (Sigma, Cat #D5025). A bone marrow biopsy from a C/G patient was obtained from a patient treated at the University of Minnesota Masonic Children's Hospital. Healthy donor T cells were obtained from Bloodworks Northwest (Seattle, WA). It was confirmed these cells lacked infectious agents (Epstein-Barr virus (EBV), human cytomegalovirus (HCMV), Hepatitis A, Hepatitis B, Hepatitis C, human herpesvirus (HHV) 6, HHV 8, human immunodeficiency virus (HIV)1, HIV2, human papillomavirus (HPV)16, HPV18, herpes simplex virus (HSV)1, HSV2, human T-lymphotropic virus (HTLV) 1, HTLV 2, and Mycoplasma sp) through IDEXX Bioanalytics (West Sacramento, CA). All specimens used in this example were obtained after written consent from patients and donors. The research was performed after approval by the FHCRC Institutional Review Board (protocol #9950). The study was conducted in accordance with the Declaration of Helsinki.

Cell lines. M07e (DSMZ, Cat #ACC104), WSU-AML (BiolVT, Cat #HCL-WSUAML-AC), and Kasumi-1 (ATCC, Cat #CRL-2724) cell lines were maintained per the manufacturer's instructions. The Kasumi-1 FOLR1+ cell line was engineered by transducing Kasumi-1 cells with a lentivirus containing the FOLR1 transgene driven by the EF1a promoter (Genecopoeia, Cat #LPP-C0250-Lvl56-050). Jurkat Nur77 reporter cells (Rosskopf et al., Oncotarget 9: 17608-17619, 2018) were maintained in RPMI supplemented with 20% FBS and 2 mM L-Glutamine.

Constructs and Lentivirus production. The MSCV-CBFA2T3-GLIS2-IRES-mCherry construct was a gift from Dr. Tanja Gruber (Department of Oncology, St. Jude Children's Research Hospital, Memphis, Tn, (Gruber et al., Cancer Cell 22, 683-697, 2012)). The C/G fusion gene from this construct and the MND promoter were inserted into pRRLhPGK-GFP lentivirus vector (Dull et al., J Virol 72: 8463-8471, 1998) as described in Smith et al. (Clin Cancer Res 26: 726-737, 2020).

CAR constructs containing IgG4 short, intermediate and long spacers are previously described in Turtle et al. (Sci Transl Med 8: 355ra116, 2016). The VL and VH sequences from Farletuzumab were used to construct the anti-FOLR1 scFv with VL/VH orientation using G4SX4 linker. The anti-FOLR1 scFv DNA fragment was human codon optimized and synthesized by IDT gBlock gene fragment and cloned into the CAR vectors with NheI and RsrII restriction sites upstream of the IgG4 spacer.

Farletuzumab scFv:
DIQLTQSPSSLSASVGDRVTITCSVSSSISSNNLHWYQQKPGKAPKPWIYGTSNLASGVPSRFS
GSGSGTDYTFTISSLQPEDIATYYCQQWSSYPYMYTFGQGTKVEIKGGGGSGGGGSGGGGS
GGGGSEVQLVESGGGVVQPGRSLRLSCSASGFTFSGYGLSWVRQAPGKGLEWVAMISSGGS
YTYYADSVKGRFAISRDNAKNTLFLQMDSLRPEDTGVYFCARHGDDPAWFAYWGQGTPVTVS
S (SEQ ID NO: 22; linker underlined).

Lentivirus particles were produced in 293T cells (ATCC, Cat #CRL-3216). 293T cells were transfected with transfer vector, viral packaging vector (psPAX2), and viral envelope vector (pMD2G) at 4:2:1 ratio using Mirus 293Trans-IT transfection agent (Mirus, Cat #MIR2700) as directed by manufacturer's protocol. Viral particles were collected each day for 4 days post transfection, filtered through 0.45 μm membrane (Thermo Fisher; Cat NAL-166-0045) and concentrated (overnight spin at 4° C., 5000 rpm) before use.

Transduction of cord blood CD34+ cells. CB samples were processed with red blood cell lysis buffer and enriched for CD34+ cells using CliniMACS CD34 MicroBeads (Miltenyi Biotec, Cat #130-017-501). CB CD34+ cells were then seeded onto retronectin (5 ug/mL, Takara, Cat #T100A)+Notch ligand Delta1 (2.5 ug/mL, (Delaney et al., Nat Med 16: 232-236, 2010)) coated plates overnight in SFEM II medium (StemCell Technologies, Cat #09650FH) containing 50 ng/mL stem cell factor (SCF, StemCell Technologies, Cat #78062), 50 ng/mL thrombopoietin (TPO, StemCell Technologies, Cat #78210) and 50 ng/mL Fms-like tyrosine kinase 3 ligand (FLT3L, StemCell Technologies, Cat #78009). Cells were transduced the following day with the C/G construct at an MOI of 200 or GFP control construct at multiplicity of infection (MOI) of 50. Transduced cells were grown on Notch ligand at 37° C. in 5% CO₂ for 6 days then sorted for GFP+ cells. Sorted GFP+ cells were either transplanted into NSG-SGM3 mice at 200,000 cells per mouse or placed in EC co-culture or myeloid promoting condition (MC, see Imren et al. (Blood 124: 3608-3612, 2014) and below) for long term culture at 75,000 cells per 6-well. In a subsequent experiment using a CB CD34+ sample from another donor (CB 2, see FIGS. 7A-7D), transduced cells were grown on Notch ligand for 2 days prior to placement in EC co-culture or MC plating at 100,000 cells per 12-well.

Long term culture of transduced cord blood CD34+ cells. Transduced cells were placed in either EC co-culture with serum free expansion medium (SFEM) II medium supplemented with 50 ng/mL SCF, 50 ng/mL TPO, 50 ng/mL FLT3L, and 100U/mL Penicillin/Streptomycin, or MC containing Iscove's Modified Dulbecco's Medium (IMDM, Gibco 12-440-053) supplemented with 15% fetal bovine serum (FBS, Corning, 35-010-CV), 100U/mL Penicillin-Streptomycin (Pen/Strep, Gibco, 15-140-122), 10 ng/mL SCF, 10 ng/mL TPO, 10 ng/mL FLT3L, 10 ng/mL IL-6 (Shenandoah Biotechnology, Cat #100-10), and 10 ng/mL IL3 (Shenandoah, Cat #100-80). For EC co-cultures, human umbilical vein endothelial cells (HUVECs) transduced with E4ORF1 construct (E4 ECs) were propagated as previously described (Walter, R. B. et al. Leukemia 28: 1969-1977, 2014; and Butler et al., Cell Stem Cell 6: 251-264, 2010). One day prior to co-culture, E4 ECs were seeded into 6-well or 12-well plates at 800,000 or 300,000 cells per well, respectively, and cultured in medium 199 (Biowhittaker #12-117Q) supplemented with FBS (20%, Hyclone, Cat #SH30088.03), endothelial mitogen (Biomedical Technologies, Cat #BT203), Heparin (Sigma, Cat #H3149), HEPES (Gibco, Cat #15630080), L-Glutamine (Gibco, Cat #25030), and Pen/Strep. After 24 hours, E4 ECs were washed with phosphate buffered saline (PBS) and cultured with transduced CB cells in media described above. Transduced CB cells in either culture condition were propagated with fresh media and E4 ECs replaced every week until cells stopped proliferating. Three-to-twenty percent of the cultures were re-plated each week for long-term culture.

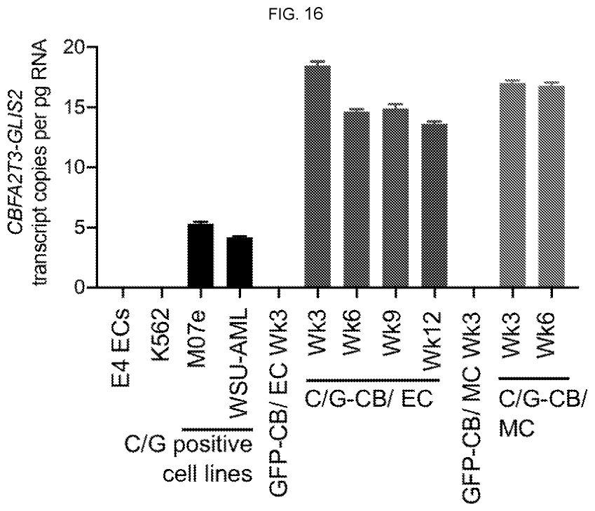

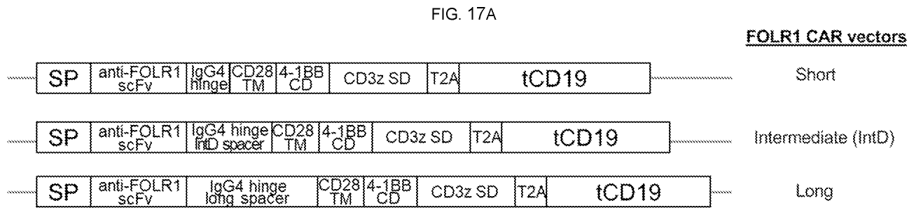

C/G and FOLR1 expression in engineered cells over weeks in culture was confirmed using RT-PCR (FIG. 16). Tranduced CB cells were sorted for GFP+ cells on an FACSAria II using FACSDiva Software (BD Biosciences). DNA and RNA from sorted cells were extracted with AllPrep DNA/RNA/miRNA Universal Kit using the QlAcube platform (QIAGEN). Expression of the fusion transcript in GFP+ cells was confirmed by RT-qPCR TaqMan assay and QuantStudio 5 real-time PCR system using the primers: Forward 5-CCCTGACGGTCATCAACCA-3 (SEQ ID NO: 114), Reverse 5-CACCATCCAAATAGCGCAGTG-3 (SEQ ID NO: 115), and TaqMan probe 5-[FAM]-CAGCGAGGACTTCCAG-[MGB]-3 (SEQ ID NO: 116). FOLR1 expression was determined using RT-qPCR TaqMan assay (Hs01124177_m1, cat #4331182).

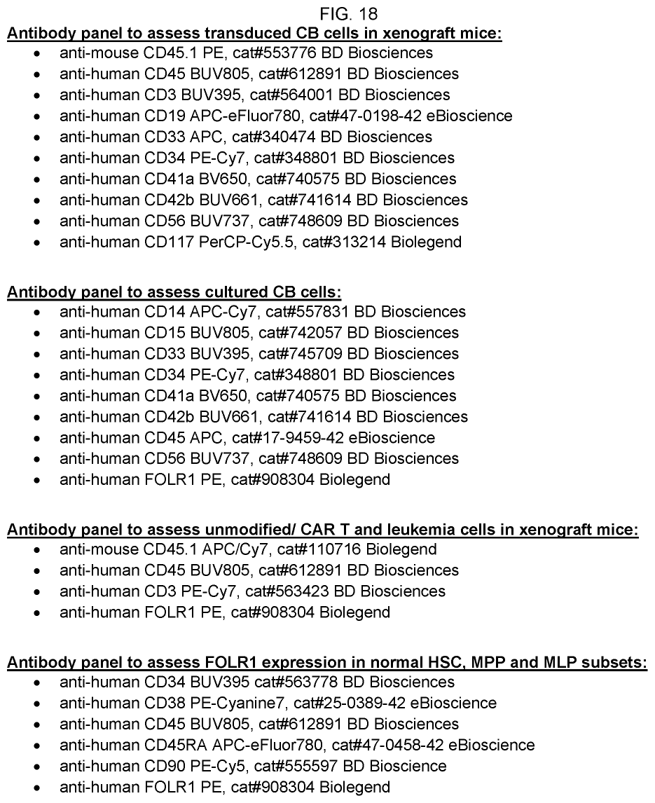

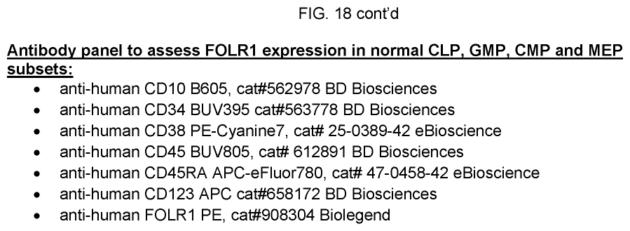

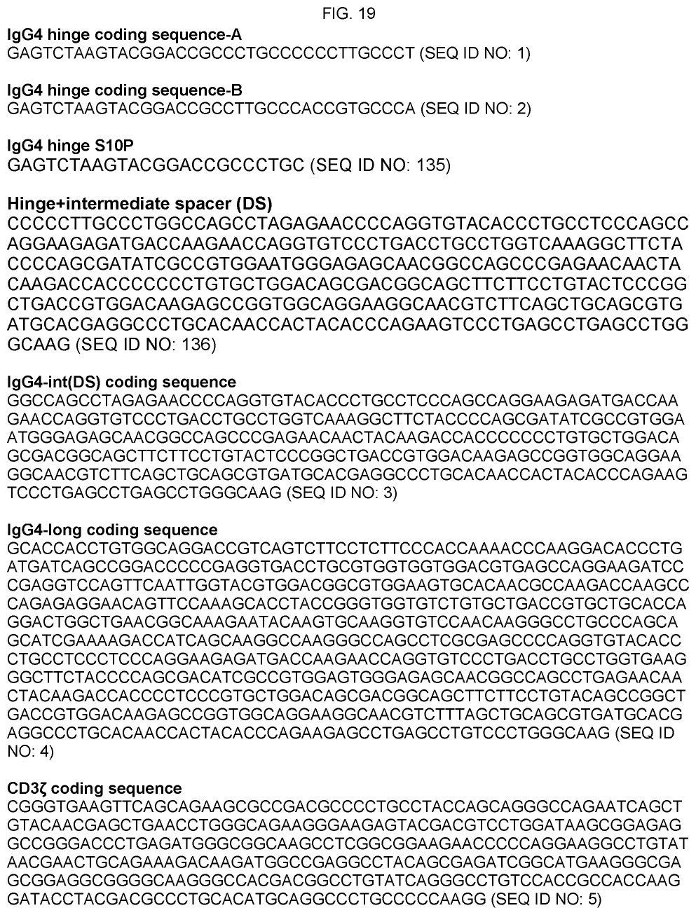

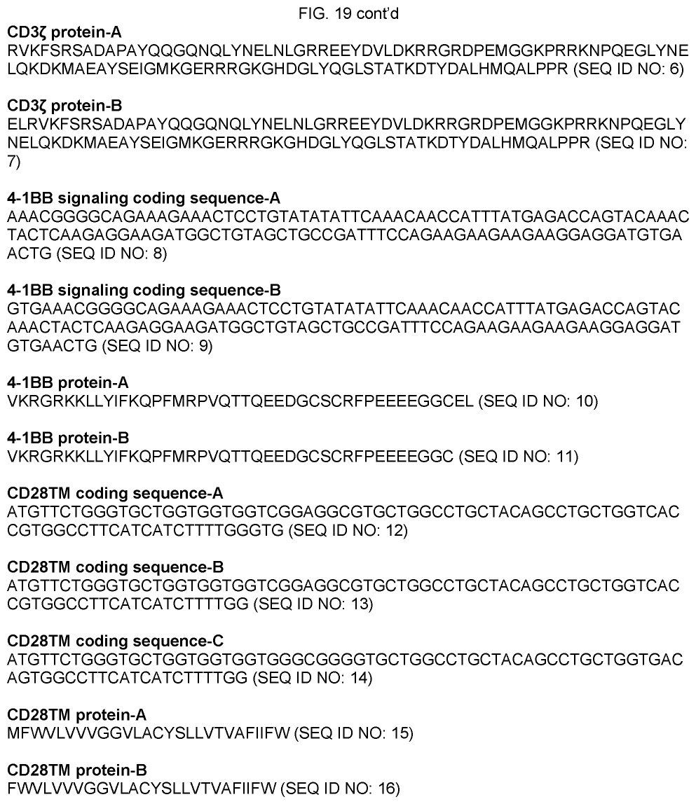

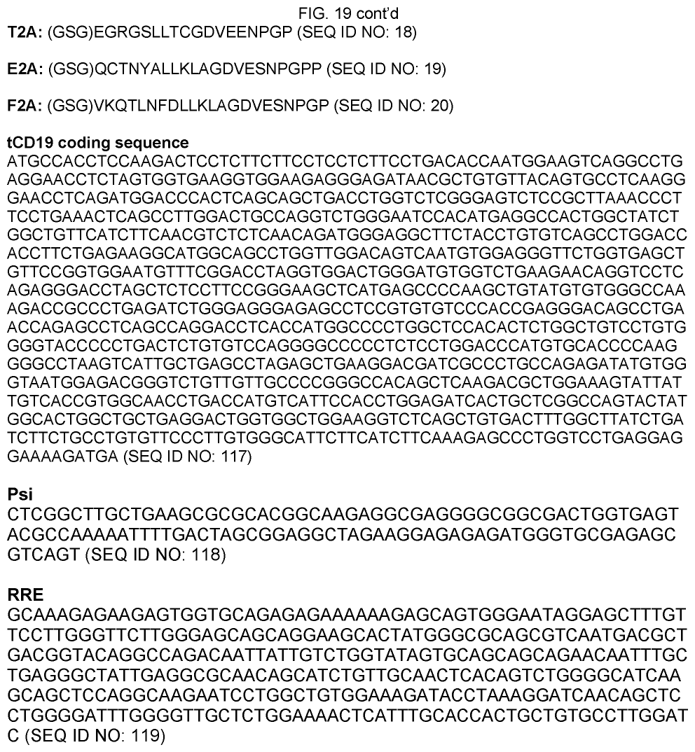

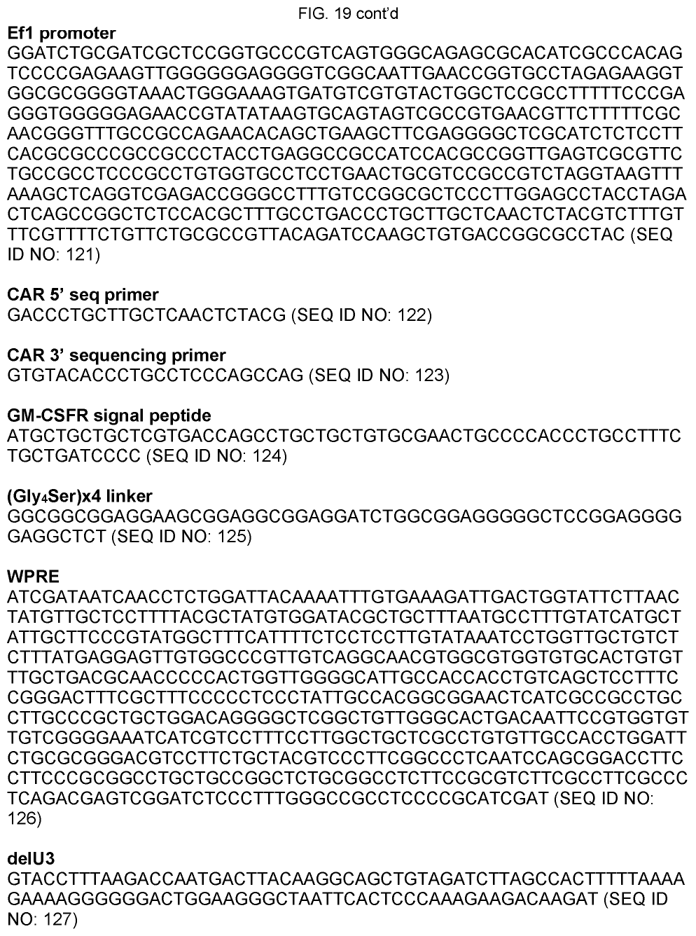

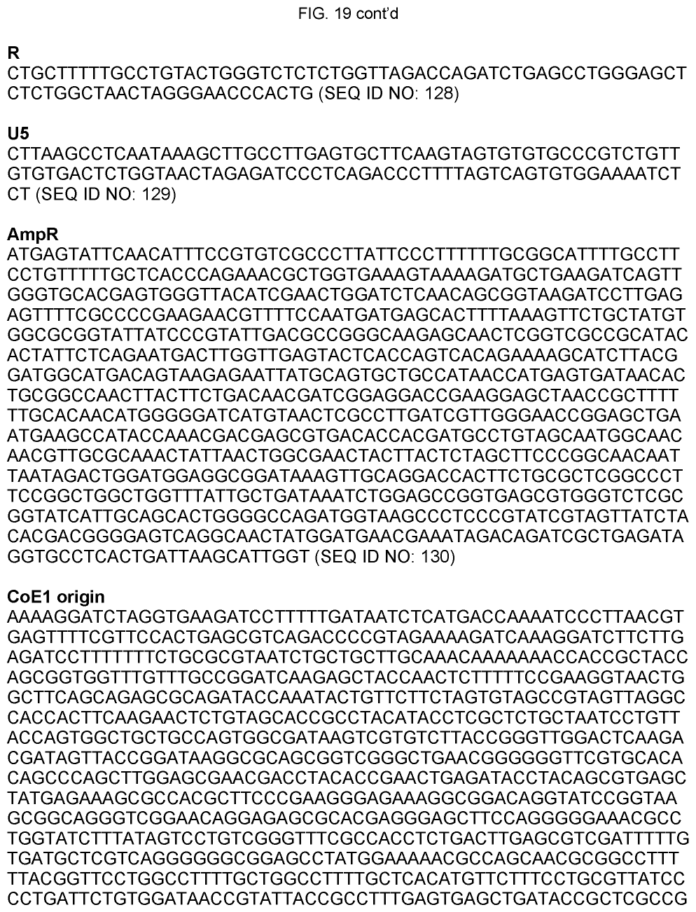

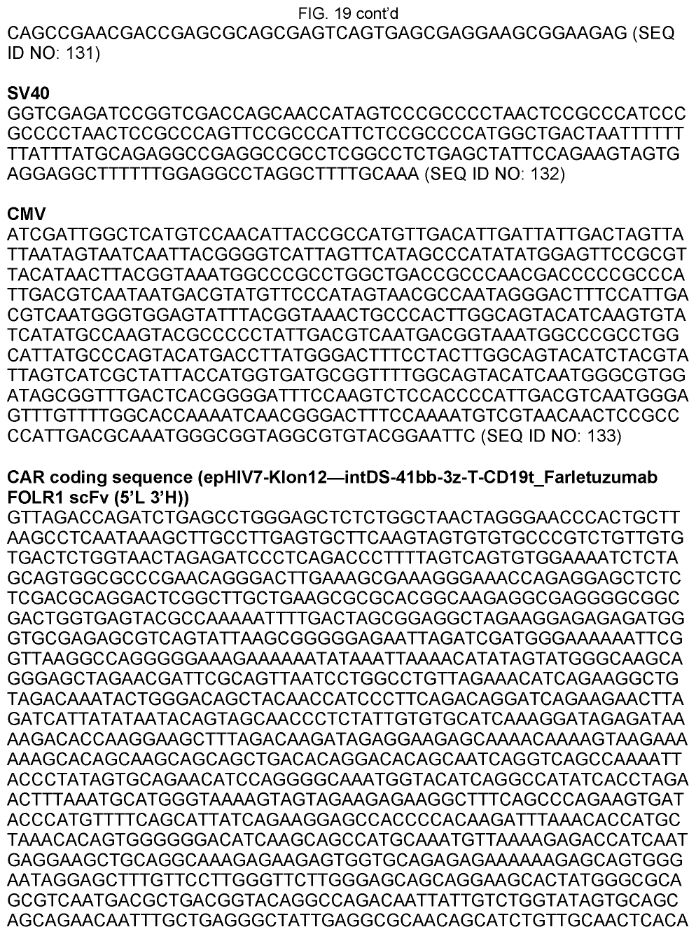

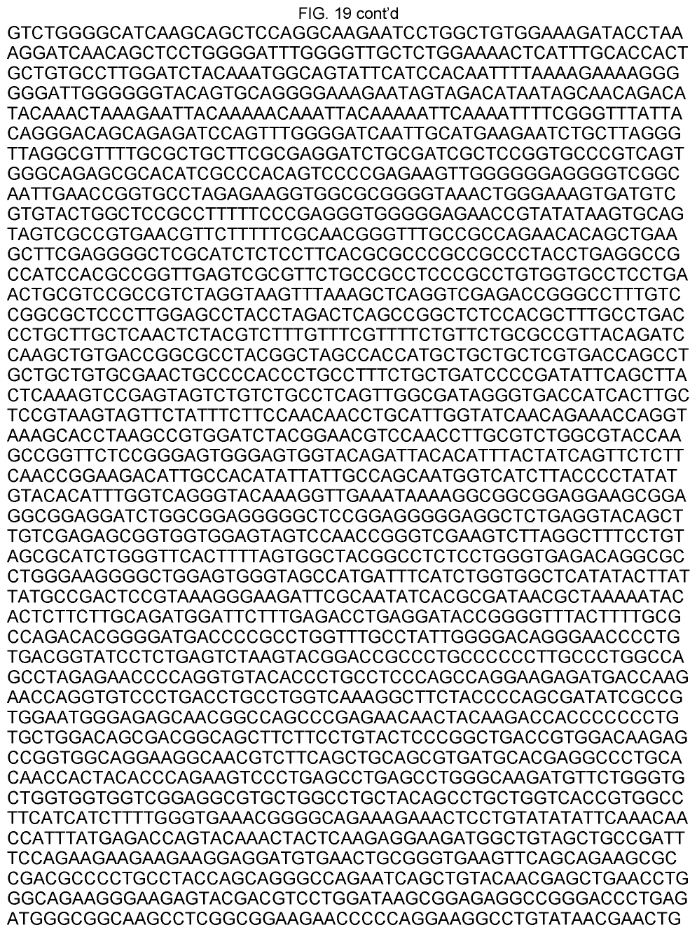

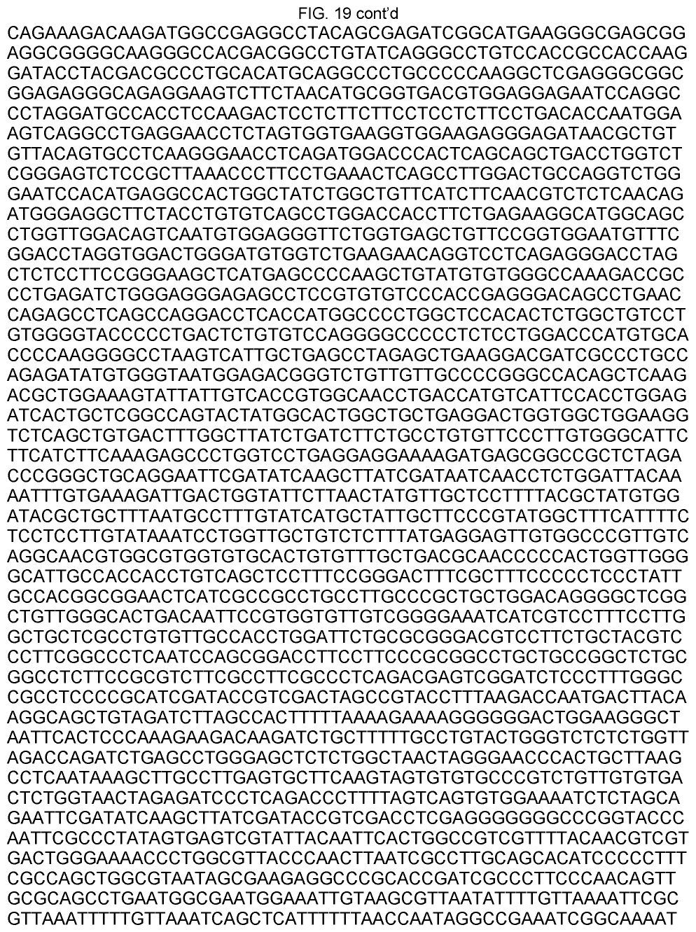

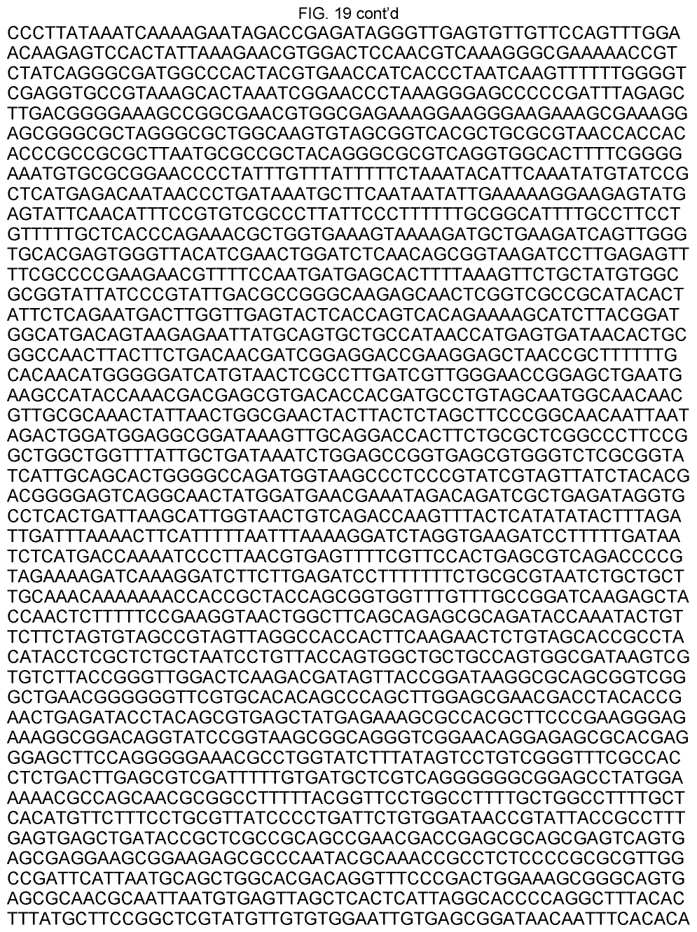

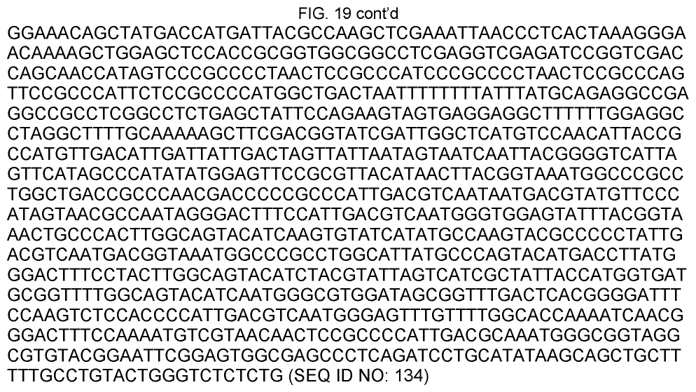

Cell surface analysis. For xenograft CB cells, mouse bone marrow, peripheral blood, spleen, and liver were harvested at necropsy and processed with red blood cell lysis buffer. Spleen and liver were processed into cell suspension with glass slides and passed through a 70-μm cell strainer. CB cells in EC co-culture and MC were harvested after vigorously pipetting to resuspend CB cells. CB cells from processed mouse tissues and cultures were washed in 2% FBS in PBS, blocked with 2% human AB serum in PBS, then stained with a cocktail of fluorescently labeled monoclonal antibodies for 20 min on ice (see FIG. 18). Labeled cells were washed with PBS and resuspended in 2% FBS/PBS prior to flow cytometric analysis. FACSymphony equipped with FACSDiva Software (BD Biosciences) was used to assess cell surface expressions and FlowJo Software was used for the analysis. Dead cells were excluded based on LIVE/DEAD™ Fixable Violet Dead Cell Stain (FVD, Invitrogen, cat #L34955). For EC co-cultures, ECs were excluded by gating on CD45+ cells or CD45+CD144− cells.

A fraction of the C/G-CB cells isolated from xenograft models or cultured in EC co-culture or MC at various timepoints were sent to Hematologics, Inc. (Seattle, WA) for assessment of the RAM immunophenotype along with C/G patient samples.

Histology and Immunocytochemistsry. Sample tissues were fixed in 10% formalin, processed into paraffin sections and stained with hematoxylin and eosin (H&E). Immunohistochemistry was performed using antibodies to ERG (EP111; Cell Marque) and CD56 (MRQ-42; Cell Marque) following citrate pretreatment and visualized with 3, 3′-diaminobenzidine (DAB) on a Ventana Bench Mark Ultra.

All tissues were examined by a board certified Hematopathologist. The bone marrow core biopsy specimen was fixed in acetic acid-zinc-formalin (AZF), decalcified, and embedded in paraffin, and sections were stained for CD56 (clone MRQ-42; Cell Marque, Rockin, California).

RNA seq analysis. RNA-sequencing Library Construction. Total RNA was extracted using the QlAcube automated system with AllPrep DNA/RNA/miRNA Universal Kits (QIAGEN, Valencia, CA, #80224) for diagnostic pediatric AML samples from peripheral blood or bone marrow, as well as, bulk healthy bone marrows, and healthy CD34+ peripheral blood samples. Total RNA from C/G-CB and GFP-CB cells in EC co-culture and MC at indicated timepoints was purified as described above. The 75 bp strand-specific paired-end mRNA libraries were prepared using the ribodepletion 2.0 protocol by the British Columbia Genome Sciences Center (BCGSC, Vancouver, BC) and sequenced on the Illumina HiSeq 2000/2500. Sequenced reads were quantified using Kallisto v0.45.0 (Bray et al., Nat Biotechnol 34: 525-527, 2016) with a GRCh38 transcriptome reference prepared using the coding and noncoding transcript annotations in in Gencode v29 and RepBase v24.01 and gene-level counts and abundances were produced using tximport v1.16.1 (Soneson et al., F1000Res 4: 1521, 2015).

Screening of C/G Fusion in patient samples. The C/G fusion transcript was detected by fragment length analysis or fusion detection algorithms STAR-fusion v1.1.0 and TransAbyss v1.4.10 (Haas et al., Genome Biol 20: 213, 2019; and Robertson et al., Nat Methods 7: 909-912, 2010). Details of the procedure are described previously (Smith et al., Clin Cancer Res 26: 726-737, 2020).

Transcriptome Analysis: Differentially expressed genes between C/G-CB and GFP-CB cells were identified using the limma voom (v3.44.3 R package) with trimmed mean of M values (TMM) normalized gene counts (Ritchie et al., Nucleic Acids Res 43: e47, 2015). Genes with absolute log 2 fold-change>1 and Benjamini-Hochberg adjusted p-values<0.05 were retained. Unsupervised hierarchical clustering was completed using the ComplexHeatmap R package (v2.4.3), utilizing Euclidean distances with the ward.D2 linkage algorithm. Log 2 transformed TMM normalized counts per million (CPM) were used as input, with a count of 1 added to avoid taking the log of zero. Hierarchical clustering of primary C/G AML samples and C/G-CB cells using a C/G transcriptome signature was carried out. The signature genes (N=1,116 genes) were defined as those within the 75^{th} percentile of absolute log 2 fold-changes and adj. p.value<0.001, when contrasting C/G fusion positive patients (N=39) against a heterogenous AML reference cohort (N=1,355). The 85^{th} percentile of this signature (N=167 genes) was used to define a C/G gene set in GSEA.

Gene-set enrichment scores were calculated using the single-sample gene-set enrichment (ssGSEA) method (GSVA v1.32.0), which transforms normalized count data from a gene by sample matrix to a gene-set by sample matrix (Hanzelmann et al., BMC Bioinformatics 14: 7, 2013). Counts were TMM normalized and log 2(x+1) transformed prior to gene-set analysis. Curated signaling and metabolic gene-sets from the KEGG database were included in the analysis (gageData v2.26.0). Significant gene-sets (Benjamini-Hochberg adjusted p-values<0.05) associated with C/G-CB cells were identified using limma v3.44.3 with the GSVA transformed gene-set by sample matrix as input.

GSEA was performed using the ‘unpaired’ comparison in the GAGE R package (v2.38.3), which tests for differential expression of gene-sets by contrasting C/G-CB against GFP-CB cells in each condition to define pathways enriched in EC co-culture versus MC. Non-redundant gene-sets were extracted for further analysis, followed by the identification of core genes that contribute to the pathway enrichment. Gene-sets from the Molecular Signatures Database (MSigDB) and the KEGG pathway database were used. Enrichment score plots for the HSC and C/G signatures were generated using the R package fgsea (v1.14.0). Log fold change values obtained from limma (contrasting C/G-CB EC week 6 against C/G-CB MC week 6) were used as a ranking metric for genes in the two signatures.

Unsupervised clustering of C/G-CB cells with pediatric AML primary diagnostic samples (N=1,033) and healthy normal bone marrows (N=68) was performed by uniform manifold approximation and projection (UMAP) using the uwot v0.1.8 R package (Leland McInnes and Melville. UMAP: Uniform ManifoldApproximation and Projection forDimension Reduction. arXiv:1802.03426, 2020). For UMAP clustering, gene counts underwent variance stabilizing transformation (VST) using the DESeq2 v1.28.1 package. Input genes for clustering (N=6,678 genes) were selected using the mean versus dispersion parametric model trend (SeqGlue v0.1) to identify genes with high variability.

Identification of fusion-specific CAR targets involves three main steps: 1) Determine the ratio of expression for AML primary samples versus healthy normal hematopoietic tissue samples (bulk normal bone marrow, N=68, in combination with CD34+ selected peripheral blood samples, N=16) from log 10 transformed normalized expression as transcripts per million, (TPM). Normalization was completed on the full gene expression matrix followed by ratio analysis on 19,901 annotated protein-coding genes for the identification of therapeutic targets. The ratio is calculated per gene from the mean expression in AML and normal tissues, where normal healthy hematopoietic tissue mean expression is the divisor, which acts as a measure of over or under expression. A normal curve is fit to the ratio values, and genes with ratios greater than +2 standard deviations were retained. This process is carried out for all heterogenous AML samples (N=1483) as a group and then repeated iteratively within AML fusion and mutation subtypes, including C/G, to ensure the inherent variability of gene expression in different fusion classes is addressed and all viable targets are identified for any given subtype. Genes are then further refined to include those with maximum expression<1.0 TPM in normal healthy hematopoietic tissue samples, and thus considered to have AML restricted expression when compared to healthy controls. 2) AML restricted genes were further selected if found to be significantly overexpressed by RNA-seq for bulk fusion positive patient samples compared to bulk healthy bone marrows and were likewise overexpressed in C/G-CB at weeks 6 and 12 in EC co-culture with an absence of expression (<1.0 TPM) in GFP-CB controls providing several candidate targets. 3). Final selection of optimal CAR-T targets was determined by the identification of candidate genes with cell surface localization potential as annotated by the Human Protein Atlas (www.proteinatlas.org/) or Jensen Lab compartments database (https://compartments.jensenlab.org/), in addition to having moderate to high expression in C/G patient samples (maximum expression≥10 TPM), expression in a majority (>75%) of patient samples, and an absence of expression in healthy hematopoietic tissues as noted in step 1 above.

Generation of human FOLR1 CAR T cells. CAR T cells were generated by transducing healthy donor T cells (Bloodworks Northwest) with lentivirus carrying the FOLR1 CAR vectors. Peripheral blood mononuclear cells from healthy donors were isolated over Lymphoprep (StemCell Technologies, Cat #07851). CD4 or CD8 T cells were isolated by negative magnetic selection using Easy Sep Human CD4+ T cell Isolation Kit II (StemCell Technologies, Cat #17952) and Easy Sep Human CD8+ T cell Isolation Kit II (StemCell Technologies, Cat #17953).

Purified T cells were cultured in CTL media [RPMI supplemented with 10% Human serum (Bloodworks Northwest), 2% L-glutamine (Gibco, Cat #25030-081 1% pen-strep (Gibco, Cat #15140-122), 0.5 M beta-mercaptoethanol (Gibco, Cat #21985-023), and 50 U/ml IL-2 (aldesleukin, Prometheus)] at 37° C. in 5% CO₂. T cells were activated with anti-CD3/CD28 beads (3:1 beads: cell, Gibco, 11131D) on Retronectin-coated plates (5 μg/mL, coated overnight at 4° C.; Takara, Cat #T100B) and transduced with CAR lentivirus (MOI=50) one day after activation via spinoculation at 800 g for 90 min at 25° C. in CTL media (+50 U/mL IL-2) supplemented with 8 ug/mL protamine sulfate. Transduction used 200,000 cells per well in 24-well plates. Transduced cells were expanded in CTL media (+50 U/mL IL-2) and separated from beads on day 5. As truncated CD19 was co-expressed with the CAR by a T2A ribosomal skip element, it was used to select for transduced cells. Transduced cells were sorted for CD19 expression [using anti-human CD19 microbeads (Miltenyi Biotec, Cat #130-050-301)] on Automacs 8-10 days post activation. Sorted cells were further expanded in CTL (+50 U/mL IL-2) media for an additional 4-6 days prior to in vitro and in vivo cytotoxicity assays.

In vitro cytotoxicity studies. Target cells (C/G-CB>9 weeks in EC co-culture, M07e, WSU-AML, Kasumi-1 FOLR1+ and Kasumi-1 parental) were split 1-2 days prior to cytotoxicity assay. Target leukemia cells were labeled with 2.5 μM carboxyfluorescein succinimidyl ester (CFSE) (Invitrogen, Cat #C34554) per the manufacturer's protocol, washed with 1×PBS, and resuspended in CTL media (without IL-2). For T cell proliferation assay, effector cells (unmodified or CAR T cells) were labeled with 2.5 μM Violet Cell Proliferation Dye (Invitrogen, Cat #C34557) washed with 1×PBS, serial diluted in CTL media (without IL-2) and combined with target cells at various effector:target (E:T) ratios in 96-well U-bottom plate. Cytotoxicity (at indicated time points) and T cell proliferation (4 days) were assessed by flow cytometry after staining cells with live/dead fixable viability dyes [FVD; Invitrogen, Cat #L34964 (cytotoxicity) or L10120 (T cell proliferation)]. Percent dead amongst target cells was assessed by gating on FVD+ amongst CFSE+ target cells. Percent specific lysis was calculated by subtracting the average of the three replicate wells containing target cells only from each well containing target and effector cells at each E:T ratio. After 24 hours of co-culture, media supernatant was assessed for IL-2, IFN-γ, and TNF-α production by Luminex microbead technology (provided by FHCRC Immune Monitoring Core).

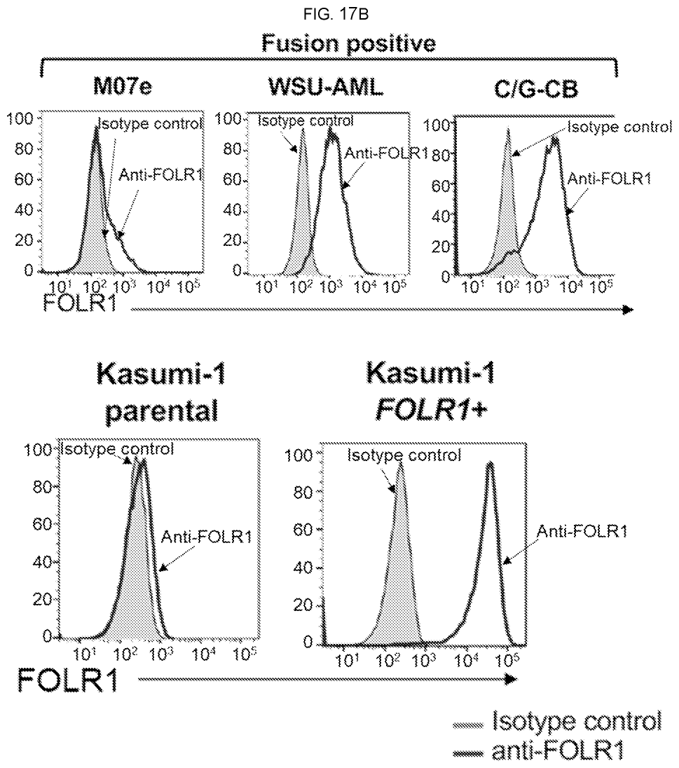

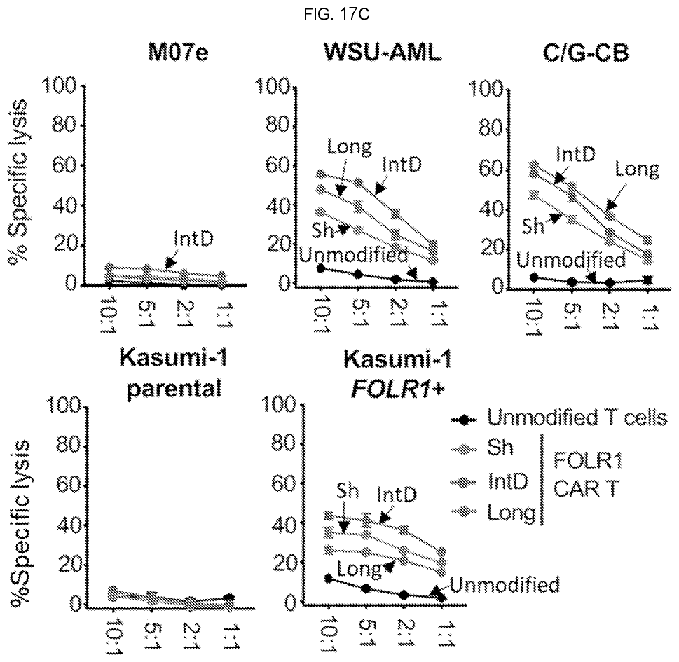

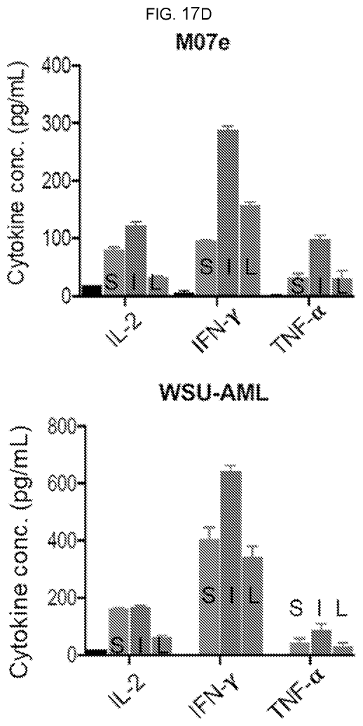

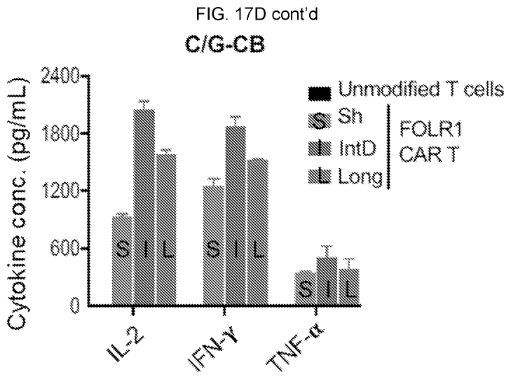

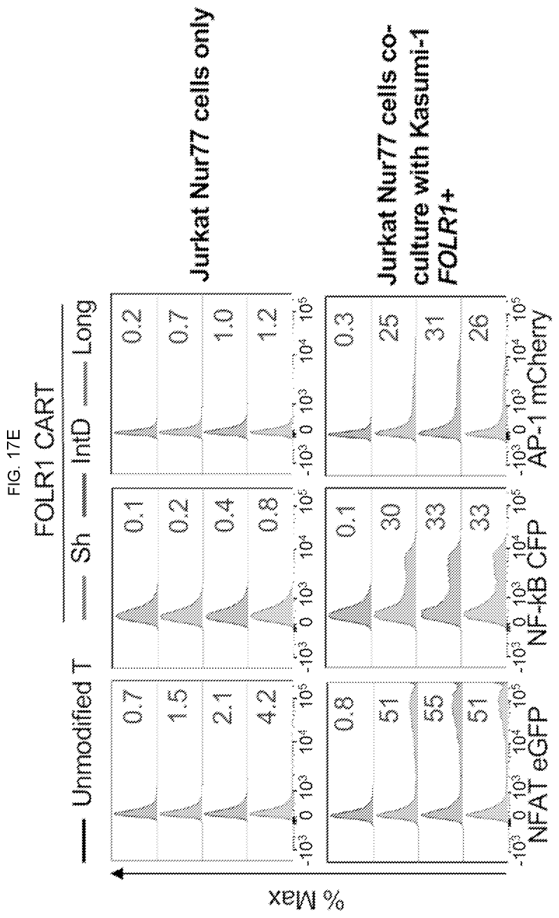

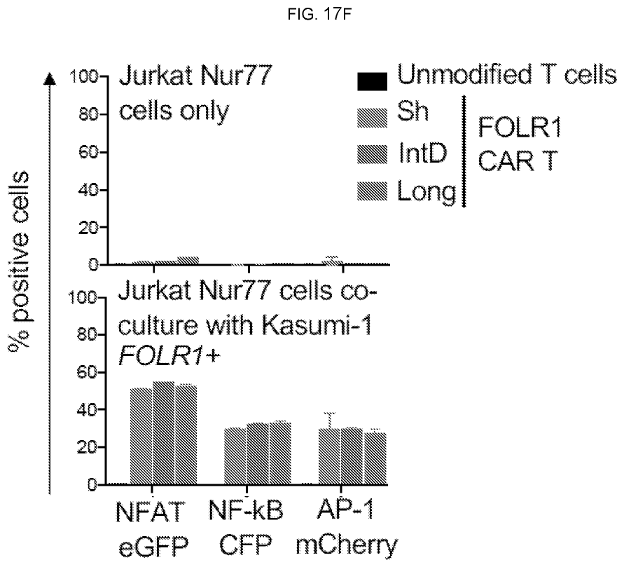

Optimization of IgG4 spacer region for efficient CAR T activity. To evaluate the therapeutic potential of targeting FOLR1, FOLR1-directed CAR were generated by fusing the single-chain variable fragment (scFv) derived from anti-FOLR1 antibody Farletuzumab to the IgG4 spacer, CD28 transmembrane, 4-1BB co-stimulatory and CD3z signaling domains (FIG. 17A). The IgG4 spacer region was optimized against fusion-positive cells lines (M0-7e and WSU-AML), C/G-CB cells, Kasumi-1 cells engineered to express FOLR1 (Kasumi-1 FOLR1+) and Kasumi-1 parental cells (FIG. 17B). Although all constructs conferred similar cytotoxicity against FOLR1+ cells, intermediate spacer CAR produced higher levels of proinflammatory cytokines (IL-2, IFN-γ and TNF-α) compared to short and long IgG4 spacers (FIGS. 17C and 17D). NFAT, NFkB and AP-1 expression were assayed in Jurkat Nur77 reporter cells (Rosskopf et al., Oncotarget 9: 17608-17619, 2018) transduced with the CAR constructs either cultured alone or co-cultured with Kasumi-1 FOLR1+ cells. None of the FOLR1 CAR constructs demonstrated tonic signaling in the absence of target binding (FIGS. 17E and 17F).

In vivo cytotoxicity studies. Target leukemia cells were transduced with mCherry/ffluciferase (C/G-CB, weeks 9-12 in EC co-culture; Plasmid #104833, Addgene) or eGFP/ffluciferase construct (WSU-AML, Kasumi-1 FOLR1+ and Kasumi-1 parental; Plasmid #104834, Addgene) and sorted for mCherry+ or GFP+ cells, respectively. Luciferase-expressing cells were injected intravenously into NSG-SGM3 (C/G-CB) at 5×10⁶ cells per mouse or NSG (WSU-AML, Kasumi-1 FOLR1+ and Kasumi-1 parental) mice at 1×10⁶ cells through the tail vein. Mice were treated with FOLR1 CAR T or unmodified T cells via tail vein intravenous injection one week following leukemia cell injection.

Leukemia burden was measured by bioluminescence imaging weekly. Leukemia burden and T cell expansion were monitored by flow cytometric analysis of mouse peripheral blood, which was drawn by retro-orbital bleeds for the indicated time points starting from the first week of T cell injection. Flow cytometric analysis of peripheral blood and tissues was performed as described elsewhere herein (FIG. 18).

Colony-forming cell assay. Following 6 and 12 weeks of culture, cells were placed in Megacult (Megacult-C, Collagen & Medium with Cytokines Stemcell Technologies, Vancouver, Canada, Cat #04961) and incubated at 37° C. in 5% CO₂ for 10-14 days. Colonies from megacult cultures were fixed in 3.7% formaldehyde, and then washed in PBS, and stained with MegaCult™-C Staining Kit for CFU-Mk (StemCell Technologies, Vancouver, Canada, Cat #04962) per the manufacturer's instructions; or were permeabilized after fixation in 0.1% Triton X-100 for 10 min, blocked in in 1% BSA in PBST(PBS+0.1% Tween-20) for 30 min, then stained with biotin-conjugated mouse anti-human CD41 (Biolegend, cat #303734) and FITC-conjugated goat anti-GFP (abcam, cat #ab6662) followed by secondary stain with Alexa 647-labeled Streptavidin (Biolegend, cat #405237) per the manufacturer's instructions, and colonies were stained with DAPI prior to imaging using the TissueFAX microscope. Mk colonies were scored based on positive staining for CD41 and enumerated.

C/G-CB and normal HPSCs after co-culture with unmodified or CAR T cells for 4 hours were placed in Methocult H4034 Optimum (Stemcell Technologies, Cat #04034). Colonies derived from erythroid (E), granulocyte-macrophage (G, M, and GM) and multipotential granulocyte, erythroid, macrophage, megakaryocyte (GEMM) progenitors were scored and enumerated after 7-10 days as directed by manufacturer's instructions.

Statistical analysis for in vitro and in vivo studies. Unpaired, two-tailed Student's t test was used to determine statistical significance for all in vitro studies. Log-rank (Mantel-Cox) test was used to compare Kaplan-Meier survival curves between experimental groups. Statistical significance is defined for p<0.05.

Data and code Availability. RNA-seq data on primary patient samples are deposited in GDC, SRA and Target Data Matrix. RNA-seq data on engineered CB are deposited in GEO. All codes used in this are publicly available.

### Example 2. Development and Preclinical Assessment of FOLR1-Directed Chimeric Antigen Receptor T Cells in CBF2AT3-GLIS2/RAM AML

Background A rare but highly aggressive type of acute myeloid leukemia (AML) that is only seen in infants with a unique immunophenotype (RAM phenotype which is characterized by positive CD56 expression, negative CD45 expression, negative CD38 expression, and negative HLA-DR expression) is caused by cryptic CBFA2T3-GLIS2 (CBF/GLIS) fusion. This infant AML is highly refractory to conventional chemotherapy with near uniform fatality despite highly intensive and myeloablative therapy (Gruber et al., Cancer Cell. 22(5):683-97, 2012). Transcriptome profiling of CBF/GLIS AML has revealed new insights into the pathogenesis of the fusion and uncovered fusion-specific molecular biomarkers that could be used for risk stratification and to inform treatment (Masetti et al., Br J Haematol. 184(3):337-47, 2019). Studying the largest cohort of these high-risk infants, several alterations were demonstrated in gene expression and transcriptional networks in these CBF/GLIS-positive patient samples that have potential for therapeutic targeting (Smith et al., Clin Cancer Res. 26(3):726-737, 2020). FOLR1, which encodes for folate receptor alpha, was highly and uniquely expressed in CBF/GLIS AML but was entirely absent in AML with other cytogenetics abnormalities and in normal hematopoietic cells. Furthermore, it was demonstrated that forced expression of CBF/GLIS enhances the proliferation and alters differentiation in cord blood (CB) CD34+ early precursors towards megakaryocytic lineage that recapitulates acute megakaryocytic leukemia seen in infants (Smith et al., Clin Cancer Res. 26(3):726-737, 2020). Of significance, FOLR1 surface expression is shown to be causally linked to CBF/GLIS-induced malignant transformation, thus making it an attractive antigen for targeted therapies against CBF/GLIS AML cells. Given that chimeric antigen receptor (CAR) T cells are extremely effective at eradicating relapsed/refractory B-cell acute lymphoblastic leukemia (B-ALL) malignancies, FOLR1-directed CAR T cells were developed for pre-clinical evaluation in CBF/GLIS AML.

Methods. A FOLR1-directed CAR was generated using anti-FOLR1 binder (Farletuzumab), IgG4 intermediate spacer and 41BB/CD3zeta signaling domains. The pre-clinical efficacy of FOLR1 CAR T cells was evaluated against CBF/GLIS AML cell lines in vitro and in vivo. CBF/GLIS AML models include CB CD34+ cells transduced with CBF/GLIS expression construct (CBF/GLIS-CB) and WSU-AML cell line. Kasumi-1 cell line was also engineered to express FOLR1 (Kasumi-1 FOLR1+) to evaluate target specificity (FIG. 17B).

Results. The target specificity of FOLR1-directed CAR T cells was tested against FOLR1-positive (CBF/GLIS-CB, WSU-AML, Kasumi-1 FOLR1+) and FOLR1-negative (Kasumi-1) cells. CD8 FOLR1 CAR T cells demonstrated cytolytic activity against FOLR1 positive but not FOLR1 negative cells (FIG. 13A). Furthermore, both CD8 and CD4 FOLR1 CAR T cells produced higher levels of IL-2, IFN-γ, and TNF-α and proliferated more robustly than did unmodified T cells when co-incubated with FOLR1 positive but not FOLR1 negative cells (FIG. 13B). These results indicate highly specific reactivity of FOLR1 CAR T cells against AML cells expressing FOLR1. Next, the in vivo efficacy of FOLR1-directed CAR T cells was investigated. In CBF/GLIS-CB, WSU-AML, and Kasumi-1 FOLR1+ xenograft models, treatment with FOLR1 CAR T cells induced leukemia clearance, while disease progression occurred in all mice that received unmodified T cells (FIG. 13D). Activity of FOLR1 CAR T cells in vivo was target specific, as they did not limit the leukemia progression nor extend the survival of Kasumi-1 xenografts (FIG. 13D).

To determine whether FOLR1 is expressed on normal hematopoietic stem and progenitor cells (HSPCs), FOLR1 expression was characterized in normal CB CD34+ samples. FOLR1 expression was entirely silent in HSPC subsets (FIG. 15C). Consistent with lack of expression, no cytolytic activity was detected against HSPCs Moreover, FOLR1 CAR T cells did not affect the self-renewal and multilineage differentiation capacity of normal HSPCs as compared to unmodified control T cells (FIG. 15E), whereas significant eradication of colonies were detected in the CBF/GLIS-CB cells (FIG. 15F).

Conclusion. In this example, it is demonstrated that FOLR1 CAR T effectively eradicates CBF/GLIS AML cells without compromising normal HSPCs, providing a promising approach for the treatment of high-risk CBF/GLIS AML. Transition of this CAR T to clinical development for infant AML is underway.

(xiii) Closing Paragraphs. The nucleic acid and amino acid sequences provided herein are shown using letter abbreviations for nucleotide bases and amino acid residues, as defined in 37 C.F.R. § 1.822 and set forth in the tables in WIPO Standard ST.25 (1998), Appendix 2, Tables 1 and 3. Only one strand of each nucleic acid sequence is shown, but the complementary strand is understood as included in embodiments where it would be appropriate.

To the extent not explicitly provided herein, coding sequences for proteins disclosed herein and protein sequences for coding sequences disclosed herein can be readily derived from one of ordinary skill in the art.

Variants of the sequences disclosed and referenced herein are also included. Guidance in determining which amino acid residues can be substituted, inserted, or deleted without abolishing biological activity can be found using computer programs well known in the art, such as DNASTAR™ (Madison, Wisconsin) software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gln), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (lie), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (non-polar): Proline (Pro), Ala, Val, Leu, lie, Phe, Met, and Trp; Group 11 (aliphatic): Gly, Ala, Val, Leu, and lie; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W.H. Freeman and Company.

In making such changes, the hydropathic index of amino acids may be considered. The importance of the hydropathic amino acid index in conferring interactive biologic function on a protein is generally understood in the art (Kyte and Doolittle, 1982, J. Mol. Biol. 157(1), 105-32). Each amino acid has been assigned a hydropathic index on the basis of its hydrophobicity and charge characteristics (Kyte and Doolittle, 1982). These values are: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glutamate (−3.5); Gln (−3.5); aspartate (−3.5); Asn (−3.5); Lys (−3.9); and Arg (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: Arg (+3.0); Lys (+3.0); aspartate (+3.0±1); glutamate (+3.0±1); Ser (+0.3); Asn (+0.2); Gln (+0.2); Gly (0); Thr (−0.4); Pro (−0.5±1); Ala (−0.5); His (−0.5); Cys (−1.0); Met (−1.3); Val (−1.5); Leu (−1.8); Ile (−1.8); Tyr (−2.3); Phe (−2.5); Trp (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like.

As indicated elsewhere, variants of gene sequences can include codon optimized variants, sequence polymorphisms, splice variants, and/or mutations that do not affect the function of an encoded product to a statistically significant degree.

Variants of the protein, nucleic acid, and gene sequences disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including (but not limited to) those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this disclosure, it will be understood that where sequence analysis software is used for analysis, the results of the analysis are based on the “default values” of the program referenced. As used herein “default values” will mean any set of values or parameters, which originally load with the software when first initialized.

Variants also include nucleic acid molecules that hybridizes under stringent hybridization conditions to a sequence disclosed herein and provide the same function as the reference sequence. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6×SSPE (20×SSPE=3M NaCl; 0.2M NaH2PO4; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

“Binds” refers to an association of a binding domain (of, for example, a CAR binding domain or a nanoparticle selected cell targeting ligand) to its cognate binding molecule with an affinity or Kₐ (i.e., an equilibrium association constant of a particular binding interaction with units of 1/M) equal to or greater than 10⁵ M⁻¹, while not significantly associating with any other molecules or components in a relevant environment sample. Binding domains may be classified as “high affinity” or “low affinity”. In particular embodiments, “high affinity” binding domains refer to those binding domains with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. In particular embodiments, “low affinity” binding domains refer to those binding domains with a Kₐ of up to 10⁷ M⁻¹, up to 10⁶ M⁻¹, up to 10⁵ M⁻¹. Alternatively, affinity may be defined as an equilibrium dissociation constant (K_{d}) of a particular binding interaction with units of M (e.g., 10⁻⁵ M to 10⁻¹³ M). In certain embodiments, a binding domain may have “enhanced affinity,” which refers to a selected or engineered binding domains with stronger binding to a cognate binding molecule than a wild type (or parent) binding domain. For example, enhanced affinity may be due to a Kₐ (equilibrium association constant) for the cognate binding molecule that is higher than the reference binding domain or due to a K_{d} (dissociation constant) for the cognate binding molecule that is less than that of the reference binding domain, or due to an off-rate (K_{off}) for the cognate binding molecule that is less than that of the reference binding domain. A variety of assays are known for detecting binding domains that specifically bind a particular cognate binding molecule as well as determining binding affinities, such as Western blot, ELISA, and BIACORE® analysis (see also, e.g., Scatchard, et al., 1949, Ann. N.Y. Acad. Sci. 51:660; and U.S. Pat. Nos. 5,283,173, 5,468,614, or the equivalent).

Unless otherwise indicated, the practice of the present disclosure can employ conventional techniques of immunology, molecular biology, microbiology, cell biology and recombinant DNA. These methods are described in the following publications. See, e.g., Sambrook, et al. Molecular Cloning: A Laboratory Manual, 2nd Edition (1989); F. M. Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant reduction in the ability to treat cancer, as described herein.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

What is claimed is:

1. A chimeric antigen receptor comprising, when expressed by a cell,
an extracellular component comprising a binding domain having the sequence as set forth in SEQ ID NO: 22 or SEQ ID NO: 23;
an intracellular component comprising an effector domain; and
a transmembrane domain linking the extracellular component to the intracellular component.

2. A targeted therapeutic molecule comprising a binding domain that binds folate receptor 1 (FOLR1), multiple EGF like domain 10 (MEGF10), heparinase-2 enzyme (HPSE2), killer cell lectin like receptor F2 (KLRF2), protocadherin-19 (PCDH19), or Fraser extracellular matrix complex subunit 1 (FRAS1).

3. The targeted therapeutic molecule of claim 2, wherein the targeted therapeutic molecule is a chimeric antigen receptor (CAR) comprising, when expressed by a cell,
an extracellular component comprising the binding domain that binds FOLR1, MEGF10, HPSE2, KLRF2, PCDH19, or FRAS1;
an intracellular component comprising an effector domain; and
a transmembrane domain linking the extracellular component to the intracellular component.

4. The targeted therapeutic molecule of claim 3, wherein the binding domain specifically binds FOLR1.

5. The targeted therapeutic molecule of claim 3, wherein the binding domain comprises a single chain variable fragment (scFv).

6. The targeted therapeutic molecule of claim 5, wherein the scFv has the sequence as set forth in SEQ ID NO: 22 or SEQ ID NO: 23.

7. The targeted therapeutic molecule of claim 4, wherein the binding domain comprises a variable heavy chain set forth in SEQ ID NO: 30 and a variable light chain set forth in SEQ ID NO: 31; a variable heavy chain set forth in SEQ ID NO: 38 and a variable light chain set forth in SEQ ID NO: 39; a variable heavy chain set forth in SEQ ID NO: 40 and a variable light chain set forth in SEQ ID NO: 41; a variable heavy chain set forth in SEQ ID NO: 48 and a variable light chain set forth in SEQ ID NO: 49; a variable heavy chain set forth in SEQ ID NO: 56 and a variable light chain set forth in SEQ ID NO: 57; a variable heavy chain set forth in SEQ ID NO: 64 and a variable light chain set forth in SEQ ID NO: 65; a variable heavy chain set forth in SEQ ID NO: 72 and a variable light chain set forth in SEQ ID NO: 73; or a variable heavy chain set forth in SEQ ID NO: 80 and a variable light chain set forth in SEQ ID NO: 81.

8. The targeted therapeutic molecule of claim 4, wherein the binding domain comprises a variable heavy chain with complementarity determining regions (CDRH) 1 as set forth in SEQ ID NO: 24, a CDRH2 as set forth in SEQ ID NO: 25, and a CDRH3 as set forth in SEQ ID NO: 26, and
a variable light chain complementarity determining region (CDRL) 1 as set forth in SEQ ID NO: 27, a CDRL2 as set forth in SEQ ID NO: 28, and a CDRL3 as set forth in SEQ ID NO: 29;
a CDRH1 as set forth in SEQ ID NO: 32, a CDRH2 as set forth in SEQ ID NO: 33, and a CDRH3 as set forth in SEQ ID NO: 34, and
a CDRL1 as set forth in SEQ ID NO: 35, a CDRL2 as set forth in SEQ ID NO: 36, and a CDRL3 as set forth in SEQ ID NO: 37;
a CDRH1 as set forth in SEQ ID NO: 42, a CDRH2 as set forth in SEQ ID NO: 43, and a CDRH3 as set forth in SEQ ID NO: 44, and
a CDRL1 as set forth in SEQ ID NO: 45, a CDRL2 as set forth in SEQ ID NO: 46, and a CDRL3 as set forth in SEQ ID NO: 47;
a CDRH1 as set forth in SEQ ID NO: 50, a CDRH2 as set forth in SEQ ID NO: 51, and a CDRH3 as set forth in SEQ ID NO: 52, and
a CDRL1 as set forth in SEQ ID NO: 53, a CDRL2 as set forth in SEQ ID NO: 54, and a CDRL3 as set forth in SEQ ID NO: 55;
a CDRH1 as set forth in SEQ ID NO: 58, a CDRH2 as set forth in SEQ ID NO: 59, and a CDRH3 as set forth in SEQ ID NO:60, and
a CDRL1 as set forth in SEQ ID NO: 61, a CDRL2 as set forth in SEQ ID NO: 62, and a CDRL3 as set forth in SEQ ID NO: 63;
a CDRH1 as set forth in SEQ ID NO: 66, a CDRH2 as set forth in SEQ ID NO: 67, and a CDRH3 as set forth in SEQ ID NO: 68, and
a CDRL1 as set forth in SEQ ID NO: 69, a CDRL2 as set forth in SEQ ID NO: 70, and a CDRL3 as set forth in SEQ ID NO: 71; and
a CDRH1 as set forth in SEQ ID NO: 74, a CDRH2 as set forth in SEQ ID NO: 75, and a CDRH3 as set forth in SEQ ID NO: 76, and
a CDRL1 as set forth in SEQ ID NO: 77, a CDRL2 as set forth in SEQ ID NO: 78, and a CDRL3 as set forth in SEQ ID NO: 79,
according to the Kabat numbering scheme.

9. The targeted therapeutic molecule of claim 3, encoded by the sequence as set forth in SEQ ID NO: 134.

10. The targeted therapeutic molecule of claim 3, wherein the binding domain specifically binds MEGF10.

11. The targeted therapeutic molecule of claim 10, wherein the binding domain comprises LS-C678634, LS-C668447, LSC497216, or PA5-76556, or a binding fragment thereof.

12. The targeted therapeutic molecule of claim 3, wherein the binding domain specifically binds HPSE2.

13. The targeted therapeutic molecule of claim 12, wherein the binding domain comprises LS-B14593, LS-C322089, LS-C378319, or HPA044603, or a binding fragment thereof.

14. The targeted therapeutic molecule of claim 3, wherein the binding domain specifically binds KLRF2.

15. The targeted therapeutic molecule of claim 14, wherein the binding domain comprises LS-C329740, LS-C203747, SAB2108513, SAB2108684, HPA055964, SAB2108320, or SAB2108355, or a binding fragment thereof.

16. The targeted therapeutic molecule of claim 3, wherein the binding domain specifically binds PCDH19.

17. The targeted therapeutic molecule of claim 16, wherein the binding domain comprises LS-C676224, LS-C496779, LS-C761991, HPA027533, or HPA001461, or a binding fragment thereof.

18. The targeted therapeutic molecule of claim 3, wherein the binding domain specifically binds FRAS1.

19. The targeted therapeutic molecule of claim 18, wherein the binding domain comprises LS-C763132, LS-B5486, LS-C754337, HPA011281, or HPA051601, or a binding fragment thereof.

20. The targeted therapeutic molecule of claim 3, wherein the extracellular component further comprises a spacer region.

21. The targeted therapeutic molecule of claim 20, wherein the spacer region comprises a long spacer region, intermediate spacer region, or short spacer region.

22. The targeted therapeutic molecule of claim 21, wherein the intermediate spacer region is 135 amino acids or less.

23. The targeted therapeutic molecule of claim 21, wherein the intermediate spacer region is 131 amino acids or less and comprises a hinge region and a CH3 domain of IgG4.

24. The targeted therapeutic molecule of claim 23, wherein the intermediate spacer region is encoded by the sequence as set forth in SEQ ID NO: 136.

25. The targeted therapeutic molecule of claim 21, wherein the intermediate spacer region is encoded by the sequence as set forth in SEQ ID NO: 3.

26. The targeted therapeutic molecule of claim 21, wherein the long spacer region is greater than 200 amino acids and comprises an IgG4 hinge, IgG4 CH3 region, and an IgG4 CH2 region.

27. The targeted therapeutic molecule of claim 21, wherein the long spacer region is encoded by the sequence as set forth in SEQ ID NO: 4.

28. The targeted therapeutic molecule of claim 21, wherein the short spacer region is less than 50 amino acids and comprises an IgG4 hinge.

29. The targeted therapeutic molecule of claim 21, wherein the short spacer region is encoded by the sequence as set forth in SEQ ID NO: 1 or SEQ ID NO: 2.

30. The targeted therapeutic molecule of claim 3, wherein the intracellular effector domain comprises all or a portion of the signaling domain of CD3ζ and 4-1BB.

31. The targeted therapeutic molecule of claim 30, wherein the CD3ζ signaling domain is encoded by the CD3ζ coding sequence as set forth in SEQ ID NO: 5.

32. The targeted therapeutic molecule of claim 30, wherein the CD3ζ signaling domain comprises the sequence as set forth in SEQ ID NO: 6 or SEQ ID NO: 7.

33. The targeted therapeutic molecule of claim 30, wherein the 4-1BB signaling domain is encoded by SEQ ID NO: 8 or SEQ ID NO: 9.

34. The targeted therapeutic molecule of claim 30, wherein the 4-1BB signaling domain comprises the sequence as set forth in SEQ ID NO: 10 or SEQ ID NO: 11.

35. The targeted therapeutic molecule of claim 3, wherein the transmembrane domain comprises a CD28 transmembrane domain.

36. The targeted therapeutic molecule of claim 35, wherein the CD28 transmembrane domain is encoded by SEQ ID NO: 12, SEQ ID NO: 13, or SEQ ID NO: 14.

37. The targeted therapeutic molecule of claim 35, wherein the CD28 transmembrane domain comprises SEQ ID NO: 15 or SEQ ID NO: 16.

38. The targeted therapeutic molecule of claim 3, further comprising a control feature selected from a tag cassette, a transduction marker, and/or a suicide switch.

39. The targeted therapeutic molecule of claim 38, wherein the transduction marker comprises a truncated CD19.

40. The targeted therapeutic molecule of claim 39, wherein the truncated CD19 is encoded by SEQ ID NO: 117.

41. The targeted therapeutic molecule of claim 3, further comprising a ribosomal skip element.

42. The targeted therapeutic molecule of claim 39, wherein the ribosomal skip element comprises T2A, P2A, E2A, or F2A.

43. The targeted therapeutic molecule of claim 41, wherein the ribosomal skip element comprises T2A.

44. The targeted therapeutic molecule of claim 43, wherein T2A is encoded by SEQ ID NO: 137.

45. A genetic construct encoding the CAR of claim 3.

46. A nanoparticle encapsulating the genetic construct of claim 45.

47. A cell genetically modified to express the CAR of claim 3.

48. The cell of claim 47, wherein the cell is an autologous cell or an allogeneic cell in reference to a subject.

49. The cell of claim 47, wherein the cell is in vivo or ex vivo.

50. The cell of claim 47, wherein the cell is a T cell, B cell, natural killer (NK) cell, NK-T cell, monocyte/macrophage, hematopoietic stem cells (HSC), or a hematopoietic progenitor cell (HPC).

51. The cell of claim 50, wherein the cell is a T cell selected from a CD3+ T cell, a CD4+ T cell, a CD8+ T cell, a central memory T cell, an effector memory T cell, and/or a naive T cell.

52. The cell of claim 50, wherein the cell is a CD8+ T cell and/or a CD4+ T cell.

53. The targeted therapeutic molecule of claim 2, wherein the binding domain is conjugated to a cytotoxic payload.

54. The targeted therapeutic of claim 2, wherein the binding domain specifically binds FOLR1.

55. The targeted therapeutic of claim 2, wherein the binding domain comprises a single chain variable fragment (scFv).

56. The targeted therapeutic of claim 55, wherein the scFv has the sequence as set forth in SEQ ID NO: 22 or SEQ ID NO: 23.

57. The targeted therapeutic of claim 54, wherein the binding domain comprises a variable heavy chain set forth in SEQ ID NO: 30 and a variable light chain set forth in SEQ ID NO: 31; a variable heavy chain set forth in SEQ ID NO: 38 and a variable light chain set forth in SEQ ID NO: 39; a variable heavy chain set forth in SEQ ID NO: 40 and a variable light chain set forth in SEQ ID NO: 41; a variable heavy chain set forth in SEQ ID NO: 48 and a variable light chain set forth in SEQ ID NO: 49; a variable heavy chain set forth in SEQ ID NO: 56 and a variable light chain set forth in SEQ ID NO: 57; a variable heavy chain set forth in SEQ ID NO: 64 and a variable light chain set forth in SEQ ID NO: 65; a variable heavy chain set forth in SEQ ID NO: 72 and a variable light chain set forth in SEQ ID NO: 73; or a variable heavy chain set forth in SEQ ID NO: 80 and a variable light chain set forth in SEQ ID NO: 81.

58. The targeted therapeutic of claim 54, wherein the binding domain comprises a variable heavy chain with complementarity determining regions (CDRH) 1 as set forth in SEQ ID NO: 24, a CDRH2 as set forth in SEQ ID NO: 25, and a CDRH3 as set forth in SEQ ID NO: 26, and
a variable light chain complementarity determining region (CDRL) 1 as set forth in SEQ ID NO: 27, a CDRL2 as set forth in SEQ ID NO: 28, and a CDRL3 as set forth in SEQ ID NO: 29;
a CDRH1 as set forth in SEQ ID NO: 32, a CDRH2 as set forth in SEQ ID NO: 33, and a CDRH3 as set forth in SEQ ID NO: 34, and
a CDRL1 as set forth in SEQ ID NO: 35, a CDRL2 as set forth in SEQ ID NO: 36, and a CDRL3 as set forth in SEQ ID NO: 37;
a CDRH1 as set forth in SEQ ID NO: 42, a CDRH2 as set forth in SEQ ID NO: 43, and a CDRH3 as set forth in SEQ ID NO: 44, and
a CDRL1 as set forth in SEQ ID NO: 45, a CDRL2 as set forth in SEQ ID NO: 46, and a CDRL3 as set forth in SEQ ID NO: 47;
a CDRH1 as set forth in SEQ ID NO: 50, a CDRH2 as set forth in SEQ ID NO: 51, and a CDRH3 as set forth in SEQ ID NO: 52, and
a CDRL1 as set forth in SEQ ID NO: 53, a CDRL2 as set forth in SEQ ID NO: 54, and a CDRL3 as set forth in SEQ ID NO: 55;
a CDRH1 as set forth in SEQ ID NO: 58, a CDRH2 as set forth in SEQ ID NO: 59, and a CDRH3 as set forth in SEQ ID NO:60, and
a CDRL1 as set forth in SEQ ID NO: 61, a CDRL2 as set forth in SEQ ID NO: 62, and a CDRL3 as set forth in SEQ ID NO: 63;
a CDRH1 as set forth in SEQ ID NO: 66, a CDRH2 as set forth in SEQ ID NO: 67, and a CDRH3 as set forth in SEQ ID NO: 68, and
a CDRL1 as set forth in SEQ ID NO: 69, a CDRL2 as set forth in SEQ ID NO: 70, and a CDRL3 as set forth in SEQ ID NO: 71; and
a CDRH1 as set forth in SEQ ID NO: 74, a CDRH2 as set forth in SEQ ID NO: 75, and a CDRH3 as set forth in SEQ ID NO: 76, and
a CDRL1 as set forth in SEQ ID NO: 77, a CDRL2 as set forth in SEQ ID NO: 78, and a CDRL3 as set forth in SEQ ID NO: 79,
according to the Kabat numbering scheme.

59. The targeted therapeutic molecule of claim 2, wherein the binding domain specifically binds MEGF10.

60. The targeted therapeutic molecule of claim 59, wherein the binding domain comprises LS-C678634, LS-C668447, LSC497216, or PA5-76556, or a binding fragment thereof.

61. The targeted therapeutic of claim 2, wherein the binding domain specifically binds HPSE2.

62. The targeted therapeutic molecule of claim 61, wherein the binding domain comprises LS-B14593, LS-C322089, LS-C378319, or HPA044603, or a binding fragment thereof.

63. The targeted therapeutic molecule of claim 2, wherein the binding domain specifically binds KLRF2.

64. The targeted therapeutic molecule of claim 63, wherein the binding domain comprises LS-C329740, LS-C203747, SAB2108513, SAB2108684, HPA055964, SAB2108320, or SAB2108355, or a binding fragment thereof.

65. The targeted therapeutic molecule of claim 2, wherein the binding domain specifically binds PCDH19.

66. The targeted therapeutic molecule of claim 65, wherein the binding domain comprises LS-C676224, LS-C496779, LS-C761991, HPA027533, or HPA001461, or a binding fragment thereof.

67. The targeted therapeutic molecule of claim 2, wherein the binding domain specifically binds FRAS1.

68. The targeted therapeutic molecule of claim 67, wherein the binding domain comprises LS-C763132, LS-B5486, LS-C754337, HPA011281, or HPA051601, or a binding fragment thereof.

69. The targeted therapeutic molecule of claim 53, wherein the cytotoxic payload comprises a cytotoxin, a cytotoxic drug, a radioisotope, or a nanoparticle.

70. The targeted therapeutic molecule of claim 69, wherein the cytotoxin comprises a holotoxin or a hemitoxin.

71. The targeted therapeutic molecule of claim 69, wherein the cytotoxic drug comprises actinomycin D, anthracycline, auristatin, calicheamicin, camptothecin, CC1065, colchicin, cytochalasin B, daunorubicin, 1-dehydrotestosterone, dihydroxy anthracinedione, dolastatin, doxorubicin, duocarmycin, elinafide, emetine, ethidium bromide, etoposide, gramicidin D, glucocorticoids, lidocaine, maytansinoid, mithramycin, mitomycin, mitoxantrone, nemorubicin, PNU-159682, procaine, propranolol, puromycin, pyrrolobenzodiazepine (PBD), taxane, taxol, tenoposide, tetracaine, trichothecene, vinblastine, vinca alkaloid, vincristine, or stereoisomers, isosteres, analogs, or derivatives thereof.

72. The targeted therapeutic molecule of claim 69, wherein the radioisotope comprises ²²⁸Ac, ¹¹¹Ag, ¹²⁴Am, ⁷⁴As, ²¹¹As, ²⁰⁹At, ¹⁹⁴Au, ¹²⁸Ba, ⁷Be, ²⁰⁶Bi, ²⁴⁵Bk, ²⁴⁶Bk, ⁷⁶Br, ¹¹C, ⁴⁷Ca, ²⁵⁴Cf, ²⁴²Cm ⁵¹Cr, ⁶⁷CU ¹⁵³Dy, ¹⁵⁷Dy, ¹⁵⁹Dy, ¹⁶⁵Dy, ¹⁶⁶Dy, 171Er, ²⁵⁰Es, ²⁵⁴Es, ¹⁴⁷Eu, ¹⁵⁷Eu, ⁵²Fe, ⁵⁹Fe, ²⁵¹Fm, ²⁵²Fm, ²⁵³Fm, ⁶⁶Ga, ⁷²Ga, ¹⁴⁶Gd, ¹⁵³Gd, ⁶⁸Ge, ¹⁷⁰Hf, ¹⁷¹Hf, ¹⁹³Hg, ¹⁹³mHg, ¹⁶⁰mHo, ¹³⁰I, ¹³¹I, ¹³⁵I, ¹¹⁴mIn, ¹⁸⁵Ir, ⁴²K, ⁴³K, ⁷⁶Kr, ⁷⁹Kr, ⁸¹mKr, ¹³²La, ²⁶²Lr, ¹⁶⁹Lu, ¹⁷⁴mLu, ¹⁷⁶mLu, ²⁵⁷Md, ²⁶⁰Md, ²⁸Mg, ⁵²Mn, ⁹⁰Mo, ²⁴Na, ⁹⁵Nb, ¹³⁸Nd, ⁵⁷Ni, ⁶⁶Ni, ²³⁴Np, ¹⁵O, ¹⁸²Os, ¹⁸⁹mOs, ¹⁹¹Os, ³²P, ²⁰¹Pb, ¹⁰¹Pd, ¹⁴³Pr, ¹⁹¹Pt, ²⁴³Pu, ²²⁵Ra, ⁸¹Rb, ¹⁸⁸Re, ¹⁰⁵Rh, ²¹¹Rn, ¹⁰³Ru, ³⁵S, ⁴⁴Sc, ⁷²Se, ¹⁵³Sm, ¹²⁵Sn, ⁹¹Sr, ¹⁷³Ta, ¹⁵⁴Tb, ¹²⁷Te, ²³⁴Th, ⁴⁵Ti, ¹⁶⁶Tm, ²³⁰U, ²³⁷U, ²⁴⁰U, ⁴⁸V, ¹⁷⁸W, ¹⁸¹W, ¹⁸⁸W, ¹²⁵Xe, ¹²⁷Xe, ¹³³Xe, ¹³³mXe, ¹³⁵Xe, ⁸⁵mY, ⁸⁶Y, ⁹⁰Y, ⁹³Y, ¹⁶⁹Yb, ¹⁷⁵Yb, ⁶⁵Zn, ⁷¹mZn, ⁸⁶Zr, ⁹⁵Zr, and/or ⁹⁷Zr.

73. The targeted therapeutic molecule of claim 69, wherein the nanoparticle comprises a metal nanoparticle, a liposome, or a polymer nanoparticle.

74. A formulation comprising cells genetically modified to express the CAR system of claim 2.

75. The formulation of claim 74, wherein the cells are T cells, natural killer cells, monocyte/macrophages, hematopoietic stem cells or hematopoietic progenitor cells.

76. The formulation of claim 75, wherein the T cells are selected from CD3 T cells, CD4 T cells, CD8 T cells, central memory T cells, effector memory T cells, and/or naive T cells.

77. The formulation of claim 75, wherein the T cells are CD4 T cells and/or CD8 T cells.

78. The formulation of claim 74, further comprising a pharmaceutically acceptable carrier.

79. A composition comprising the targeted therapeutic of claim 54 and a pharmaceutically acceptable carrier.

80. A method of treating a subject in need thereof comprising administering a therapeutically effective amount of the formulation of claim 74 and/or the composition of claim 79 to the subject thereby treating the subject in need thereof.

81. The method of claim 80, wherein the subject in need thereof has cancer.

82. The method of claim 81, wherein the cancer comprises cancer cells expressing FOLR1, MEGF10, HPSE2, KLRF2, PCDH19, or FRAS1.

83. The method of claim 81, wherein the cancer comprises leukemia.

84. The method of claim 83, wherein the leukemia is acute myeloid leukemia (AML).

85. The method of claim 84, wherein the AML comprises CBFA2T3/GLIS2 AML.

86. The method of claim 81, wherein the cancer comprises cancer cells expressing FOLR1.

87. The method of claim 86, wherein the cancer comprises leukemia, peritoneal cancer, fallopian tube cancer, ovarian cancer, endometrial cancer, cervical cancer, breast cancer, bladder cancer, renal cell carcinoma, pituitary tumors, lung cancer, uterine cancer, squamous cell carcinoma, ureter cancer, urethral cancer, osteosarcoma, or transitional cell carcinoma.

88. The method of claim 87, wherein the cancer is metastatic.

89. The method of claim 87, wherein the ovarian cancer comprises epithelial ovarian cancer.

90. The method of claim 87, wherein the breast cancer comprises triple-negative breast cancer or HER2-breast cancer.

91. The method of claim 87, wherein the lung cancer comprises lung adenocarcinoma or epithelial lung cancer such as non-small cell lung cancer.

92. The method of claim 80, wherein the formulation comprises autologous cells or allogeneic cells.

93. A method of treating a subject with CBFA2T3/GLIS2 acute myeloid leukemia (AML) comprising administering a therapeutically effective amount of the formulation of claim 74 and/or the composition of claim 79 to the subject thereby treating the subject with the CBFA2T3/GLIS2 AML.

94. The method of claim 93, wherein the formulation comprises autologous cells or allogeneic cells.
